SEQUENCE LISTING (6 sequences)

SEQ ID NO: 1 - DNA, 3848 bp, Homo Sapiens
acctgcagcgcgaggcgcgccgctccaggcggcatcgcagggctgggccggcgcggcctggggaccccgggctccggaggccatgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgacgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtagaggaacaatttagttttaaggacttcatccctccacctatccctaacaggctgtagattaccaaaacaagattaatttcatcactaaaagaaaatctattatcaactgctgcttcaccagacttttctctagaagctgtctgcgtttactcttgttttcaaagggacttttgtaaaatcaaatcatcctgtcacaaggcaggaggagctgataatgaactttattggagcattgatctgcatccaaggccttctcaggctggcttgagtgaattgtgtacctgaagtacagtatattcttgtaaatacataaaacaaaagcattttgctaaggagaagctaatatgattttttaagtctatgttttaaaataatatgtaaatttttcagctatttagtgatatattttatgggtgggaataaaatttctactacagaattgcccattattgaattatttacatggtataattagggcaagtcttaactggagttcacgaaccccctgaaattgtgcacccatagccacctacacattccttccagagcacgtgtgcttttaccccaagatacaaggaatgtgtaggcagctatggttgtcacagcctaagatttctgcaacaacaggggttgtattgggggaagtttataatgaataggtgttctaccataaagagtaatacatcacctagacactttggcggccttcccagactcagggccagtcagaagtaacatggaggattagtattttcaataaagttactcttgtccccacaaaaaaa

SEQ ID NO: 2 - protein, 993 aa, Homo Sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 3 - DNA, 1056 bp, Homo Sapiens
tctctggctgtcacccggcttggccccttccacacccaactggggcaagcctgacccggcgacaggaggcatgaggggcccccggccgaaatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactgacctggccaaggcctcatcctggggaggatactgaggcacacagaggggagtcaccagccagaggatgcatagcctggacacagaggaagttggctagaggccggtcccttccttgggcccctctcattccctccccagaatggaggcaacgccagaatccagcaccggccccatttacccaactctgtacaaagcccttgtccccatgaaattgtatataaatcatccttttctaccaaaaaaaaaaaaaaaaaaaaa

SEQ ID NO: 4 - protein, 235 aa, Homo Sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 5 - protein, 209 aa, Homo Sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 6 - protein, 515 aa, Homo Sapiens
NQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDN

(19) United States
(12) Patent Application Publication
(10) Pub. No.: US 2013/0288373 A1
(43) Pub. Date: Oct. 31, 2013

(54) CRYSTAL STRUCTURE OF FLT3 LIGAND-RECEPTOR COMPLEX

(71) Applicants: Kenneth Verstraete, Gent (BE); Savvas Savvides, Kortrijk (BE)

(72) Inventors: Kenneth Verstraete, Gent (BE); Savvas Savvides, Kortrijk (BE)

(21) Appl. No.: 13/996,118

(22) PCT Filed: Dec. 20, 2011
(86) PCT No.: PCT/EP2011/073335

§ 371 Date: Jun. 20, 2013

(30) Foreign Application Priority Data
Dec. 20, 2010 (EP) 10196039.1

(51) Int. Cl. G01N 33/50; G06F 19/16

(52) U.S. Cl. CPC G01N 33/5044; G06F 19/16
USPC 435/375; 436/501; 530/389.6; 530/300; 703/11

(57) ABSTRACT

The present invention relates to the crystal structure of Flt3 and its cognate ligand FL. In particular, the binding interface of Flt3 and its cognate ligand FL has been determined. The present invention also relates to the applicability in modulating Flt3 activity. Methods for the identification as well as the rational design of mediators of Flt3 signaling are disclosed. In an aspect the method comprises a step of employing the atomic coordinates representing the three-dimensional structure of Flt3 and/or FL. In another aspect, the method comprises a step of contacting a candidate ligand with an Flt3 polypeptide comprising the FL binding site or alternatively an FL polypeptide comprising the Flt3 binding site, after which binding is determined

### TECHNICAL FIELD

The present invention generally relates to structural studies of the Flt3 receptor tyrosine kinase. In particular, the present invention relates to the crystal structure of Flt3 in complex with its cognate ligand FL. The present invention also relates to the applicability in modulating Flt3 activity. Methods for the identification as well as the rational design of agonistic or antagonistic modulators of Flt3 signaling are disclosed.

### BACKGROUND

Hematopoiesis is a finely regulated process during which diverse cell types originating from a limited and self-renewing population of hematopoietic stem cells (HSC) are stimulated to proliferate and differentiate to create the cellular repertoire that sustains the mammalian hematopoietic and immune systems (Metcalf, 2008). The hematopoietic pathway is orchestrated by intracellular signaling pathways, which are initiated via the activation of hematopoietic receptors by their cognate cytokine ligands at the cell surface (Bryder 2006; Li and Li, 2006; Metcalf, 2007; Ross and Li, 2006).

The Fms-like tyrosine kinase receptor 3 (Flt3), is the most recent addition to the diverse family of hematopoietic receptors (Matthews 1991; Rosnet 1991). Flt3 is activated on HSC and early myeloid and lymphoid progenitors by its cognate ligand (FL) (Lyman, 1993; Hannum, 1994), to initiate downstream signaling via the PI3K/AKT and the RAS/RAF/MEK/ERK pathways (Parcells, 2006; Stirewalt, 2003). Consistent with the narrow expression profile of Flt3 in the bone marrow environment, signaling via the Flt3 ligand/receptor complex primarily impacts early hematopoiesis, particularly the proliferation and development of HSC and B-cell progenitors (Stirewalt, 2003; Kikushige, 2008). In recent years Flt3 and FL emerged as potent regulators of dendritic cell (DC) development and homeostasis (Waskow, 2008; Onai, 2007; Liu, 2009; Liu and Nussenweig, 2010; Schmid, 2010), and DC-mediated natural killer cell activation (Eidenschenk, 2010; Guimond, 2010), thereby gaining an important role at the interface of innate and acquired immunity and in cancer immunotherapy (Antonysamy and Thomson, 2000; Dong, 2002; Fong, 2001; Karsunky, 2003; Wu and Liu, 2007). Notably, Flt3/FL-driven DC generation yields both classical- and plasmacytoid DC from bone-marrow progenitors regardless of myeloid or lymphoid commitment, a property that is currently unmatched by any other receptor/cytokine system relevant for DC physiology (Schmid, 2010).

Flt3 is together with the prototypic platelet-derived growth factor receptor (PDGFR), colony-stimulating factor 1 receptor (CSF-1R), and KIT (Robinson, 2000; Grassot, 2006) a class III receptor tyrosine kinase III (RTKIII). Thus, Flt3 has been predicted to be organized into a modular structure featuring an extracellular segment with 5 immunoglobulin (Ig)-like domains (residues 27-543), a single transmembrane helix (TM, residues 544-563), a cytoplasmic juxtamembrane domain (JM, residues 572-603) and a split intracellular kinase module (residues 604-958). The RTKIII family is closely related to the RTKV family of vascular endothelial growth factor receptors (VEGFR), which have 7 extracellular Ig-like domains. The hallmark of RTKIII/V signaling lies in the dimerization of the extracellular receptor segments upon binding of their respective cytokine ligands, followed by intermolecular autophosphorylation and activation of the intracellular kinase domains (Turner, 1996; Kiyoi, 1998; Hubbard and Miller, 2007; Lemmon and Schlessinger, 2010).

Besides the outspoken role of Flt3 signaling in hematopoiesis and immune system development, overexpression of wild type or oncogenic forms of Flt3 have been implicated in a number hematopoietic malignancies (Stirewalt and Radich, 2003; Sanz, 2010), and inflammatory disorders (Dehlin, 2008). In particular, internal tandem duplication (ITD) in the JM region or point mutations in the kinase activation loop occur in 35% of patients with Acute Myeloid Leukemia (AML) resulting in constitutive activation of the receptor and uncontrolled proliferation of hematopoietic precursors (Kiyoi, 1998; Stirewalt and Radich, 2003; Reindl, 2006; Parcells, 2006; Frohling, 2007). Such mutation fingerprints have established Flt3 as the predominant prognostic factor in AML cases (Eklund, 2010), and have rationalized targeting of Flt3 in a clinical setting (Sanz, 2009; Parcells, 2006; Sternberg and Licht, 2005; Stirewalt, 2003; Kindler, 2010).

Although the cellular and physiological role of the Flt3 ligand-receptor interaction has been featured prominently in the biomedical literature over the last two decades, the Flt3 signaling complex has remained uncharacterized at the molecular and structural level. Such insights are the missing link in exposing the structural and functional diversity of RTKIII/V extracellular complexes, and would help provide a nearly complete picture of the entire Flt3 signaling complex given the available structure of the Flt3 intracellular kinase domains (Griffith, 2004). A recent flurry of studies of RTKIII/V extracellular complexes led to a structural paradigm for RTKIII/V activation, whereby the receptors bind via their N-terminal Ig-like domains to the activating dimeric cytokine and concomitantly make homotypic contacts between their membrane-proximal domains. A universal feature of all characterized RTKIII/V complexes thus far is that the cytokine-binding epitope is distributed equally between extracellular domains 2 and 3 covering ˜2000 Å² of surface area, and that homotypic receptor-receptor interactions are mediated by a few but well-conserved residues found in the membrane-proximal domains (domain 4 in RTKIII and domain 7 in RTKV). Nonetheless, Flt3 appears to be an outlier among RTKIII/V receptors due to several unique features in its extracellular segment (Lyman, 1993; Maroc, 1993), thus raising the question whether the current structural paradigm could be extrapolated to Flt3. Notably, Flt3 exhibits intragenic homology relating extracellular domains 1 and 4, and domains 2 and 5, indicative of an ancient internal duplication event during evolution. Furthermore, Flt3 has an N-terminal sequence of 50 amino acids preceding ectodomain 1 that shows no similarity to other proteins, and contains 12 additional cysteines that are not present in any of the homologous receptors.

Rational drug design for modulating Flt3-mediated signaling is hampered by the lack of structural information of the Flt3-receptor, in particular the Flt3 ligand-receptor interaction. It is therefore an object of the present invention to provide such structural information. In particular, identification of the binding site of Flt3 for its cognate ligand FL is instructive in screening, identifying and designing for ligands of Flt3 and FL which can be used to modulate Flt3 signaling.

### SUMMARY

The present inventors have resolved the crystal structure of Flt3 bound to its cognate ligand. Surprisingly, and contrary to expectations, the inventors have identified a particular compact Flt3/FL binding interface. Flt3 employs a single and very compact ligand-binding epitope contributed exclusively by Ig-like domain 3 (D3), without engaging in homotypic interactions with its tandem receptor in the complex. This combination of features is completely unexpected because it deviates drastically from the current paradigm for extracellular activation of RTKIII receptors. More specifically, it was expected that Flt3 would collectively employ ectodomains D1-D3 to bind to its cognate cytokine, and that this interaction would be accompanied by homotypic interactions in the membrane-proximal domains D4-D5. The resolved crystal structure proves otherwise. As such, the Flt3 receptor is the only helical cytokine receptor that does not use more than one interaction site to bind its cognate ligand. In addition, FL is identified as the only helical cytokine that does not use any helix-helix groove to engage its receptor. Moreover, FL uses a preformed binding epitope to bind to the receptor subregion of the extracellular Flt3 domain.

Previous predictions identified a much larger region of the extracellular signaling complex as crucial for ligand binding and Flt3 activation. This hampered rational design of novel drugs targeting this large domain as it was not clear which regions were the most important. With the new data set, the binding epitope has been identified and turns out to be compact making it an interesting target for drug design. Also, it is clear now how FL interacts with this epitope, making blocking strategies of the ligand also a possibility, next to blocking its extracellular epitope (receptor blocking strategy versus ligand blocking strategy).

Aberrant Flt3 signaling is caused by oncogenic forms of the receptor or by overexpression of the wild type receptor. Furthermore, autocrine signaling loops seem to play an important role in leukogenesis (Zheng, 2004). Currently known strategies to modulate Flt3 signaling are mainly focused on targeting and inhibiting the intracellular tyrosine kinase domain with the use of tyrosine kinsase inhibitors (TKI). However, primary and secondary acquired resistance severely compromise long-term and durable efficacy of these inhibitors as a therapeutic strategy. Therefore, a major contribution of the present invention over the art includes the identification of a compact Flt3/FL binding interface, making it a very attractive target useful for protein-based therapeutic strategies aiming at blocking the binding of the cognate ligand FL to the Flt3 extracellular domain, or alternatively activating Flt3 signaling with FL mimetic ligands. Such strategies would lead to deactivation or activation, respectively, of downstream pathways affecting hematopoietic cell proliferation and DC homeostasis/activity.

Accordingly, in an aspect, the invention relates to a method for identifying or designing a ligand which modulates Flt3 signaling, comprising the step of employing a three dimensional structure represented by a set of atomic coordinates presented in Table 3, or a subset thereof, or atomic coordinates which deviate from those in Table 3, or a subset thereof, by RMSD over protein backbone atoms by no more than 3 Å.

In an embodiment, said method further comprises the step of structure-based identification and/or design of a ligand based on the interaction of said ligand with the 3D structure represented by the atomic coordinates presented in Table 3, or a subset thereof, or atomic coordinates which deviate from those in Table 3, or a subset thereof, by RMSD over protein backbone atoms by no more than 3 Å.

In another embodiment, said method is a computer-implemented method, said computer comprising an inputting device, a processor, a user interface, and an outputting device, wherein said method comprises the steps of:
a) generating a three-dimensional structure of atomic coordinates presented in Table 3, or a subset thereof, or atomic coordinates which deviate from those in Table 3, or a subset thereof, by RMSD over protein backbone atoms by no more than 3 Å;
b) fitting the structure of step a) with the structure of a candidate ligand by computational modeling;
c) selecting a ligand that possesses energetically favorable interactions with the structure of step a).

In an embodiment, said fitting comprises superimposing the structure of step a) with the structure of said candidate ligand. In another embodiment, said modeling comprises docking modeling.

In a further embodiment, said ligand of step c) can bind to at least 1 amino acid residue of the structure of step a) without steric interference.

In another aspect, the invention relates to a method for identifying a ligand which modulates Flt3 signaling, comprising the steps of:
a) providing a candidate ligand;
b1) providing a polypeptide comprising a region of at least 5 consecutive amino acid residues of amino acid residues 245-345 of Flt3; or
b2) providing a polypeptide comprising a region of at least 5 consecutive amino acid residues of amino acid residues 5-20 of FL;
c) contacting said candidate ligand with said polypeptide of step b1) or step b2);
d) determining the binding of said candidate ligand with said region of step b1) or step b2); and
e) identifying said candidate ligand as a ligand which modulates Flt3 signaling if binding between said candidate ligand and said region of step b1) or step b2) is detected.

In a further aspect, the invention relates to an in vitro method for modulating Flt3 signaling, comprising the steps of:
a) providing a composition comprising an Flt3 polyprotein; and
b) contacting said composition with a ligand as identified or designed according to the methods as described herein.

In yet another aspect, the invention relates to the use of a polypeptide comprising a region of at least 5 consecutive amino acid residues of amino acid residues 245-345 of Flt3, a polypeptide comprising a region of at least 5 consecutive amino acid residues of amino acid residues 5-20 of FL, and/or the atomic coordinates presented in Table 3, or a subset thereof, or atomic coordinates which deviate from those in Table 3, or a subset thereof, by RMSD over protein backbone atoms by no more than 3 Å for designing and/or identifying a ligand which modulates Flt3 signaling.

In an embodiment, the ligand which is designed and/or identified according to the methods as described herein is an antagonist, which is preferably selected from the group consisting of an Alphabody™, a Nanobody®, an antibody, or a small molecule.

In an aspect, the invention also relates to an Alphabody™, a Nanobody®, an antibody, or a small molecule which binds to the region comprised within amino acid residues 245-345 of Flt3, or which binds to the region comprised within amino acid residues 5-20 of FL.

In a further aspect, the invention relates to a polypeptide comprising at most 200 consecutive amino acid residues of Flt3, wherein said polypeptide comprises at least 5 consecutive amino acid residues of amino acid residues 245-345 of FL.

In another aspect, the invention relates to a polypeptide comprising at most 50 consecutive amino acid residues of FL, wherein said polypeptide comprises at least 5 consecutive amino acid residues of amino acid residues 5-20 of FL.

In an aspect, the invention also relates to a ligand as designed and/or identified according to the methods as described herein, for use as a modulator of Flt3 signaling.

A further aspect of the invention relates to a computer system comprising:
a) a database containing the atomic coordinates presented in Table 3, or a subset thereof, or atomic coordinates which deviate from those in Table 3, or a subset thereof, by RMSD over protein backbone atoms by no more than 3 Å, stored on a computer readable storage medium; and
b) a user interface to view the information.

### DESCRIPTION OF THE DRAWINGS

FIG. 1. High-Affinity Complex Formation Between FL and Flt3 Ectodomain Variants.

(A-B) Isolation of Flt3D1-D5:FL and Flt3D1-D4:FL by size-exclusion chromatography (SEC). Also shown are Coomassie-stained SDS-PAGE strips corresponding to the peak fraction of the isolated complexes. The elution profiles of the complexes are characterized by large shifts to a single, faster migrating peak corresponding to the respective complex. (C) Size-exclusion chromatography on the Flt3D1-D3:FL mixture at the end of an ITC experiment, showing that a large amount of Flt3D1-D3 remains in the unbound form. Identical elution profiles were obtained in standard SEC experiments as well, in the presence of a large molar excess of FL. (D-F) Binding isotherms and thermodynamic parameters of FL binding to Flt3 ectodomains obtained by Isothermal Titration calorimetry (ITC). All experiments were performed by titrating recombinant Flt3 extracellular domains with FL.

FIG. 2 Crystal Structure of the Flt3D1-D4:FL Complex.

(A) Domain organization of the Flt3 extracellular segment. The five Ig-like domains of Flt3 (D1: residues 79-161, D2: residues 167-244, D3: residues 245-345, D4: residues 348-434 and D5: residues 435-533) are shown as colored boxes: D1 is colored in yellow, D2 in blue, D3 in green, D4 in orange and D5 in gray. N-linked glycosylation sites are indicated by blue diamonds. Partially occupied glycosylation sites are indicated with an asterisk. Also shown is the disulfide bond network in Flt3D1-D4 as determined by mass-spectrometry. The putative disulfide bridges in Flt3D5 are shown as dashed lines, based on homology with Flt3D2 and KITD5. (B) Overall structure of the Flt3D1-D4:FL complex. The crystal structure of the Flt3D14:FL complex is shown in ribbon representation with the twofold symmetry axis of FL oriented along the vertical axis of the plane. FL is colored in magenta, while the different domains of Flt3D1-4 follow the same coloring scheme as in panel A. Disulfide bridges are shown as yellow spheres and N-linked glycans as green sticks. The structural panels to the right show FL in ribbon representation and the receptor in surface representation. A 90° rotation of the main figure along the horizontal axis of the plane allows a clear view on the symmetry of the FL-Flt3D2-D3 subcomplex, whereas a 90° rotation along the vertical axis of the plane shows how FL is bound by the membrane-distal tip of D3. This view also clearly shows the asymmetric projection of the two Flt3D1 away from the core of the complex.

FIG. 3 The Flt3-FL Binding Interface.

(A) Close-up view of the Flt3-FL binding interface. FL is colored in green, Flt3D3 in grey and Flt3D2 in orange. Residues that constitute the cytokine-receptor interface are labeled and shown as sticks protruding from spheres centered at their C-alpha positions. FL residues are colored in yellow and Flt3 residues are colored in green. The receptor-binding epitope on FL is almost entirely contained in the N-terminal loop (8-13) preceding helix A (see also the inset). At the receptor site, the residues involved in ligand binding are located in the BC loop and strands D and E, and in the DE loop (see also panel B). Residue D180, in the AB loop of Flt3D2 might interact with S13 of FL, but is the only residue from Flt3D2 that could possibly contact FL. (B) The unusual Flt3D2-Flt3D3 interface. Flt3D2-D4 is shown as a C trace coloured in red. The ligand is shown in ribbon representation and is coloured green. Flt3D2 is tightly packed against Flt3D3 burying ˜1000 Å2. The residues that participate in the hydrophobic interface are labeled and their sidechains are shown as black sticks. Disulfide bonds in the two receptor domains are labeled and shown as ball and sticks (yellow). (C) Structure-based alignment of diverse FL sequences. A comparison of the FL sequences from a wide variety of species shows that the PISSXF-segment (residues 10-15) within the N-terminal loop is strictly conserved (coloured in red). A complete alignment can be found in Supplementary FIG. 3. (D) Structural comparison of bound versus the unbound FL. FL undergoes a domain tilt by 6° about its dimer interface upon receptor binding, while the receptor-binding epitope remains virtually unchanged upon receptor binding (shown in red).

FIG. 4 The Flt3D3-Flt3D4 Elbow and the Absence of Receptor Homotypic Contacts in the Flt3:FL Complex.

(A) The Flt3D3-Flt3D4 elbow. Flt3D3 (partially shown) and Flt3D4 are shown in ribbon representations. The -strands of Flt3D4 are labelled as A-G. The locations of the atypical disulfide bridges in Flt3D4 (Cys368-Cys407 and Cys381-Cys391) are indicated. Residues mediating hydrophobic interactions between Flt3D3 and Flt3D4 are shown as green sticks (F261, V345, F349 and Y376). Residues in the Flt3D3-Flt3D4 linker are shown as yellow spheres centered at their C-positions (E346-G348). The side-chains of residues that mediate the contacts between the AA′ loop of Flt3D3 and the C′E loop of Flt3D4 could not be modelled due to the low resolution of our analysis. The EF-loop of Flt3D4 which constitutes the ‘tyrosine corner’ around Y416 (green sticks) is shown in orange. (B) KITD3-KITD4 orientation in the KIT:SCF complex. Homotypic contacts between tandem ectodomain 4 modules in the KIT-SCF complex are mediated by salt bridges, formed by R381 and E386 (green sticks), which reside on the EF loops (orange) of the interacting domains (PDB entry 2E9W). The residues that make up the hydrophobic KITD3-KITD4 interface (L222, V308, F312 and F340) are shown as green sticks. Residues in the KITD3-KITD4 linker region (D309-G311) are shown as yellow spheres. (C) Flt3D4 displays an atypical EF-loop within the RTKIII/V family. A sequence comparison shows that the pair of residues mediating the homotypic contacts in KITD4 and VEGFR-2D7 is well conserved in the corresponding domains of all RTKIII/V members but not in Flt3D4. (D) Sequence conservation of residues involved at the D3-D4 interface in KIT and Flt3. A sequence comparison between human and murine Flt3 and KIT sequences reveals that the residues in the Flt3D3-Flt3D4 linker region and those participating in the hydrophobic Flt3D3-Flt3D4 interface are strongly conserved in the homologous KIT receptor.

FIG. 5 Architecture of the Complete Flt3 Ectodomain Complex.

Surface representations of the full length Flt3 ectodomain complex. FL is coloured in magenta, D2 in blue, D3 in green, D4 in orange and D5. The central view shows the complex with the two-fold axis of FL oriented vertically in the plane of the paper. The left panel shows a view corresponding to a 45° rotation along the vertical axis, while the right panel shows a view at a 90° rotation along the horizontal axis. Whereas domains D2, D3 and D4 essentially follow the P2-symmetry of FL, domains 5 and 1 display varying degrees of plasticity. Like the Flt3D1-D4:FL complex, the Flt3D1-D5:FL complex is devoid of homotypic interactions as the tandem membrane-proximal modules Flt3D4-D5 remain separated by 20 Å.

FIG. 6 Comparison of Representative Extracellular Complexes for all Members of the RTKIII/V Family.

The structures shown represent the architecture of receptor-cytokine complexes for the different members of the RTKIII/V family: From left to right: human Flt3:FL (this study), human KIT:SCF (PDB 2E9W), murine CS-1R:CSF-1 (PDB 3EJJ), hPDGFR:PDGF (PDB 3MJG) and human VEGFR2:VEGF (PDB 2X1X). The dimeric ligands are colored in magenta. Receptor ectodomains are coloured as follows: D1 in pale yellow, D2 in blue, D3 in green, D4 in orange and D5 in grey.

FIG. 7 Asymmetric Unit of the D1-4 and D1-5 Complex.

(A) The asymmetric unit of Flt3D1-D4:FL complex crystals. The Flt3D1-D4:FL complex crystallized in spacegroup P21 with two complexes in the asymmetric unit (asu). The two helical ligands in the different complexes (chains A-B and chains C-D) make extensive interactions in the asu. The receptor chains are labeled E, F, H and G. No density was visible for domains D1 of receptor chains G and H. D4 of chain G was also not modelled because of its weak density. (B) The asymmetric unit of Flt3D1-D5:FL complex crystals. Like the Flt3D1-D4:FL complex, the Flt3D1-D5:FL complex crystallized in spacegroup P21 with two complexes in the assymetric unit (asu). The contacts between the two complexes are entirely mediated by the two ligands (chains A-B and chains C-D). The Flt3 receptor chains are labeled E, F, H and G. The structure was refined by rigid-body refinement in autoBuster 2.8 using the FL promoters (residues 3-132), Flt3D1 (residues 79-161), Flt3D2-D3 (residues 167-345), Flt3D4 (residues 348-434) and Flt3D5 (residues 437-529) as rigid bodies. D1 of chain F was not modelled because of its weak density.

FIG. 8 Final Quality of the Density Map for the D14 Complex.

(A) Stereo diagram illustrating the quality of the final 2Fo-Fc electron density map to 4.2 Å resolution (contoured at 1) for the Flt3D1-D4:FL complex. The figure is centered on the Flt3D2-D3 interface and junction, with the final model for Flt3D2 (left) and Flt3D3 (right) displayed in ribbon representation (blue). The N-linked NAG glycan residue modeled at Asn306 is shown in sticks (magenta). (B) Phase improvement by density modification based on a partial model of the Flt3D1-D4:FL complex consisting of only FL and Flt3D3. The electron density is contoured at 1. The final model for domains 3 and 4 in one of the receptor chains in the Flt3D1-D4-FL complex structure is shown in ribbon representation. N-linked glycans are shown in stick representation (magenta). This electron density map was obtained by applying NCS-averaging and solvent flattering protocols as implemented in PARROT1, and proved to be crucial early in the structure determination process providing the complete electron density trace for domain 4.

FIG. 9 Interspecies Comparison of the Flt3 Ligand (FL) Sequence.

Sequence numbering and secondary structure assignment are according to the determined structure of human Flt3 ligand (pdb 1ETE). Strictly conserved residues in the included FL sequences are shaded. Residues shown to interact with the receptor (according to the present invention) are marked with an asterix. The sequences were retrieved from the NCBI and Ensembl databases: Homo sapiens (NP_{—}001450.2), Mus musculus (NP_{—}038548.3), Rattus norvegicus (XP_{—}002725623.1), Papio cynocephalus (AAO72538.1), Felis catus (NP_{—}001009842.1), Ailuropoda melanoleuca (XP_{—}002917887.1), Canis lupus familiaris (NP_{—}001003350.1), Pteropus vampyrus (ENSPVAT00000010957), Ovis aries (NP_{—}001072128.1), Bos taurus (NP_{—}851373.1), Sus scrofa (ACZ63257.1), Sorex araneus (ENSSARP00000002887), Cavia porcellus (ENSCPOP00000020385), Monodelphis domestica (XP_{—}001379894), Xenopus tropicalis (XP_{—}002938571.1)

FIG. 10 Structural Characterization of the Flt3D1-5:FL Complex by Negative-Staining Electron Microscopy and SAXS Analysis of the Flt3D1-D5:FL Complex.

(A) The displayed gallery of 100 class averages of the Flt3D1-D5:FL complex allows to recognise features corresponding to projections of the crystal structure at different orientations, notably the slightly open horseshoe ring structure with well defined individual IgG domains. (B) The crystal structure of the Flt3D1-5-FL complex was refined as a rigid-body model against the experimental scattering curve obtained by SAXS. Fitting of the theoretical scattering curve calculated from the refined model (inset) to the experimental scattering curve shows a good agreement (X²=2.5).

FIG. 11 Mapping of Non-Synonymous Sequence Variants Identified in the Flt3 Ectodomain of AML Patients.

While the majority of oncogenic alterations in the Flt3 gene are located in the JM and TKD regions, several mutations in the extracellular domains have recently been identified in AML patients2, 3. Expression of Flt3 carrying a mutation at position 451 (S451F) in BaF3 cells resulted in cytokine-independent proliferation and constitutive Flt3 autophosphorylation, demonstrating the oncogenic potential of this sequence variant. S451 is located at the solvent exposed site of strand B in the membrane proximal domain 5. Although the D324N variant did not result in ligand independent activation it is associated with a higher risk of myeloid leukemias3. D324 is located in the EF-loop of domain 3. The possible role for all other sequence variants (T167A, V194M, Y364H) in leukemogenesis has not yet been demonstrated.

FIG. 12 Sequence Listing.

### DETAILED DESCRIPTION

Before the present method and products of the invention are described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilized and structural or logical changes may be made without departing from the scope of the present invention. The following detailed description, therefore, is not to be taken in a limiting sense, and the scope of the present invention is defined by the appended claims.

Standard techniques commonly used in molecular biology are well known in the art, such as described in Sambrook et al., Molecular Cloning: A Laboratory Manual, Cold Spring Harbor Laboratory Press, New York (1989); Ausubel et al., Current Protocols in Molecular Biology, John Wiley and Sons, Baltimore, Md. (1989); Perbal, A Practical Guide to Molecular Cloning, John Wiley & Sons, New York (1988); Watson et al., Recombinant DNA, Scientific American Books, New York; Birren et al (eds) Genome Analysis: A Laboratory Manual Series, Vols. 1-4 Cold Spring Harbor Laboratory Press, New York (1998).

As used herein, “Flt3” refers to fms-like tyrosine kinase receptor-3 (Entrez Gene ID of the human orthologue: 2322; NCBI reference mRNA sequence: NM_{—}004119.2 (SEQ ID NO: 1); NCBI reference protein sequence: NP_{—}004110.2 (SEQ ID NO: 2)). Unless explicitly indicated otherwise, all Flt3 amino acid residue positions referred to herein correspond to the amino acid residue positions as indicated in SEQ ID NO: 2. SEQ ID NO: 6 is a polypeptide consisting of a subset of contiguous amino acid residues of SEQ ID NO: 2, corresponding to the extracellular domain of Flt3, in particular Ig-like domains D1 to D5 (amino acid residues 27-541 of SEQ ID NO: 2). According to the invention, the Flt3 nucleotide and protein sequences referred to herein relate to Flt3 sequences originating from any organism, i.e. all orthologues of Flt3. Preferably, the Flt3 nucleotide and protein sequences referred to herein are from mammalian origin. Particularly preferred Flt3 sequences are human.

As used herein, “FL” refers to fms-like tyrosine kinase receptor-3 ligand (Entrez Gene ID of the human orthologue: 2323; NCBI reference mRNA sequence: NM_{—}001459.2 (SEQ ID NO: 3); NCBI reference protein sequence: NP_{—}001450.2 (SEQ ID NO: 4)). Amino acid residue positions 1 to 26 correspond to the signal peptide of FL. SEQ ID NO: 5 represents human mature FL in which the signal peptide is removed. Unless explicitly indicated otherwise, all FL amino acid residue positions referred to herein correspond to the amino acid residue positions as indicated in SEQ ID NO: 5. According to the invention, the FL nucleotide and protein sequences referred to herein relate to FL sequences originating from any organism, i.e. all orthologues of FL. Preferably, the FL nucleotide and protein sequences referred to herein are from mammalian origin. Particularly preferred FL sequences are human.

As used herein, the term “ligand” refers to a substance that is able to bind to and form a complex with a biomolecule to serve a biological purpose. The binding occurs by intermolecular forces, such as ionic bonds, hydrogen bonds and van der Waals forces. The docking (association) is usually, and preferably, reversible (dissociation). According to the invention, the ligand referred to herein is a ligand of Flt3 or a ligand of FL. As the ligands according to the present invention are able to modulate Flt3 signaling, the term “ligand” can be used interchangeably with the term “modulator”. In an embodiment, the ligand according to the invention are characterized by a dissociation constant (K_{d}) for its substrate (Flt3 or FL) of at most 10⁻⁵ M, preferably at most 10⁻⁶ M, at most 10⁻⁷ M, at most 10⁻⁸ M, at most 10⁻⁹ M, or at most 10⁻¹⁰ M.

As used herein, the term “binding site” or “binding interface” relates to the respective regions on either of two components where binding takes place. This region typically includes amino acid residues which are directly involved in binding and participate in non-covalent intermolecular interactions. This region may also include amino acid residues which are not directly involved in binding or participate in non-covalent intermolecular interactions, but which are merely interspersed between interacting amino acid residues, and/or provide a structural, special, energetic or other function. The term “binding site” or “binding interface” also refers to an area which determines an exclusion zone or competition zone of a component for two ligands with the same binding site. According to the present invention, the Flt3/FL binding interface or Flt3 and FL binding sites comprises or consists of amino acid residues 240-350, in particular D3, more in particular amino acid residues 245-345, even more in particular amino acid residues 279-311 of Flt3 and amino acid residues 5-20, in particular 5-18, 8-18, 5-15, or 8-15 of FL, preferably 5-15.

As used herein, the term “ligand which modulates Flt3 signaling” or “modulator of Flt3 signaling” refers to a ligand or modulator which is capable of influencing, regulating and/or otherwise altering Flt3 signaling. As such, contacting the ligand or modulator according to the present invention with its substrate results in a measurable effect on Flt3 signaling. Such effects can be for instance partial or full activation of Flt3 signaling, enhancement of Flt3 signaling, reduction of Flt3 signaling or partial or full inhibition of Flt3 signaling. Flt3 signaling is well documented in the art. Flt3 is a class III receptor tyrosine kinase, which activation resides in activation of the intracellular kinase domains by phosphorylation upon ligand binding. These phosphorylation events initiate downstream signaling via the PI3K/AKT and the RAS/RAF/MEK/ERK pathways. Modulation of Flt3 signaling can be easily and routinely evaluated for instance by measurement of a change in intracellular Flt3 phosphorylation or any of the downstream components. By means of example, and without limitation, Flt3 activation can be evaluated by measurement of tyrosine phosphorylation status (such as Y958 or Y969) by means of phospho-specific Flt3 antibodies, which are known in the art. By extension, modulation of Flt3 signaling can also be evaluated based on a specific biological event or outcome. It is known that Flt3 signaling is a potent regulator mechanism of for instance dendritic cell (DC) development and homeostasis and DC-mediated natural killer cell (NKC) activation. Therefore, a modulator of Flt3 signaling may also be evaluated or identified based on for instance measurement of DC proliferation, development, homeostasis or NKC activation.

The ligand according to the present invention can be of any chemical class of molecules, such as, without limitation, a naturally occurring or non-natural occurring protein, nucleic acid, hapten, lipid, carbohydrate, as well as chimeras and/or derivatives thereof, in monomeric, polymeric or conjugated forms. In a preferred embodiment, the ligand is an Alphabody™ (Complix, Belgium) a Nanobody® (Ablynx, Belgium), an antibody, or a small molecule, preferably an Alphabody™.

Antibodies, methods for obtaining antibodies, methods for screening antibodies are known in the art, and will not be detailed further. By means of further guidance, and without limitation, full length antibodies as well as functional fragments thereof, such as Fab, Fab′, (Fab′)₂, or Fv fragments, can be used as ligands to be identified or designed according to the invention. Also single chain antibodies (SCA) can be used.

Nanobodies® are antibody fragments consisting of a single monomeric variable antibody domain. These antibody-derived proteins contain the unique structural and functional properties of naturally-occurring heavy-chain antibodies. Originally derived from camelidae, these heavy-chain antibodies contain a single variable domain (VHH) and two constant domains (CH2 and CH3). The VHH domain is a perfectly stable polypeptide harbouring the full antigen-binding capacity of the original heavy-chain antibody. The isolated VHH domain is called a Nanobody®, and is described for instance in WO 94/04678, which is incorporated herein in its entirety by reference. In addition to sharing various common structural and functional features with conventional antibodies, in particular high target specificity, high affinity for their target, and low inherent toxicity, Nanobodies® offer several additional advantages. Due to their small size (about 1/10^{th} of conventional antibodies), like small molecule drugs they have the opportunity to inhibit enzymes and readily access receptor clefts. Furthermore, Nanobodies® are extremely stable, have the potential to be administered by means other than injection, and are easy to manufacture. These characteristics make Nanobodies® a versatile tool for drug development.

Accordingly, the invention also relates to a Nanobody® as identified or designed according to the methods as described herein.

Alphabodies™ are single-chain, triple-stranded coiled coil proteins with a molecular weight of between 10 and 14 kDa (10 to 15 times smaller than antibodies). Alphabodies™ are described in EP 2 188 303, EP 2 161 278 and WO 2010/066740 which are incorporated herein in their entirety by reference. Alphabodies™ can bind with high affinity to a wide range of molecular targets and display various beneficial characteristics as therapeutic drugs. Due to their unique structural properties, Alphabodies™ can bind to certain types of targets that are not easily accessible to antibodies or other types of protein scaffolds. Because of their small size, Alphabodies™ have a superior tissue penetration potential as compared to larger protein therapeutics, such as conventional antibodies. Despite their small size however, and simple structure, Alphabodies™ can display more than one antigen binding site on their surface; this means that a single Alphabody™ domain can display multi-specific target binding, a feature hardly achievable with antibodies or other known protein scaffolds. Furthermore, Alphabodies™ are extremely stable (melting temperature of >120° C.), can be autoclaved, can be lyophilized, and are highly resistant to various proteases. These properties allow the development of different formulations and alternative modes of administration (such as topical or pulmonary). Additional advantages of Alphabodies™ include the ease with which the in vivo half-life can be modulated (e.g. by standard techniques such as PEGylation) as well as the ease of production (e.g. by E. coli fermentation). Like Nanobodies®, these characteristics make Alphabodies™ a versatile tool for drug development. A particularly advantageous property of Alphabodies™ is their structural similarity with the cognate ligand of Flt3, FL (helical-shaped protein scaffolds), which makes this type of moieties excellent candidates for the design of non-naturally occurring ligands for Flt3. Accordingly, the invention also relates to a Alphabody™ as identified or designed according to the methods as described herein.

As used herein, the term “small molecule” refers to a low molecular weight organic, inorganic, or organometallic compound typically having a molecular weight of less than about 1000, as is generally known in the art. Small molecules can occur naturally (such as neurotransmitters, (steroid) hormones, etc.) or can be chemically synthesized. Most conventional pharmaceuticals, such as for instance aspirin, are small molecules. By means of example, small molecules include, but are not limited to, mono- or oligo-saccharides, -peptides, peptidomimetics, primary or secondary metabolites, etc. Small molecules can be of any chemical class, such as, without limitation, alcohols, ethers, esters, aldehydes, ketons, acids, amines, amides, etc. and can be chemically modified. Small molecule libraries offer a good source of small molecules for use in screening for particular activity. Methods for generating small molecule libraries are for instance disclosed in WO9424314. Various types of small molecule libraries can be obtained from commercial sources, such as, for instance, from ChemBridge (San Diego, Calif., USA).

As used herein, the term “crystal” refers to an ordered state of matter, in particular a structure (such as a three dimensional (3D) solid aggregate) in which the plane faces intersect at definite angles and in which there is a regular structure (such as internal structure) of the constituent chemical species. The term “crystal” refers in particular to a solid physical crystal form such as an experimentally prepared crystal.

Proteins, by their nature are difficult to purify to homogeneity. Even highly purified proteins may be chronically heterogeneous due to modifications, the binding of ligands or a host of other effects. In addition, proteins are crystallized from generally complex solutions that may include not only the target molecule but also buffers, salts, precipitating agents, water and any number of small binding proteins. It is important to note that protein crystals are composed not only of protein, but also of a large percentage of solvents molecules, in particular water. These may vary from 30 to even 90%. Protein crystals may accumulate greater quantities and a diverse range of impurities which cannot be listed here or anticipated in detail. Frequently, heterogeneous masses serve as nucleation centers and the crystals simply grow around them. The skilled person knows that some crystals diffract better than others. Crystals vary in size from a barely observable 20 μm to 1 or more mm. Crystals useful for X-ray analysis are typically single, 0.05 mm or larger, and free of cracks and defects.

As used herein, the term “atomic coordinates” refers to a set of values which define the position of one or more atoms with reference to a system of axes. This term refers to the information of the three dimensional organization of the atoms contributing to a protein structure. The final map containing the atomic coordinates of the constituents of the crystal may be stored on a data carrier; typically the data is stored in PDB format or in x-plor format, both of which are known to the person skilled in the art. However, crystal coordinates may as well be stored in simple tables or text formats. The PDB format is organized according to the instructions and guidelines given by the Research Collaboratory for structural Bioinformatics. It will be understood by those skilled in the art that atomic coordinates may be varied, without affecting significantly the accuracy of models derived therefrom. Thus, although the invention provides a very accurate definition of a preferred atomic structure, it will be understood that minor variations are envisaged and the claims are intended to encompass such variations. The invention also relates to subsets of atomic coordinates as described herein, as well as the use of subsets in the methods as described herein. In a preferred embodiment, said subsets comprise or consist of the Flt3/FL binding interface, the FL binding site on Flt3, or the Flt3 binding site on FL. Particularly preferred subsets of the atomic coordinates as described herein are subsets comprising or consisting of atomic coordinates of atoms 1 to 681 of Table 3 or atoms 1 to 687 of Table 3 for atomic coordinates corresponding to Flt3; or atoms 688 to 1698 of Table 3 for atomic coordinates corresponding to FL. In other preferred embodiments, a subset of atomic coordinates may comprise or consist of atomic coordinates of atoms 227 to 456 of Table 3 for atomic coordinates corresponding to Flt3; or atoms 709 to 818 of Table 3 for atomic coordinates corresponding to FL; or a combination of both. In yet other preferred embodiments, the subsets of atomic coordinates may comprise or consist of atomic coordinates of atoms of Table 3, corresponding to any of the amino acid regions (of Flt3 and/or FL) as disclosed herein.

The term “root mean square deviation” (rmsd) is used as a means of comparing two closely related structures and relates to a deviation in the distance between related atoms of the two structures after structurally minimizing this distance in a superposition. Related proteins with closely related structures will be characterized by relatively low RMSD values whereas larger differences will result in an increase of the RMSD value.

As used herein, the terms “% identical” and “% homologous” in the context of polynucleic acid sequences or polypeptide sequences refer to the similarity between two sequences, preferably expressed as a percentage of identical nucleic acids or amino acids between two sequences after alignment of these sequences. Alignments and percentages of identity can be performed and calculated with various different programs and algorithms known in the art. Preferred alignment algorithms include BLAST (Altschul, 1990; available for instance at the NCBI website) and Clustal (reviewed in Chema, 2003; available for instance at the EBI website). Preferably, BLAST is used to calculate the percentage of identity between two sequences.

In an aspect, the invention relates to a crystal comprising Flt3, in particular the extracellular domain of Flt3. In an embodiment, said extracellular domain is at least 80%, preferably at least 85%, 90%, or 95% identical or homologous to SEQ ID NO: 6. In another embodiment, said extracellular domain has the sequence of SEQ ID NO: 6. The invention further relates to a crystal comprising Flt3, in particular the extracellular domain of Flt3, and a ligand. Preferably, said ligand is FL. In an embodiment, said ligand is at least 80%, preferably at least 85%, 90%, or 95% identical or homologous to SEQ ID NO: 5. In yet another embodiment, said ligand has the sequence of SEQ ID NO: 5.

The invention also relates to a crystal comprising a fragment of the extracellular domain of Flt3. The invention further relates to a crystal comprising a fragment of the extracellular domain of Flt3, and a ligand, preferably FL. Said fragment of the extracellular domain of Flt3 is extracellular domain (D) D1, D2, D3, D4, or D5, preferably D3. In an embodiment, said fragment of the extracellular domain of Flt3 is amino acid residues 79-161, 167-244, 245-345, 348-434, or 435-533, preferably 245-345. In another embodiment, said fragment of the extracellular domain of Flt3 is at least 80%, preferably at least 85%, 90%, or 95% identical or homologous to amino acid residues 79-161, 167-244, 245-345, 348-434, or 435-533 of SEQ ID NO 2, preferably 245-345.

The crystal of the invention preferably effectively diffracts x-rays for the determination of the atomic coordinates of the protein to a resolution better than 6 Å. More preferably the three dimensional structure determinations can be determined with a resolution of more than 5 Å, such as more than 4 Å or most preferably about 3.5 Å using the crystals according to the invention.

In a further embodiment, said crystal comprises a three-dimensional (3D) crystal structure characterized by the atomic coordinates in Table 3, or a subset thereof. Preferred subsets define one or more of the extracellular domains D1, D2, D3, D4, and/or D5 of Flt3. It will be understood that any reference herein, as well as in other aspects and embodiments of the invention as disclosed herein, to the atomic coordinates or subset of the atomic coordinates shown in Table 3 shall include, unless specified otherwise, atomic coordinates having a root mean square deviation of backbone atoms of not more than 3 Å, preferably not more than 2.5 Å, preferably not more than 1.5 Å, even more preferably not more than 1 Å, when superimposed on the corresponding backbone atoms described by the atomic coordinates shown in Table 3. Preferred variants are those in which the root mean square deviation (RMSD) of the x, y and z co-ordinates for all backbone atoms other than hydrogen is less than 1.5 Å (preferably less than 1 Å, 0.7 Å or less than 0.3 Å) compared with the coordinates given in Table 3. It will be readily appreciated by those skilled in the art that a 3D rigid body rotation and/or translation of the atomic coordinates does not alter the structure of the molecule concerned. In a highly preferred embodiment, the crystal has the atomic coordinates as shown in Table 3.

A person skilled in the art will appreciate that a set of atomic coordinates determined by X-ray crystallography is not without standard error. Accordingly, any set of structure coordinates for a crystal as described herein that has a root mean square deviation of protein backbone atoms of less than 0.75 Å when superimposed (using backbone atoms) on the atomic coordinates listed in Table 3 shall be considered identical.

The present invention also relates to the atomic coordinates of a crystal as described herein that substantially conforms to the atomic coordinates listed in Table 3. Accordingly, in an aspect, the invention relates to a set of atomic coordinates as shown in Table 3, or a subset thereof of both or either, in which the coordinates define a three dimensional structure of (the extracellular domain of) Flt3 and/or FL. The invention also relates to atomic coordinates which deviate from those in Table 3, or a subset thereof, by RMSD over protein backbone atoms by no more than 3 Å.

A structure that “substantially conforms” to a given set of atomic coordinates is a structure wherein at least about 50% of such structure has an RMSD of less than about 1.5 Å for the backbone atoms in secondary structure elements in each domain, and more preferably, less than about 1.3 Å for the backbone atoms in secondary structure elements in each domain, and, in increasing preference, less than about 1.0 Å, less than about 0.7 Å, less than about 0.5 Å, and most preferably, less than about 0.3 Å for the backbone atoms in secondary structure elements in each domain.

In a more preferred embodiment, a structure that substantially conforms to a given set of atomic coordinates is a structure wherein at least about 75% of such structure has the recited RMSD value, and more preferably, at least about 90% of such structure has the recited RMSD value, and most preferably, about 100% of such structure has the recited RMSD value. In an even more preferred embodiment, the above definition of “substantially conforms” can be extended to include atoms of amino acid side chains. As used herein, the phrase “common amino acid side chains” refers to amino acid side chains that are common to both the structure which substantially conforms to a given set of atomic coordinates and the structure that is actually represented by such atomic coordinates.

Those of skill in the art will understand that a set of structure coordinates for a protein or protein complex or a portion thereof, is a relative set of points that define a shape in three dimensions. Thus, it is possible that an entirely different set of coordinates could define a similar or identical shape. The variations in coordinates may be generated by mathematical manipulations of the structure coordinates. For example, the structure coordinates set forth in Table 3 could be manipulated by crystallographic permutations of the structure coordinates, fractionalization or matrix operations to sets of the structure coordinates or any combination of the above.

Various computational analyses are used to determine whether a molecular complex or a portion thereof is sufficiently similar to all or parts of the structure of the extracellular domain of IR described above. Such analyses may be carried out in current software applications, such as the Molecular Similarity program of QUANTA (Molecular Simulations Inc., San Diego, Calif.) version 4.1.

The Molecular Similarity program permits comparisons between different structures, different conformations of the same structure, and different parts of the same structure. Comparisons typically involve calculation of the optimum translations and rotations required such that the root mean square difference of the fit over the specified pairs of equivalent atoms is an absolute minimum. This number is given in angstroms (Å).

Accordingly, structural coordinates of an (extracellular domain of) Flt3, or fragments thereof and/or FL within the scope of the present invention include structural coordinates related to the atomic coordinates listed in Table 3 by whole body translations and/or rotations. Accordingly, RMSD values listed herein assume that at least the backbone atoms of the structures are optimally superimposed which may require translation and/or rotation to achieve the required optimal fit from which to calculate the RMSD value. A three dimensional structure of an Flt3 and/or FL polypeptide or region thereof which substantially conforms to a specified set of atomic coordinates can be modeled by a suitable modeling computer program such as MODELER (Sali & Blundell, 1993), as implemented in the Insight II Homology software package (Insight II (97.0), MSI, San Diego), using information, for example, derived from the following data: (1) the amino acid sequence of the human Flt3 (extracellular domain) and/or FL; (2) the amino acid sequence of the related portion(s) of the protein represented by the specified set of atomic coordinates having a three dimensional configuration; and, (3) the atomic coordinates of the specified three dimensional configuration. A 3D structure of such polypeptides which substantially conforms to a specified set of atomic coordinates can also be calculated by a method such as molecular replacement, which is described in detail below.

In another aspect, the invention relates to the use of a crystal as defined herein for determining the 3D structure of (the extracellular domain) of Flt3, or fragments thereof, and/or FL, or fragments thereof, as well as a method for determining the 3D structure of (the extracellular domain) of Flt3, or fragments thereof, and/or FL, or fragments thereof, by means of said crystal.

In a further aspect, the invention relates to a three-dimensional structure obtained by or obtainable by the crystal as described herein.

In a further aspect, the invention relates to the use of the atomic coordinates as described in Table 3, or a subset thereof, or atomic coordinates which deviate from those in Table 3, or a subset thereof, by RMSD over protein backbone atoms by no more than 3 Å, for identifying and/or designing a modulator of Flt3 signaling, for identifying and/or designing a ligand of Flt3 or for identifying and/or designing a ligand of FL.

In a further aspect, the invention relates to a method for identifying and/or designing a modulator of Flt3 signaling, for identifying and/or designing a ligand of Flt3 or for identifying and/or designing a ligand of FL, comprising structure-based identification and/or design of a ligand based on the interaction of said ligand with the 3D structure represented by the atomic coordinates of Table 3, or a subset thereof, or atomic coordinates which deviate from those in Table 3, or a subset thereof, by RMSD over protein backbone atoms by no more than 3 Å. Said subset preferably comprises or consists of the Flt3/FL binding interface, the FL binding site of Flt3 and/or the Flt3 binding site of FL, as described herein.

Structure coordinates/atomic coordinates are typically loaded onto a machine readable-medium for subsequent computational manipulation. Thus models and/or atomic coordinates are advantageously stored on machine-readable media, such as magnetic or optical media and random-access or read-only memory, including tapes, diskettes, hard disks, CD-ROMs and DVDs, flash memory cards or chips, servers and the internet. The machine is typically a computer. Accordingly, in an aspect, the invention relates to a machine- or computer-readable data storage medium comprising a data storage material encoded with the structure coordinates, or at least a portion of the structure coordinates set forth in Table 3. Thus, in accordance with the present invention, the structure coordinates of (the extracellular domain of) Flt3, or fragments thereof and/or FL, or fragments thereof, can be stored in a machine- or computer-readable storage medium. Such data may be used for a variety of purposes, such as drug discovery and X-ray crystallographic analysis of protein crystal. Accordingly, the invention also relates to a computer-readable media comprising the three-dimensional structure of the crystal as described herein. The invention further relates to a computer-readable media comprising the atomic coordinates of Table 3, or a subset thereof, or atomic coordinates which deviate from those in Table 3, or a subset thereof, by RMSD over protein backbone atoms by no more than 3 Å.

The storage medium may be local to a computer as described above, or the storage medium may be located in a net-worked storage medium including the internet, to which remote accessibility is possible.

The structure coordinates/atomic coordinates may be used in a computer to generate a representation, e.g. an image, of the three-dimensional structure of the IR ectodomain crystal which can be displayed by the computer and/or represented in an electronic file.

The structure coordinates/atomic coordinates and models derived therefrom may also be used for a variety of purposes such as drug discovery, biological reagent (binding protein) selection and X-ray crystallographic analysis of other protein crystals. Accordingly, in an aspect, the invention relates to the use of the crystal, the atomic coordinates or the computer-readable media as described herein for the identification and the design of ligands of Flt3 and/or FL. In another aspect, the invention relates to methods for identifying or designing ligands of Flt3 and/or FL by means of the crystal, the atomic coordinates or the computer-readable media as described herein. Alternatively, the invention also relates to the use of the crystal, the atomic coordinates or the computer-readable media as described herein for the identification of the binding-site for a ligand on Flt3 and/or FL. In another aspect, the invention relates to methods for identifying the binding-site for a ligand on Flt3 and/or FL by means of the crystal, the atomic coordinates or the computer-readable media as described herein.

Modulators of Flt3 signaling can be identified or designed with various computer-implemented modeling algorithms known in the art. As used herein, the term “modeling” includes the quantitative and qualitative analysis of molecular structure and/or function based on atomic structural information and interaction models. The term “modeling” includes conventional numeric-based molecular dynamic and energy minimization models, interactive computer graphic models, modified molecular mechanics models, distance geometry and other structure-based constraint models. Molecular modeling techniques can be applied to the atomic coordinates as described herein or a subset thereof to derive a range of 3D models and to investigate the structure of binding sites, such as the binding sites of potential ligands. Such modeling methods are developed to design or select chemical entities that possess stereochemical complementary to particular target regions. By “stereochemical complementarity” is meant that the compound or a portion thereof makes a sufficient number of energetically favourable contacts with the target region as to have a net reduction of free energy on binding to the receptor. It is preferred that the stereochemical complementarity is such that the compound has a dissociation constant (K_{d}) for its substrate (Flt3 or FL) of at most 10⁻⁵ M, preferably at most 10⁻⁶ M, at most 10⁻⁷ M, at most 10⁻⁸ M, at most 10⁻⁹ M, or at most 10⁻¹⁰ M. It will be appreciated that it is not necessary that the complementarity between chemical entities and the receptor site extend over all residues of the target site in order to modulate Flt3 signaling.

Modeling and docking software that can be used for the identification or design of ligands is well known in the art and includes, without limitation DOCK, FLEXR, GOLD, FLO, FRED, GLIDE, LIGFIT, MOE, MVP, QUANTA, INSIGHT, SYBYL, AMBER, CHARMM, GRID, MCSS, AUTODOCK, CAVEAT, MACCS-3D, HOOK. By means of example, and without limitation, the following approach may be used to identify and/or design ligands. Ligands are in silico directly docked from a three-dimensional structural database, to the target site, using mostly, but not exclusively, geometric criteria to assess the goodness-of-fit of a particular molecule to the site. This approach is illustrated by Kuntz et al. (1982) and Ewing et al. (2001), the contents of which are hereby incorporated by reference, whose algorithm for ligand design is implemented in a commercial software package, DOCK version 4.0, distributed by the Regents of the University of California and further described in a document, provided by the distributor, which is entitled “Overview of the DOCK program suite” the contents of which are hereby incorporated by reference. Ligands identified on the basis of geometric parameters, can then be modified to satisfy criteria associated with chemical complementarity, such as hydrogen bonding, ionic interactions and Van der Waals interactions. The scoring functions may include, but are not limited to force-field scoring functions (affinities estimated by summing Van der Waals and electrostatic interactions of all atoms in the complex between the target site and the ligand), empirical scoring functions (counting the number of various interactions, for instance number of hydrogen bonds, hydrophobic-hydrophobic contacts and hydrophilic-hydrophobic contacts, between the target site and the ligand), and knowledge based scoring functions (with basis on statistical findings of intermolecular contacts involving certain types of atoms or functional groups). Scoring functions involving terms from any of the two of the mentioned scoring functions may also be combined into a single function used in database virtual screening of chemical libraries. Different scoring functions can be employed to rank and select the best molecule from a database. See for example Bohm & Stahl (1999). The software package FlexX, marketed by Tripos Associates, Inc. (St. Louis, Mo.) is another program that can be used in this direct docking approach (see Rarey et al., 1996).

Once a ligand has been designed or identified, the efficiency with which the ligand may bind to the target site can be tested and optimized by computational evaluation. An effective ligand must preferably demonstrate a relatively small difference in energy between its bound and free states (i.e., a small deformation energy of binding). Thus, the most efficient ligand should preferably be designed with a deformation energy of binding of not greater than about 10 kcal/mole, preferably, not greater than 7 kcal/mole.

A compound designed or identified as binding to a target site may be further computationally optimized so that in its bound state it would preferably lack repulsive electrostatic interaction with the target protein. Such non-complementary (e.g., electrostatic) interactions include repulsive charge-charge, dipole-dipole and charge-dipole interactions. Specifically, the sum of all electrostatic interactions between ligand and the target site, preferably make a neutral or favorable contribution to the enthalpy of binding. Once an Flt3- or FL-binding ligand has been optimally selected or designed, as described above, substitutions may then be made in some of its atoms or side groups to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to the target site by the same computer methods described above.

The identification and/or design methods may be implemented in hardware or software, or a combination of both. However, preferably, the methods are implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design. Each program is preferably implemented in a high level procedural or object-oriented programming language to communicate with a computer system. However, the programs can be implemented in assembly or machine language, if desired. In any case, the language may be compiled or interpreted language. Accordingly, the invention relates to a computer system comprising:
a) a database containing information on the three dimensional structure of the crystal as described herein, stored on a computer readable storage medium; and
b) a user interface to view the information.

In an embodiment, said database contains the atomic coordinates presented in Table 3, or a subset thereof, or atomic coordinates which deviate from those in Table 3, or a subset thereof, by RMSD over protein backbone atoms by no more than 3 Å, stored on a computer readable storage medium. Said subset preferably comprises or consists of the Flt3/FL binding interface, the FL binding site of Flt3 and/or the Flt3 binding site of FL, as described herein.

In an aspect, the invention relates to a method of identifying or designing a ligand which modulates Flt3 signaling, a ligand of (the region comprised within amino acid residues 240-350, preferably 245-345 of) Flt3 or a ligand of (the region comprised within amino acid residues 5-20 of) FL, comprising the step of employing a three dimensional structure of the crystal as described herein or the atomic coordinates as described herein, or a subset thereof or atomic coordinates which deviate from those in Table 3, or a subset thereof, by RMSD over protein backbone atoms by no more than 3 Å.

In an embodiment, said method further comprises the step of structure-based identification and/or design of a ligand based on the interaction of said ligand with the 3D structure represented by the atomic coordinates of Table 3, or a subset thereof, or atomic coordinates which deviate from those in Table 3, or a subset thereof, by RMSD over protein backbone atoms by no more than 3 Å.

In an embodiment, said method is a computer-implemented method, said computer preferably comprising an inputting device, a processor, a user interface, and/or an outputting device. Said inputting device may comprise for instance a CD-rom driver, a USB-port, a keyboard. Said processor may comprise hardware and software (such as the modeling algorithms and programs as described herein). Said user interface may comprise a computer screen. Said outputting device may comprise a printer.

In an embodiment, said method, comprises the steps of:
a) generating a three-dimensional structure of atomic coordinates presented in Table 3, or a subset thereof, or atomic coordinates which deviate from those in Table 3, or a subset thereof, by RMSD over protein backbone atoms by no more than 3 Å;
b) fitting the structure of step a) with the structure of a candidate ligand by computational modeling;
c) selecting a ligand that possesses energetically favorable interactions with the structure of step a).

In an embodiment, said fitting comprises superimposing the structure of step a) with the structure of said candidate ligand. In another embodiment, said modeling comprises docking modeling. In a further embodiment, said ligand of step c) can bind to at least 1 amino acid residue, such as at least 2, 3, 4, 5, 6, 7, or 8 amino acid residues of the structure of step a) without steric interference.

It will be understood by the skilled person that generating the structures, as well as the modeling and fitting operations as described above may be performed with the algorithms, programs and platforms as disclosed in the present specification.

In an aspect, the invention also relates to a method for identifying modulators of Flt3 signaling. In particular, the invention relates to a method for identifying a ligand which modulates Flt3 signaling, comprising the steps of:
a) providing a candidate ligand;
b1) providing a polypeptide comprising or consisting of a region of at least 5 consecutive amino acid residues of amino acid residues 240-350, preferably 245-345 of Flt3; or
b2) providing a polypeptide comprising or consisting of a region of at least 5 consecutive amino acid residues of amino acid residues 5-20 of FL;
c) contacting said candidate ligand with said polypeptide of step b1) or step b2);
d) determining the binding of said candidate ligand with said region of step b1) or step b2); and
e) identifying said candidate ligand as a ligand which modulates Flt3 signaling if binding between said candidate ligand and said region of step b1) or step b2) is detected.

The invention also relates to a method for identifying a ligand of Flt3, comprising the steps of:
a) providing a candidate ligand;
b) providing a polypeptide comprising or consisting of a region of at least 5 consecutive amino acid residues of amino acid residues 240-350, preferably 245-345 of Flt3;
c) contacting said candidate ligand with said polypeptide of step b);
d) determining the binding of said candidate ligand with said region of step b); and
e) identifying said candidate ligand as a ligand which modulates Flt3 signaling if binding between said candidate ligand and said region of step b) detected.

The invention also relates to a method for identifying a ligand of Flt3 which binds to the FL binding site, in particular the region of Flt3 comprised within of consisting of amino acid residues 240-350, preferably 245-345, the method comprising the steps of:
a) providing a candidate ligand;
b) providing a polypeptide comprising or consisting of said FL binding site or said region,
c) contacting said candidate ligand with said polypeptide;
d) determining the binding of said candidate ligand with said region;
e) identifying said candidate ligand as a ligand of Flt3 if binding is detected.

The invention further relates to a method for identifying a ligand of FL, comprising the steps of:
a) providing a candidate ligand;
b) providing a polypeptide comprising or consisting of a region of at least 5 consecutive amino acid residues of amino acid residues 5-20 of FL;
c) contacting said candidate ligand with said polypeptide of step b);
d) determining the binding of said candidate ligand with said region of step b); and
e) identifying said candidate ligand as a ligand which modulates Flt3 signaling if binding between said candidate ligand and said region of step b) detected.

The invention also relates to a method for identifying a ligand of FL that binds to the Flt3 binding site, in particular the region of FL comprised within or consisting of amino acid residues 5-20, the method comprising the steps of:
a) providing a candidate ligand;
b) providing a polypeptide comprising or consisting of said Flt3 binding site or said region,
c) contacting said candidate ligand with said polypeptide;
d) determining the binding of said candidate ligand with said region;
e) identifying said candidate ligand as a ligand of FL if binding is detected.

According to an aspect of the invention, a candidate ligand is brought into contact with any one of the above indicated polypeptides or fragments of Flt3 or FL, after which binding between said candidate ligand and said polypeptides or fragments of Flt3 or FL is evaluated. In a particularly preferred embodiment, the binding between said candidate ligand and the respective region of Flt3 or FL which constitutes the Flt3/FL binding interface is determined. Methods for identifying interactions between compounds, such as interactions between proteins, are well known in the art, and will not be detailed further. By means of example, and without limitation, interactions can be evaluated by techniques such as pull-down, co-immunoprecipitation, yeast two-hybrid, bimolecular fluorescence complementation (BiFC), affinity electrophoresis, label transfer, phage display, ELISA, RIA, in-vivo crosslinking, tandem affinity purification (TAP), chemical crosslinking, dual polarisation interferometry (DPI), surface plasmon resonance (SPR), static light scattering (SLS), dynamic light scattering (DLS or QELS), fluorescence polarization/anisotropy, fluorescence correlation spectroscopy, fluorescence resonance energy transfer (FRET), EMSA, NMR, isothermal titration calorimetry (ITC). Particularly preferred techniques include competition or displacement assays, which are well known in the art. Briefly, a known ligand (such as (a fragment of) FL or Flt3) competes with the candidate ligand for binding. Either one or both of the known or candidate ligand can be labeled for ease of (differential) detection. Different types of labels are well known in the art, such as labels which allow fluorescent detection or affinity purification. Typically, a dilution series of candidate or known ligand is incubated with the binding partner and with fixed concentration of known or candidate ligand. Concentration-dependent changes in the detection of binding of the known or candidate ligand identifies candidate ligands as effective ligands. An alternative technique to validate candidate ligands comprises on the one hand incubating the candidate ligand with a wild type binding partner or fragment thereof (Flt3 or FL) and on the other hand incubating the candidate ligand with a mutated binding partner or fragment thereof (Flt3 or FL), wherein the mutated Flt3 or FL comprises at least one mutation in the respective binding domain of Flt3 or FL which constitutes the Flt3/FL binding interface. It will be understood by a person skilled in the art that preferred mutations constitute non-conservative mutations.

Accordingly, in an aspect, the invention relates to a method for identifying a ligand of Flt3, comprising the steps of
a) providing a candidate ligand;
b1) providing a first polypeptide comprising or consisting of a region of at least 5 consecutive amino acid residues of amino acid residues 240-350, preferably 245-345 of Flt3;
b2) providing a second polypeptide comprising or consisting of said region, wherein at least one amino acid residue of amino acid residues 240-350, preferably 245-345 is mutated;
c) contacting said candidate ligand with said polypeptide of step b1) or step b2);
d) determining the binding of said candidate ligand with said region of step b1) and step b2); and
e) identifying said candidate ligand as a ligand of Flt3 if binding between said candidate ligand and said region of step b1) is detected and if no binding between said candidate ligand and said region (or polypeptide) of step b2) is detected.

Particularly preferred amino acid residues to be mutated on Flt3 comprise one or more of amino acid residues at position 279, 281, 301, 302, 303, 307, 309, and 311. Accordingly, in an embodiment, the invention relates to a method as describes above, wherein said at least 5 consecutive amino acid residues comprise one or more of amino acid residues at position 279, 281, 301, 302, 303, 307, 309, and 311 of which one or more is mutated. In a further aspect, the invention relates to an Flt3 (isolated) polypeptide or a fragment thereof (such as D3, or a fragment corresponding to amino acid residues 245-345 of SEQ ID NO: 2), as well as the polynucleic acid sequences encoding these polypeptides, wherein at least one of the amino acid residues, or the corresponding nucleotide(s) in the polynucleic acid sequence encoding said polypeptide, comprised within the FL binding domain is mutated. In an embodiment, one or more amino acid residue, or the corresponding nucleotide(s), comprises within amino acid residues 240-350, preferably 245-345, more preferably 279-311 is mutated. In a preferred embodiment, one or more of amino acids 279, 280, 281, 301, 302, 303, 307, 309, or 311 is mutated.

In a further aspect, the invention relates to a method for identifying a ligand of FL, comprising the steps of
a) providing a candidate ligand;
b1) providing a first polypeptide comprising or consisting of a region of at least 5 consecutive amino acid residues of amino acid residues 5-20 of FL;
b2) providing a second polypeptide comprising or consisting of said region wherein at least one amino acid residue of amino acid residues 5-20 is mutated;
c) contacting said candidate ligand with said polypeptide of step b1) or step b2);
d) determining the binding of said candidate ligand with said region of step b1) and step b2); and
e) identifying said candidate ligand as a ligand of FL if binding between said candidate ligand and said region of step b1) is detected and if no binding between said candidate ligand and said region (or polypeptide) of step b2) is detected.

Particularly preferred amino acid residues to be mutated on FL comprise one or more of amino acid residues at position 8, 9, 10, 11, 12, 13, 14, and 15. Accordingly, in an embodiment, the invention relates to a method as describes above, wherein said at least 5 consecutive amino acid residues comprise one or more of amino acid residues at position 8, 9, 10, 11, 12, 13, 14, and 15 of which one or more is mutated.

Underlying the present invention is the surprising finding that the binding interface of Flt3 and its cognate ligand FL comprises a subset of extracellular domain 3 (D3) of Flt3 (comprised within amino acid residues 240-350, preferably 245-345 of Flt3) and an N-terminal part of FL (comprised within amino acid residues 5-20 of FL). Accordingly, in an aspect, the present invention relates to a method for the identification of ligands which modulate (or modulators) of Flt3 signaling, wherein said ligands or modulators are capable of binding to the respective binding site of Flt3 or FL which contribute to the Flt3/FL binding interface. In another aspect, the invention relates to a method for the identification of ligands of Flt3, wherein said ligands are capable of binding to the binding site of Flt3 which contributes to the Flt3/FL binding interface. In a further aspect, the invention relates to a method for the identification of ligands of FL, wherein said ligands are capable of binding to the binding site of FL which contributes to the Flt3/FL binding interface.

According to an aspect of the invention, the methods as described herein for identifying a ligand of Flt3 or a ligand which modulates Flt3 signaling comprise a step of providing a polypeptide or the atomic coordinates of Table 3, or a subset thereof, or atomic coordinates which deviate from those in Table 3, or a subset thereof, by RMSD over protein backbone atoms by no more than 3 Å comprising a region of at least 5 consecutive amino acid residues of amino acid residues 240-350, preferably 245-345, more preferably 279-311 of Flt3. In an embodiment, said polypeptide or atomic coordinates comprises a region of at least 5 consecutive amino acid residues of amino acid residues 240-350, preferably 245-345, more preferably 279-311 of a protein which is at least 80%, preferably at least 85%, 90%, or 95% identical or homologous to SEQ ID NO: 2. In another embodiment, said polypeptide or atomic coordinates comprises a region of at least 5 consecutive amino acid residues of amino acid residues 240-350, preferably 245-345, more preferably 279-311 of SEQ ID NO: 2. In an embodiment, said polypeptide or atomic coordinates comprises at least 5 consecutive amino acid residues of amino acid residues 250-350, 250-340, 260-350, 260-340, 270-340, 270-330, 270-320, 275-320, 275-315, or 279-311 of Flt3, of SEQ ID NO: 2, or of a protein which is at least 80%, preferably at least 85%, 90%, or 95% identical or homologous to SEQ ID NO: 2. In another embodiment, said polypeptide or atomic coordinates comprises 6, 7, 8, 9, 10, 11, 12, 13, 14, 15, 16, 17, 18, 19, 20, 25, 30, 35, 40, 45, 50, 60, 70, 80, 90, or 100 or at least the recited number of consecutive amino acid residues of the region comprised within any of the amino acid residues 250-350, 260-350, 250-340, 260-340, 270-340, 270-330, 270-320, 275-320, 275-315, or 279-311 of Flt3, of SEQ ID NO: 2, or of a protein which is at least 80%, preferably at least 85%, 90%, or 95% identical or homologous to SEQ ID NO: 2. In a further embodiment, said polypeptide or atomic coordinates consists of extracellular domain 3 (D3) of Flt3. In another embodiment, said polypeptide or atomic coordinates consists of a fragment of D3 of Flt3, wherein said fragment of D3 comprises at least 5 consecutive amino acid residues of amino acid residues 240-350, preferably 245-345, more preferably 279-311 of Flt3, of SEQ ID NO: 2, or of a protein which is at least 80%, preferably at least 85%, 90%, or 95% identical or homologous to SEQ ID NO: 2. In an embodiment, said fragment of D3 comprises at least 5 consecutive amino acid residues of amino acid residues 260-350, 250-340, 260-340, 270-340, 270-330, 270-320, 275-320, 275-315, or 279-311 of Flt3, of SEQ ID NO: 2, or of a protein which is at least 80%, preferably at least 85%, 90%, or 95% identical or homologous to SEQ ID NO: 2. In another embodiment, said fragment of D3 comprises 6, 7, 8, 9, 10, 11, 12, 13, 14, 15, 16, 17, 18, 19, 20, 25, 30, 35, 40, 45, 50, 60, 70, 80, 90, or 100 or at least the recited number of consecutive amino acid residues of the region comprised within amino acid residues 250-350, 250-340, 260-350, 260-340, 270-340, 270-330, 270-320, 275-320, 275-315, or 279-311 of Flt3, of SEQ ID NO: 2, or of a protein which is at least 80%, preferably at least 85%, 90%, or 95% identical or homologous to SEQ ID NO: 2. In yet another embodiment, said polypeptide or atomic coordinates consists of amino acid residues 245-345 of Flt3, more preferably 279-311 of SEQ ID NO: 2, or of a protein which is at least 80%, preferably at least 85%, 90%, or 95% identical or homologous to SEQ ID NO: 2.

In an aspect, the invention also specifically relates to the (isolated) Flt3 polypeptide sequences as well as the as the (isolated) polynucleic acid sequences encoding said polypeptide sequences as described herein. In a preferred embodiment, said Flt3 polypeptide sequence comprises at most 200 amino acid residues, preferably at most 175, 150, 125, or 100 amino acid residues. A particularly preferred Flt3 polypeptide according to an embodiment of the invention comprises D3 as the sole Flt3-derived polypeptide fragment or is at least 80%, preferably at least 85%, 90%, or 95% identical or homologous to amino acid residues 245-345 of SEQ ID NO: 2.

In an aspect, the invention relates to the use of the polypeptides and/or fragments thereof or atomic coordinates as described herein for designing and/or identifying a ligand which modulates Flt3 signaling.

In a further aspect, the invention also relates to the use of the polypeptides and/or fragments thereof or atomic coordinates as described herein for designing and/or identifying a ligand of Flt3, in particular, the FL-binding region of Flt3. As these polypeptide fragments or atomic coordinates of Flt3 comprise the FL binding site, these fragments may be used to inhibit Flt3 signaling. Accordingly, in an aspect, the invention relates to the use of said fragment as an antagonist of Flt3 signaling, as well as a method for antagonizing Flt3 signaling by using said fragments.

According to another aspect of the invention, the methods as described herein for identifying a ligand of FL or a ligand which modulates Flt3 signaling comprises a step of providing a polypeptide or atomic coordinates comprising a region of at least 5 consecutive amino acid residues of amino acid residues 5-20 of FL. In an embodiment, said polypeptide comprises a region of at least 5 consecutive amino acid residues of amino acid residues 5-20 of a protein which is at least 80%, preferably at least 85%, 90%, or 95% identical or homologous to SEQ ID NO: 5. In another embodiment, said polypeptide or atomic coordinates comprises a region of at least 5 consecutive amino acid residues of amino acid residues 5-20 of SEQ ID NO: 5. In an embodiment, said polypeptide or atomic coordinates comprises at least 5 consecutive amino acid residues of amino acid residues 5-19, 5-18, 5-17, 5-16, 5-15, 6-20, 6-19, 6-18, 6-17, 6-16, 6-15, 7-20, 7-19, 7-18, 7-17, 7-16, 7-15, 8-20, 8-19, 8-18, 8-17, 8-16, 8-15 of FL, of SEQ ID NO: 5, preferably 8-15 or 5-15 of FL or SEQ ID NO: 5, or of a protein which is at least 80%, preferably at least 85%, 90%, or 95% identical or homologous to SEQ ID NO: 5. In another embodiment, said polypeptide or atomic coordinates consists of a fragment of at most 50 consecutive amino acid residues of FL, wherein said fragment comprises at least 5 consecutive amino acid residues of amino acid residues 5-19, 5-18, 5-17, 5-16, 5-15, 6-20, 6-19, 6-18, 6-17, 6-16, 6-15, 7-20, 7-19, 7-18, 7-17, 7-16, 7-15, 8-20, 8-19, 8-18, 8-17, 8-16, 8-15 of FL, of SEQ ID NO: 5, preferably 8-15 or 5-15 of FL or SEQ ID NO: 5, or of a protein which is at least 80%, preferably at least 85%, 90%, or 95% identical or homologous to SEQ ID NO: 5. In another embodiment, said polypeptide or atomic coordinates comprises 6, 7, 8, 9, 10, 11, 12, 13, 14, 15 or 16 or at least the recited number of consecutive amino acid residues of the region comprised within any of the amino acid residues 5-19, 5-18, 5-17, 5-16, 5-15, 6-20, 6-19, 6-18, 6-17, 6-16, 6-15, 7-20, 7-19, 7-18, 7-17, 7-16, 7-15, 8-20, 8-19, 8-18, 8-17, 8-16, 8-15 of FL, of SEQ ID NO: 5, preferably 8-15 or 5-15 of FL or SEQ ID NO: 5, or of a protein which is at least 80%, preferably at least 85%, 90%, or 95% identical or homologous to SEQ ID NO: 5. In a further embodiment, said polypeptide or atomic coordinates consists of amino acid residues 8-15 of FL, of SEQ ID NO: 5, or of a protein which is at least 80%, preferably at least 85%, 90%, or 95% identical or homologous to SEQ ID NO: 5.

In an aspect, the invention also specifically relates to the FL (isolated) polypeptide sequences as well as the as the (isolated) polynucleic acid sequences encoding said polypeptide sequences as described herein. In a preferred embodiment, said FL polypeptide sequence comprises at most 50 amino acid residues, preferably at most 40, 30, 20, or 10 amino acid residues. A particularly preferred FL polypeptide according to an embodiment of the is at least 80%, preferably at least 85%, 90%, or 95% identical or homologous to amino acid residues 5-20, more preferably 8-18 of SEQ ID NO: 5.

In an aspect, the invention also relates to the use of the polypeptides and/or fragments thereof or atomic coordinates as described herein for designing and/or identifying a ligand of FL, in particular, the Flt3-binding region of FL. As these polypeptide fragments of FL comprise the Flt3 binding site, these fragments may be used to inhibit Flt3 signaling. Accordingly, in an aspect, the invention relates to the use of said fragment as an antagonist of Flt3 signaling, as well as a method for antagonizing Flt3 signaling by using said fragments.

It will be appreciated by a skilled person that the Flt3 and/or FL polypeptides and polynucleotides as described herein can be fused to heterologous polypeptide or polynucleotide sequences. As used herein, the term “heterologous polypeptide” and “heterologous polynucleotide” relate to polypeptides or polynucleotides which are not derived from or originate from Flt3 or FL. Examples of such heterologous sequences include for instance tags, such as tags for detection and/or isolation and/or immobilization and/or reporter tags, etc.

The invention also relates to polypeptide and polynucleic acid sequences comprising or encoding the herein described respective region of Flt3 or FL which constitutes the Flt3/FL binding interface, as well as the full length Flt3 or FL or fragments thereof wherein one or more of the amino acid residues of the respective region of Flt3 or FL which constitutes the Flt3/FL binding interface, or the corresponding nucleotide(s) in the polynucleic acid encoding the polypeptides, are mutated.

A person skilled in the art will appreciate that the polynucleic acids disclosed herein can be cloned in a vector, with techniques which are well known in the art (such as PCR amplification or restriction digests). Accordingly, in an aspect, the invention relates to vector comprising a polynucleic acid as described herein. In an embodiment, said vector is an expression vector, such as a eukaryotic or prokaryotic expression vector. Vectors in general and eukaryotic or prokaryotic expression vectors in particular are well known in the art and hence will not be detailed further. In an aspect, the invention also relates to a host cell comprising a polynucleic acid or a vector as described herein. Suitable host cells include prokaryotic and eukaryotic host cells, such as bacteria, yeast, insect cells and mammalian cells. Methods for transiently or stably introducing polynucleic acids in these host cells (such as transformation, infection, electroporation, transfection), as well as methods for expressing polypeptides encoded by these polynucleic acids (inducible or constitutive) are well known in the art. The invention in an aspect also relates to the use of these host cells for the expression of the polypeptides as disclosed herein, as well as methods for expressing the polypeptides as disclosed herein by use of these host cells.

The invention also relates to ligands of Flt3 or FL, preferably ligands which bind to the respective domains of Flt3 or FL constituting the Flt3/FL binding interface. As Flt3 and FL are known binding partners and hence per se ligands of each other, the full length FL and Flt3 polypeptides are hereby explicitly disclaimed as ligands.

In an aspect, the invention relates to a ligand which is identified by the methods as described herein. In an embodiment, said ligand is an Alphabody™, a Nanobody®, an antibody, or a small molecule, preferably an Alphabody™.

In another aspect, the invention relates to a ligand which binds to the FL binding site of Flt3. In an embodiment, said ligand is an Alphabody™, a Nanobody®, an antibody, or a small molecule, preferably an Alphabody™.

In another aspect, the invention relates to a ligand which binds to the Flt3 binding site of FL. In an embodiment, said ligand is an Alphabody™, a Nanobody®, an antibody, or a small molecule, preferably an Alphabody™.

In a further aspect, the invention relates to the ligands as described herein for use in modulating Flt3 signaling or for use as a medicament. In a further aspect, the invention relates to the use of the ligands as described herein for the manufacture of a medicament. In a further aspect, the invention relates to a method for treating diseases or disorders characterized by abnormal Flt3 signaling with a ligand as described herein. Diseases or disorders characterized by abnormal Flt3 signaling can be caused by a lack of or insufficient Flt3 signaling or alternatively can be caused by inappropriate or increased Flt3 signaling.

In an embodiment, the ligand as described herein may be coupled to a therapeutic compound or drug, and hence may function as a drug-delivery vehicle. Accordingly, in an aspect, the invention relates to a ligand as described herein for use in drug delivery, wherein said ligand is coupled to said drug.

In an embodiment, the ligand according to the present invention is a ligand which modulates or interferes with Flt3 dimerization. The ligand according to this embodiment is a monovalent ligand which completely or partially prevents ligand-mediated dimerization of Flt3 receptors. In an embodiment, the ligand according to the present invention is a ligand which modulates or interferes with Flt3/FL binding. The ligand according to this embodiment completely or partially prevents binding of the cognate ligand FL to Flt3. In another embodiment, the ligand according to the present invention is a ligand which modulates Flt3 (kinase) activation. The ligand according to this embodiment completely or partially alters Flt3 phosphorylation. In a further embodiment, the ligand according to the present invention is a therapeutical agent. The ligand according to this embodiment modulates Flt3 signaling such that a biological effect results in a therapeutic application. In another embodiment, the ligand according to the present invention is an agonist or an antagonist of Flt3 signaling. An agonist according to this embodiment completely or partially activates or enhances Flt3 signaling. An antagonist according to this embodiment completely or partially inhibits or reduces Flt3 signaling. It is to be understood that the ligand according to the invention exerts its function either on Flt3 (if it is a ligand of Flt3) or on FL (if it is a ligand of FL).

In an embodiment, the invention relates to a ligand as described herein for use in modulating Flt3 signaling. Preferred indications which benefit from Flt3 modulation include cancer, precancerous state, autoimmune diseases (such as rheumatoid arthritis), transplantation or grafting, inflammation, immunomodulation, musculo-skeletal disorders (in particular bone disorders such as characterized by abnormal bone resorption), angiogenesis, ophthalmological disorders (such as diabetic macular edema and macular degeneration), apoptosis, cell cycle regulation, dermatological abnormalities (such as dermal fibrosis, mastocytis and psoriasis), CNS disorders (such as multiple sclerosis). In a preferred embodiment, the invention relates to a ligand as described herein for use in treating cancer. In another preferred embodiment, the invention relates to a ligand as described herein for use in treating autoimmune diseases, preferably rheumatoid arthritis, psoriasis or multiple sclerosis. In yet another preferred embodiment, the invention relates to a ligand as described herein for use in cell or organ transplantation. Said ligand is preferably administered prior to, during and/or after transplantation.

In another embodiment, the invention relates to a ligand as described herein for use in any of:
a) treating cancer, said cancer not being characterized by increased Flt3 signaling, if said ligand is an Flt3 agonist;
b) treating cancer, said cancer being characterized by increased Flt3 signaling, if said ligand is an Flt3 antagonist;
c) treating autoimmune diseases if said ligand is an Flt3 antagonist;
d) stimulating an immune response if said ligand is an Flt3 agonist;
e) suppressing an immune response if said ligand is an Flt3 antagonist, such as in the case of transplantation;
f) expansion of dendritic cells if said ligand is an Flt3 agonist;
g) activating Flt3 signaling if said ligand is an agonist;
h) suppressing Flt3 signaling if said ligand is an antagonist.

Cancer treatment which benefits from Flt3 antagonists relates to cancers characterized by an inappropriately increased Flt3 signaling, such as acute myeloid leukemia (AML), bile duct cancer, bladder cancer, brain tumors (in particular (anaplastic) astrocytoma or glioblastoma), breast cancer, uterine cancer, leukemia (in particular (chronic) lymphocytic or myelogenous leukemia, colon cancer, colorectal cancer, stomach cancer, head and neck cancer (in particular squamous cell carcinoma), hematological malignancies (in particular (systemic) mastocytosis or myoproliferative diseases), kidney cancer (in particular urothelial or renal cell carcinoma), liver cancer (in particular hepatocellular carcinoma), lymphoma, melanoma, mesothelioma, multiple myeloma, neoplasia, neuroendocrine tumors (in particular advanced pancreatic neuroendocrine tumors), lung cancer (in particular non-small cell lung cancer), ovarial cancer, pancreatic cancer, prostate cancer, sarcoma or thyroid cancer. In a preferred embodiment, the invention relates to a ligand which is an antagonist designed and/or identified as described herein, for use in treating acute myeloid leukemia. On the other hand, cancer treatment which benefits from Flt3 agonists relates to cancers which are not characterized by an inappropriately increased Flt3 signaling. Particularly beneficial applications of Flt3 agonists relate to immunotherapy in such cancers. In particular, Flt3 signaling is involved in DC homeostasis and DC-mediated activation of NK cells. Hence, activation of Flt3 signaling by Flt3 agonists in DC cells leads to DC proliferation and expansion in aiding immunotherapy. It will be appreciated by the skilled person that, FL as the cognate ligand of Flt3, dimerizes and as a consequence thereof brings Flt3 individual receptors in close proximity upon interaction of an FL dimer with two Flt3 receptors. Such association of Flt3 receptors will lead to intermolecular Flt3 phosphorylation and downstream signaling. Accordingly, a ligand which functions as an agonist preferably is bivalent with respect to Flt3 binding. Alternatively, two monovalent ligands may be coupled to each other to mimic a bivalent ligand. Such ligands may be coupled covalently, for instance by linker or hinge regions, or non-covalently, for instance by self-association or dimerization.

The invention also relates to medicaments or compositions comprising or consisting of the ligands as described herein. In a preferred embodiment, the invention relates to such medicaments or compositions, wherein said ligand is identified according to the methods as described herein. In an embodiment, said compositions are pharmaceutical compositions comprising a ligand as described herein and one or more pharmaceutically acceptable excipients, such as without limitation buffers (such as for instance isotonic saline solutions or PBS), salts, stabilizers, solubilizers, coating agents, emulgators, etc. Pharmaceutical compositions or medicaments containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications. Routes of administration include topical, parenteral, intramuscular, oral, intravenous, intra-peritoneal, intranasal inhalation, lung inhalation, intradermal or intra-articular. Due to the high stability of Nanobodies® and Alphabodies™, oral administration of medicaments comprising Nanobodies® and Alphabodies™ as described herein is preferred.

The invention further relates to such compositions for use as a medicament. The invention further relates to the use of such compositions for the manufacture of a medicament. The invention further relates to a method of treatment by using such compositions.

The invention also relates to a method for modulating Flt3 signaling, comprising the steps of:
a) providing (a composition comprising) an Flt3 polyprotein; and
b) contacting said (composition comprising an) Flt3 polyprotein with a ligand as described herein.

In an embodiment, said method is an in vitro method, wherein said Flt3 polypeptide is provided in an isolated form from an individual, such as an isolated cancer cell, DC, etc.

The invention also relates to a method for determining a mutation in the ligand-binding region of Flt3, comprising the step of determining one or more mutation in the region corresponding to amino acid residues 240-350, preferably 245-345 of Flt3 and/or the polynucleic acid encoding said region. Preferably, said region comprises or consists of amino acid residues 240-350, in particular D3, more in particular amino acid residues 245-345, even more in particular amino acid residues 279-311 of Flt3. Particularly preferred Flt3 mutations comprise mutations of amino acid residues at positions 279, 280, 281, 301, 302, 303, 307, 309, or 311.

In another aspect, the invention relates to a method for diagnosing a disorder which is characterized by aberrant Flt3 signaling, the method comprising the step of determining one or more mutation in the region corresponding to amino acid residues 240-350, preferably 245-345 of Flt3 and/or the polynucleic acid encoding said region. Preferably said method is an in vitro method. Accordingly, said mutation(s) is (are) detected in a sample isolated from an individual.

In a further aspect, the invention relates to the use of a ligand as designed or identified according to any one of the methods defined herein, as a modulator of Flt3 signaling.

The invention will now be illustrated by means of the following examples, which do not limit the scope of the invention in any way.

### EXAMPLES

### Example 1

### Preparation of Recombinant Human Flt3 and Flt3-FL Complexes

cDNA encoding human Flt3 ectodomain variants, Flt3D1(27-161), Flt3D12 (27-244), Flt3D13(27-346), Flt3D14(27-434) and Flt3D15(27-541) were cloned in the mammalian expression vectors, pHLsec (Aricescu) and pcDNA4/TO (Invitrogen), which contained a p-phosphatase secretion signal and a C-terminal hexahistine tag.

Transient protein expression in HEK293T was carried out as previously described (Aricescu, 2006). Briefly, confluent cells, grown in tissue culture flasks or roller bottles (Greiner Bio-One) were transfected with purified plasmid DNA (Plasmid Mega Kit, Qiagen) mixed with 25 kDa branched polyethylenimine (Aldrich) and allowed to secrete the recombinant protein for 5-7 days in serum free medium, in the presence of kifunensine.

The pcDNA4/TO constructs were used to establish stable secreting cell lines in HEK293S GnTI−/− cells, as follows. 70% confluent cells were transfected with the plasmid-DNA according the calcium phosphate precipitation method. Stably transfected clones were selected using Zeocine (Invitrogen) at a concentration of 200 μg/mL, and allowed to grow for 3 weeks. Individual colonies were picked up with trypsin-soaked pieces of filter paper, expanded and subsequently tested for their protein expression. The presence of the recombinant protein in the medium was detected by Western blot analysis using a anti-His(C-term)-antibody coupled to horseradish peroxidase (Invitrogen). For large scale expression experiments, the medium of 50 confluent 175 cm2 tissue culture flasks was replaced with serum-free DMEM-F12 medium containing 5 mM sodium butyrate (Sigma) and 2 μg/mL tetracycline to induce protein expression.

The receptor variants were purified using IMAC: conditioned medium (1-3 liter) was applied to a Talon column (Clontech) with a bed volume of 20 mL, washed and eluted using imidazole. The proteins were further purified by gel-filtration chromatography on a Superdex 200 column (GE Healthcare). Ligand-receptor complexes were formed by adding excess molar amounts of recombinant FL (Verstraete, 2009) to purified receptor ectodomains, followed by purification by gel filtration chromatography.

### Example 2

### Mapping of Disulfide Bridges and Glycosylation Sites by Mass Spectrometry

Gel slices containing recombinant Flt3D1-D5 obtained from Coomassie-stained polyacrylamide gels were digested with trypsin (Promega) as previously described (Vanrobaeys, 2003). After digestion overnight at 37° C., the digestion mixture were dried and redissolved in 20 ml 0.1% formic acid. One microliter of the digestion mixture was mixed with an equal volume of 3 mg/ml a-cyano hydroxycinnamic acid (Sigma) in 50% acetronitrile/0.1% TFA and was subsequently subjected to mass spectrometric analyses on a 4800 plus TOF/TOF analyzer (Applied Biosystems).

About 75 pmoles of purified recombinant Flt3D1-D5 were dissolved in 20 mM Tris-HCl, pH 8.0, and digested with trypsin (Promega), Glu-C and Asp-N endoproteinases (Sigma) at E/S= 1/35. After incubation at 37° C. overnight, 1 ml of the digestion mixture were mixed with 5 ml of 3 mg/ml α-cyano hydroxycinnamic acid (Sigma) in 50% acetronitrile/0.1% TFA prior to mass spectrometric analyses as described earlier. The remaining volume of the digestion mixture was applied on a Spheri-5 PTC-C18 column (220×2.1 mm, Higgins Analytical) at a flow rate of 100 ml/min. Reversed phase chromatography of peptide mixture was performed on an Ettan LC (Amersham Biosciences) with on-line 96-well plate Frac-950 fractionator set at 20 ml/min. One microliter of the collected fractions was mixed with an equal volume of 3 mg/ml a-cyano hydroxycinnamic acid as described earlier. The results are depicted in Table 1.

TABLE 1
Mapping of disulfide bridges and N-linked glycosylation
sites in the Flt3 ectodomain by mass-spectrometry.

| | | | |
|---|---|---|---|
| measured | calculated | sequence | |
| mass | mass | positions | remarks |

Asp-N peptides

| | | | |
|---|---|---|---|
| 2013.3 | 2013.09 | 248-265* | No glycosylation at #250 |
| 2081.56 | 2081.28 | 96-118 | Glycosylation at #100, disulfide |
| | | | bridge between #103 and #114 |
| 3486.61 | 3486.78 | 29-43 | Glycosylation at #43, disulfide bridge |
| | | | between #35 and #65 |
| | | 62-76* | |
| 4156.83 | 4156.11 | 29-43 | Glycosylation at #43, disulfide bridge |
| | | | between #35 and #65 |
| | | 62-83 | |

Glu-C peptides

| | | | |
|---|---|---|---|
| 1499.56 | 1499.61 | 196-204 | Disulfide bridge between #199 |
| | | | and #206 |
| | | 205-208 | |
| 1628.6 | 1628.65 | 196-204 | Disulfide bridge between #199 |
| | | | and #206 |
| | | 205-209* | |
| 1573.53 | 1573.72 | 347-357 | Glycosylation at #351 and #354 |
| 1777.52 | 1777.78 | 347-360* | Glycosylation at #351 or #354 |
| 1980.58 | 1980.86 | 347-360* | Glycosylation at #351 and #354 |
| 4018.67 | 4018.32** | 25-44* | Glycosylation at #43, disulfide bridge |
| | | | between #35 and #65 |
| | | 63-77* | |
| (4134.96) | 4133.35** | 25-44* | Glycosylation at #43, disulfide bridge |
| | | | between #35 and #65 |
| | | 62-77 | |
| (4263.21) | 4262.39** | 24-44* | Glycosylation at #43, disulfide bridge |
| | | | between #35 and #65 |
| | | 62-77 | |
| 5559.91 | 5560.18** | 25-58 | Glycosylation at #43, disulfide bridge |
| | | | between #35 and #65 |
| | | 62-77 | |

Tryptic peptides

| | | | |
|---|---|---|---|
| 1735.62 | 1735.76 | 381-387 | Disulfide bridge between #381 |
| | | | and #392 |
| | | 389-395 | |
| 1775.74 | 1775.71 | 323-334 | Glycosylation at #323, disulfide |
| | | | bridge between #330 and #272 |
| | | 272-273 | |
| 2187.03 | 1983.98 | 491-508 | Glycosylation at #502 |
| 2214.78 | 2214.95 | 317-307 | Glycosylation at #306*** |
| 2485.03 | 2281.98 | 467-485 | Glycosylation at #473 |
| 3542.46 | 3542.78 | 133-161 | Glycosylation at #151 |
| 3811.3 | 3811.62 | 348-372 | Glycosylation at #351 or #354, |
| | | | disulfide bridge between #368 |
| | | | and #407 |
| | | 406-410 | |
| 4014.43 | 4014.7 | 348-372 | Glycosylation at #351 and #354, |
| | | | disulfide bridge between #368 |
| | | | and #407 |
| | | 406-410 | |
| (5332.29) | 5333.06** | 176-215* | Disulfide bridges between #184-#231 |
| | | | and #232-#241 (from X-ray data) |
| | | 231-234 | |
| | | 240-243 | |
| 6614.06 | 6614.55** | 176-215* | Disulfide bridges between #184-#231 |
| | | | and #232-#241 (from X-ray data) |
| | | 220-234* | |
| | | 240-243 | |
| 8998.44 | 8998.13** | 35-41 | Glycosylation at 100***, disulfide |
| | | | bridges between #35-#65 and |
| | | | #103-#114 |
| | | 50-108 | |
| | | 109-123 | |

*Peptide containing in-complete or on-specific cleavage
**Averaged values
***contains also very small amount of fucose (+146 Da)

### Example 3

### Crystallography of Flt3:FL Complexes

Purified recombinant Flt3D1-D4: FL (5 mg/mL in 10 mM Hepes pH 7.4, 100 mM NaCl) and Flt3D1-D5: FL (8 mg/mL in 10 mM Hepes pH 7.4, 100 mM NaCl) complexes were used to carry out an extensive crystallization screen based on 1 μL crystallization droplets (0.5 μL protein sample and 0.5 μL crystallization condition) equilibrated in sitting- and hanging-drop geometry over 250 μL reservoirs containing a given crystallization condition. This led to the identification of multiple lead conditions that typically combined 0.1-0.2 M monovalent or divalent salts, pH 6-7.5, and 10-20% PEG of various molecular weights.

Diffraction quality crystals of Flt3_{D1-D4}:FL and Flt3_{D1-D5}:FL could be grown over the course of several days as rectangular rods measuring 0.1×0.1×0.3 mm from both lead conditions using the vapor-diffusion method based on the ‘sitting drop’ geometry as follows: for each complex, crystallization droplets consisting of 1 μL protein sample (Flt3_{D1-D4}:FL at 5 mg/mL in 10 mM Hepes pH 7.4, 150 mM NaCl; Flt3_{D1-D5}:FL at 5 mg/mL in 10 mM Hepes pH 7.4, 150 mM NaCl) were mixed with 1 μL reservoir solution (Flt3_{D1-D4}:FL: 100 mM MgCl₂, 50 mM MES pH 6.5, 11-13% w/v PEG 4000; Flt3_{D1-D5}:FL: 200 mM lithium citrate, 100 mM Tris pH 7.0, 12-14% w/v PEG 3350) and were equilibrated against 0.5 mL reservoir solution. For data collection under cryogenic conditions (100 K), single crystals were flash cooled—with the help of a nylon loop—in liquid nitrogen after brief serial incubations (typically 1-2 minutes per step) in mother liquor containing a gradually higher percentage of cryoprotectant (PEG 400 for Flt3_{D1-D4}:FL and glycerol for Flt3_{D1-D5}:FL). The optimal concentration of the cryoprotectant was 20% v/v for both crystal types.

Diffraction experiments were conducted on the X06SA (PXI) and X06DA (PXIII) beamlines at the Swiss Light Source (Paul Scherrer Institute, Villigen, Switzerland) and the 1D23-1 beamline at the ESRF (Grenoble, France). All data were integrated and scaled using the XDS suite (Kabsch, 2010).

The structure of Flt3D1-D4: FL was determined by maximum-likelihood molecular replacement as implemented in the program suite PHASER (McCoy et al., 2007), using the structure of human FL as search model (PDB entry 1ETE, Savvides 2000). Following density modification employing solvent flattening and 4-fold ncs-averaging via the program PARROT (Cowtan, 2010), the electron density maps revealed contiguous density for domains 2 and 3 of the Flt3 ectodomain. Model (re)building was carried out manually in electron density maps after density modification, using the program COOT (Emsley, 2010), and in the later stages via a combination of automated methods as implemented in the program BUCCANEER (Cowtan 2006). Chain tracing was facilitated by mapping of the disulfide bridges and glycosylation sites in Flt3 by mass-spectrometry. Crystallographic refinement and structure validation was carried out using PHENIX (Adams, 2010) and Buster-TNT (Blanc, 2004). The structure of Flt3D1-D5: FL was determined by maximum-likelihood molecular replacement as implemented in the program suite PHASER (McCoy et al., 2007), using the structure of the Flt3D2-D3: FL subcomplex as determined in the Flt3D1-D4: FL complex. The remaining domains were placed via additional rounds of molecular replacement and manual placement in electron density maps after density modification. Due to the low resolution of the analysis we only applied rigid-body refinement to optimize model placement.

### Example 4

### Small-Angle X-Ray Scattering

Data were collected at beamline X33 at DESY, Hamburg. The measurements were carried out at 298 K, within a momentum transfer range of 0.01 Å-1<s<0.45 Å-1 where s=4π sin(θ)/λ and 2θ is the scattering angle. All samples were measured at several solute concentrations ranging from 0.5 to 6 mg/ml in 50 mM NaPO4 pH 7.40, 100 mM NaCl, with intermittent buffer solution (50 mM NaPO4, pH 7.40, 100 mM NaCl). To monitor radiation damage, consecutive 30 sec. exposures at the highest protein concentration were compared. The data were processed using standard procedures, corrected for buffer contribution and extrapolated to infinite dilution using the program PRIMUS20. The radius of gyration Rg and forward scattering I(O), the maximum particle dimension Dmax and the distance distribution function p(r) were evaluated using the program GNOM21. The molecular masses of the different constructs were calculated by comparison with the reference bovine serum albumin (BSA) samples. The scattering patterns from the high-resolution models were calculated using the program CRYSOL22. Constrained rigid-body refinement runs were carried out in SASREF723. Rigid-body refinement of the unliganded receptors was carried out under P1 symmetry; refinement convergence was optimal with specified ambiguous distance contacts at the D3-D3* and D4-D4* interfaces. Rigid-body refinement of the hCSF-1L:hCSF-1RD1-D3 complex was carried out with twofold symmetry imposed.

### Example 5

### Electron Microscopy

For preparation of negatively stained Flt3D1-D5/FL complex, purified complex at ˜0.05 mg/mL in PBS buffer was applied to the clear side of carbon on a carbon-mica interface and stained with 2% (w/v) uranyl acetate. Images were recorded under low-dose conditions with a JEOL 1200 EX II microscope at 100 kV and at nominal 40000× magnification. Selected negatives were then digitized on a Zeiss scanner (Photoscan TD) at a step size of 14 micrometer giving a pixel size of 3.5 Å at the specimen level. Using the boxer routine of the EMAN image processing software (Ludtke, 1999), 25134 subframes of 96×96 pixels containing individual Flt3D1-D5/FL complex particles were selected interactively, CTF-corrected with CTFFIND3 (Mindell and Grigorieff, 2003) and bsoft (Heymann et al., 2008), and low-path-filtered at 15 Å with Imagic-5. Subsequent data processing was performed with Imagic-5 software package (van Heel et al., 1996) The translationally centered data set was subjected to multivariate statistical analysis and classification that provided a set of references for multireference alignment. Class averages obtained after several cycles of multireference alignment, multivariate statistical analysis and classification were compared to projections of the SAXS model of the Flt3D1-D5/FL complex (FIG. 10).

### Example 6

### Isothermal Titration Calorimetry

Experiments were carried out using a VP-ITC MicroCalorimeter (MicroCal, MA) at 37° C., and data were analyzed using the Origin ITC analysis software package supplied by MicroCal. Purified recombinant Flt3 ectodomain constructs and FL were dialyzed overnight against 10 mM Tris-HCl, pH 7.4, at 4° C. Protein concentrations were measured spectrophotometrically at 280 nm using calculated theoretical extinction coefficients and all solutions were extensively degassed prior to use. The sample was stirred at a speed of 400 rpm throughout. The thermal titration data were fit to the “one binding site model”, and apparent molar reaction enthalpy (ΔH°), apparent entropy (ΔS°), association constant (Ka) and stoichiometry of binding (N) were determined. Several titrations were performed to evaluate reproducibility.

### Example 7

### Isolation of Recombinant Flt3 Ectodomain Complexes and Thermodynamic Binding Profile of Complex Formation

A series of constructed recombinant Flt3 ectodomains was constructed (Flt3D1-D5, Flt3D1-D4, Flt3D1-D3, Flt3D1-D2, and Flt3D1) based on intron/exon boundaries and sequence alignments with homologous receptors. The constructs were produced via transient protein expression in human embryonic kidney 293T cells. Faced with prohibitively poor protein yields (100-200 μg per liter of media) tetracycline-inducible cell lines were established in HEK293S cells deficient in N-acetylglucosaminyltransferase I (HEK293S GnTI−/−) (Reeves 2006) that could secrete the target ectodomain variants with limited and homogeneous glycosylation to mg amounts. The yields and expression levels for both Flt3D1-D3 and Flt3D1-D2 were much lower than for all other constructs, and the two constructs suffered from significant solubility and stability problems, especially Flt3D1-D2.

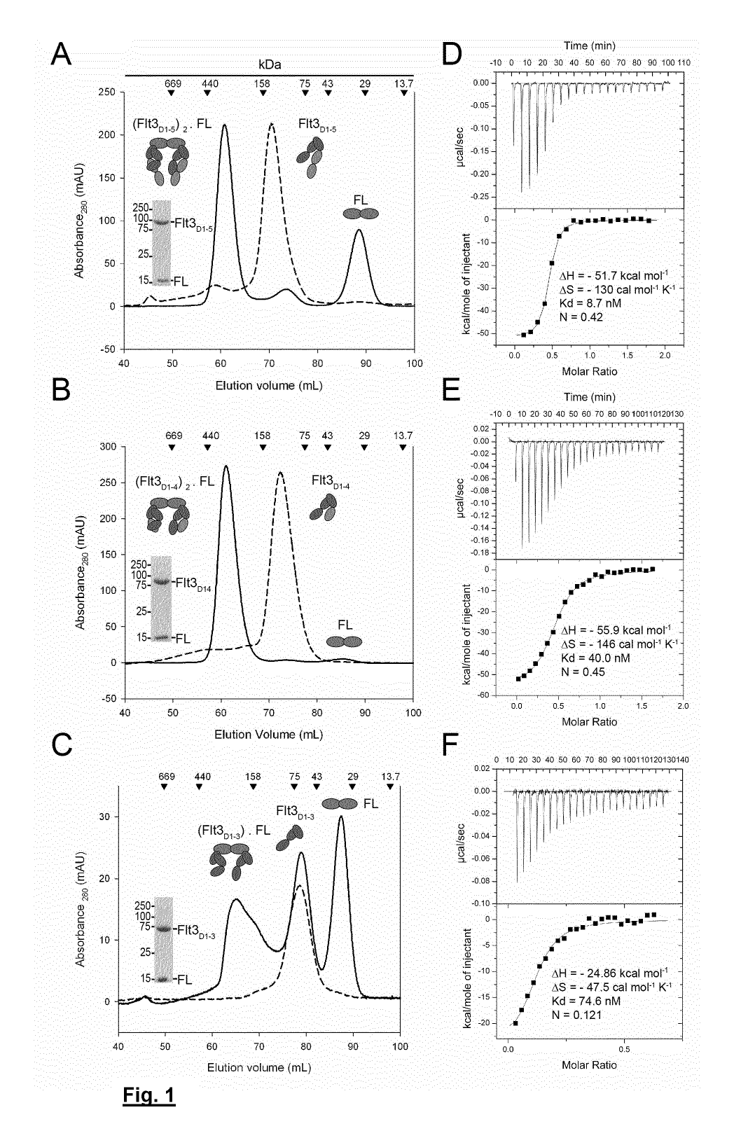

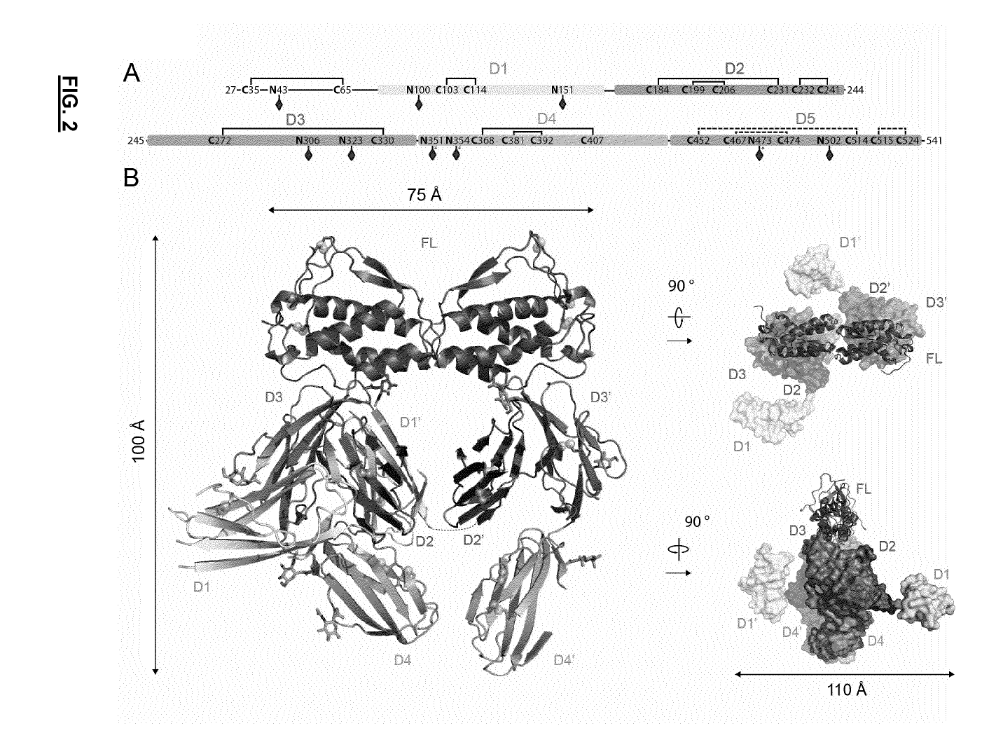

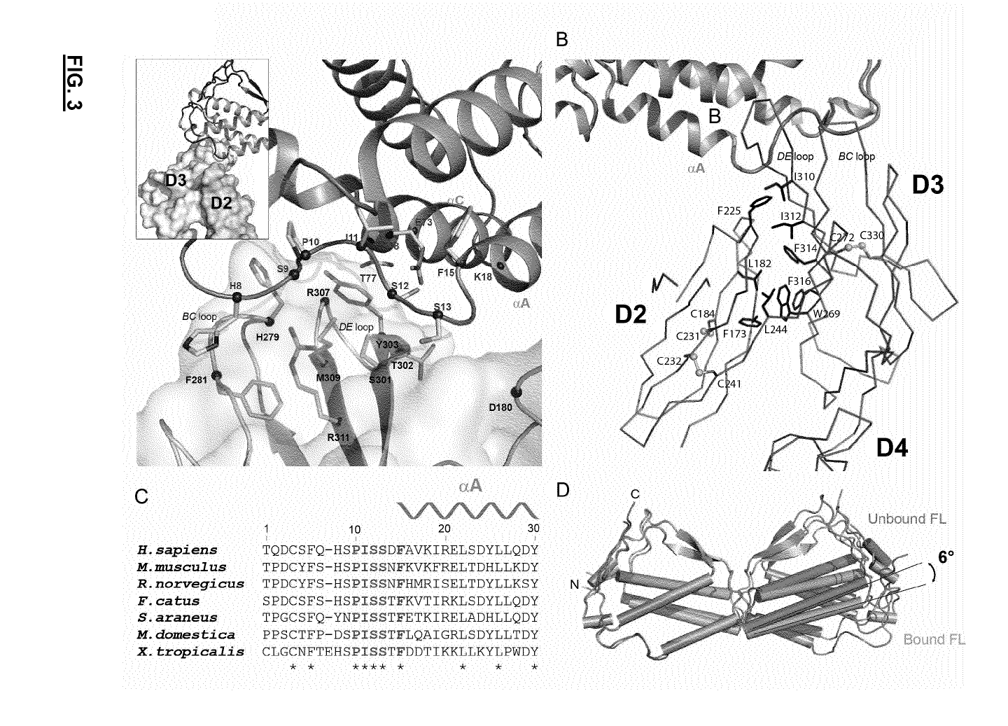

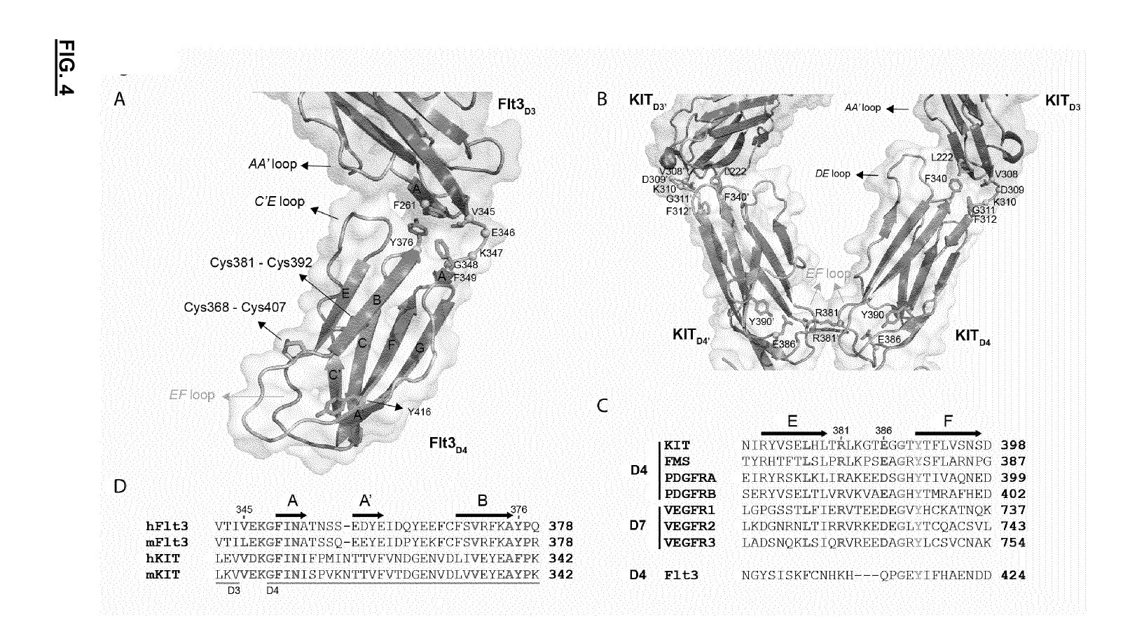

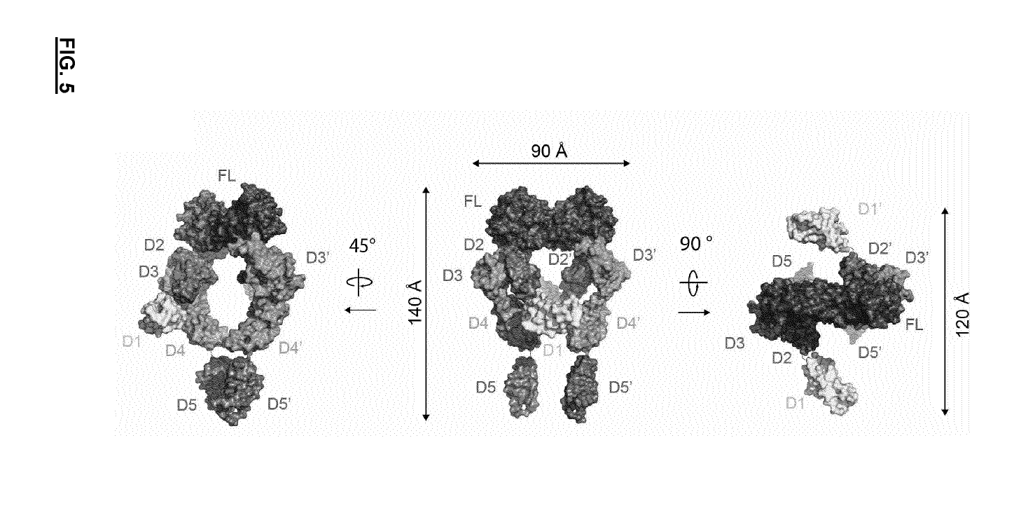

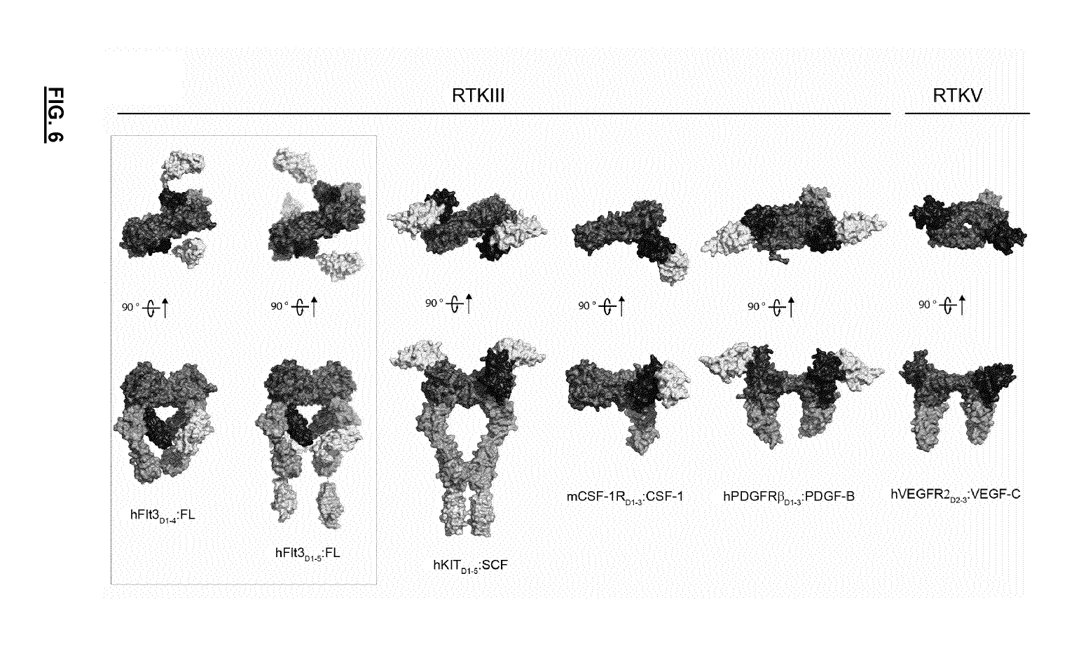

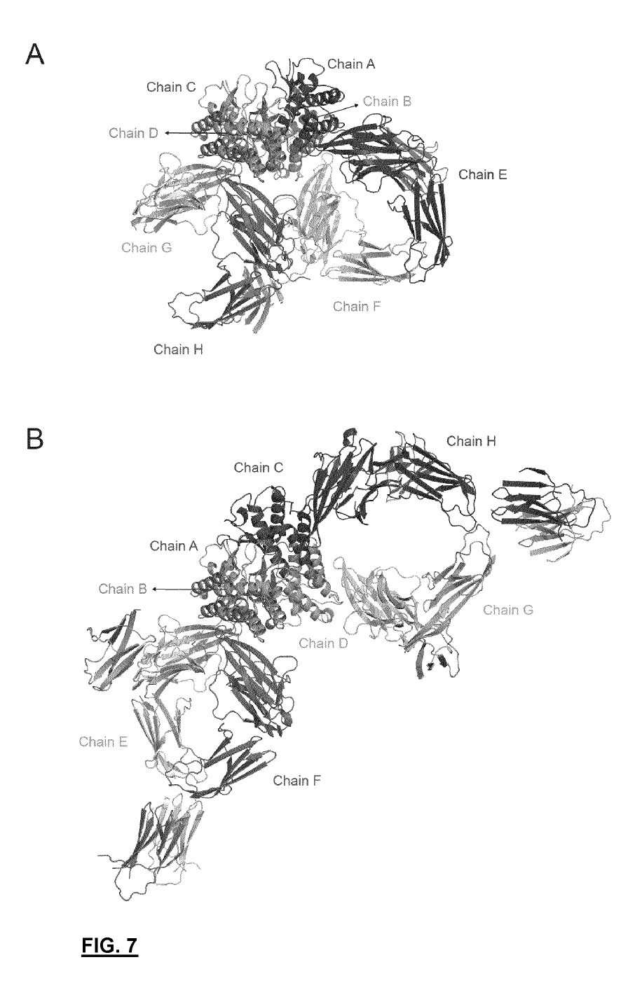

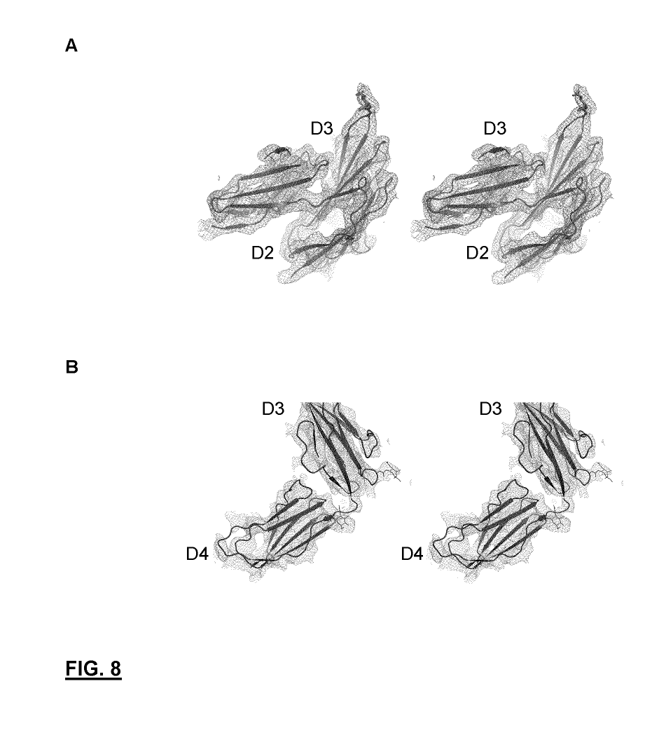

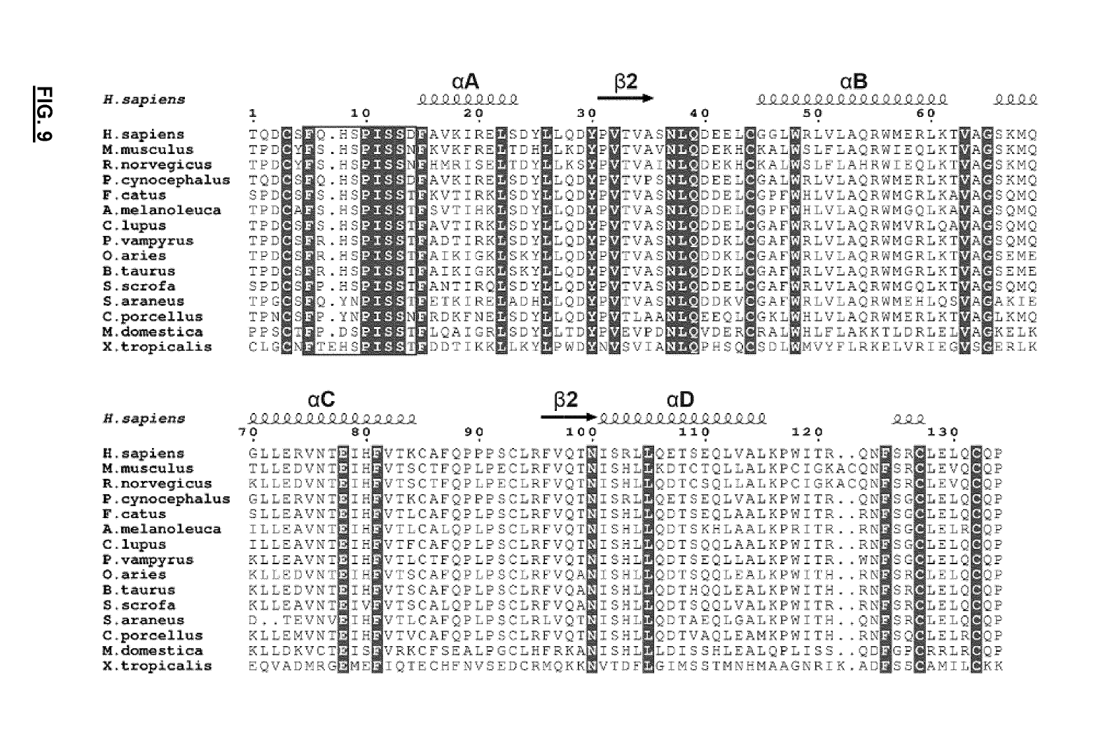

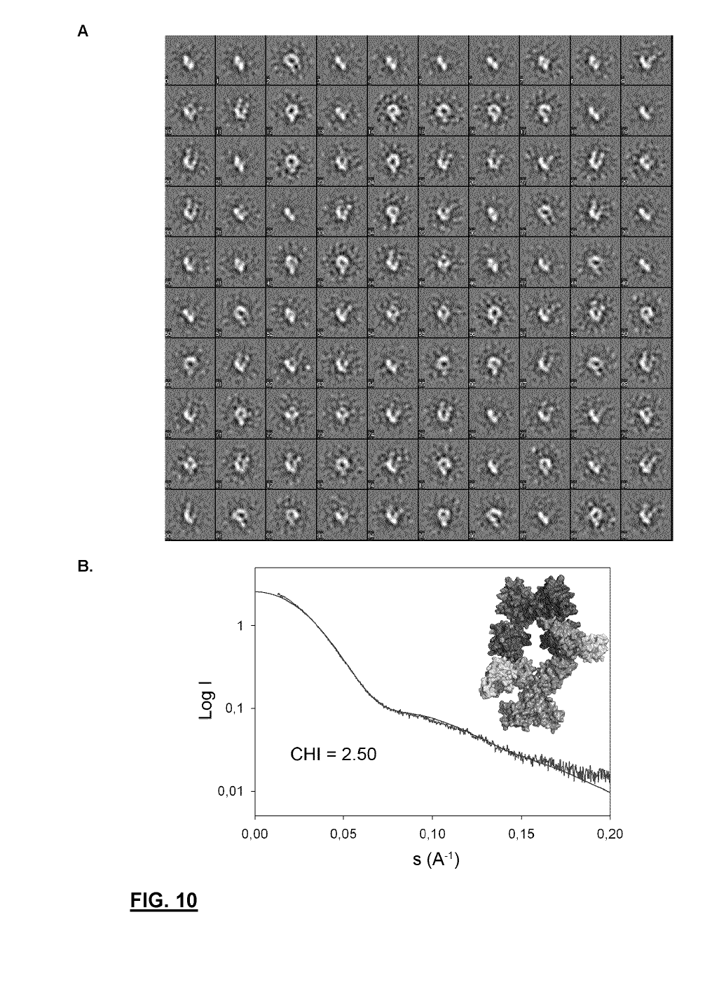

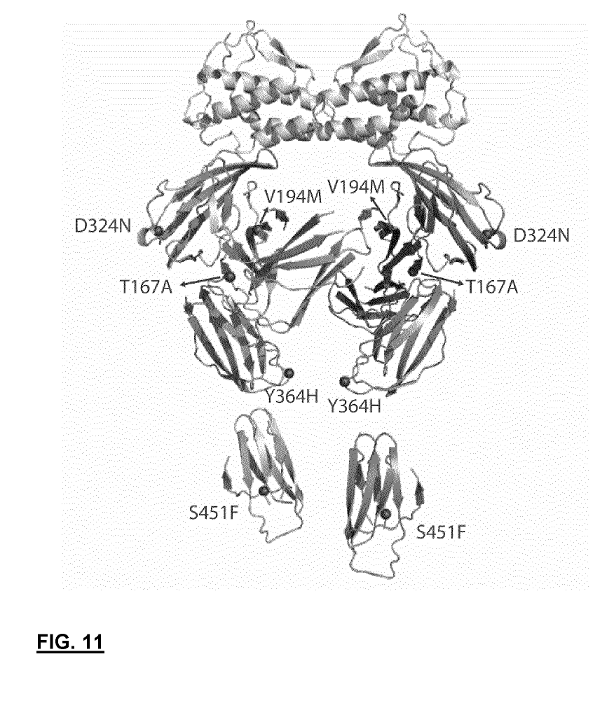

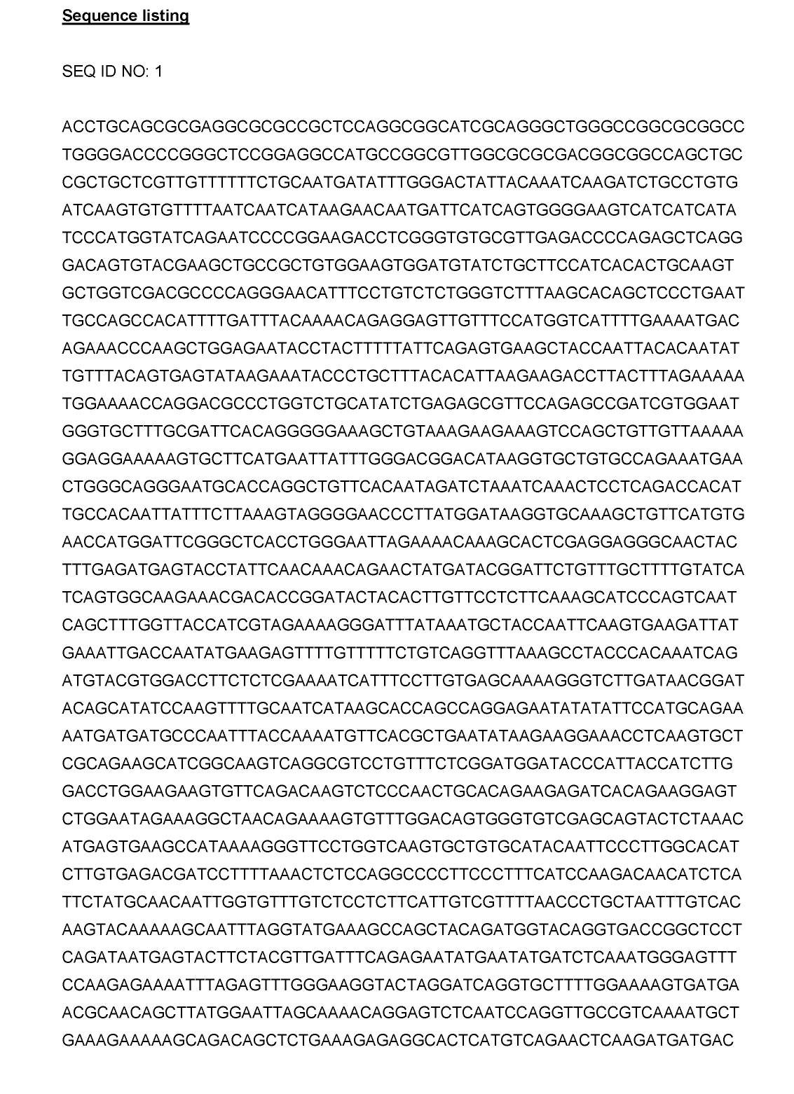

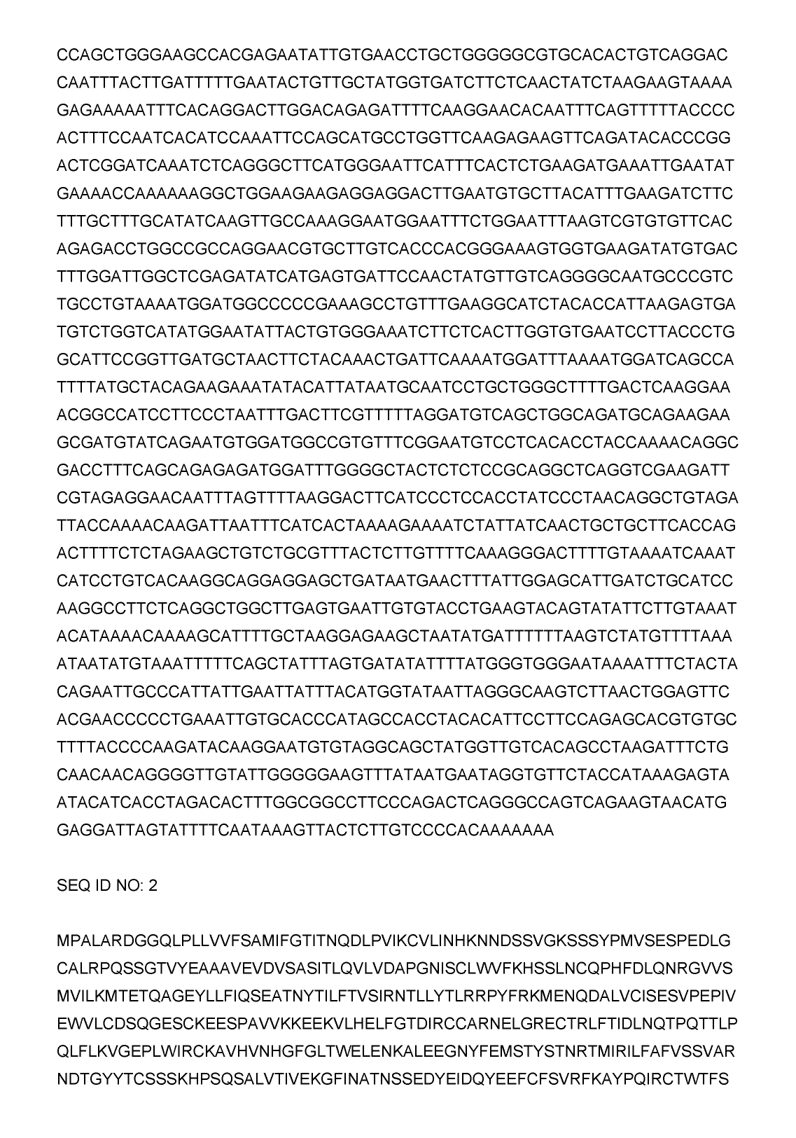

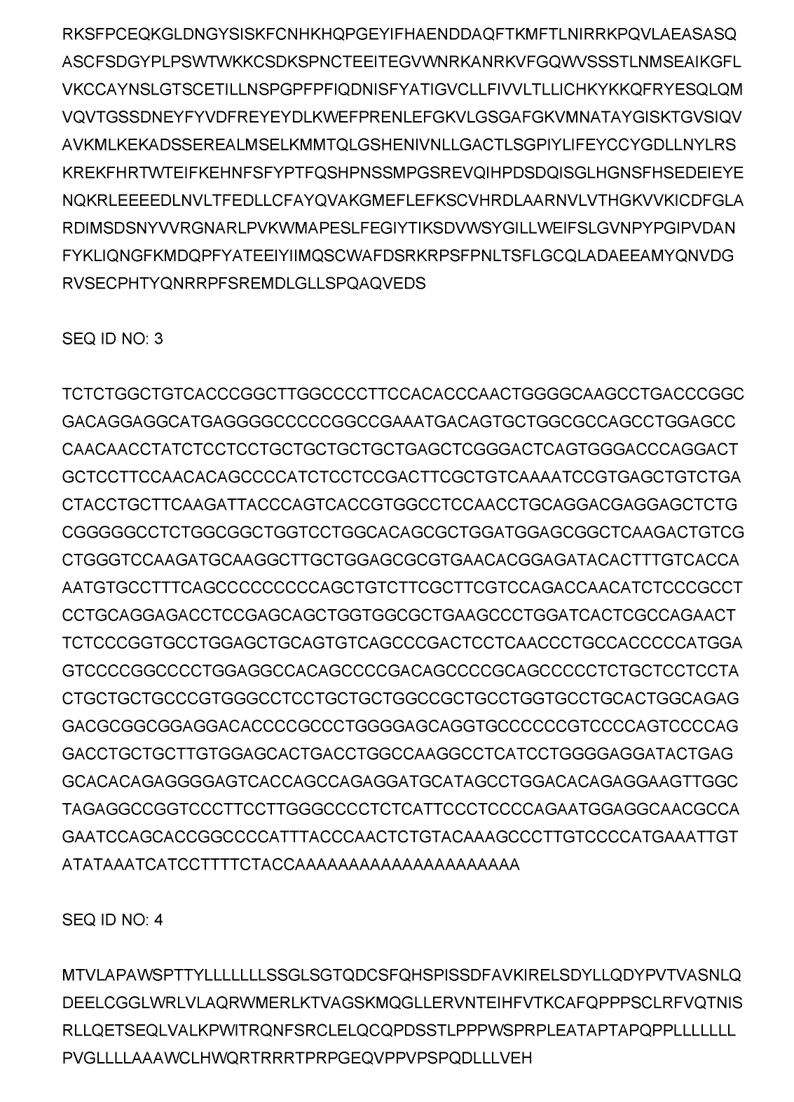

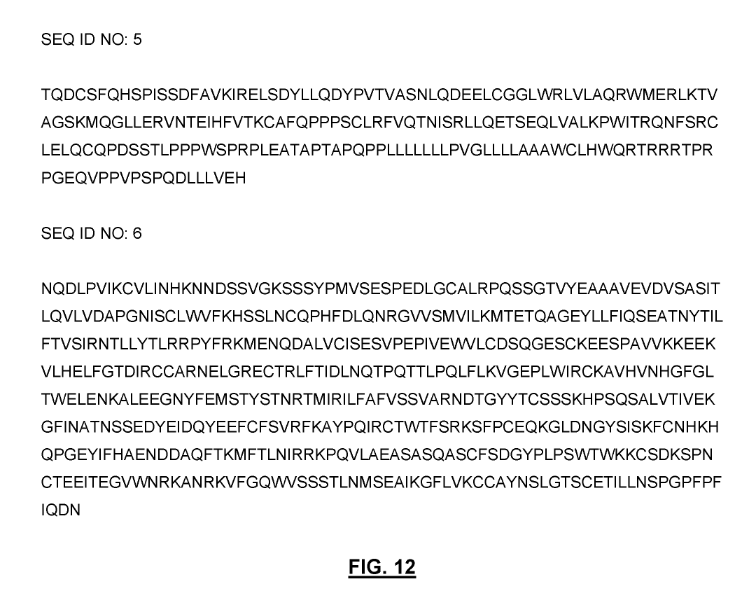

High-affinity stoichiometric complexes of purified glycosylated Flt3D1-D5, Flt3D1-D4, and Flt3D1-D3 with recombinant human FL produced in E. coli (Verstraete 2009) consistent with bivalent binding of FL to each of the ectodomain constructs were initially established by analytical size-exclusion chromatography, and subsequent batches for structural studies were obtained via preparative size-exclusion chromatography in the presence of excess molar amounts of purified FL (FIG. 1A-C). The observed elution profiles for all three ectodomain complexes were indicative of ligand-induced receptor dimerization. In contrast to our preparations of Flt3D1-D5 and Flt3D1-D4, preparations of recombinant Flt3D1-D3 consistently contained a significant portion of receptor that was incapable of binding the ligand (FIG. 10). On the other hand, we were not able to observe complex formation for Flt3D1, and Flt3D1-D2, providing the first direct evidence that these ectodomain constructs do not carry a high-affinity ligand binding site.

To quantify the thermodynamics, stoichiometry and affinity of extracellular complexes and to dissect the contribution of individual ectodomain modules to complex formation, isothermal titration calorimetry (ITC) was employed. This led to a number of consensus observations (FIG. 1D-F). A first consensus is that all three characterized complexes exhibit high-affinity binding characterized by a strongly exothermic enthalpic term coupled to an entropic penalty (FIG. 1D-F). Secondly, FL exhibits bivalent binding to both Flt3D1-D5 and Flt3D1-D4 (N=0.5, 2 molecules of Flt3 to 1 FL). Furthermore, the sequential exclusion of the membrane-proximal domains Flt3D4 and Flt3D5 leads to a very modest decrease in affinity (Kd [Flt3D1-D5:FL]=8.7 nM; Kd [Flt3D1-D4:FL]=40 nM; Kd [Flt3D1-D3:FL]=70 nM) (FIG. 1D-F). Taken together, the data indicate that the membrane-proximal module Flt3D4-D5 does not contribute to the overall stability of the complex. It is noted that the inherent instability of recombinant Flt3D1-D3 (FIG. 10) is the likely reason for the observed deviation in stoichiometry of binding for the Flt3D1-D3:FL complex in the ITC measurements. Nonetheless, the data clearly show that Flt3D1-D3 is capable of engaging in a high-affinity interaction just like the larger ectodomain constructs. Upon adjusting the Flt3 concentration to the approximate active receptor fraction (˜50%) estimated via the chromatographic elution profile of complex formation, a stoichiometry and thermodynamic signature consistent with that of the other two complexes is detected (FIG. 1F).

### Example 8

### Crystal Structure of the Flt3D1-D4:FL Complex

Highly pure and monodisperse preparations of Flt3D1-D4:FL complex yielded crystals of appreciable size (typically 0.2×0.1×0.25 mm), which diffracted synchrotron X-rays anisotropically to 4.5-5.5 Å resolution. The crystals exhibited great variation in diffraction quality even within the same crystal. Trimming of the N-glycosylation via treatment with endoglycosidase H, in an effort to the improve crystal quality resulted in receptor-ligand preparations with drastically reduced solubility and stability, which proved inadequate for crystal optimization. Optimization of the crystal cryoprotection protocol and a large scale screening of Flt3D1-D4:FL complex crystals resulted in a dataset to 4.3 Å resolution (Table 2).

TABLE 2
X-ray data collection and refinement statistics
(the values in parentheses refer to the highest resolution shell)

| | Flt3_{D1-D4}:FL | Flt3_{D1-D5}:FL |
|---|---|---|
| Data collection | | |
| Source | ESRF ID23-1 | ESRF ID23-1 |
| Wavelength | 0.9714 | 1.0762 |
| (Å) | | |
| Detector | ADSC-Q315R | ADSC-Q315R |
| Resolution (Å) | 40.00-4.3 (4.45-4.30) | 40.00-7.8 (8-7.8) |
| Space group | P2₁ | P2₁ |
| Unit cell | a = 103.89 | a = 124.74 |
| parameters | b = 146.26 | b = 153.51 |
| | c = 105.95 | c = 133.85 |
| | α = γ = 90°, β = 109.7° | α = γ = 90°, β = 94.6° |
| Wilson B (Å²) | 135 | 436 |
| Unique | 20184 (1942) | 5575 (374) |
| reflections | | |
| Redundancy | 3.8 (3.8) | 3.1 (3.0) |
| Completeness | 98.8 (98.9) | 95.3 (90.8) |
| (%) | | |
| Rₘₑₐₛ (%)^{a} | 10.8 (75.9) | 12.5 (75.9) |
| Average //σ(/) | 12.04 (2.08) | 8.8 (1.8) |
| Refinement | | |
| Resolution (Å) | 40.00-4.3 (4.53-4.30) | 35-7.8 (8.7-7.8) |
| Reflections | 19172/1010 (2622/141) | 5090/565 (1437/159) |
| (working set/ | | |
| test set) | | |
| R_{work}, R_{free} | 0.258/0.286 (0.270/0.268) | 0.367/0.350 (0.336/0.334) |
| R.m.s. | | |
| deviations | | |
| Bonds (Å) | 0.010 | n.a. |
| Angles (°) | 1.16 | n.a. |
| Average | 174 | 370 |
| ADP (Å²) | | |
| Ramachandran | | |
| analysis (%) | | |
| Favorable | 97.7 | n.a. |
| Outliers | 2.3 | n.a. |
| Protein Data | | |
| Bank access | | |
| code | | |

^{a}Rₘₑₐₛ = Σₕ√nₕ/(nₕ − 1) ΣₕΣᵢ|/(h, i) − </(h)>|/ΣₕΣᵢ/(h, i), where nₕ is the multiplicity, /(h, i) is the intensity of the i^{th} measurement of reflection h, and </(h)> is the average value over multiple measurements.

In the first phase of the structure determination process, molecular replacement solutions for FL and Flt3D3 were found using the crystal structure of FL (Savvides et al., 2000) and a homology model for Flt3D3 based on the third extracellular domain of KIT (Yuzawa et al., 2007). This showed the presence of two Flt3D1-D4:FL complexes in the crystal asymmetric unit (FIG. 7). Electron density modification exploiting the presence of improper 4-fold non-crystallographic symmetry and the high solvent content of the crystals, allowed us to select the correct MR solution for a homology model of Flt3D4 based on domain 4 of KIT, and to manually place a model for Flt3D2 based on domain 5 of KIT. In the later stages of model building and refinement, the core structure of Flt3D1 was modeled for one of the two receptor complexes. To facilitate chain tracing we determined the atypical disulfide-bond network of Flt3 as well as the actual number of N-linked glycosylation sites in extracellular Flt3 by mass-spectrometry. It was confirmed that all nine N-linked glycosylation sites are at least partially occupied and that all cysteines present in Flt3D1-D4 are engaged in disulfide-bond formation (FIG. 2A).

### Example 9

### Overall Structure of the Flt3 D1-D4:FL Complex

The structure of the Flt3D1-D4:FL complex was found to be unlike any of the structurally characterized RTKIII/V complexes to date and was found to be characterized by a number of surprising features (FIG. 2B). The FL-Flt3D1-D4 extracellular complex can be described as a moderately open horseshoe ring structure measuring 100 Å×75 Å×110 Å, comprising FL, Flt3D2, Flt3D3 and Flt3D4. FL is bound bivalently by two receptor molecules and is accommodated by a binding epitope contributed exclusively by Flt3D3. Flt3D2 leans against the concave side of Flt3D3 and is stowed underneath FL in the ring opening without engaging in interactions with the cytokine ligand. Intriguingly, the apparent two-fold symmetry of the complex about the FL dimer interface only holds for the FL:Flt3D2-D3 subcomplex, as both Flt3D1 and Flt3D4 adopt asymmetric orientations compared to their tandem modules in the complex (FIG. 2B). Remarkably, Flt3D4 does not engage in any obvious homotypic interactions as seen in the KIT structure (Yuzawa 2007). The N-terminal Flt3D1 exhibits significant disorder and domain plasticity manifested by at least two different orientations about the D1-D2 linker region (residues 162-166), and protrudes perpendicularly away from the plane of the ring assembly at the level of Flt3D2 without making any interactions with the rest of the complex. Our electron density maps (FIG. 8) allowed us to model only the core of the Flt3D1 structure (residues 79-161), but residual positive difference electron density extending away from the N-terminus of the model suggested that the atypical 50 amino acid module of Flt3D1 is likely associated with the core domain structure.

### Example 10

### Flt3 Employs a Remarkably Compact Cytokine-Binding Epitope

Perhaps the most unanticipated feature of the Flt3D1-D4:FL complex is that the ligand-binding epitope is exclusively contributed by Flt3D3 (FIG. 3A). This module is a member of the “I-set” Ig domains and is structurally homologous to extracellular domain 3 of KIT (Liu 2007; Yuzawa 2007) and FMS (Chen 2008), featuring 8 β-strands making up the ABED and A′FGC β-sheets.

However, the topology of Flt3D3 is unusual such that the polypeptide chain extending from Flt3D2 forms the N-terminal A strand in Flt3D3 (residues 246-249) by complementing strand B in a parallel fashion, while the AA′ loop of Flt3D3 (residues 250-258) adopts an extended conformation. Flt3D2, which in all other RTKIII/V complexes contributes roughly half of the ligand-binding epitope, packs against the hydrophobic patch projected by the ABED-face of Flt3D3 centered around Trp269 burying ˜1000 Å2 (FIG. 3B). Flt3D2 is homologous to KITD5 and is a member of the C2 subset of IgSF (ABED/CFG topology), but contains an additional solvent-exposed disulfide bridging strands F and G. Although the AB and EF loops of Flt3D2 point in the direction of the ligand they generally remain too far to engage in any interactions. The FL binding epitope on Flt3D3 engages in extensive interactions with the N-terminal loop of FL leading to αA (residues 8-13) and Lys18 on αA, and is mainly contributed by the BC loop of Flt3D3 (residues 279-280) and strands D (residues 301-303). Additional interactions are mediated by the DE loop of Flt3D3 (residues 307) which contacts a small patch on the C-terminal region of helix αC of FL defined by residues 73 and 78. Therefore, the Flt3 ligand-receptor interaction results in a single, highly polar contact site covering merely ˜900 Å2 of buried surface area.

### Example 11

### FL Plasticity Upon Receptor Binding

Comparison of FL in its unbound (Savvides 2000) and now in its receptor-bound form revealed that the cytokine ligand does not undergo any significant local structural changes at its receptor binding epitope (FIG. 3D). This is contrary to what has been observed in Stem Cell Factor in complex with KIT, whereby the cytokine ligand undergoes a cascade of structural rearrangements (Liu 2007, Yuzawa 2007). However, the two FL subunits display a hinge-like rigid-body rearrangement about the dimer interface, resulting in an increase in the tilt angle between the two promoters by 5-6° (FIG. 3D). A similar motion was previously observed for human Stem Cell Factor (SCF) binding to KIT (Yuzawa 2007), although SCF already appears to have significant variability in the receptor-free form as shown by the range of intersubunit tilt angles (2° to 6°) in two independent crystal structures of SCF (1EXZ and 1SCF).

### Example 12

### The Flt3D3-Flt3D4 Domain Elbow and the Absence of Homotypic Receptor Interactions

A second striking feature of the Flt3D1-D4:FL complex is the absence of any obvious specific homotypic receptor interactions. Based on the current paradigm of RTKIII activation, such interactions are mediated by Ig-like domain 4. While Flt3D4 points to its tandem Flt3D4′ in the complex, the two receptor domains stay clearly away from each other and deviate from the two-fold symmetry of the complex. The inability of Flt3D4 to engage in homotypic interactions may also explain the observed disorder for this part of the structure, as a only a complete Flt3D4-Flt3D4′ tandem could reliably be modeled and refined in only one of the two complexes in the asymmetric unit of the crystal, whereas the second could only place one of the two domains.

Closer inspection of Flt3D4 topology and sequence revealed that Flt3D4 does not possess the conserved structure-sequence fingerprints seen in all other RTKIII/V homologues for this domain. For instance, Flt3D4 has two additional disulfide bridges, a solvent exposed cross-strand disulfide bridge connecting strands B and E, and a second connecting its unusual C′E loop with strand C. Most importantly, Flt3D4 displays an EF-loop which drastically differs both in structure and sequence from all homologues (FIG. 4). The EF-loop constitutes the conserved ‘tyrosine corner’ motif in I-set Ig-domains (Harpaz and Chothia), and has been shown to mediate homotypic interactions in the case of KITD4 and VEGFRD7.

Structural comparisons of the two independent Flt3D1-D4:FL complexes in the crystal asymmetric unit revealed slight orientational plasticity of Flt3D4 about the Flt3D3-Flt3D4 linker region. This stretch of residues and the A strand of Flt3D4 are strongly conserved in Flt3 and KIT and other RTKIIIs, suggesting a common functional role. Indeed, a comparison of KIT in the bound and unbound form showed that the KITD3-KITD4 linker region acts as a hinge to reorient KITD4 for homotypic interactions upon ligand binding. However, the orientational flexibility of Flt3D4 appears to be restricted by a core of hydrophobic interactions mediated by Phe261 (A′ strand of Flt3D3), Val345 (Flt3D3-Flt3D4 linker), Phe349 (A strand of Flt3D4) and Tyr376 (BC loop of Flt3D4), as well as additional interactions between the AA′ loop of Flt3D3 and the C′E loop of Flt3D4 (FIG. 4). It thus appears that the domain elbow defined by Flt3D3 and Flt3D4 in cytokine-bound Flt3 is preserved in the ligand-free receptor.

### Example 13

### Architecture of the Complete Flt3 Extracellular Signaling Complex

Structural studies of the complete extracellular complex of Flt3 (Flt3D1-D5:FL) were pursued via a combined approach involving X-ray crystallography, negative-stain electron microscopy (EM), and Small-angle X-ray Scattering (SAXS).

Crystals of Flt3D1-D5:FL grew reproducibly from a number of crystallization conditions but proved to be of low diffraction quality. Despite repeated efforts to improve diffraction quality via reduction of glycosylation and by applying several crystal manipulation techniques, only a complete data set to 7.8 Å resolution was obtained (Table 3). Nonetheless, this data set proved sufficient to elucidate the architecture of the complete extracellular Flt3 signaling complex by molecular replacement based on the Flt3D2-D3: FL subcomplex as refined in the Flt3D1-D4:FL crystal structure. All remaining receptor domains in the two complexes in the crystal asymmetric unit, including a conservative homology model of Flt3D5 derived from the structure of human KIT, were subsequently placed into electron density and optimized by rigid-body refinement protocols (Yuzawa 2007) (Table 3).

In the full-length ectodomain complex the core structure observed in Flt3D1-D4:FL is mounted onto two membrane-proximal Flt3D5 facing each other to form an assembly resembling a hollow tennis racket (140×75×110 Å) (FIG. 5, 6). Remarkably, the asymmetry exhibited by the tandem Flt3D4 modules in Flt3D1-D4:FL is not present in the complete extracellular complex. Instead, the two Flt3D4 segments face each other symmetrically according to the 2-fold symmetry of the Flt3D2-D3:FL core structure and approach to about 20 Å from each other. While this inter-receptor separation is maintained at the ensuing Flt3D5 modules, the apparent two-fold symmetry breaks down. Furthermore, the asymmetric projection of the N-terminal Flt3D1 domains perpendicularly our of the plane of the racket head occurs in a manner analogous to what was observed in the Flt3D1-D4:FL complex.

Complementary studies of the full-length signaling complex by negative-stain EM and by SAXS in solution corroborated the overall structural features revealed by the crystal structure (FIG. 10). In retrospect, the inherent flexibility and asymmetry of the Flt3 complexes, coupled to the extensive receptor glycosylation might explain why structural studies of extracellular complexes of Flt3 have proved so challenging.

### Example 14

### Flt3 Agonist Ligand Identification

cDNA encoding full length human Flt3 is cloned in the mammalian expression vectors, pcDNA4/TO (Invitrogen).

Transient protein expression in HEK293T is carried out as previously described (Aricescu, 2006). Briefly, confluent cells, grown in tissue culture flasks or roller bottles (Greiner Bio-One) are transfected with purified plasmid DNA (Plasmid Mega Kit, Qiagen) by means of Ca-phosphate transfection method, essentially as described in Kingston et al. (2003). Flt3 expression is induced according to the manufacturer's instructions.

A dilution series of candidate ligand is added to the culture medium in a concentration ranging between 0.01 and 1000 ng/ml for 15 minutes.

Cells are lysed in Laemmli lysis buffer and subjected to Western blot analysis. Flt3 phosphorylation is evaluated with Phospho-FLT3 (Tyr591) Antibody #3461 (Cell Signaling). Data are normalized for total Flt3 expression levels with FLT3 (8F2) Rabbit mAb #3462 (Cell Signaling).

Candidate ligands are identified as Flt3 agonists if capable to induce Flt3 phosphorylation. EC50 values give information about the strength of the agonist.

### Example 15

### Flt3 Antagonist Ligand Identification

cDNA encoding full length human Flt3 is cloned in the mammalian expression vectors, pcDNA4/TO (Invitrogen).

Transient protein expression in HEK293T is carried out as previously described (Aricescu, 2006). Briefly, confluent cells, grown in tissue culture flasks or roller bottles (Greiner Bio-One) are transfected with purified plasmid DNA (Plasmid Mega Kit, Qiagen) by means of Ca-phosphate transfection method, essentially as described in Kingston et al. (2003). Flt3 expression is induced according to the manufacturer's instructions.

Human recombinant FL (hFLT3L #8924, Cell Signaling) is added to the culture medium in a concentration ranging between 0.1 and 100 ng/ml for 15 minutes. For each FL concentration, a dilution series of candidate ligand (0.01-1000 ng/ml) is concomitantly added for the same time.

Cells are lysed in Laemmli lysis buffer and subjected to Western blot analysis. Flt3 phosphorylation is evaluated with Phospho-FLT3 (Tyr591) Antibody #3461 (Cell Signaling). Data are normalized for total Flt3 expression levels with FLT3 (8F2) Rabbit mAb #3462 (Cell Signaling).

Candidate ligands are identified as Flt3 antagonists if capable to decrease FL-induced Flt3 phosphorylation relative to the Flt3 phosphorylation which is induced by FL. EC50 values give information about the strength of the antagonist.

### Example 16

### Expansion of Dendritic Cells

Cells having the CD34+ phenotype are isolated with a CD34 specific monoclonal antibody.

The CD34+ cells which are selected then are cultured in McCoy's enhanced media with 20 ng/ml each of GM-CSF, 1L-4, TNF-a (negative control); 20 ng/ml each of GM-CSF, 1L-4, TNF-a, and 100 ng/ml FL (positive control); and 20 ng/ml each of GM-CSF, 1L-4, TNF-a, and 0.01-1000 ng/ml candidate Flt3 ligand (experimental setup). The culture is continued for approximately two weeks at 37° C. in 10% C02 in humid air. Cells then are sorted by flow cytometry for CDIa+ and HLA-DR+ expression.

Candidate ligands are identified as Flt3 agonists if capable to expand dendritic cells. EC50 values give information about the strength of the agonist.

### Example 17

### Cell Proliferation Assay

Monocytic human leukemic OCI-AML3 and THP-1 cell lines, which express the wild type Flt3 receptor and proliferate in response to FL, are purchased from the German Collection of Microorganisms and Cell Cultures (DSMZ) (Braunschweig, Germany). OCI-AML3 cells are cultured in alpha-MEM with nucleosides (Gibco, Karlsruhe, Germany) and THP-1 cells are cultured in RPMI1640 (Gibco, Karlsruhe, Germany), with both media supplemented with 10% (v/v) heat-inactivated fetal calf serum (FCS) (Gibco, Karlsruhe, Germany) and 1% (v/v) Penicillin/Streptomycin (PAA Laboratories, Pasching, Austria) at 37° C. and 5% CO2, in a humidified atmosphere. Recombinant human FL (rhFL) produced in insect cells is used as a positive control (from R&D Systems; Minneapolis, Minn., USA).

The proliferation behavior of cells is assessed using the CellTiter 96® Aqueous One Solution Cell Proliferation Assay kit from Promega (Madison, Wis., USA) according to manufacturer's recommendations. In brief, 5,000 cells are seeded per well of a 96-well-plate in medium with 1% FCS (starvation medium) with or without the addition of a candidate modulator of Flt3 signaling or rhFL and cultured for 70 h at 37° C. and 5% CO2, in a humidified atmosphere. After adding the CellTiter 96® aqueous one solution reagent, cells are incubated for further 2 h at 37° C. and 5% CO2. Absorbance is recorded at 450 nm in an Anthos htII spectrometer (Anthos Labtec Instruments, Wals, Austria). Each assay is performed in triplicate in at least three independent experiments.

Flt3 modulators are identified by their propensity to stimulate cell proliferation.

Modulation of Flt3 signaling is verified by Flt3 modulator-dependent phosphorylation of the Flt3 receptor and the downstream signaling molecule MEK via Western blot analysis, using mouse monoclonal anti-phospho FLT3 antibody, rabbit polyclonal anti phospho-MEK1/2 antibody, and mouse monoclonal anti-MEK1/2 antibody (Cell Signaling Technology, Danvers, Mass., USA); mouse monoclonal anti-human FLT3 antibody (R&D Systems); and mouse monoclonal anti-GAPDH antibody (Abcam, Cambridge, UK).

### Example 18

### Screening for Ligands

Screening for ligands of Flt3 or FL is carried out by phage display, essentially as described in Clackson & Lowman (2004). DNA encoding candidate ligands, preferably Alphabodies™ or Nanobodies®, is cloned into the pIII or pVIII gene of bacteriophage M13 in a phagmid vector, and transformed into E. coli. Viral production initiates upon coinfection of E. coli with helper phages. In this way, a phage library is established.

Full length Flt3 or FL protein is immobilized on a solid substrate and incubated with the phage library, preferably via avidin/biotin coupling. The substrate is washed by which non-bound phages are removed. Retained phages are eluted and used to infect E. coli. After amplification, the phagmid containing the DNA sequence of the candidate ligand is extracted and the DNA sequence of the candidate ligand is determined. It will be clear to the person skilled in the art that multiple consecutive cycles of infection may be performed after each elution step in order to gradually enrich the final population of phages containing strongly binding candidate ligands.

### REFERENCES

Adams P D, Afonine P V, Bunkóczi G, Chen V B, Davis I W, Echols N, Headd J J, Hung L W, Kapral G J, Grosse-Kunstleve R W, McCoy A J, Moriarty N R, Oeffner R, Read R J, Richardson D C, Richardson J S, Terwilliger T C, Zwart P H., 2010. PHENIX: a comprehensive Python-based system for macromolecular structure solution. Acta Crystallogr D Biol Crystallogr, 66(Pt 2):213-21.
Altschul S F, Gish W, Miller W, Myers E W, Lipman D J. 1990. Basic local alignment search tool. J Mol Biol., 215(3):403-10.
Antonysamy, M. A. and Thomson, A. W., 2000. Flt3 ligand (FL) and its influence on immune reactivity. Cytokine, 12(2): 87-100.
Blanc E, Roversi P, Vonrhein C, Flensburg C, Lea S M, Bricogne G., 2004. Refinement of severely incomplete structures with maximum likelihood in BUSTER-TNT. Acta Crystallogr D Biol Crystallogr, 60(Pt 12 Pt 1):2210-21
Bourgeas R, Basse M J, Morelli X, Roche P., 2010. Atomic analysis of protein-protein interfaces with known inhibitors: the 2P21 database. PLoS One, 5(3):e9598.
Braden B C, Dall'Acqua W, Eisenstein E, Fields B A, Goldbaum F A, Malchiodi E L, Mariuzza R A, Schwarz F P, Ysern X, Poljak R J., 1995. Protein motion and lock and key complementarity in antigen-antibody reactions. Pharm Acta Helv, 69(4):225-30.
Bryder, D., Rossi, D. J. and Weissman, I. L., 2006. Hematopoietic stem cells: the paradigmatic tissue-specific stem cell. Am J Pathol, 169(2): 338-46.
Chen, X., Liu, H., Focia, P. J., Shim, A. H. and He, X., 2008. Structure of macrophage colony stimulating factor bound to FMS: diverse signaling assemblies of class III receptor tyrosine kinases. Proc Natl Acad Sci USA, 105(47): 18267-72.
Chema R, Sugawara H, Koike T, Lopez R, Gibson T J, Higgins D G, Thompson J D. 2003. Multiple sequence alignment with the Clustal series of programs. Nucleic Acids Res., 31(13):3497-500.
Clackson T, Lowman H B. 2004. Phage display: a practical approach. Oxford University Press, 332 pages.
Cowtan K., 2006. The Buccaneer software for automated model building. 1. Tracing protein chains. Acta Crystallogr D Biol Crystallog, 62(Pt 9):1002-11. Epub 2006 Aug. 19.
Cowtan K., 2010. Recent developments in classical density modification. Acta Crystallogr D Biol Crystallogr, 66(Pt 4):470-8.
Dehlin M, Bokarewa M, Rottapel R, Foster S J, Magnusson M, Dahlberg L E, Tarkowski A., 2008. Intra-articular fms-like tyrosine kinase 3 ligand expression is a driving force in induction and progression of arthritis. PLoS One, 3(11):e3633.
Dong, J., McPherson, C. M. and Stambrook, P. J., 2002. Flt-3 ligand: a potent dendritic cell stimulator and novel antitumor agent. Cancer Biol Ther, 1(5): 486-9.
Emsley P, Lohkamp B, Scott W G, Cowtan K., 2010. Features and development of Coot. Acta Crystallogr D Biol Crystallogr, 66(Pt 4):486-501.
Eklund E A., 2010. Genomic analysis of acute myeloid leukemia: potential for new prognostic indicators. Curr Opin Hematol, 17(2):75-8.
Eidenschenk C, Crozat K, Krebs P, Arens R, Popkin D, Arnold C N, Blasius A L, Benedict C A, Moresco E M, Xia Y, Beutler B., 2010. Flt3 permits survival during infection by rendering dendritic cells competent to activate NK cells. Proc Natl Acad Sci USA., 107(21):9759-64.
Ewing T J, Makino S, Skillman A G, Kuntz I D. 2001. DOCK 4.0: search strategies for automated molecular docking of flexible molecule databases. J Comput Aided Mol Des, 15(5):411-28.
Fong, L. et al., 2001. Altered peptide ligand vaccination with Flt3 ligand expanded dendritic cells for tumor immunotherapy. Proc Natl Acad Sci USA, 98(15): 8809-14.
Frohling, S. et al., 2007. Identification of driver and passenger mutations of FLT3 by high-throughput DNA sequence analysis and functional assessment of candidate alleles. Cancer Cell, 12(6): 501-13.
Guimond M, Freud A G, Mao H C, Yu J, Blaser B W, Leong J W, Vandeusen J B, Dorrance A, Zhang J, Mackall C L, Caligiuri M A., 2010. In vivo role of Flt3 ligand and dendritic cells in NK cell homeostasis. J Immunol, 184(6):2769-75.
Graddis, T. J. et al., 1998. Structure-function analysis of FLT3 ligand-FLT3 receptor interactions using a rapid functional screen. J Biol Chem, 273(28): 17626-33.
Grassot J, Gouy M, Perriere G, Mouchiroud G, 2006. Origin and molecular evolution of receptor tyrosine kinases with immunoglobulin-like domains. Mol Biol Evol, 23(6):1232-41.
Griffith J, Black J, Faerman C, Swenson L, Wynn M, Lu F, Lippke J, Saxena K., 2004. The structural basis for autoinhibition of FLT3 by the juxtamembrane domain. Mol Cell, 13(2):169-78.
Hannum C et al., 1994. Ligand for FLT3/FLK2 receptor tyrosine kinase regulates growth of haematopoietic stem cells and is encoded by variant RNAs. Nature, 368(6472):643-8.
Harpaz Y, Chothia C., 1994. Many of the immunoglobulin superfamily domains in cell adhesion molecules and surface receptors belong to a new structural set which is close to that containing variable domains. J Mol Biol, 238(4):528-39.
Heymann, J. B., Cardone, G., Winkler, D. C. and Steven, A. C. (2008) Computational resources for cryo-electron tomography in Bsoft. J. Struct. Biol. 161(3), 232-242).
Janin J, Bahadur R P, Chakrabarti P., 2008. Protein-protein interaction and quaternary structure. Protein-protein interaction and quaternary structure. Q Rev Biophys, 41(2):133-80.
Hubbard S R, Miller W T., 2007. Receptor tyrosine kinases: mechanisms of activation and signaling. Curr Opin Cell Biol, 19(2):117-23.
Karsunky, H., Merad, M., Cozzio, A., Weissman, I. L. and Manz, M. G., 2003. Flt3 ligand regulates dendritic cell development from Flt3+ lymphoid and myeloid-committed progenitors to Flt3+ dendritic cells in vivo. J Exp Med, 198(2): 305-13.
Kikushige Y et al. Human Flt3 is expressed at the hematopoietic stem cell and the granulocyte/macrophage progenitor stages to maintain cell survival. J Immunol, 180(11):7358-67.
Kiyoi, H. et al., 1998. Internal tandem duplication of the FLT3 gene is a novel modality of elongation mutation which causes constitutive activation of the product. Leukemia, 12(9): 1333-7.
Kindler T, Lipka D B, Fischer T., 2010. FLT3 as a therapeutic target in AML: still challenging after all these years. Blood, August 12. [Epub ahead of print].
Kingston R E, Chen C A, Rose J K. 2003. Calcium phosphate transfection. Curr Protoc Mol Biol. Chapter 9:Unit 9.1.
Konarev, P. V., Volkov, V. V., Sokolova, A. V., Koch, M. H. J. & Svergun, D. I. PRIMUS: a Windows PC-based system for small-angle scattering data analysis. Journal of Applied Crystallography 36, 1277-1282 (2003).
Kuntz I D, Blaney J M, Oatley S J, Langridge R, Ferrin T E. 1982. A geometric approach to macromolecule-ligand interactions. J Mol Biol., 161(2):269-88.
Lavagna, C., Marchetto, S., Birnbaum, D. and Rosnet, 0., 1995. Identification and characterization of a functional murine FLT3 isoform produced by exon skipping. J Biol Chem, 270(7): 3165-71.
Lemmon, M. A., Pinchasi, D., Zhou, M., Lax, I. and Schlessinger, J., 1997. Kit receptor dimerization is driven by bivalent binding of stem cell factor. J Biol Chem, 272(10): 6311-7.
Lemmon M A, Schlessinger J., 2010. Cell signaling by receptor tyrosine kinases. Cell, 141(7):1117-34.
Leppanen, V. M. et al., 2010. Structural determinants of growth factor binding and specificity by VEGF receptor 2. Proc Natl Acad Sci USA, 107(6): 2425-30.
Li, Z. and Li, L., 2006. Understanding hematopoietic stem-cell microenvironments. Trends Biochem Sci, 31(10): 589-95.
Lin H, Lee E, Hestir K, Leo C, Huang M, Bosch E, Halenbeck R, Wu G, Zhou A, Behrens D, Hollenbaugh D, Linnemann T, Qin M, Wong J, Chu K, Doberstein S K, Williams L T., 2008. Discovery of a cytokine and its receptor by functional screening of the extracellular proteome. Science, 320(5877):807-11.
Liu, H., Chen, X., Focia, P. J. and He, X., 2007. Structural basis for stem cell factor-KIT signaling and activation of class III receptor tyrosine kinases. EMBO J, 26(3): 891-901.
Liu K, Nussenzweig M C., 2010. Origin and development of dendritic cells. Immunol Rev, 234(1):45-54.
Liu K, et al. 2009. In vivo analysis of dendritic cell development and homeostasis. Science, 324(5925):392-7.
Ludtke, S. J., Baldwin, P. R. and Chiu, W., 1999. EMAN: semiautomated software for high-resolution single-particle reconstructions. J Struct Biol, 128(1): 82-97.
Lyman, S. D. et al., 1993. Characterization of the protein encoded by the flt3 (flk2) receptor-like tyrosine kinase gene. Oncogene, 8(4): 815-22.
Maroc, N. et al., 1993. Biochemical characterization and analysis of the transforming potential of the FLT3/FLK2 receptor tyrosine kinase. Oncogene, 8(4): 909-18.
Matthews, W., Jordan, C. T., Wiegand, G. W., Pardoll, D. and Lemischka, I. R., 1991. A receptor tyrosine kinase specific to hematopoietic stem and progenitor cell-enriched populations. Cell, 65(7): 1143-52.
McCoy, A. J. et al., 2007. Phaser crystallographic software. J Appl Crystallogr, 40(Pt 4): 658-674.
Metcalf, D., 2007. On hematopoietic stem cell fate. Immunity, 26(6): 669-73.
Metcalf D., 2008. Hematopoietic cytokines. Blood, 111(2):485-91.
Mindell, J. A. and Grigorieff, N., 2003. Accurate determination of local defocus and specimen tilt in electron microscopy. J Struct Biol, 142(3): 334-47.
Oates J, King G, Dixon A M., 2010. Strong oligomerization behavior of PDGFbeta receptor transmembrane domain and its regulation by the juxtamembrane regions. Biochim Biophys Acta, 1798(3):605-15.
Oefner C, D'Arcy A, Winkler F K, Eggimann B, Hosang M., 1992. Crystal structure of human platelet-derived growth factor BB. EMBO J, 11(11):3921-6.
Onai N, Obata-Onai A, Schmid M A, Manz MG., 2007. Flt3 in regulation of type I interferon-producing cell and dendritic cell development. Ann NY Acad Sci. 2, 1106:253-61.
Pandit J, Bohm A, Jancarik J, Halenbeck R, Koths K, Kim SH., 1992. Three-dimensional structure of dimeric human recombinant macrophage colony-stimulating factor. Science, 258(5086):1358-62.
Parcells, B. W., Ikeda, A. K., Simms-Waldrip, T., Moore, T. B. and Sakamoto, K. M., 2006. FMS-like tyrosine kinase 3 in normal hematopoiesis and acute myeloid leukemia. Stem Cells, 24(5): 1174-84.
Petoukhov, M. V. & Svergun, D. I. Global rigid body modeling of macromolecular complexes against small-angle scattering data. Biophysical Journal 89, 1237-1250 (2005).
Rarey M, Wefing S, Lengauer T. 1996. Placement of medium-sized molecular fragments into active sites of proteins. J Comput Aided Mol Des., 10(1):41-54.
Reindl C, Bagrintseva K, Vempati S, Schnittger S, Ellwart J W, Wenig K, Hopfner K P, Hiddemann W, Spiekermann K., 2006. Point mutations in the juxtamembrane domain of FLT3 define a new class of activating mutations in AML. Blood, 107(9):3700-7.
Robinson S, Mosley R L, Parajuli P, Pisarev V, Sublet J, Ulrich A, Talmadge J., 2000. Comparison of the hematopoietic activity of flt-3 ligand and granulocyte-macrophage colony-stimulating factor acting alone or in combination. J Hematother Stem Cell Res, 9(5):711-20.
Rosnet, O., Marchetto, S., deLapeyriere, O. and Birnbaum, D., 1991. Murine Flt3, a gene encoding a novel tyrosine kinase receptor of the PDGFR/CSF1R family. Oncogene, 6(9): 1641-50.
Ross, J. and Li, L., 2006. Recent advances in understanding extrinsic control of hematopoietic stem cell fate. Curr Opin Hematol, 13(4): 237-42.
Ruch C, Skiniotis G, Steinmetz M O, Walz T, Ballmer-Hofer K., 2007. Structure of a VEGF-VEGF receptor complex determined by electron microscopy. Nat Struct Mol Biol, 14(3):249-50.
Sanz M, Burnett A, Lo-Coco F, Löwenberg B., 2009. FLT3 inhibition as a targeted therapy for acute myeloid leukemia. Curr Opin Oncol., 21(6):594-600.
Savvides, S. N., Boone, T. and Andrew Karplus, P., 2000. Flt3 ligand structure and unexpected commonalities of helical bundles and cystine knots. Nat Struct Biol, 7(6): 486-91.
Schmid M A, Kingston D, Boddupalli S, Manz M G, 2010. Instructive cytokine signals in dendritic cell lineage commitment. Immunol Rev, 234(1):32-44.
Shim, A. H. et al., 2010. Structures of a platelet-derived growth factor/propeptide complex and a platelet-derived growth factor/receptor complex. Proc Natl Acad Sci USA, 107(25): 11307-12.
Sternberg D W, Licht J D., 2005. Therapeutic intervention in leukemias that express the activated fms-like tyrosine kinase 3 (FLT3): opportunities and challenges. Curr Opin Hematol, 12(1):7-13.
Stirewalt, D. L. and Radich, J. P., 2003. The role of FLT3 in haematopoietic malignancies. Nat Rev Cancer, 3(9): 650-65.
Stroud R M, Wells J A, 2004. Mechanistic diversity of cytokine receptor signaling across cell membranes. Sci STKE, 2004(231):re7.
Sundberg E J., 2009. Structural basis of antibody-antigen interactions. Methods Mol Biol, 524:23-36.
Svergun, D. I. Determination of the Regularization Parameter in Indirect-Transform Methods Using Perceptual Criteria. Journal of Applied Crystallography 25, 495-503 (1992).
Svergun, D., Barberato, C. & Koch, M. H. J. CRYSOL—A program to evaluate x-ray solution scattering of biological macromolecules from atomic coordinates. Journal of Applied Crystallography 28, 768-773 (1995).
Turner, A. M., Lin, N. L., Issarachai, S., Lyman, S. D. and Broudy, V. C., 1996. FLT3 receptor expression on the surface of normal and malignant human hematopoietic cells. Blood, 88(9): 3383-90.
van Heel, M., Harauz, G., Orlova, E. V., Schmidt, R. and Schatz, M., 1996. A new generation of the IMAGIC image processing system. J Struct Biol, 116(1): 17-24.
Vanrobaeys F, Devreese B, Lecocq E, Rychlewski L, De Smet L, Van Beeumen J., 2003. Proteomics of the dissimilatory iron-reducing bacterium Shewanella oneidensis MR-1, using a matrix-assisted laser desorption/ionization-tandem-time of flight mass spectrometer. Proteomics, 3(11):2249-57.
Verstraete, K. et al., 2009. Efficient production of bioactive recombinant human Flt3 ligand in E. coli. Protein J, 28(2): 57-65.
Waskow C et al. The receptor tyrosine kinase Flt3 is required for dendritic cell development in peripheral lymphoid tissues. Nat Immunol, 9(6):676-83.
Wu, L. and Liu, Y. J., 2007. Development of dendritic-cell lineages. Immunity, 26(6): 741-50.
Yuzawa, S. et al., 2007. Structural basis for activation of the receptor tyrosine kinase KIT by stem cell factor. Cell, 130(2): 323-34.

TABLE 3
Atomic coordinates

| | | | | | | | | | | | |
|---|---|---|---|---|---|---|---|---|---|---|---|
| ATOM | 1 | N | PHE | B | 245 | −16.843 | 30.204 | −81.964 | 1.00 | 161.52 | N |
| ATOM | 2 | CA | PHE | B | 245 | −16.008 | 31.221 | −82.589 | 1.00 | 161.52 | C |
| ATOM | 3 | C | PHE | B | 245 | −15.016 | 31.792 | −81.597 | 1.00 | 161.52 | C |
| ATOM | 4 | O | PHE | B | 245 | −15.390 | 32.078 | −80.462 | 1.00 | 161.52 | O |
| ATOM | 5 | CB | PHE | B | 245 | −16.837 | 32.332 | −83.225 | 1.00 | 161.52 | C |
| ATOM | 6 | CG | PHE | B | 245 | −16.014 | 33.145 | −84.190 | 1.00 | 161.52 | C |
| ATOM | 7 | CD1 | PHE | B | 245 | −15.862 | 32.740 | −85.510 | 1.00 | 161.52 | C |
| ATOM | 8 | CD2 | PHE | B | 245 | −15.357 | 34.294 | −83.771 | 1.00 | 161.52 | C |
| ATOM | 9 | CE1 | PHE | B | 245 | −15.080 | 33.484 | −86.401 | 1.00 | 161.52 | C |
| ATOM | 10 | CE2 | PHE | B | 245 | −14.581 | 35.040 | −84.659 | 1.00 | 161.52 | C |
| ATOM | 11 | CZ | PHE | B | 245 | −14.447 | 34.632 | −85.969 | 1.00 | 161.52 | C |
| ATOM | 12 | N | THR | B | 246 | −13.746 | 31.924 | −82.017 | 1.00 | 159.24 | N |
| ATOM | 13 | CA | THR | B | 246 | −12.687 | 32.414 | −81.145 | 1.00 | 159.24 | C |
| ATOM | 14 | C | THR | B | 246 | −11.776 | 33.427 | −81.844 | 1.00 | 159.24 | C |
| ATOM | 15 | O | THR | B | 246 | −11.549 | 33.330 | −83.046 | 1.00 | 159.24 | O |
| ATOM | 16 | CB | THR | B | 246 | −11.875 | 31.234 | −80.609 | 1.00 | 159.24 | C |
| ATOM | 17 | N | ILE | B | 247 | −11.270 | 34.404 | −81.063 | 1.00 | 158.96 | N |
| ATOM | 18 | CA | ILE | B | 247 | −10.340 | 35.475 | −81.440 | 1.00 | 158.96 | C |
| ATOM | 19 | C | ILE | B | 247 | −9.153 | 35.378 | −80.483 | 1.00 | 158.96 | C |
| ATOM | 20 | O | ILE | B | 247 | −9.288 | 35.730 | −79.304 | 1.00 | 158.96 | O |
| ATOM | 21 | CB | ILE | B | 247 | −11.025 | 36.879 | −81.400 | 1.00 | 158.96 | C |
| ATOM | 22 | CG1 | ILE | B | 247 | −12.145 | 36.998 | −82.450 | 1.00 | 158.96 | C |
| ATOM | 23 | CG2 | ILE | B | 247 | −10.001 | 38.022 | −81.541 | 1.00 | 158.96 | C |
| ATOM | 24 | CD1 | ILE | B | 247 | −12.974 | 38.289 | −82.374 | 1.00 | 158.96 | C |
| ATOM | 25 | N | ASP | B | 248 | −8.012 | 34.863 | −80.964 | 1.00 | 161.81 | N |
| ATOM | 26 | CA | ASP | B | 248 | −6.845 | 34.693 | −80.105 | 1.00 | 161.81 | C |
| ATOM | 27 | C | ASP | B | 248 | −5.915 | 35.888 | −80.180 | 1.00 | 161.81 | C |
| ATOM | 28 | O | ASP | B | 248 | −5.301 | 36.142 | −81.218 | 1.00 | 161.81 | O |
| ATOM | 29 | CB | ASP | B | 248 | −6.080 | 33.401 | −80.429 | 1.00 | 161.81 | C |
| ATOM | 30 | N | LEU | B | 249 | −5.821 | 36.629 | −79.068 | 1.00 | 166.16 | N |
| ATOM | 31 | CA | LEU | B | 249 | −4.897 | 37.751 | −78.935 | 1.00 | 166.16 | C |
| ATOM | 32 | C | LEU | B | 249 | −3.627 | 37.157 | −78.324 | 1.00 | 166.16 | C |
| ATOM | 33 | O | LEU | B | 249 | −3.396 | 37.134 | −77.098 | 1.00 | 166.16 | O |
| ATOM | 34 | CB | LEU | B | 249 | −5.489 | 38.928 | −78.145 | 1.00 | 166.16 | C |
| ATOM | 35 | CG | LEU | B | 249 | −6.842 | 39.445 | −78.639 | 1.00 | 166.16 | C |
| ATOM | 36 | CD1 | LEU | B | 249 | −7.336 | 40.567 | −77.769 | 1.00 | 166.16 | C |
| ATOM | 37 | CD2 | LEU | B | 249 | −6.778 | 39.890 | −80.086 | 1.00 | 166.16 | C |
| ATOM | 38 | N | ASN | B | 250 | −2.913 | 36.504 | −79.237 | 1.00 | 170.32 | N |
| ATOM | 39 | CA | ASN | B | 250 | −1.670 | 35.759 | −79.150 | 1.00 | 170.32 | C |
| ATOM | 40 | C | ASN | B | 250 | −1.326 | 35.382 | −80.602 | 1.00 | 170.32 | C |
| ATOM | 41 | O | ASN | B | 250 | −0.226 | 35.692 | −81.068 | 1.00 | 170.32 | O |
| ATOM | 42 | CB | ASN | B | 250 | −1.808 | 34.531 | −78.228 | 1.00 | 170.32 | C |
| ATOM | 43 | N | GLN | B | 251 | −2.327 | 34.822 | −81.350 | 1.00 | 174.18 | N |
| ATOM | 44 | CA | GLN | B | 251 | −2.235 | 34.375 | −82.754 | 1.00 | 174.18 | C |
| ATOM | 45 | C | GLN | B | 251 | −1.929 | 35.517 | −83.737 | 1.00 | 174.18 | C |
| ATOM | 46 | O | GLN | B | 251 | −2.239 | 36.685 | −83.469 | 1.00 | 174.18 | O |
| ATOM | 47 | CB | GLN | B | 251 | −3.533 | 33.663 | −83.193 | 1.00 | 174.18 | C |
| ATOM | 48 | N | THR | B | 252 | −1.313 | 35.149 | −84.882 | 1.00 | 176.76 | N |
| ATOM | 49 | CA | THR | B | 252 | −0.954 | 36.050 | −85.978 | 1.00 | 176.76 | C |
| ATOM | 50 | C | THR | B | 252 | −2.227 | 36.490 | −86.711 | 1.00 | 176.76 | C |
| ATOM | 51 | O | THR | B | 252 | −3.092 | 35.647 | −86.977 | 1.00 | 176.76 | O |
| ATOM | 52 | CB | THR | B | 252 | 0.042 | 35.373 | −86.927 | 1.00 | 176.76 | C |
| ATOM | 53 | N | PRO | B | 253 | −2.369 | 37.801 | −87.025 | 1.00 | 178.90 | N |
| ATOM | 54 | CA | PRO | B | 253 | −3.594 | 38.274 | −87.694 | 1.00 | 178.90 | C |
| ATOM | 55 | C | PRO | B | 253 | −3.889 | 37.571 | −89.023 | 1.00 | 178.90 | C |
| ATOM | 56 | O | PRO | B | 253 | −2.975 | 37.261 | −89.794 | 1.00 | 178.90 | O |
| ATOM | 57 | CB | PRO | B | 253 | −3.327 | 39.766 | −87.911 | 1.00 | 178.90 | C |
| ATOM | 58 | CG | PRO | B | 253 | −2.317 | 40.129 | −86.878 | 1.00 | 178.90 | C |
| ATOM | 59 | CD | PRO | B | 253 | −1.445 | 38.921 | −86.747 | 1.00 | 178.90 | C |
| ATOM | 60 | N | GLN | B | 254 | −5.188 | 37.295 | −89.259 | 1.00 | 181.38 | N |
| ATOM | 61 | CA | GLN | B | 254 | −5.712 | 36.639 | −90.459 | 1.00 | 181.38 | C |
| ATOM | 62 | C | GLN | B | 254 | −5.953 | 37.674 | −91.546 | 1.00 | 181.38 | C |
| ATOM | 63 | O | GLN | B | 254 | −6.766 | 38.585 | −91.357 | 1.00 | 181.38 | O |
| ATOM | 64 | CB | GLN | B | 254 | −7.016 | 35.867 | −90.146 | 1.00 | 181.38 | C |
| ATOM | 65 | N | THR | B | 255 | −5.250 | 37.538 | −92.683 | 1.00 | 182.34 | N |
| ATOM | 66 | CA | THR | B | 255 | −5.407 | 38.437 | −93.822 | 1.00 | 182.34 | C |
| ATOM | 67 | C | THR | B | 255 | −6.691 | 38.043 | −94.572 | 1.00 | 182.34 | C |
| ATOM | 68 | O | THR | B | 255 | −6.651 | 37.592 | −95.720 | 1.00 | 182.34 | O |
| ATOM | 69 | CB | THR | B | 255 | −4.152 | 38.410 | −94.688 | 1.00 | 182.34 | C |
| ATOM | 70 | N | THR | B | 256 | −7.834 | 38.190 | −93.868 | 1.00 | 184.42 | N |
| ATOM | 71 | CA | THR | B | 256 | −9.206 | 37.894 | −94.300 | 1.00 | 184.42 | C |
| ATOM | 72 | C | THR | B | 256 | −10.237 | 38.692 | −93.453 | 1.00 | 184.42 | C |
| ATOM | 73 | O | THR | B | 256 | −11.316 | 39.021 | −93.971 | 1.00 | 184.42 | O |
| ATOM | 74 | CB | THR | B | 256 | −9.484 | 36.382 | −94.215 | 1.00 | 184.42 | C |
| ATOM | 75 | N | LEU | B | 257 | −9.875 | 39.024 | −92.165 | 1.00 | 184.95 | N |
| ATOM | 76 | CA | LEU | B | 257 | −10.694 | 39.717 | −91.149 | 1.00 | 184.95 | C |
| ATOM | 77 | C | LEU | B | 257 | −12.019 | 38.954 | −91.013 | 1.00 | 184.95 | C |
| ATOM | 78 | O | LEU | B | 257 | −13.043 | 39.423 | −91.518 | 1.00 | 184.95 | O |
| ATOM | 79 | CB | LEU | B | 257 | −10.912 | 41.203 | −91.489 | 1.00 | 184.95 | C |
| ATOM | 80 | N | PRO | B | 258 | −11.951 | 37.714 | −90.448 | 1.00 | 185.16 | N |
| ATOM | 81 | CA | PRO | B | 258 | −13.126 | 36.809 | −90.385 | 1.00 | 185.16 | C |
| ATOM | 82 | C | PRO | B | 258 | −14.563 | 37.396 | −90.425 | 1.00 | 185.16 | C |
| ATOM | 83 | O | PRO | B | 258 | −14.914 | 38.304 | −89.668 | 1.00 | 185.16 | O |
| ATOM | 84 | CB | PRO | B | 258 | −12.890 | 36.061 | −89.077 | 1.00 | 185.16 | C |
| ATOM | 85 | CG | PRO | B | 258 | −11.353 | 36.030 | −88.933 | 1.00 | 185.16 | C |
| ATOM | 86 | CD | PRO | B | 258 | −10.761 | 37.038 | −89.883 | 1.00 | 185.16 | C |
| ATOM | 87 | N | GLN | B | 259 | −15.397 | 36.833 | −91.335 | 1.00 | 185.06 | N |
| ATOM | 88 | CA | GLN | B | 259 | −16.799 | 37.206 | −91.538 | 1.00 | 185.06 | C |
| ATOM | 89 | C | GLN | B | 259 | −17.700 | 36.134 | −90.934 | 1.00 | 185.06 | C |
| ATOM | 90 | O | GLN | B | 259 | −17.851 | 35.060 | −91.519 | 1.00 | 185.06 | O |
| ATOM | 91 | CB | GLN | B | 259 | −17.112 | 37.413 | −93.034 | 1.00 | 185.06 | C |
| ATOM | 92 | N | LEU | B | 260 | −18.254 | 36.407 | −89.735 | 1.00 | 185.40 | N |
| ATOM | 93 | CA | LEU | B | 260 | −19.160 | 35.495 | −89.017 | 1.00 | 185.40 | C |
| ATOM | 94 | C | LEU | B | 260 | −20.556 | 35.544 | −89.645 | 1.00 | 185.40 | C |
| ATOM | 95 | O | LEU | B | 260 | −20.988 | 36.618 | −90.070 | 1.00 | 185.40 | O |
| ATOM | 96 | CB | LEU | B | 260 | −19.236 | 35.867 | −87.523 | 1.00 | 185.40 | C |
| ATOM | 97 | CG | LEU | B | 260 | −18.869 | 34.778 | −86.509 | 1.00 | 185.40 | C |
| ATOM | 98 | CD1 | LEU | B | 260 | −18.618 | 35.382 | −85.139 | 1.00 | 185.40 | C |
| ATOM | 99 | CD2 | LEU | B | 260 | −19.957 | 33.711 | −86.410 | 1.00 | 185.40 | C |
| ATOM | 100 | N | PHE | B | 261 | −21.257 | 34.396 | −89.721 | 1.00 | 183.44 | N |
| ATOM | 101 | CA | PHE | B | 261 | −22.579 | 34.364 | −90.348 | 1.00 | 183.44 | C |
| ATOM | 102 | C | PHE | B | 261 | −23.600 | 33.597 | −89.535 | 1.00 | 183.44 | C |
| ATOM | 103 | O | PHE | B | 261 | −23.268 | 32.538 | −88.992 | 1.00 | 183.44 | O |
| ATOM | 104 | CB | PHE | B | 261 | −22.480 | 33.743 | −91.749 | 1.00 | 183.44 | C |
| ATOM | 105 | CG | PHE | B | 261 | −22.169 | 34.723 | −92.856 | 1.00 | 183.44 | C |
| ATOM | 106 | CD1 | PHE | B | 261 | −23.186 | 35.269 | −93.628 | 1.00 | 183.44 | C |
| ATOM | 107 | CD2 | PHE | B | 261 | −20.854 | 35.089 | −93.139 | 1.00 | 183.44 | C |
| ATOM | 108 | CE1 | PHE | B | 261 | −22.896 | 36.158 | −94.669 | 1.00 | 183.44 | C |
| ATOM | 109 | CE2 | PHE | B | 261 | −20.565 | 35.997 | −94.165 | 1.00 | 183.44 | C |
| ATOM | 110 | CZ | PHE | B | 261 | −21.587 | 36.522 | −94.927 | 1.00 | 183.44 | C |
| ATOM | 111 | N | LEU | B | 262 | −24.850 | 34.123 | −89.457 | 1.00 | 178.10 | N |
| ATOM | 112 | CA | LEU | B | 262 | −25.926 | 33.414 | −88.743 | 1.00 | 178.10 | C |
| ATOM | 113 | C | LEU | B | 262 | −27.323 | 33.949 | −89.107 | 1.00 | 178.10 | C |
| ATOM | 114 | O | LEU | B | 262 | −27.464 | 35.134 | −89.419 | 1.00 | 178.10 | O |
| ATOM | 115 | CB | LEU | B | 262 | −25.726 | 33.364 | −87.203 | 1.00 | 178.10 | C |
| ATOM | 116 | CG | LEU | B | 262 | −25.617 | 34.646 | −86.359 | 1.00 | 178.10 | C |
| ATOM | 117 | CD1 | LEU | B | 262 | −25.841 | 34.322 | −84.898 | 1.00 | 178.10 | C |
| ATOM | 118 | CD2 | LEU | B | 262 | −24.246 | 35.286 | −86.467 | 1.00 | 178.10 | C |
| ATOM | 119 | N | LYS | B | 263 | −28.342 | 33.051 | −89.113 | 1.00 | 171.62 | N |
| ATOM | 120 | CA | LYS | B | 263 | −29.725 | 33.393 | −89.453 | 1.00 | 171.62 | C |
| ATOM | 121 | C | LYS | B | 263 | −30.414 | 34.127 | −88.307 | 1.00 | 171.62 | C |
| ATOM | 122 | O | LYS | B | 263 | −30.245 | 33.729 | −87.147 | 1.00 | 171.62 | O |
| ATOM | 123 | CB | LYS | B | 263 | −30.508 | 32.141 | −89.830 | 1.00 | 171.62 | C |
| ATOM | 124 | N | VAL | B | 264 | −31.192 | 35.203 | −88.632 | 1.00 | 168.89 | N |
| ATOM | 125 | CA | VAL | B | 264 | −31.897 | 36.018 | −87.609 | 1.00 | 168.89 | C |
| ATOM | 126 | C | VAL | B | 264 | −32.683 | 35.108 | −86.636 | 1.00 | 168.89 | C |
| ATOM | 127 | O | VAL | B | 264 | −33.477 | 34.274 | −87.077 | 1.00 | 168.89 | O |
| ATOM | 128 | CB | VAL | B | 264 | −32.806 | 37.124 | −88.226 | 1.00 | 168.89 | C |
| ATOM | 129 | CG1 | VAL | B | 264 | −33.831 | 37.668 | −87.226 | 1.00 | 168.89 | C |
| ATOM | 130 | CG2 | VAL | B | 264 | −31.971 | 38.266 | −88.778 | 1.00 | 168.89 | C |
| ATOM | 131 | N | GLY | B | 265 | −32.417 | 35.278 | −85.335 | 1.00 | 167.11 | N |
| ATOM | 132 | CA | GLY | B | 265 | −33.026 | 34.509 | −84.250 | 1.00 | 167.11 | C |
| ATOM | 133 | C | GLY | B | 265 | −32.031 | 33.612 | −83.537 | 1.00 | 167.11 | C |
| ATOM | 134 | O | GLY | B | 265 | −32.213 | 33.326 | −82.347 | 1.00 | 167.11 | O |
| ATOM | 135 | N | GLU | B | 266 | −30.960 | 33.163 | −84.272 | 1.00 | 162.99 | N |
| ATOM | 136 | CA | GLU | B | 266 | −29.887 | 32.293 | −83.765 | 1.00 | 162.99 | C |
| ATOM | 137 | C | GLU | B | 266 | −29.001 | 33.033 | −82.716 | 1.00 | 162.99 | C |
| ATOM | 138 | O | GLU | B | 266 | −28.962 | 34.268 | −82.715 | 1.00 | 162.99 | O |
| ATOM | 139 | CB | GLU | B | 266 | −29.032 | 31.754 | −84.929 | 1.00 | 162.99 | C |
| ATOM | 140 | N | PRO | B | 267 | −28.299 | 32.324 | −81.802 | 1.00 | 159.20 | N |
| ATOM | 141 | CA | PRO | B | 267 | −27.500 | 33.037 | −80.793 | 1.00 | 159.20 | C |
| ATOM | 142 | C | PRO | B | 267 | −26.048 | 33.295 | −81.214 | 1.00 | 159.20 | C |
| ATOM | 143 | O | PRO | B | 267 | −25.320 | 32.371 | −81.581 | 1.00 | 159.20 | O |
| ATOM | 144 | CB | PRO | B | 267 | −27.572 | 32.118 | −79.571 | 1.00 | 159.20 | C |
| ATOM | 145 | CG | PRO | B | 267 | −27.950 | 30.755 | −80.117 | 1.00 | 159.20 | C |
| ATOM | 146 | CD | PRO | B | 267 | −28.240 | 30.867 | −81.587 | 1.00 | 159.20 | C |
| ATOM | 147 | N | LEU | B | 268 | −25.621 | 34.559 | −81.130 | 1.00 | 156.12 | N |
| ATOM | 148 | CA | LEU | B | 268 | −24.258 | 34.932 | −81.484 | 1.00 | 156.12 | C |
| ATOM | 149 | C | LEU | B | 268 | −23.305 | 34.584 | −80.357 | 1.00 | 156.12 | C |
| ATOM | 150 | O | LEU | B | 268 | −23.648 | 34.786 | −79.198 | 1.00 | 156.12 | O |
| ATOM | 151 | CB | LEU | B | 268 | −24.195 | 36.423 | −81.799 | 1.00 | 156.12 | C |
| ATOM | 152 | CG | LEU | B | 268 | −22.825 | 37.011 | −82.115 | 1.00 | 156.12 | C |
| ATOM | 153 | CD1 | LEU | B | 268 | −22.205 | 36.363 | −83.351 | 1.00 | 156.12 | C |
| ATOM | 154 | CD2 | LEU | B | 268 | −22.910 | 38.517 | −82.283 | 1.00 | 156.12 | C |
| ATOM | 155 | N | TRP | B | 269 | −22.111 | 34.073 | −80.694 | 1.00 | 154.61 | N |
| ATOM | 156 | CA | TRP | B | 269 | −21.097 | 33.679 | −79.713 | 1.00 | 154.61 | C |
| ATOM | 157 | C | TRP | B | 269 | −19.679 | 33.957 | −80.203 | 1.00 | 154.61 | C |
| ATOM | 158 | O | TRP | B | 269 | −19.236 | 33.344 | −81.178 | 1.00 | 154.61 | O |
| ATOM | 159 | CB | TRP | B | 269 | −21.240 | 32.192 | −79.379 | 1.00 | 154.61 | C |
| ATOM | 160 | CG | TRP | B | 269 | −22.337 | 31.905 | −78.407 | 1.00 | 154.61 | C |
| ATOM | 161 | CD1 | TRP | B | 269 | −23.600 | 31.482 | −78.696 | 1.00 | 154.61 | C |
| ATOM | 162 | CD2 | TRP | B | 269 | −22.271 | 32.033 | −76.983 | 1.00 | 154.61 | C |
| ATOM | 163 | CE2 | TRP | B | 269 | −23.527 | 31.648 | −76.469 | 1.00 | 154.61 | C |
| ATOM | 164 | CE3 | TRP | B | 269 | −21.259 | 32.410 | −76.088 | 1.00 | 154.61 | C |
| ATOM | 165 | NE1 | TRP | B | 269 | −24.328 | 31.341 | −77.538 | 1.00 | 154.61 | N |
| ATOM | 166 | CZ2 | TRP | B | 269 | −23.797 | 31.619 | −75.102 | 1.00 | 154.61 | C |
| ATOM | 167 | CZ3 | TRP | B | 269 | −21.541 | 32.416 | −74.738 | 1.00 | 154.61 | C |
| ATOM | 168 | CH2 | TRP | B | 269 | −22.796 | 32.026 | −74.256 | 1.00 | 154.61 | C |
| ATOM | 169 | N | ILE | B | 270 | −18.958 | 34.869 | −79.518 | 1.00 | 152.78 | N |
| ATOM | 170 | CA | ILE | B | 270 | −17.587 | 35.230 | −79.898 | 1.00 | 152.78 | C |
| ATOM | 171 | C | ILE | B | 270 | −16.665 | 35.244 | −78.671 | 1.00 | 152.78 | C |
| ATOM | 172 | O | ILE | B | 270 | −16.753 | 36.159 | −77.858 | 1.00 | 152.78 | O |
| ATOM | 173 | CB | ILE | B | 270 | −17.514 | 36.590 | −80.647 | 1.00 | 152.78 | C |
| ATOM | 174 | CG1 | ILE | B | 270 | −18.825 | 36.995 | −81.330 | 1.00 | 152.78 | C |
| ATOM | 175 | CG2 | ILE | B | 270 | −16.365 | 36.616 | −81.615 | 1.00 | 152.78 | C |
| ATOM | 176 | CD1 | ILE | B | 270 | −19.492 | 38.096 | −80.641 | 1.00 | 152.78 | C |
| ATOM | 177 | N | ARG | B | 271 | −15.779 | 34.244 | −78.550 | 1.00 | 154.11 | N |
| ATOM | 178 | CA | ARG | B | 271 | −14.831 | 34.155 | −77.438 | 1.00 | 154.11 | C |
| ATOM | 179 | C | ARG | B | 271 | −13.596 | 34.942 | −77.748 | 1.00 | 154.11 | C |
| ATOM | 180 | O | ARG | B | 271 | −13.067 | 34.838 | −78.844 | 1.00 | 154.11 | O |
| ATOM | 181 | CB | ARG | B | 271 | −14.448 | 32.699 | −77.135 | 1.00 | 154.11 | C |
| ATOM | 182 | N | CYS | B | 272 | −13.119 | 35.718 | −76.798 | 1.00 | 157.73 | N |
| ATOM | 183 | CA | CYS | B | 272 | −11.899 | 36.459 | −77.018 | 1.00 | 157.73 | C |
| ATOM | 184 | C | CYS | B | 272 | −10.874 | 36.045 | −75.991 | 1.00 | 157.73 | C |
| ATOM | 185 | O | CYS | B | 272 | −10.980 | 36.438 | −74.826 | 1.00 | 157.73 | O |
| ATOM | 186 | CB | CYS | B | 272 | −12.152 | 37.954 | −76.988 | 1.00 | 157.73 | C |
| ATOM | 187 | SG | CYS | B | 272 | −10.724 | 38.929 | −77.497 | 1.00 | 157.73 | S |
| ATOM | 188 | N | LYS | B | 273 | −9.897 | 35.218 | −76.415 | 1.00 | 153.79 | N |
| ATOM | 189 | CA | LYS | B | 273 | −8.863 | 34.668 | −75.529 | 1.00 | 153.79 | C |
| ATOM | 190 | C | LYS | B | 273 | −7.558 | 35.410 | −75.659 | 1.00 | 153.79 | C |
| ATOM | 191 | O | LYS | B | 273 | −6.873 | 35.293 | −76.675 | 1.00 | 153.79 | O |
| ATOM | 192 | CB | LYS | B | 273 | −8.632 | 33.166 | −75.785 | 1.00 | 153.79 | C |
| ATOM | 193 | N | ALA | B | 274 | −7.208 | 36.168 | −74.630 | 1.00 | 150.31 | N |
| ATOM | 194 | CA | ALA | B | 274 | −5.960 | 36.908 | −74.637 | 1.00 | 150.31 | C |
| ATOM | 195 | C | ALA | B | 274 | −4.994 | 36.257 | −73.694 | 1.00 | 150.31 | C |
| ATOM | 196 | O | ALA | B | 274 | −5.395 | 35.865 | −72.595 | 1.00 | 150.31 | O |
| ATOM | 197 | CB | ALA | B | 274 | −6.200 | 38.350 | −74.249 | 1.00 | 150.31 | C |
| ATOM | 198 | N | VAL | B | 275 | −3.733 | 36.091 | −74.120 | 1.00 | 150.55 | N |
| ATOM | 199 | CA | VAL | B | 275 | −2.768 | 35.439 | −73.228 | 1.00 | 150.55 | C |
| ATOM | 200 | C | VAL | B | 275 | −1.668 | 36.402 | −72.855 | 1.00 | 150.55 | C |
| ATOM | 201 | O | VAL | B | 275 | −1.009 | 36.931 | −73.744 | 1.00 | 150.55 | O |
| ATOM | 202 | CB | VAL | B | 275 | −2.163 | 34.124 | −73.776 | 1.00 | 150.55 | C |
| ATOM | 203 | CG1 | VAL | B | 275 | −1.945 | 33.124 | −72.643 | 1.00 | 150.55 | C |
| ATOM | 204 | CG2 | VAL | B | 275 | −3.011 | 33.517 | −74.896 | 1.00 | 150.55 | C |
| ATOM | 205 | N | HIS | B | 276 | −1.436 | 36.607 | −71.555 | 1.00 | 151.82 | N |
| ATOM | 206 | CA | HIS | B | 276 | −0.391 | 37.529 | −71.118 | 1.00 | 151.82 | C |
| ATOM | 207 | C | HIS | B | 276 | 0.424 | 36.934 | −69.966 | 1.00 | 151.82 | C |
| ATOM | 208 | O | HIS | B | 276 | −0.028 | 35.971 | −69.350 | 1.00 | 151.82 | O |
| ATOM | 209 | CB | HIS | B | 276 | −1.020 | 38.871 | −70.726 | 1.00 | 151.82 | C |
| ATOM | 210 | CG | HIS | B | 276 | −0.044 | 39.953 | −70.365 | 1.00 | 151.82 | C |
| ATOM | 211 | CD2 | HIS | B | 276 | 0.936 | 40.522 | −71.106 | 1.00 | 151.82 | C |
| ATOM | 212 | ND1 | HIS | B | 276 | −0.047 | 40.538 | −69.107 | 1.00 | 151.82 | N |
| ATOM | 213 | CE1 | HIS | B | 276 | 0.918 | 41.442 | −69.125 | 1.00 | 151.82 | C |
| ATOM | 214 | NE2 | HIS | B | 276 | 1.539 | 41.468 | −70.305 | 1.00 | 151.82 | N |
| ATOM | 215 | N | VAL | B | 277 | 1.631 | 37.509 | −69.693 | 1.00 | 153.56 | N |
| ATOM | 216 | CA | VAL | B | 277 | 2.595 | 37.118 | −68.643 | 1.00 | 153.56 | C |
| ATOM | 217 | C | VAL | B | 277 | 2.040 | 37.425 | −67.227 | 1.00 | 153.56 | C |
| ATOM | 218 | O | VAL | B | 277 | 1.822 | 36.493 | −66.447 | 1.00 | 153.56 | O |
| ATOM | 219 | CB | VAL | B | 277 | 3.969 | 37.798 | −68.852 | 1.00 | 153.56 | C |
| ATOM | 220 | CG1 | VAL | B | 277 | 5.001 | 37.253 | −67.872 | 1.00 | 153.56 | C |
| ATOM | 221 | CG2 | VAL | B | 277 | 4.449 | 37.633 | −70.288 | 1.00 | 153.56 | C |
| ATOM | 222 | N | ASN | B | 278 | 1.864 | 38.722 | −66.886 | 1.00 | 153.90 | N |
| ATOM | 223 | CA | ASN | B | 278 | 1.299 | 39.153 | −65.604 | 1.00 | 153.90 | C |
| ATOM | 224 | C | ASN | B | 278 | −0.215 | 39.186 | −65.730 | 1.00 | 153.90 | C |
| ATOM | 225 | O | ASN | B | 278 | −0.724 | 39.187 | −66.845 | 1.00 | 153.90 | O |
| ATOM | 226 | CB | ASN | B | 278 | 1.845 | 40.525 | −65.214 | 1.00 | 153.90 | C |
| ATOM | 227 | N | HIS | B | 279 | −0.943 | 39.220 | −64.615 | 1.00 | 155.49 | N |
| ATOM | 228 | CA | HIS | B | 279 | −2.406 | 39.284 | −64.676 | 1.00 | 155.49 | C |
| ATOM | 229 | C | HIS | B | 279 | −2.883 | 40.689 | −65.076 | 1.00 | 155.49 | C |
| ATOM | 230 | O | HIS | B | 279 | −4.072 | 40.897 | −65.331 | 1.00 | 155.49 | O |
| ATOM | 231 | CB | HIS | B | 279 | −3.014 | 38.898 | −63.325 | 1.00 | 155.49 | C |
| ATOM | 232 | CG | HIS | B | 279 | −2.900 | 39.982 | −62.304 | 1.00 | 155.49 | C |
| ATOM | 233 | CD2 | HIS | B | 279 | −3.685 | 41.069 | −62.108 | 1.00 | 155.49 | C |
| ATOM | 234 | ND1 | HIS | B | 279 | −1.854 | 40.013 | −61.399 | 1.00 | 155.49 | N |
| ATOM | 235 | CE1 | HIS | B | 279 | −2.045 | 41.100 | −60.672 | 1.00 | 155.49 | C |
| ATOM | 236 | NE2 | HIS | B | 279 | −3.128 | 41.775 | −61.072 | 1.00 | 155.49 | N |
| ATOM | 237 | N | GLY | B | 280 | −1.959 | 41.642 | −65.037 | 1.00 | 157.26 | N |
| ATOM | 238 | CA | GLY | B | 280 | −2.209 | 43.043 | −65.338 | 1.00 | 157.26 | C |
| ATOM | 239 | C | GLY | B | 280 | −2.559 | 43.329 | −66.781 | 1.00 | 157.26 | C |
| ATOM | 240 | O | GLY | B | 280 | −1.692 | 43.702 | −67.577 | 1.00 | 157.26 | O |
| ATOM | 241 | N | PHE | B | 281 | −3.843 | 43.144 | −67.110 | 1.00 | 158.55 | N |
| ATOM | 242 | CA | PHE | B | 281 | −4.461 | 43.393 | −68.408 | 1.00 | 158.55 | C |
| ATOM | 243 | C | PHE | B | 281 | −5.933 | 43.050 | −68.338 | 1.00 | 158.55 | C |
| ATOM | 244 | O | PHE | B | 281 | −6.337 | 42.180 | −67.564 | 1.00 | 158.55 | O |
| ATOM | 245 | CB | PHE | B | 281 | −3.777 | 42.619 | −69.555 | 1.00 | 158.55 | C |
| ATOM | 246 | CG | PHE | B | 281 | −4.106 | 41.149 | −69.693 | 1.00 | 158.55 | C |
| ATOM | 247 | CD1 | PHE | B | 281 | −4.795 | 40.675 | −70.802 | 1.00 | 158.55 | C |
| ATOM | 248 | CD2 | PHE | B | 281 | −3.687 | 40.232 | −68.735 | 1.00 | 158.55 | C |
| ATOM | 249 | CE1 | PHE | B | 281 | −5.069 | 39.312 | −70.944 | 1.00 | 158.55 | C |
| ATOM | 250 | CE2 | PHE | B | 281 | −3.972 | 38.868 | −68.875 | 1.00 | 158.55 | C |
| ATOM | 251 | CZ | PHE | B | 281 | −4.650 | 38.418 | −69.985 | 1.00 | 158.55 | C |
| ATOM | 252 | N | GLY | B | 282 | −6.716 | 43.763 | −69.129 | 1.00 | 161.67 | N |
| ATOM | 253 | CA | GLY | B | 282 | −8.154 | 43.579 | −69.261 | 1.00 | 161.67 | C |
| ATOM | 254 | C | GLY | B | 282 | −8.512 | 43.370 | −70.716 | 1.00 | 161.67 | C |
| ATOM | 255 | O | GLY | B | 282 | −7.623 | 43.316 | −71.570 | 1.00 | 161.67 | O |
| ATOM | 256 | N | LEU | B | 283 | −9.804 | 43.227 | −71.010 | 1.00 | 164.54 | N |
| ATOM | 257 | CA | LEU | B | 283 | −10.302 | 43.036 | −72.374 | 1.00 | 164.54 | C |
| ATOM | 258 | C | LEU | B | 283 | −11.608 | 43.801 | −72.580 | 1.00 | 164.54 | C |
| ATOM | 259 | O | LEU | B | 283 | −12.352 | 44.008 | −71.613 | 1.00 | 164.54 | O |
| ATOM | 260 | CB | LEU | B | 283 | −10.516 | 41.548 | −72.657 | 1.00 | 164.54 | C |
| ATOM | 261 | CG | LEU | B | 283 | −9.332 | 40.766 | −73.162 | 1.00 | 164.54 | C |
| ATOM | 262 | CD1 | LEU | B | 283 | −8.565 | 40.167 | −72.022 | 1.00 | 164.54 | C |
| ATOM | 263 | CD2 | LEU | B | 283 | −9.794 | 39.647 | −74.038 | 1.00 | 164.54 | C |
| ATOM | 264 | N | THR | B | 284 | −11.900 | 44.216 | −73.828 | 1.00 | 167.63 | N |
| ATOM | 265 | CA | THR | B | 284 | −13.121 | 44.959 | −74.120 | 1.00 | 167.63 | C |
| ATOM | 266 | C | THR | B | 284 | −13.636 | 44.649 | −75.491 | 1.00 | 167.63 | C |
| ATOM | 267 | O | THR | B | 284 | −12.857 | 44.428 | −76.418 | 1.00 | 167.63 | O |
| ATOM | 268 | CB | THR | B | 284 | −12.889 | 46.459 | −73.980 | 1.00 | 167.63 | C |
| ATOM | 269 | N | TRP | B | 285 | −14.966 | 44.665 | −75.615 | 1.00 | 174.19 | N |
| ATOM | 270 | CA | TRP | B | 285 | −15.708 | 44.420 | −76.851 | 1.00 | 174.19 | C |
| ATOM | 271 | C | TRP | B | 285 | −16.307 | 45.717 | −77.417 | 1.00 | 174.19 | C |
| ATOM | 272 | O | TRP | B | 285 | −16.684 | 46.590 | −76.630 | 1.00 | 174.19 | O |
| ATOM | 273 | CB | TRP | B | 285 | −16.839 | 43.410 | −76.591 | 1.00 | 174.19 | C |
| ATOM | 274 | CG | TRP | B | 285 | −16.436 | 41.963 | −76.675 | 1.00 | 174.19 | C |
| ATOM | 275 | CD1 | TRP | B | 285 | −16.478 | 41.040 | −75.670 | 1.00 | 174.19 | C |
| ATOM | 276 | CD2 | TRP | B | 285 | −16.004 | 41.258 | −77.849 | 1.00 | 174.19 | C |
| ATOM | 277 | CE2 | TRP | B | 285 | −15.763 | 39.919 | −77.468 | 1.00 | 174.19 | C |
| ATOM | 278 | CE3 | TRP | B | 285 | −15.778 | 41.634 | −79.187 | 1.00 | 174.19 | C |
| ATOM | 279 | NE1 | TRP | B | 285 | −16.070 | 39.812 | −76.136 | 1.00 | 174.19 | N |
| ATOM | 280 | CZ2 | TRP | B | 285 | −15.305 | 38.957 | −78.372 | 1.00 | 174.19 | C |
| ATOM | 281 | CZ3 | TRP | B | 285 | −15.321 | 40.680 | −80.080 | 1.00 | 174.19 | C |
| ATOM | 282 | CH2 | TRP | B | 285 | −15.079 | 39.363 | −79.668 | 1.00 | 174.19 | C |
| ATOM | 283 | N | GLU | B | 286 | −16.445 | 45.824 | −78.770 | 1.00 | 177.27 | N |
| ATOM | 284 | CA | GLU | B | 286 | −17.026 | 47.009 | −79.435 | 1.00 | 177.27 | C |
| ATOM | 285 | C | GLU | B | 286 | −17.712 | 46.688 | −80.788 | 1.00 | 177.27 | C |
| ATOM | 286 | O | GLU | B | 286 | −17.348 | 45.687 | −81.412 | 1.00 | 177.27 | O |
| ATOM | 287 | CB | GLU | B | 286 | −15.929 | 48.062 | −79.675 | 1.00 | 177.27 | C |
| ATOM | 288 | N | LEU | B | 287 | −18.675 | 47.567 | −81.261 | 1.00 | 180.30 | N |
| ATOM | 289 | CA | LEU | B | 287 | −19.319 | 47.440 | −82.593 | 1.00 | 180.30 | C |
| ATOM | 290 | C | LEU | B | 287 | −18.998 | 48.702 | −83.437 | 1.00 | 180.30 | C |
| ATOM | 291 | O | LEU | B | 287 | −18.682 | 49.752 | −82.859 | 1.00 | 180.30 | O |
| ATOM | 292 | CB | LEU | B | 287 | −20.853 | 47.210 | −82.508 | 1.00 | 180.30 | C |
| ATOM | 293 | CG | LEU | B | 287 | −21.549 | 46.424 | −83.654 | 1.00 | 180.30 | C |
| ATOM | 294 | CD1 | LEU | B | 287 | −22.777 | 45.730 | −83.148 | 1.00 | 180.30 | C |
| ATOM | 295 | CD2 | LEU | B | 287 | −22.005 | 47.330 | −84.790 | 1.00 | 180.30 | C |
| ATOM | 296 | N | GLU | B | 288 | −19.056 | 48.586 | −84.795 | 1.00 | 182.72 | N |
| ATOM | 297 | CA | GLU | B | 288 | −18.803 | 49.668 | −85.766 | 1.00 | 182.72 | C |
| ATOM | 298 | C | GLU | B | 288 | −19.762 | 50.845 | −85.542 | 1.00 | 182.72 | C |
| ATOM | 299 | O | GLU | B | 288 | −20.946 | 50.759 | −85.889 | 1.00 | 182.72 | O |
| ATOM | 300 | CB | GLU | B | 288 | −18.921 | 49.155 | −87.214 | 1.00 | 182.72 | C |
| ATOM | 301 | N | ASN | B | 289 | −19.243 | 51.924 | −84.909 | 1.00 | 183.79 | N |
| ATOM | 302 | CA | ASN | B | 289 | −19.955 | 53.152 | −84.534 | 1.00 | 183.79 | C |
| ATOM | 303 | C | ASN | B | 289 | −21.165 | 52.855 | −83.600 | 1.00 | 183.79 | C |
| ATOM | 304 | O | ASN | B | 289 | −21.973 | 53.751 | −83.358 | 1.00 | 183.79 | O |
| ATOM | 305 | CB | ASN | B | 289 | −20.386 | 53.952 | −85.774 | 1.00 | 183.79 | C |
| ATOM | 306 | N | LYS | B | 290 | −21.264 | 51.617 | −83.050 | 1.00 | 185.08 | N |
| ATOM | 307 | CA | LYS | B | 290 | −22.341 | 51.213 | −82.143 | 1.00 | 185.08 | C |
| ATOM | 308 | C | LYS | B | 290 | −21.786 | 50.680 | −80.825 | 1.00 | 185.08 | C |
| ATOM | 309 | O | LYS | B | 290 | −20.818 | 49.890 | −80.805 | 1.00 | 185.08 | O |
| ATOM | 310 | CB | LYS | B | 290 | −23.266 | 50.173 | −82.786 | 1.00 | 185.08 | C |
| ATOM | 311 | N | ALA | B | 291 | −22.409 | 51.149 | −79.716 | 1.00 | 187.65 | N |
| ATOM | 312 | CA | ALA | B | 291 | −22.084 | 50.775 | −78.335 | 1.00 | 187.65 | C |
| ATOM | 313 | C | ALA | B | 291 | −22.750 | 49.446 | −78.009 | 1.00 | 187.65 | C |
| ATOM | 314 | O | ALA | B | 291 | −23.982 | 49.335 | −78.047 | 1.00 | 187.65 | O |
| ATOM | 315 | CB | ALA | B | 291 | −22.527 | 51.862 | −77.359 | 1.00 | 187.65 | C |
| ATOM | 316 | N | LEU | B | 292 | −21.919 | 48.432 | −77.736 | 1.00 | 187.87 | N |
| ATOM | 317 | CA | LEU | B | 292 | −22.353 | 47.070 | −77.484 | 1.00 | 187.87 | C |
| ATOM | 318 | C | LEU | B | 292 | −23.057 | 46.887 | −76.151 | 1.00 | 187.87 | C |
| ATOM | 319 | O | LEU | B | 292 | −22.657 | 47.483 | −75.143 | 1.00 | 187.87 | O |
| ATOM | 320 | CB | LEU | B | 292 | −21.152 | 46.127 | −77.550 | 1.00 | 187.87 | C |
| ATOM | 321 | CG | LEU | B | 292 | −21.255 | 45.013 | −78.579 | 1.00 | 187.87 | C |
| ATOM | 322 | CD1 | LEU | B | 292 | −19.884 | 44.508 | −78.959 | 1.00 | 187.87 | C |
| ATOM | 323 | CD2 | LEU | B | 292 | −22.140 | 43.867 | −78.077 | 1.00 | 187.87 | C |
| ATOM | 324 | N | GLU | B | 293 | −24.103 | 46.025 | −76.159 | 1.00 | 188.78 | N |
| ATOM | 325 | CA | GLU | B | 293 | −24.865 | 45.625 | −74.980 | 1.00 | 188.78 | C |
| ATOM | 326 | C | GLU | B | 293 | −23.971 | 44.672 | −74.190 | 1.00 | 188.78 | C |
| ATOM | 327 | O | GLU | B | 293 | −24.002 | 43.448 | −74.397 | 1.00 | 188.78 | O |
| ATOM | 328 | CB | GLU | B | 293 | −26.218 | 44.992 | −75.364 | 1.00 | 188.78 | C |
| ATOM | 329 | N | GLU | B | 294 | −23.101 | 45.278 | −73.331 | 1.00 | 189.56 | N |
| ATOM | 330 | CA | GLU | B | 294 | −22.117 | 44.629 | −72.443 | 1.00 | 189.56 | C |
| ATOM | 331 | C | GLU | B | 294 | −22.824 | 43.800 | −71.342 | 1.00 | 189.56 | C |
| ATOM | 332 | O | GLU | B | 294 | −22.150 | 43.160 | −70.521 | 1.00 | 189.56 | O |
| ATOM | 333 | CB | GLU | B | 294 | −21.161 | 45.672 | −71.823 | 1.00 | 189.56 | C |
| ATOM | 334 | N | GLY | B | 295 | −24.170 | 43.826 | −71.369 | 1.00 | 186.66 | N |
| ATOM | 335 | CA | GLY | B | 295 | −25.084 | 43.052 | −70.532 | 1.00 | 186.66 | C |
| ATOM | 336 | C | GLY | B | 295 | −25.528 | 41.813 | −71.295 | 1.00 | 186.66 | C |
| ATOM | 337 | O | GLY | B | 295 | −26.710 | 41.455 | −71.314 | 1.00 | 186.66 | O |
| ATOM | 338 | N | ASN | B | 296 | −24.543 | 41.182 | −71.962 | 1.00 | 181.79 | N |
| ATOM | 339 | CA | ASN | B | 296 | −24.590 | 39.991 | −72.802 | 1.00 | 181.79 | C |
| ATOM | 340 | C | ASN | B | 296 | −23.135 | 39.436 | −72.946 | 1.00 | 181.79 | C |
| ATOM | 341 | O | ASN | B | 296 | −22.923 | 38.373 | −73.550 | 1.00 | 181.79 | O |
| ATOM | 342 | CB | ASN | B | 296 | −25.217 | 40.357 | −74.153 | 1.00 | 181.79 | C |
| ATOM | 343 | N | TYR | B | 297 | −22.147 | 40.177 | −72.350 | 1.00 | 175.21 | N |
| ATOM | 344 | CA | TYR | B | 297 | −20.700 | 39.924 | −72.307 | 1.00 | 175.21 | C |
| ATOM | 345 | C | TYR | B | 297 | −20.254 | 39.395 | −70.925 | 1.00 | 175.21 | C |
| ATOM | 346 | O | TYR | B | 297 | −20.660 | 39.949 | −69.900 | 1.00 | 175.21 | O |
| ATOM | 347 | CB | TYR | B | 297 | −19.935 | 41.215 | −72.645 | 1.00 | 175.21 | C |
| ATOM | 348 | N | PHE | B | 298 | −19.397 | 38.335 | −70.911 | 1.00 | 170.91 | N |
| ATOM | 349 | CA | PHE | B | 298 | −18.893 | 37.678 | −69.690 | 1.00 | 170.91 | C |
| ATOM | 350 | C | PHE | B | 298 | −17.370 | 37.445 | −69.690 | 1.00 | 170.91 | C |
| ATOM | 351 | O | PHE | B | 298 | −16.779 | 37.101 | −70.719 | 1.00 | 170.91 | O |
| ATOM | 352 | CB | PHE | B | 298 | −19.607 | 36.337 | −69.465 | 1.00 | 170.91 | C |
| ATOM | 353 | CG | PHE | B | 298 | −19.245 | 35.641 | −68.173 | 1.00 | 170.91 | C |
| ATOM | 354 | CD1 | PHE | B | 298 | −19.689 | 36.135 | −66.948 | 1.00 | 170.91 | C |
| ATOM | 355 | CD2 | PHE | B | 298 | −18.448 | 34.505 | −68.175 | 1.00 | 170.91 | C |
| ATOM | 356 | CE1 | PHE | B | 298 | −19.345 | 35.496 | −65.744 | 1.00 | 170.91 | C |
| ATOM | 357 | CE2 | PHE | B | 298 | −18.126 | 33.857 | −66.976 | 1.00 | 170.91 | C |
| ATOM | 358 | CZ | PHE | B | 298 | −18.565 | 34.362 | −65.767 | 1.00 | 170.91 | C |
| ATOM | 359 | N | GLU | B | 299 | −16.767 | 37.570 | −68.497 | 1.00 | 163.47 | N |
| ATOM | 360 | CA | GLU | B | 299 | −15.335 | 37.446 | −68.279 | 1.00 | 163.47 | C |
| ATOM | 361 | C | GLU | B | 299 | −14.935 | 36.359 | −67.282 | 1.00 | 163.47 | C |
| ATOM | 362 | O | GLU | B | 299 | −15.356 | 36.372 | −66.126 | 1.00 | 163.47 | O |
| ATOM | 363 | CB | GLU | B | 299 | −14.770 | 38.782 | −67.787 | 1.00 | 163.47 | C |
| ATOM | 364 | N | MET | B | 300 | −14.049 | 35.462 | −67.734 | 1.00 | 159.67 | N |
| ATOM | 365 | CA | MET | B | 300 | −13.421 | 34.382 | −66.956 | 1.00 | 159.67 | C |
| ATOM | 366 | C | MET | B | 300 | −11.918 | 34.540 | −67.008 | 1.00 | 159.67 | C |
| ATOM | 367 | O | MET | B | 300 | −11.399 | 35.071 | −67.991 | 1.00 | 159.67 | O |
| ATOM | 368 | CB | MET | B | 300 | −13.787 | 32.999 | −67.504 | 1.00 | 159.67 | C |
| ATOM | 369 | CG | MET | B | 300 | −15.224 | 32.656 | −67.377 | 1.00 | 159.67 | C |
| ATOM | 370 | SD | MET | B | 300 | −15.589 | 30.997 | −67.964 | 1.00 | 159.67 | S |
| ATOM | 371 | CE | MET | B | 300 | −15.225 | 31.157 | −69.714 | 1.00 | 159.67 | C |
| ATOM | 372 | N | SER | B | 301 | −11.206 | 34.054 | −65.999 | 1.00 | 155.29 | N |
| ATOM | 373 | CA | SER | B | 301 | −9.753 | 34.162 | −66.004 | 1.00 | 155.29 | C |
| ATOM | 374 | C | SER | B | 301 | −9.134 | 33.024 | −65.243 | 1.00 | 155.29 | C |
| ATOM | 375 | O | SER | B | 301 | −9.706 | 32.618 | −64.241 | 1.00 | 155.29 | O |
| ATOM | 376 | CB | SER | B | 301 | −9.312 | 35.492 | −65.399 | 1.00 | 155.29 | C |
| ATOM | 377 | OG | SER | B | 301 | −9.744 | 35.631 | −64.057 | 1.00 | 155.29 | O |
| ATOM | 378 | N | THR | B | 302 | −7.988 | 32.480 | −65.713 | 1.00 | 154.59 | N |
| ATOM | 379 | CA | THR | B | 302 | −7.263 | 31.431 | −64.967 | 1.00 | 154.59 | C |
| ATOM | 380 | C | THR | B | 302 | −5.744 | 31.589 | −65.215 | 1.00 | 154.59 | C |
| ATOM | 381 | O | THR | B | 302 | −5.318 | 32.423 | −66.029 | 1.00 | 154.59 | O |
| ATOM | 382 | CB | THR | B | 302 | −7.807 | 29.990 | −65.196 | 1.00 | 154.59 | C |
| ATOM | 383 | CG2 | THR | B | 302 | −7.310 | 29.345 | −66.476 | 1.00 | 154.59 | C |
| ATOM | 384 | OG1 | THR | B | 302 | −7.451 | 29.177 | −64.073 | 1.00 | 154.59 | O |
| ATOM | 385 | N | TYR | B | 303 | −4.939 | 30.813 | −64.492 | 1.00 | 154.51 | N |
| ATOM | 386 | CA | TYR | B | 303 | −3.498 | 30.891 | −64.618 | 1.00 | 154.51 | C |
| ATOM | 387 | C | TYR | B | 303 | −2.900 | 29.746 | −65.465 | 1.00 | 154.51 | C |
| ATOM | 388 | O | TYR | B | 303 | −3.509 | 28.679 | −65.613 | 1.00 | 154.51 | O |
| ATOM | 389 | CB | TYR | B | 303 | −2.883 | 30.919 | −63.234 | 1.00 | 154.51 | C |
| ATOM | 390 | CG | TYR | B | 303 | −3.004 | 32.264 | −62.561 | 1.00 | 154.51 | C |
| ATOM | 391 | CD1 | TYR | B | 303 | −1.911 | 33.111 | −62.460 | 1.00 | 154.51 | C |
| ATOM | 392 | CD2 | TYR | B | 303 | −4.200 | 32.672 | −61.984 | 1.00 | 154.51 | C |
| ATOM | 393 | CE1 | TYR | B | 303 | −2.003 | 34.334 | −61.805 | 1.00 | 154.51 | C |
| ATOM | 394 | CE2 | TYR | B | 303 | −4.307 | 33.897 | −61.334 | 1.00 | 154.51 | C |
| ATOM | 395 | CZ | TYR | B | 303 | −3.202 | 34.723 | −61.242 | 1.00 | 154.51 | C |
| ATOM | 396 | OH | TYR | B | 303 | −3.291 | 35.928 | −60.597 | 1.00 | 154.51 | O |
| ATOM | 397 | N | SER | B | 304 | −1.703 | 29.997 | −66.034 | 1.00 | 159.68 | N |
| ATOM | 398 | CA | SER | B | 304 | −0.941 | 29.082 | −66.896 | 1.00 | 159.68 | C |
| ATOM | 399 | C | SER | B | 304 | 0.554 | 29.023 | −66.459 | 1.00 | 159.68 | C |
| ATOM | 400 | O | SER | B | 304 | 0.938 | 29.767 | −65.551 | 1.00 | 159.68 | O |
| ATOM | 401 | CB | SER | B | 304 | −1.068 | 29.531 | −68.350 | 1.00 | 159.68 | C |
| ATOM | 402 | OG | SER | B | 304 | −0.201 | 28.842 | −69.235 | 1.00 | 159.68 | O |
| ATOM | 403 | N | THR | B | 305 | 1.377 | 28.133 | −67.104 | 1.00 | 164.27 | N |
| ATOM | 404 | CA | THR | B | 305 | 2.824 | 27.856 | −66.906 | 1.00 | 164.27 | C |
| ATOM | 405 | C | THR | B | 305 | 3.414 | 28.516 | −65.639 | 1.00 | 164.27 | C |
| ATOM | 406 | O | THR | B | 305 | 3.108 | 28.056 | −64.537 | 1.00 | 164.27 | O |
| ATOM | 407 | CB | THR | B | 305 | 3.651 | 28.223 | −68.154 | 1.00 | 164.27 | C |
| ATOM | 408 | CG2 | THR | B | 305 | 3.762 | 27.073 | −69.126 | 1.00 | 164.27 | C |
| ATOM | 409 | OG1 | THR | B | 305 | 3.093 | 29.367 | −68.806 | 1.00 | 164.27 | O |
| ATOM | 410 | N | ASN | B | 306 | 4.237 | 29.575 | −65.786 | 1.00 | 167.32 | N |
| ATOM | 411 | CA | ASN | B | 306 | 4.798 | 30.272 | −64.635 | 1.00 | 167.32 | C |
| ATOM | 412 | C | ASN | B | 306 | 4.031 | 31.561 | −64.390 | 1.00 | 167.32 | C |
| ATOM | 413 | O | ASN | B | 306 | 4.201 | 32.524 | −65.140 | 1.00 | 167.32 | O |
| ATOM | 414 | CB | ASN | B | 306 | 6.294 | 30.533 | −64.811 | 1.00 | 167.32 | C |
| ATOM | 415 | CG | ASN | B | 306 | 7.202 | 29.402 | −64.374 | 1.00 | 167.32 | C |
| ATOM | 416 | ND2 | ASN | B | 306 | 6.618 | 28.204 | −64.045 | 1.00 | 167.32 | N |
| ATOM | 417 | OD1 | ASN | B | 306 | 8.437 | 29.575 | −64.351 | 1.00 | 167.32 | O |
| ATOM | 418 | N | ARG | B | 307 | 3.135 | 31.551 | −63.372 | 1.00 | 162.29 | N |
| ATOM | 419 | CA | ARG | B | 307 | 2.278 | 32.669 | −62.959 | 1.00 | 162.29 | C |
| ATOM | 420 | C | ARG | B | 307 | 1.769 | 33.506 | −64.183 | 1.00 | 162.29 | C |
| ATOM | 421 | O | ARG | B | 307 | 1.696 | 34.735 | −64.130 | 1.00 | 162.29 | O |
| ATOM | 422 | CB | ARG | B | 307 | 3.012 | 33.547 | −61.930 | 1.00 | 162.29 | C |
| ATOM | 423 | N | THR | B | 308 | 1.423 | 32.798 | −65.277 | 1.00 | 160.17 | N |
| ATOM | 424 | CA | THR | B | 308 | 0.907 | 33.314 | −66.552 | 1.00 | 160.17 | C |
| ATOM | 425 | C | THR | B | 308 | −0.611 | 33.484 | −66.422 | 1.00 | 160.17 | C |
| ATOM | 426 | O | THR | B | 308 | −1.225 | 32.808 | −65.600 | 1.00 | 160.17 | O |
| ATOM | 427 | CB | THR | B | 308 | 1.290 | 32.341 | −67.700 | 1.00 | 160.17 | C |
| ATOM | 428 | CG2 | THR | B | 308 | 1.266 | 32.987 | −69.084 | 1.00 | 160.17 | C |
| ATOM | 429 | OG1 | THR | B | 308 | 2.575 | 31.760 | −67.450 | 1.00 | 160.17 | O |
| ATOM | 430 | N | MET | B | 309 | −1.221 | 34.373 | −67.219 | 1.00 | 158.25 | N |
| ATOM | 431 | CA | MET | B | 309 | −2.664 | 34.580 | −67.130 | 1.00 | 158.25 | C |
| ATOM | 432 | C | MET | B | 309 | −3.362 | 34.532 | −68.484 | 1.00 | 158.25 | C |
| ATOM | 433 | O | MET | B | 309 | −2.910 | 35.147 | −69.461 | 1.00 | 158.25 | O |
| ATOM | 434 | CB | MET | B | 309 | −2.981 | 35.901 | −66.433 | 1.00 | 158.25 | C |
| ATOM | 435 | CG | MET | B | 309 | −3.815 | 35.723 | −65.197 | 1.00 | 158.25 | C |
| ATOM | 436 | SD | MET | B | 309 | −5.488 | 36.353 | −65.422 | 1.00 | 158.25 | S |
| ATOM | 437 | CE | MET | B | 309 | −6.009 | 36.486 | −63.724 | 1.00 | 158.25 | C |
| ATOM | 438 | N | ILE | B | 310 | −4.471 | 33.780 | −68.522 | 1.00 | 154.55 | N |
| ATOM | 439 | CA | ILE | B | 310 | −5.329 | 33.634 | −69.689 | 1.00 | 154.55 | C |
| ATOM | 440 | C | ILE | B | 310 | −6.625 | 34.305 | −69.337 | 1.00 | 154.55 | C |
| ATOM | 441 | O | ILE | B | 310 | −7.145 | 34.065 | −68.237 | 1.00 | 154.55 | O |
| ATOM | 442 | CB | ILE | B | 310 | −5.550 | 32.151 | −70.120 | 1.00 | 154.55 | C |
| ATOM | 443 | CG1 | ILE | B | 310 | −4.275 | 31.301 | −70.013 | 1.00 | 154.55 | C |
| ATOM | 444 | CG2 | ILE | B | 310 | −6.172 | 32.056 | −71.522 | 1.00 | 154.55 | C |
| ATOM | 445 | CD1 | ILE | B | 310 | −4.543 | 29.867 | −69.601 | 1.00 | 154.55 | C |
| ATOM | 446 | N | ARG | B | 311 | −7.151 | 35.147 | −70.246 | 1.00 | 154.13 | N |
| ATOM | 447 | CA | ARG | B | 311 | −8.437 | 35.804 | −70.016 | 1.00 | 154.13 | C |
| ATOM | 448 | C | ARG | B | 311 | −9.403 | 35.537 | −71.166 | 1.00 | 154.13 | C |
| ATOM | 449 | O | ARG | B | 311 | −9.041 | 35.687 | −72.333 | 1.00 | 154.13 | O |
| ATOM | 450 | CB | ARG | B | 311 | −8.282 | 37.311 | −69.779 | 1.00 | 154.13 | C |
| ATOM | 451 | CG | ARG | B | 311 | −7.849 | 37.678 | −68.361 | 1.00 | 154.13 | C |
| ATOM | 452 | CD | ARG | B | 311 | −8.413 | 39.026 | −67.908 | 1.00 | 154.13 | C |
| ATOM | 453 | NE | ARG | B | 311 | −7.549 | 39.711 | −66.931 | 1.00 | 154.13 | N |
| ATOM | 454 | CZ | ARG | B | 311 | −7.690 | 39.653 | −65.606 | 1.00 | 154.13 | C |
| ATOM | 455 | NH1 | ARG | B | 311 | −8.666 | 38.931 | −65.064 | 1.00 | 154.13 | N + 1 |
| ATOM | 456 | NH2 | ARG | B | 311 | −6.851 | 40.308 | −64.814 | 1.00 | 154.13 | N |
| ATOM | 457 | N | ILE | B | 312 | −10.618 | 35.103 | −70.822 | 1.00 | 154.45 | N |
| ATOM | 458 | CA | ILE | B | 312 | −11.697 | 34.838 | −71.764 | 1.00 | 154.45 | C |
| ATOM | 459 | C | ILE | B | 312 | −12.723 | 35.931 | −71.604 | 1.00 | 154.45 | C |
| ATOM | 460 | O | ILE | B | 312 | −13.346 | 36.019 | −70.547 | 1.00 | 154.45 | O |
| ATOM | 461 | CB | ILE | B | 312 | −12.357 | 33.437 | −71.573 | 1.00 | 154.45 | C |
| ATOM | 462 | CG1 | ILE | B | 312 | −11.356 | 32.281 | −71.700 | 1.00 | 154.45 | C |
| ATOM | 463 | CG2 | ILE | B | 312 | −13.573 | 33.247 | −72.516 | 1.00 | 154.45 | C |
| ATOM | 464 | CD1 | ILE | B | 312 | −11.974 | 30.898 | −71.430 | 1.00 | 154.45 | C |
| ATOM | 465 | N | LEU | B | 313 | −12.932 | 36.744 | −72.637 | 1.00 | 158.55 | N |
| ATOM | 466 | CA | LEU | B | 313 | −13.961 | 37.776 | −72.586 | 1.00 | 158.55 | C |
| ATOM | 467 | C | LEU | B | 313 | −14.915 | 37.534 | −73.731 | 1.00 | 158.55 | C |
| ATOM | 468 | O | LEU | B | 313 | −14.729 | 38.098 | −74.809 | 1.00 | 158.55 | O |
| ATOM | 469 | CB | LEU | B | 313 | −13.345 | 39.174 | −72.646 | 1.00 | 158.55 | C |
| ATOM | 470 | CG | LEU | B | 313 | −14.212 | 40.276 | −72.049 | 1.00 | 158.55 | C |
| ATOM | 471 | CD1 | LEU | B | 313 | −13.637 | 40.772 | −70.723 | 1.00 | 158.55 | C |
| ATOM | 472 | CD2 | LEU | B | 313 | −14.356 | 41.438 | −73.007 | 1.00 | 158.55 | C |
| ATOM | 473 | N | PHE | B | 314 | −15.885 | 36.626 | −73.541 | 1.00 | 162.00 | N |
| ATOM | 474 | CA | PHE | B | 314 | −16.769 | 36.316 | −74.658 | 1.00 | 162.00 | C |
| ATOM | 475 | C | PHE | B | 314 | −17.975 | 37.225 | −74.690 | 1.00 | 162.00 | C |
| ATOM | 476 | O | PHE | B | 314 | −18.410 | 37.727 | −73.659 | 1.00 | 162.00 | O |
| ATOM | 477 | CB | PHE | B | 314 | −17.196 | 34.837 | −74.699 | 1.00 | 162.00 | C |
| ATOM | 478 | CG | PHE | B | 314 | −17.853 | 34.281 | −73.468 | 1.00 | 162.00 | C |
| ATOM | 479 | CD1 | PHE | B | 314 | −19.193 | 34.539 | −73.198 | 1.00 | 162.00 | C |
| ATOM | 480 | CD2 | PHE | B | 314 | −17.159 | 33.436 | −72.615 | 1.00 | 162.00 | C |
| ATOM | 481 | CE1 | PHE | B | 314 | −19.809 | 34.012 | −72.058 | 1.00 | 162.00 | C |
| ATOM | 482 | CE2 | PHE | B | 314 | −17.774 | 32.909 | −71.475 | 1.00 | 162.00 | C |
| ATOM | 483 | CZ | PHE | B | 314 | −19.099 | 33.192 | −71.208 | 1.00 | 162.00 | C |
| ATOM | 484 | N | ALA | B | 315 | −18.485 | 37.460 | −75.893 | 1.00 | 165.31 | N |
| ATOM | 485 | CA | ALA | B | 315 | −19.677 | 38.256 | −76.139 | 1.00 | 165.31 | C |
| ATOM | 486 | C | ALA | B | 315 | −20.733 | 37.353 | −76.703 | 1.00 | 165.31 | C |
| ATOM | 487 | O | ALA | B | 315 | −20.407 | 36.457 | −77.489 | 1.00 | 165.31 | O |
| ATOM | 488 | CB | ALA | B | 315 | −19.369 | 39.383 | −77.103 | 1.00 | 165.31 | C |
| ATOM | 489 | N | PHE | B | 316 | −21.993 | 37.549 | −76.300 | 1.00 | 170.05 | N |
| ATOM | 490 | CA | PHE | B | 316 | −23.037 | 36.673 | −76.805 | 1.00 | 170.05 | C |
| ATOM | 491 | C | PHE | B | 316 | −24.387 | 37.395 | −76.959 | 1.00 | 170.05 | C |
| ATOM | 492 | O | PHE | B | 316 | −24.750 | 38.202 | −76.109 | 1.00 | 170.05 | O |
| ATOM | 493 | CB | PHE | B | 316 | −23.142 | 35.423 | −75.900 | 1.00 | 170.05 | C |
| ATOM | 494 | CG | PHE | B | 316 | −24.447 | 35.145 | −75.196 | 1.00 | 170.05 | C |
| ATOM | 495 | CD1 | PHE | B | 316 | −25.460 | 34.421 | −75.829 | 1.00 | 170.05 | C |
| ATOM | 496 | CD2 | PHE | B | 316 | −24.658 | 35.581 | −73.889 | 1.00 | 170.05 | C |
| ATOM | 497 | CE1 | PHE | B | 316 | −26.665 | 34.139 | −75.169 | 1.00 | 170.05 | C |
| ATOM | 498 | CE2 | PHE | B | 316 | −25.869 | 35.314 | −73.230 | 1.00 | 170.05 | C |
| ATOM | 499 | CZ | PHE | B | 316 | −26.865 | 34.591 | −73.874 | 1.00 | 170.05 | C |
| ATOM | 500 | N | VAL | B | 317 | −25.131 | 37.082 | −78.042 | 1.00 | 169.64 | N |
| ATOM | 501 | CA | VAL | B | 317 | −26.475 | 37.627 | −78.302 | 1.00 | 169.64 | C |
| ATOM | 502 | C | VAL | B | 317 | −27.478 | 36.488 | −78.167 | 1.00 | 169.64 | C |
| ATOM | 503 | O | VAL | B | 317 | −27.303 | 35.456 | −78.816 | 1.00 | 169.64 | O |
| ATOM | 504 | CB | VAL | B | 317 | −26.594 | 38.330 | −79.674 | 1.00 | 169.64 | C |
| ATOM | 505 | CG1 | VAL | B | 317 | −28.008 | 38.852 | −79.902 | 1.00 | 169.64 | C |
| ATOM | 506 | CG2 | VAL | B | 317 | −25.577 | 39.455 | −79.799 | 1.00 | 169.64 | C |
| ATOM | 507 | N | SER | B | 318 | −28.506 | 36.667 | −77.317 | 1.00 | 170.99 | N |
| ATOM | 508 | CA | SER | B | 318 | −29.536 | 35.665 | −77.046 | 1.00 | 170.99 | C |
| ATOM | 509 | C | SER | B | 318 | −30.198 | 35.224 | −78.341 | 1.00 | 170.99 | C |
| ATOM | 510 | O | SER | B | 318 | −30.098 | 34.056 | −78.711 | 1.00 | 170.99 | O |
| ATOM | 511 | CB | SER | B | 318 | −30.568 | 36.213 | −76.065 | 1.00 | 170.99 | C |
| ATOM | 512 | N | SER | B | 319 | −30.823 | 36.169 | −79.046 | 1.00 | 173.70 | N |
| ATOM | 513 | CA | SER | B | 319 | −31.464 | 35.957 | −80.339 | 1.00 | 173.70 | C |
| ATOM | 514 | C | SER | B | 319 | −31.083 | 37.128 | −81.228 | 1.00 | 173.70 | C |
| ATOM | 515 | O | SER | B | 319 | −31.377 | 38.273 | −80.865 | 1.00 | 173.70 | O |
| ATOM | 516 | CB | SER | B | 319 | −32.979 | 35.829 | −80.187 | 1.00 | 173.70 | C |
| ATOM | 517 | N | VAL | B | 320 | −30.361 | 36.862 | −82.351 | 1.00 | 175.37 | N |
| ATOM | 518 | CA | VAL | B | 320 | −29.890 | 37.923 | −83.262 | 1.00 | 175.37 | C |
| ATOM | 519 | C | VAL | B | 320 | −31.024 | 38.488 | −84.135 | 1.00 | 175.37 | C |
| ATOM | 520 | O | VAL | B | 320 | −32.059 | 37.846 | −84.318 | 1.00 | 175.37 | O |
| ATOM | 521 | CB | VAL | B | 320 | −28.678 | 37.533 | −84.146 | 1.00 | 175.37 | C |
| ATOM | 522 | CG1 | VAL | B | 320 | −27.453 | 37.235 | −83.300 | 1.00 | 175.37 | C |
| ATOM | 523 | CG2 | VAL | B | 320 | −29.002 | 36.383 | −85.092 | 1.00 | 175.37 | C |
| ATOM | 524 | N | ALA | B | 321 | −30.802 | 39.703 | −84.671 | 1.00 | 178.14 | N |
| ATOM | 525 | CA | ALA | B | 321 | −31.713 | 40.430 | −85.565 | 1.00 | 178.14 | C |
| ATOM | 526 | C | ALA | B | 321 | −30.905 | 41.352 | −86.518 | 1.00 | 178.14 | C |
| ATOM | 527 | O | ALA | B | 321 | −29.697 | 41.153 | −86.666 | 1.00 | 178.14 | O |
| ATOM | 528 | CB | ALA | B | 321 | −32.723 | 41.231 | −84.750 | 1.00 | 178.14 | C |
| ATOM | 529 | N | ARG | B | 322 | −31.563 | 42.335 | −87.170 | 1.00 | 178.72 | N |
| ATOM | 530 | CA | ARG | B | 322 | −30.913 | 43.280 | −88.079 | 1.00 | 178.72 | C |
| ATOM | 531 | C | ARG | B | 322 | −29.890 | 44.145 | −87.344 | 1.00 | 178.72 | C |
| ATOM | 532 | O | ARG | B | 322 | −28.777 | 44.316 | −87.841 | 1.00 | 178.72 | O |
| ATOM | 533 | CB | ARG | B | 322 | −31.961 | 44.172 | −88.753 | 1.00 | 178.72 | C |
| ATOM | 534 | N | ASN | B | 323 | −30.269 | 44.645 | −86.141 | 1.00 | 181.16 | N |
| ATOM | 535 | CA | ASN | B | 323 | −29.494 | 45.513 | −85.237 | 1.00 | 181.16 | C |
| ATOM | 536 | C | ASN | B | 323 | −28.150 | 44.907 | −84.783 | 1.00 | 181.16 | C |
| ATOM | 537 | O | ASN | B | 323 | −27.210 | 45.670 | −84.527 | 1.00 | 181.16 | O |
| ATOM | 538 | CB | ASN | B | 323 | −30.332 | 45.881 | −83.987 | 1.00 | 181.16 | C |
| ATOM | 539 | CG | ASN | B | 323 | −31.599 | 46.651 | −84.304 | 1.00 | 181.16 | C |
| ATOM | 540 | ND2 | ASN | B | 323 | −32.422 | 46.993 | −83.338 | 1.00 | 181.16 | N |
| ATOM | 541 | OD1 | ASN | B | 323 | −31.933 | 46.897 | −85.460 | 1.00 | 181.16 | O |
| ATOM | 542 | N | ASP | B | 324 | −28.061 | 43.554 | −84.685 | 1.00 | 179.55 | N |
| ATOM | 543 | CA | ASP | B | 324 | −26.871 | 42.812 | −84.229 | 1.00 | 179.55 | C |
| ATOM | 544 | C | ASP | B | 324 | −25.792 | 42.602 | −85.330 | 1.00 | 179.55 | C |
| ATOM | 545 | O | ASP | B | 324 | −24.635 | 42.334 | −84.980 | 1.00 | 179.55 | O |
| ATOM | 546 | CB | ASP | B | 324 | −27.275 | 41.463 | −83.617 | 1.00 | 179.55 | C |
| ATOM | 547 | CG | ASP | B | 324 | −28.237 | 41.604 | −82.455 | 1.00 | 179.55 | C |
| ATOM | 548 | OD1 | ASP | B | 324 | −27.903 | 42.327 | −81.488 | 1.00 | 179.55 | O |
| ATOM | 549 | OD2 | ASP | B | 324 | −29.331 | 41.025 | −82.525 | 1.00 | 179.55 | O + 1 |
| ATOM | 550 | N | THR | B | 325 | −26.152 | 42.744 | −86.635 | 1.00 | 175.46 | N |
| ATOM | 551 | CA | THR | B | 325 | −25.201 | 42.640 | −87.750 | 1.00 | 175.46 | C |
| ATOM | 552 | C | THR | B | 325 | −24.241 | 43.864 | −87.708 | 1.00 | 175.46 | C |
| ATOM | 553 | O | THR | B | 325 | −24.717 | 45.002 | −87.583 | 1.00 | 175.46 | O |
| ATOM | 554 | CB | THR | B | 325 | −25.957 | 42.532 | −89.079 | 1.00 | 175.46 | C |
| ATOM | 555 | N | GLY | B | 326 | −22.918 | 43.614 | −87.743 | 1.00 | 171.84 | N |
| ATOM | 556 | CA | GLY | B | 326 | −21.892 | 44.663 | −87.701 | 1.00 | 171.84 | C |
| ATOM | 557 | C | GLY | B | 326 | −20.437 | 44.241 | −87.539 | 1.00 | 171.84 | C |
| ATOM | 558 | O | GLY | B | 326 | −20.130 | 43.053 | −87.469 | 1.00 | 171.84 | O |
| ATOM | 559 | N | TYR | B | 327 | −19.525 | 45.229 | −87.479 | 1.00 | 168.24 | N |
| ATOM | 560 | CA | TYR | B | 327 | −18.086 | 45.021 | −87.300 | 1.00 | 168.24 | C |
| ATOM | 561 | C | TYR | B | 327 | −17.723 | 44.987 | −85.804 | 1.00 | 168.24 | C |
| ATOM | 562 | O | TYR | B | 327 | −17.661 | 46.038 | −85.157 | 1.00 | 168.24 | O |
| ATOM | 563 | CB | TYR | B | 327 | −17.289 | 46.115 | −88.028 | 1.00 | 168.24 | C |
| ATOM | 564 | N | TYR | B | 328 | −17.492 | 43.777 | −85.256 | 1.00 | 164.98 | N |
| ATOM | 565 | CA | TYR | B | 328 | −17.159 | 43.565 | −83.839 | 1.00 | 164.98 | C |
| ATOM | 566 | C | TYR | B | 328 | −15.663 | 43.482 | −83.623 | 1.00 | 164.98 | C |
| ATOM | 567 | O | TYR | B | 328 | −14.964 | 42.824 | −84.394 | 1.00 | 164.98 | O |
| ATOM | 568 | CB | TYR | B | 328 | −17.816 | 42.283 | −83.301 | 1.00 | 164.98 | C |
| ATOM | 569 | CG | TYR | B | 328 | −19.318 | 42.351 | −83.109 | 1.00 | 164.98 | C |
| ATOM | 570 | CD1 | TYR | B | 328 | −19.880 | 42.281 | −81.840 | 1.00 | 164.98 | C |
| ATOM | 571 | CD2 | TYR | B | 328 | −20.181 | 42.398 | −84.203 | 1.00 | 164.98 | C |
| ATOM | 572 | CE1 | TYR | B | 328 | −21.263 | 42.296 | −81.660 | 1.00 | 164.98 | C |
| ATOM | 573 | CE2 | TYR | B | 328 | −21.564 | 42.417 | −84.034 | 1.00 | 164.98 | C |
| ATOM | 574 | CZ | TYR | B | 328 | −22.100 | 42.355 | −82.762 | 1.00 | 164.98 | C |
| ATOM | 575 | OH | TYR | B | 328 | −23.460 | 42.388 | −82.594 | 1.00 | 164.98 | O |
| ATOM | 576 | N | THR | B | 329 | −15.169 | 44.122 | −82.562 | 1.00 | 162.52 | N |
| ATOM | 577 | CA | THR | B | 329 | −13.740 | 44.089 | −82.283 | 1.00 | 162.52 | C |
| ATOM | 578 | C | THR | B | 329 | −13.439 | 43.917 | −80.807 | 1.00 | 162.52 | C |
| ATOM | 579 | O | THR | B | 329 | −14.062 | 44.547 | −79.938 | 1.00 | 162.52 | O |
| ATOM | 580 | CB | THR | B | 329 | −13.014 | 45.319 | −82.840 | 1.00 | 162.52 | C |
| ATOM | 581 | CG2 | THR | B | 329 | −13.525 | 46.641 | −82.259 | 1.00 | 162.52 | C |
| ATOM | 582 | OG1 | THR | B | 329 | −11.607 | 45.175 | −82.601 | 1.00 | 162.52 | O |
| ATOM | 583 | N | CYS | B | 330 | −12.426 | 43.085 | −80.550 | 1.00 | 158.77 | N |
| ATOM | 584 | CA | CYS | B | 330 | −11.939 | 42.796 | −79.213 | 1.00 | 158.77 | C |
| ATOM | 585 | C | CYS | B | 330 | −10.555 | 43.417 | −79.027 | 1.00 | 158.77 | C |
| ATOM | 586 | O | CYS | B | 330 | −9.658 | 43.178 | −79.844 | 1.00 | 158.77 | O |
| ATOM | 587 | CB | CYS | B | 330 | −11.919 | 41.292 | −78.960 | 1.00 | 158.77 | C |
| ATOM | 588 | SG | CYS | B | 330 | −11.389 | 40.836 | −77.291 | 1.00 | 158.77 | S |
| ATOM | 589 | N | SER | B | 331 | −10.386 | 44.217 | −77.960 | 1.00 | 160.47 | N |
| ATOM | 590 | CA | SER | B | 331 | −9.110 | 44.858 | −77.660 | 1.00 | 160.47 | C |
| ATOM | 591 | C | SER | B | 331 | −8.725 | 44.620 | −76.212 | 1.00 | 160.47 | C |
| ATOM | 592 | O | SER | B | 331 | −9.559 | 44.784 | −75.320 | 1.00 | 160.47 | O |
| ATOM | 593 | CB | SER | B | 331 | −9.174 | 46.351 | −77.956 | 1.00 | 160.47 | C |
| ATOM | 594 | N | SER | B | 332 | −7.465 | 44.213 | −75.983 | 1.00 | 162.45 | N |
| ATOM | 595 | CA | SER | B | 332 | −6.937 | 43.960 | −74.643 | 1.00 | 162.45 | C |
| ATOM | 596 | C | SER | B | 332 | −5.987 | 45.080 | −74.205 | 1.00 | 162.45 | C |
| ATOM | 597 | O | SER | B | 332 | −5.417 | 45.779 | −75.049 | 1.00 | 162.45 | O |
| ATOM | 598 | CB | SER | B | 332 | −6.243 | 42.605 | −74.574 | 1.00 | 162.45 | C |
| ATOM | 599 | N | SER | B | 333 | −5.851 | 45.265 | −72.873 | 1.00 | 163.81 | N |
| ATOM | 600 | CA | SER | B | 333 | −5.009 | 46.301 | −72.269 | 1.00 | 163.81 | C |
| ATOM | 601 | C | SER | B | 333 | −3.552 | 46.123 | −72.679 | 1.00 | 163.81 | C |
| ATOM | 602 | O | SER | B | 333 | −2.955 | 47.053 | −73.222 | 1.00 | 163.81 | O |
| ATOM | 603 | CB | SER | B | 333 | −5.140 | 46.296 | −70.747 | 1.00 | 163.81 | C |
| ATOM | 604 | N | LYS | B | 334 | −2.998 | 44.925 | −72.473 | 1.00 | 164.27 | N |
| ATOM | 605 | CA | LYS | B | 334 | −1.620 | 44.667 | −72.849 | 1.00 | 164.27 | C |
| ATOM | 606 | C | LYS | B | 334 | −1.572 | 43.831 | −74.146 | 1.00 | 164.27 | C |
| ATOM | 607 | O | LYS | B | 334 | −0.676 | 42.993 | −74.286 | 1.00 | 164.27 | O |
| ATOM | 608 | CB | LYS | B | 334 | −0.862 | 43.995 | −71.687 | 1.00 | 164.27 | C |
| ATOM | 609 | N | HIS | B | 335 | −2.515 | 44.076 | −75.111 | 1.00 | 165.44 | N |
| ATOM | 610 | CA | HIS | B | 335 | −2.561 | 43.346 | −76.394 | 1.00 | 165.44 | C |
| ATOM | 611 | C | HIS | B | 335 | −3.158 | 44.162 | −77.573 | 1.00 | 165.44 | C |
| ATOM | 612 | O | HIS | B | 335 | −3.831 | 45.167 | −77.325 | 1.00 | 165.44 | O |
| ATOM | 613 | CB | HIS | B | 335 | −3.327 | 42.021 | −76.244 | 1.00 | 165.44 | C |
| ATOM | 614 | CG | HIS | B | 335 | −2.530 | 40.991 | −75.524 | 1.00 | 165.44 | C |
| ATOM | 615 | CD2 | HIS | B | 335 | −2.742 | 40.427 | −74.314 | 1.00 | 165.44 | C |
| ATOM | 616 | ND1 | HIS | B | 335 | −1.315 | 40.547 | −76.020 | 1.00 | 165.44 | N |
| ATOM | 617 | CE1 | HIS | B | 335 | −0.844 | 39.707 | −75.115 | 1.00 | 165.44 | C |
| ATOM | 618 | NE2 | HIS | B | 335 | −1.673 | 39.594 | −74.074 | 1.00 | 165.44 | N |
| ATOM | 619 | N | PRO | B | 336 | −2.926 | 43.745 | −78.859 | 1.00 | 165.68 | N |
| ATOM | 620 | CA | PRO | B | 336 | −3.482 | 44.508 | −79.998 | 1.00 | 165.68 | C |
| ATOM | 621 | C | PRO | B | 336 | −4.900 | 44.085 | −80.406 | 1.00 | 165.68 | C |
| ATOM | 622 | O | PRO | B | 336 | −5.199 | 42.888 | −80.453 | 1.00 | 165.68 | O |
| ATOM | 623 | CB | PRO | B | 336 | −2.499 | 44.208 | −81.127 | 1.00 | 165.68 | C |
| ATOM | 624 | CG | PRO | B | 336 | −1.970 | 42.838 | −80.815 | 1.00 | 165.68 | C |
| ATOM | 625 | CD | PRO | B | 336 | −2.124 | 42.592 | −79.334 | 1.00 | 165.68 | C |
| ATOM | 626 | N | SER | B | 337 | −5.755 | 45.069 | −80.748 | 1.00 | 165.23 | N |
| ATOM | 627 | CA | SER | B | 337 | −7.141 | 44.836 | −81.162 | 1.00 | 165.23 | C |
| ATOM | 628 | C | SER | B | 337 | −7.230 | 43.949 | −82.422 | 1.00 | 165.23 | C |
| ATOM | 629 | O | SER | B | 337 | −6.367 | 44.054 | −83.298 | 1.00 | 165.23 | O |
| ATOM | 630 | CB | SER | B | 337 | −7.849 | 46.162 | −81.417 | 1.00 | 165.23 | C |
| ATOM | 631 | N | GLN | B | 338 | −8.252 | 43.051 | −82.484 | 1.00 | 164.33 | N |
| ATOM | 632 | CA | GLN | B | 338 | −8.537 | 42.151 | −83.626 | 1.00 | 164.33 | C |
| ATOM | 633 | C | GLN | B | 338 | −10.037 | 42.126 | −83.876 | 1.00 | 164.33 | C |
| ATOM | 634 | O | GLN | B | 338 | −10.828 | 42.212 | −82.926 | 1.00 | 164.33 | O |
| ATOM | 635 | CB | GLN | B | 338 | −7.986 | 40.736 | −83.408 | 1.00 | 164.33 | C |
| ATOM | 636 | N | SER | B | 339 | −10.439 | 42.040 | −85.142 | 1.00 | 162.18 | N |
| ATOM | 637 | CA | SER | B | 339 | −11.858 | 42.150 | −85.424 | 1.00 | 162.18 | C |
| ATOM | 638 | C | SER | B | 339 | −12.419 | 41.085 | −86.341 | 1.00 | 162.18 | C |
| ATOM | 639 | O | SER | B | 339 | −11.687 | 40.443 | −87.090 | 1.00 | 162.18 | O |
| ATOM | 640 | CB | SER | B | 339 | −12.147 | 43.512 | −86.038 | 1.00 | 162.18 | C |
| ATOM | 641 | OG | SER | B | 339 | −11.268 | 44.499 | −85.524 | 1.00 | 162.18 | O |
| ATOM | 642 | N | ALA | B | 340 | −13.757 | 40.941 | −86.288 | 1.00 | 163.31 | N |
| ATOM | 643 | CA | ALA | B | 340 | −14.585 | 40.029 | −87.075 | 1.00 | 163.31 | C |
| ATOM | 644 | C | ALA | B | 340 | −15.936 | 40.678 | −87.355 | 1.00 | 163.31 | C |
| ATOM | 645 | O | ALA | B | 340 | −16.484 | 41.354 | −86.484 | 1.00 | 163.31 | O |
| ATOM | 646 | CB | ALA | B | 340 | −14.772 | 38.715 | −86.338 | 1.00 | 163.31 | C |
| ATOM | 647 | N | LEU | B | 341 | −16.474 | 40.495 | −88.560 | 1.00 | 165.75 | N |
| ATOM | 648 | CA | LEU | B | 341 | −17.734 | 41.148 | −88.908 | 1.00 | 165.75 | C |
| ATOM | 649 | C | LEU | B | 341 | −18.880 | 40.163 | −88.996 | 1.00 | 165.75 | C |
| ATOM | 650 | O | LEU | B | 341 | −18.867 | 39.253 | −89.827 | 1.00 | 165.75 | O |
| ATOM | 651 | CB | LEU | B | 341 | −17.604 | 41.937 | −90.217 | 1.00 | 165.75 | C |
| ATOM | 652 | CG | LEU | B | 341 | −16.809 | 43.254 | −90.143 | 1.00 | 165.75 | C |
| ATOM | 653 | CD1 | LEU | B | 341 | −15.323 | 43.055 | −90.486 | 1.00 | 165.75 | C |
| ATOM | 654 | CD2 | LEU | B | 341 | −17.415 | 44.309 | −91.052 | 1.00 | 165.75 | C |
| ATOM | 655 | N | VAL | B | 342 | −19.879 | 40.368 | −88.125 | 1.00 | 166.66 | N |
| ATOM | 656 | CA | VAL | B | 342 | −21.097 | 39.569 | −87.986 | 1.00 | 166.66 | C |
| ATOM | 657 | C | VAL | B | 342 | −22.113 | 39.962 | −89.078 | 1.00 | 166.66 | C |
| ATOM | 658 | O | VAL | B | 342 | −22.572 | 41.106 | −89.131 | 1.00 | 166.66 | O |
| ATOM | 659 | CB | VAL | B | 342 | −21.692 | 39.698 | −86.555 | 1.00 | 166.66 | C |
| ATOM | 660 | CG1 | VAL | B | 342 | −23.050 | 39.015 | −86.444 | 1.00 | 166.66 | C |
| ATOM | 661 | CG2 | VAL | B | 342 | −20.734 | 39.133 | −85.518 | 1.00 | 166.66 | C |
| ATOM | 662 | N | THR | B | 343 | −22.452 | 38.991 | −89.936 | 1.00 | 167.53 | N |
| ATOM | 663 | CA | THR | B | 343 | −23.398 | 39.132 | −91.025 | 1.00 | 167.53 | C |
| ATOM | 664 | C | THR | B | 343 | −24.585 | 38.222 | −90.784 | 1.00 | 167.53 | C |
| ATOM | 665 | O | THR | B | 343 | −24.459 | 37.011 | −90.546 | 1.00 | 167.53 | O |
| ATOM | 666 | CB | THR | B | 343 | −22.728 | 38.864 | −92.354 | 1.00 | 167.53 | C |
| ATOM | 667 | N | ILE | B | 344 | −25.748 | 38.829 | −90.837 | 1.00 | 171.98 | N |
| ATOM | 668 | CA | ILE | B | 344 | −27.006 | 38.154 | −90.615 | 1.00 | 171.98 | C |
| ATOM | 669 | C | ILE | B | 344 | −27.527 | 37.638 | −91.972 | 1.00 | 171.98 | C |
| ATOM | 670 | O | ILE | B | 344 | −27.315 | 38.273 | −93.006 | 1.00 | 171.98 | O |
| ATOM | 671 | CB | ILE | B | 344 | −27.953 | 39.140 | −89.854 | 1.00 | 171.98 | C |
| ATOM | 672 | CG1 | ILE | B | 344 | −28.166 | 38.722 | −88.384 | 1.00 | 171.98 | C |
| ATOM | 673 | CG2 | ILE | B | 344 | −29.259 | 39.467 | −90.569 | 1.00 | 171.98 | C |
| ATOM | 674 | CD1 | ILE | B | 344 | −27.075 | 39.243 | −87.392 | 1.00 | 171.98 | C |
| ATOM | 675 | N | VAL | B | 345 | −28.144 | 36.454 | −91.966 | 1.00 | 176.21 | N |
| ATOM | 676 | CA | VAL | B | 345 | −28.704 | 35.867 | −93.185 | 1.00 | 176.21 | C |
| ATOM | 677 | C | VAL | B | 345 | −30.200 | 35.563 | −92.999 | 1.00 | 176.21 | C |
| ATOM | 678 | O | VAL | B | 345 | −30.677 | 35.276 | −91.871 | 1.00 | 176.21 | O |
| ATOM | 679 | CB | VAL | B | 345 | −27.939 | 34.632 | −93.748 | 1.00 | 176.21 | C |
| ATOM | 680 | CG1 | VAL | B | 345 | −26.675 | 35.055 | −94.480 | 1.00 | 176.21 | C |
| ATOM | 681 | CG2 | VAL | B | 345 | −27.633 | 33.587 | −92.674 | 1.00 | 176.21 | C |
| ATOM | 682 | N | GLU | B | 346 | −30.925 | 35.677 | −94.135 | 1.00 | 178.80 | N |
| ATOM | 683 | CA | GLU | B | 346 | −32.356 | 35.436 | −94.288 | 1.00 | 178.80 | C |
| ATOM | 684 | C | GLU | B | 346 | −32.610 | 33.983 | −94.695 | 1.00 | 178.80 | C |
| ATOM | 685 | O | GLU | B | 346 | −33.632 | 33.409 | −94.314 | 1.00 | 178.80 | O |
| ATOM | 686 | CB | GLU | B | 346 | −32.953 | 36.401 | −95.323 | 1.00 | 178.80 | C |
| TER | 687 | | GLU | B | 346 | | | | | | |
| ATOM | 688 | N | THR | A | 1 | 5.321 | 54.347 | −52.494 | 1.00 | 185.01 | N |
| ATOM | 689 | CA | THR | A | 1 | 5.559 | 52.904 | −52.695 | 1.00 | 185.01 | C |
| ATOM | 690 | C | THR | A | 1 | 4.970 | 52.120 | −51.488 | 1.00 | 185.01 | C |
| ATOM | 691 | O | THR | A | 1 | 5.217 | 50.918 | −51.336 | 1.00 | 185.01 | O |
| ATOM | 692 | CB | THR | A | 1 | 7.056 | 52.621 | −52.911 | 1.00 | 185.01 | C |
| ATOM | 693 | N | GLN | A | 2 | 4.183 | 52.835 | −50.641 | 1.00 | 180.26 | N |
| ATOM | 694 | CA | GLN | A | 2 | 3.446 | 52.356 | −49.469 | 1.00 | 180.26 | C |
| ATOM | 695 | C | GLN | A | 2 | 1.944 | 52.225 | −49.832 | 1.00 | 180.26 | C |
| ATOM | 696 | O | GLN | A | 2 | 1.080 | 52.324 | −48.959 | 1.00 | 180.26 | O |
| ATOM | 697 | CB | GLN | A | 2 | 3.665 | 53.304 | −48.267 | 1.00 | 180.26 | C |
| ATOM | 698 | N | ASP | A | 3 | 1.646 | 52.010 | −51.131 | 1.00 | 174.34 | N |
| ATOM | 699 | CA | ASP | A | 3 | 0.285 | 51.826 | −51.612 | 1.00 | 174.34 | C |
| ATOM | 700 | C | ASP | A | 3 | 0.061 | 50.363 | −51.975 | 1.00 | 174.34 | C |
| ATOM | 701 | O | ASP | A | 3 | 1.013 | 49.581 | −52.010 | 1.00 | 174.34 | O |
| ATOM | 702 | CB | ASP | A | 3 | −0.025 | 52.747 | −52.792 | 1.00 | 174.34 | C |
| ATOM | 703 | N | CYS | A | 4 | −1.205 | 49.987 | −52.205 | 1.00 | 170.58 | N |
| ATOM | 704 | CA | CYS | A | 4 | −1.605 | 48.619 | −52.524 | 1.00 | 170.58 | C |
| ATOM | 705 | C | CYS | A | 4 | −2.976 | 48.619 | −53.170 | 1.00 | 170.58 | C |
| ATOM | 706 | O | CYS | A | 4 | −3.971 | 48.863 | −52.498 | 1.00 | 170.58 | O |
| ATOM | 707 | CB | CYS | A | 4 | −1.593 | 47.758 | −51.261 | 1.00 | 170.58 | C |
| ATOM | 708 | SG | CYS | A | 4 | −2.029 | 46.023 | −51.540 | 1.00 | 170.58 | S |
| ATOM | 709 | N | SER | A | 5 | −3.030 | 48.351 | −54.465 | 1.00 | 169.59 | N |
| ATOM | 710 | CA | SER | A | 5 | −4.276 | 48.306 | −55.223 | 1.00 | 169.59 | C |
| ATOM | 711 | C | SER | A | 5 | −4.051 | 47.507 | −56.482 | 1.00 | 169.59 | C |
| ATOM | 712 | O | SER | A | 5 | −2.911 | 47.415 | −56.947 | 1.00 | 169.59 | O |
| ATOM | 713 | CB | SER | A | 5 | −4.740 | 49.713 | −55.580 | 1.00 | 169.59 | C |
| ATOM | 714 | OG | SER | A | 5 | −3.864 | 50.295 | −56.533 | 1.00 | 169.59 | O |
| ATOM | 715 | N | PHE | A | 6 | −5.124 | 46.944 | −57.050 | 1.00 | 166.76 | N |
| ATOM | 716 | CA | PHE | A | 6 | −5.011 | 46.128 | −58.253 | 1.00 | 166.76 | C |
| ATOM | 717 | C | PHE | A | 6 | −5.940 | 46.647 | −59.320 | 1.00 | 166.76 | C |
| ATOM | 718 | O | PHE | A | 6 | −7.147 | 46.740 | −59.092 | 1.00 | 166.76 | O |
| ATOM | 719 | CB | PHE | A | 6 | −5.313 | 44.657 | −57.931 | 1.00 | 166.76 | C |
| ATOM | 720 | CG | PHE | A | 6 | −4.504 | 44.124 | −56.772 | 1.00 | 166.76 | C |
| ATOM | 721 | CD1 | PHE | A | 6 | −3.361 | 43.363 | −56.990 | 1.00 | 166.76 | C |
| ATOM | 722 | CD2 | PHE | A | 6 | −4.860 | 44.421 | −55.459 | 1.00 | 166.76 | C |
| ATOM | 723 | CE1 | PHE | A | 6 | −2.594 | 42.897 | −55.915 | 1.00 | 166.76 | C |
| ATOM | 724 | CE2 | PHE | A | 6 | −4.089 | 43.968 | −54.388 | 1.00 | 166.76 | C |
| ATOM | 725 | CZ | PHE | A | 6 | −2.964 | 43.204 | −54.622 | 1.00 | 166.76 | C |
| ATOM | 726 | N | GLN | A | 7 | −5.374 | 46.987 | −60.497 | 1.00 | 163.88 | N |
| ATOM | 727 | CA | GLN | A | 7 | −6.110 | 47.520 | −61.655 | 1.00 | 163.88 | C |
| ATOM | 728 | C | GLN | A | 7 | −6.991 | 46.436 | −62.292 | 1.00 | 163.88 | C |
| ATOM | 729 | O | GLN | A | 7 | −8.166 | 46.688 | −62.565 | 1.00 | 163.88 | O |
| ATOM | 730 | CB | GLN | A | 7 | −5.140 | 48.116 | −62.699 | 1.00 | 163.88 | C |
| ATOM | 731 | N | HIS | A | 8 | −6.422 | 45.235 | −62.505 | 1.00 | 159.70 | N |
| ATOM | 732 | CA | HIS | A | 8 | −7.101 | 44.085 | −63.095 | 1.00 | 159.70 | C |
| ATOM | 733 | C | HIS | A | 8 | −7.144 | 42.968 | −62.064 | 1.00 | 159.70 | C |
| ATOM | 734 | O | HIS | A | 8 | −6.228 | 42.877 | −61.249 | 1.00 | 159.70 | O |
| ATOM | 735 | CB | HIS | A | 8 | −6.393 | 43.655 | −64.389 | 1.00 | 159.70 | C |
| ATOM | 736 | CG | HIS | A | 8 | −6.235 | 44.774 | −65.383 | 1.00 | 159.70 | C |
| ATOM | 737 | CD2 | HIS | A | 8 | −5.180 | 45.598 | −65.604 | 1.00 | 159.70 | C |
| ATOM | 738 | ND1 | HIS | A | 8 | −7.268 | 45.136 | −66.237 | 1.00 | 159.70 | N |
| ATOM | 739 | CE1 | HIS | A | 8 | −6.801 | 46.147 | −66.957 | 1.00 | 159.70 | C |
| ATOM | 740 | NE2 | HIS | A | 8 | −5.547 | 46.453 | −66.621 | 1.00 | 159.70 | N |
| ATOM | 741 | N | SER | A | 9 | −8.228 | 42.155 | −62.074 | 1.00 | 154.92 | N |
| ATOM | 742 | CA | SER | A | 9 | −8.541 | 41.065 | −61.129 | 1.00 | 154.92 | C |
| ATOM | 743 | C | SER | A | 9 | −7.425 | 40.033 | −60.991 | 1.00 | 154.92 | C |
| ATOM | 744 | O | SER | A | 9 | −7.248 | 39.212 | −61.895 | 1.00 | 154.92 | O |
| ATOM | 745 | CB | SER | A | 9 | −9.830 | 40.353 | −61.531 | 1.00 | 154.92 | C |
| ATOM | 746 | N | PRO | A | 10 | −6.686 | 40.027 | −59.855 | 1.00 | 150.62 | N |
| ATOM | 747 | CA | PRO | A | 10 | −5.606 | 39.043 | −59.689 | 1.00 | 150.62 | C |
| ATOM | 748 | C | PRO | A | 10 | −6.133 | 37.708 | −59.186 | 1.00 | 150.62 | C |
| ATOM | 749 | O | PRO | A | 10 | −5.368 | 36.748 | −59.045 | 1.00 | 150.62 | O |
| ATOM | 750 | CB | PRO | A | 10 | −4.690 | 39.707 | −58.667 | 1.00 | 150.62 | C |
| ATOM | 751 | CG | PRO | A | 10 | −5.594 | 40.487 | −57.822 | 1.00 | 150.62 | C |
| ATOM | 752 | CD | PRO | A | 10 | −6.765 | 40.918 | −58.683 | 1.00 | 150.62 | C |
| ATOM | 753 | N | ILE | A | 11 | −7.441 | 37.660 | −58.911 | 1.00 | 146.11 | N |
| ATOM | 754 | CA | ILE | A | 11 | −8.132 | 36.464 | −58.472 | 1.00 | 146.11 | C |
| ATOM | 755 | C | ILE | A | 11 | −8.739 | 35.810 | −59.711 | 1.00 | 146.11 | C |
| ATOM | 756 | O | ILE | A | 11 | −9.515 | 36.446 | −60.433 | 1.00 | 146.11 | O |
| ATOM | 757 | CB | ILE | A | 11 | −9.172 | 36.820 | −57.395 | 1.00 | 146.11 | C |
| ATOM | 758 | CG1 | ILE | A | 11 | −8.481 | 37.471 | −56.186 | 1.00 | 146.11 | C |
| ATOM | 759 | CG2 | ILE | A | 11 | −9.972 | 35.580 | −56.987 | 1.00 | 146.11 | C |
| ATOM | 760 | CD1 | ILE | A | 11 | −9.327 | 38.396 | −55.412 | 1.00 | 146.11 | C |
| ATOM | 761 | N | SER | A | 12 | −8.347 | 34.555 | −59.977 | 1.00 | 142.13 | N |
| ATOM | 762 | CA | SER | A | 12 | −8.834 | 33.808 | −61.129 | 1.00 | 142.13 | C |
| ATOM | 763 | C | SER | A | 12 | −10.236 | 33.272 | −60.869 | 1.00 | 142.13 | C |
| ATOM | 764 | O | SER | A | 12 | −10.688 | 33.277 | −59.728 | 1.00 | 142.13 | O |
| ATOM | 765 | CB | SER | A | 12 | −7.889 | 32.657 | −61.448 | 1.00 | 142.13 | C |
| ATOM | 766 | OG | SER | A | 12 | −8.167 | 31.538 | −60.624 | 1.00 | 142.13 | O |
| ATOM | 767 | N | SER | A | 13 | −10.921 | 32.805 | −61.924 | 1.00 | 139.08 | N |
| ATOM | 768 | CA | SER | A | 13 | −12.252 | 32.214 | −61.818 | 1.00 | 139.08 | C |
| ATOM | 769 | C | SER | A | 13 | −12.118 | 30.762 | −61.319 | 1.00 | 139.08 | C |
| ATOM | 770 | O | SER | A | 13 | −12.832 | 30.356 | −60.402 | 1.00 | 139.08 | O |
| ATOM | 771 | CB | SER | A | 13 | −12.990 | 32.270 | −63.161 | 1.00 | 139.08 | C |
| ATOM | 772 | OG | SER | A | 13 | −13.289 | 33.582 | −63.611 | 1.00 | 139.08 | O |
| ATOM | 773 | N | ASP | A | 14 | −11.156 | 30.009 | −61.881 | 1.00 | 137.46 | N |
| ATOM | 774 | CA | ASP | A | 14 | −10.945 | 28.594 | −61.600 | 1.00 | 137.46 | C |
| ATOM | 775 | C | ASP | A | 14 | −9.926 | 28.348 | −60.490 | 1.00 | 137.46 | C |
| ATOM | 776 | O | ASP | A | 14 | −9.169 | 27.378 | −60.566 | 1.00 | 137.46 | O |
| ATOM | 777 | CB | ASP | A | 14 | −10.510 | 27.875 | −62.888 | 1.00 | 137.46 | C |
| ATOM | 778 | N | PHE | A | 15 | −9.926 | 29.185 | −59.443 | 1.00 | 135.86 | N |
| ATOM | 779 | CA | PHE | A | 15 | −8.999 | 29.006 | −58.323 | 1.00 | 135.86 | C |
| ATOM | 780 | C | PHE | A | 15 | −9.483 | 27.901 | −57.394 | 1.00 | 135.86 | C |
| ATOM | 781 | O | PHE | A | 15 | −8.676 | 27.258 | −56.722 | 1.00 | 135.86 | O |
| ATOM | 782 | CB | PHE | A | 15 | −8.832 | 30.317 | −57.542 | 1.00 | 135.86 | C |
| ATOM | 783 | CG | PHE | A | 15 | −10.051 | 30.729 | −56.756 | 1.00 | 135.86 | C |
| ATOM | 784 | CD1 | PHE | A | 15 | −10.346 | 30.136 | −55.533 | 1.00 | 135.86 | C |
| ATOM | 785 | CD2 | PHE | A | 15 | −10.900 | 31.713 | −57.230 | 1.00 | 135.86 | C |
| ATOM | 786 | CE1 | PHE | A | 15 | −11.479 | 30.508 | −54.807 | 1.00 | 135.86 | C |
| ATOM | 787 | CE2 | PHE | A | 15 | −12.023 | 32.101 | −56.495 | 1.00 | 135.86 | C |
| ATOM | 788 | CZ | PHE | A | 15 | −12.301 | 31.498 | −55.285 | 1.00 | 135.86 | C |
| ATOM | 789 | N | ALA | A | 16 | −10.811 | 27.707 | −57.337 | 1.00 | 133.89 | N |
| ATOM | 790 | CA | ALA | A | 16 | −11.448 | 26.716 | −56.479 | 1.00 | 133.89 | C |
| ATOM | 791 | C | ALA | A | 16 | −11.138 | 25.319 | −56.977 | 1.00 | 133.89 | C |
| ATOM | 792 | O | ALA | A | 16 | −11.240 | 24.344 | −56.235 | 1.00 | 133.89 | O |
| ATOM | 793 | CB | ALA | A | 16 | −12.949 | 26.950 | −56.459 | 1.00 | 133.89 | C |
| ATOM | 794 | N | VAL | A | 17 | −10.740 | 25.231 | −58.233 | 1.00 | 130.93 | N |
| ATOM | 795 | CA | VAL | A | 17 | −10.433 | 23.967 | −58.841 | 1.00 | 130.93 | C |
| ATOM | 796 | C | VAL | A | 17 | −9.027 | 23.551 | −58.457 | 1.00 | 130.93 | C |
| ATOM | 797 | O | VAL | A | 17 | −8.817 | 22.388 | −58.131 | 1.00 | 130.93 | O |
| ATOM | 798 | CB | VAL | A | 17 | −10.676 | 24.000 | −60.361 | 1.00 | 130.93 | C |
| ATOM | 799 | CG1 | VAL | A | 17 | −11.739 | 25.029 | −60.746 | 1.00 | 130.93 | C |
| ATOM | 800 | CG2 | VAL | A | 17 | −9.407 | 24.164 | −61.184 | 1.00 | 130.93 | C |
| ATOM | 801 | N | LYS | A | 18 | −8.081 | 24.504 | −58.437 | 1.00 | 132.98 | N |
| ATOM | 802 | CA | LYS | A | 18 | −6.692 | 24.233 | −58.081 | 1.00 | 132.98 | C |
| ATOM | 803 | C | LYS | A | 18 | −6.570 | 23.852 | −56.616 | 1.00 | 132.98 | C |
| ATOM | 804 | O | LYS | A | 18 | −5.620 | 23.165 | −56.245 | 1.00 | 132.98 | O |
| ATOM | 805 | CB | LYS | A | 18 | −5.809 | 25.450 | −58.382 | 1.00 | 132.98 | C |
| ATOM | 806 | N | ILE | A | 19 | −7.535 | 24.274 | −55.792 | 1.00 | 135.42 | N |
| ATOM | 807 | CA | ILE | A | 19 | −7.520 | 23.990 | −54.361 | 1.00 | 135.42 | C |
| ATOM | 808 | C | ILE | A | 19 | −8.127 | 22.624 | −54.103 | 1.00 | 135.42 | C |
| ATOM | 809 | O | ILE | A | 19 | −7.710 | 21.933 | −53.174 | 1.00 | 135.42 | O |
| ATOM | 810 | CB | ILE | A | 19 | −8.224 | 25.098 | −53.552 | 1.00 | 135.42 | C |
| ATOM | 811 | CG1 | ILE | A | 19 | −7.597 | 26.455 | −53.861 | 1.00 | 135.42 | C |
| ATOM | 812 | CG2 | ILE | A | 19 | −8.150 | 24.809 | −52.044 | 1.00 | 135.42 | C |
| ATOM | 813 | CD1 | ILE | A | 19 | −8.245 | 27.604 | −53.171 | 1.00 | 135.42 | C |
| ATOM | 814 | N | ARG | A | 20 | −9.111 | 22.235 | −54.917 | 1.00 | 141.42 | N |
| ATOM | 815 | CA | ARG | A | 20 | −9.749 | 20.934 | −54.794 | 1.00 | 141.42 | C |
| ATOM | 816 | C | ARG | A | 20 | −8.778 | 19.888 | −55.295 | 1.00 | 141.42 | C |
| ATOM | 817 | O | ARG | A | 20 | −8.769 | 18.768 | −54.797 | 1.00 | 141.42 | O |
| ATOM | 818 | CB | ARG | A | 20 | −11.068 | 20.896 | −55.581 | 1.00 | 141.42 | C |
| ATOM | 819 | N | GLU | A | 21 | −7.935 | 20.270 | −56.260 | 1.00 | 145.72 | N |
| ATOM | 820 | CA | GLU | A | 21 | −6.944 | 19.373 | −56.826 | 1.00 | 145.72 | C |
| ATOM | 821 | C | GLU | A | 21 | −5.814 | 19.152 | −55.849 | 1.00 | 145.72 | C |
| ATOM | 822 | O | GLU | A | 21 | −5.315 | 18.034 | −55.759 | 1.00 | 145.72 | O |
| ATOM | 823 | CB | GLU | A | 21 | −6.420 | 19.916 | −58.148 | 1.00 | 145.72 | C |
| ATOM | 824 | CG | GLU | A | 21 | −7.133 | 19.312 | −59.346 | 1.00 | 145.72 | C |
| ATOM | 825 | CD | GLU | A | 21 | −7.273 | 20.227 | −60.555 | 1.00 | 145.72 | C |
| ATOM | 826 | OE1 | GLU | A | 21 | −6.230 | 20.699 | −61.072 | 1.00 | 145.72 | O |
| ATOM | 827 | OE2 | GLU | A | 21 | −8.424 | 20.449 | −61.007 | 1.00 | 145.72 | O + 1 |
| ATOM | 828 | N | LEU | A | 22 | −5.415 | 20.201 | −55.110 | 1.00 | 147.14 | N |
| ATOM | 829 | CA | LEU | A | 22 | −4.363 | 20.083 | −54.107 | 1.00 | 147.14 | C |
| ATOM | 830 | C | LEU | A | 22 | −4.871 | 19.221 | −52.977 | 1.00 | 147.14 | C |
| ATOM | 831 | O | LEU | A | 22 | −4.189 | 18.292 | −52.553 | 1.00 | 147.14 | O |
| ATOM | 832 | CB | LEU | A | 22 | −3.935 | 21.461 | −53.585 | 1.00 | 147.14 | C |
| ATOM | 833 | CG | LEU | A | 22 | −3.155 | 21.473 | −52.275 | 1.00 | 147.14 | C |
| ATOM | 834 | CD1 | LEU | A | 22 | −1.800 | 20.894 | −52.445 | 1.00 | 147.14 | C |
| ATOM | 835 | CD2 | LEU | A | 22 | −3.012 | 22.852 | −51.747 | 1.00 | 147.14 | C |
| ATOM | 836 | N | SER | A | 23 | −6.087 | 19.514 | −52.517 | 1.00 | 151.36 | N |
| ATOM | 837 | CA | SER | A | 23 | −6.723 | 18.790 | −51.435 | 1.00 | 151.36 | C |
| ATOM | 838 | C | SER | A | 23 | −6.881 | 17.299 | −51.755 | 1.00 | 151.36 | C |
| ATOM | 839 | O | SER | A | 23 | −6.873 | 16.483 | −50.839 | 1.00 | 151.36 | O |
| ATOM | 840 | CB | SER | A | 23 | −8.077 | 19.407 | −51.131 | 1.00 | 151.36 | C |
| ATOM | 841 | OG | SER | A | 23 | −8.804 | 18.608 | −50.217 | 1.00 | 151.36 | O |
| ATOM | 842 | N | ASP | A | 24 | −6.989 | 16.935 | −53.038 | 1.00 | 156.26 | N |
| ATOM | 843 | CA | ASP | A | 24 | −7.142 | 15.538 | −53.448 | 1.00 | 156.26 | C |
| ATOM | 844 | C | ASP | A | 24 | −5.890 | 14.695 | −53.227 | 1.00 | 156.26 | C |
| ATOM | 845 | O | ASP | A | 24 | −5.969 | 13.469 | −53.278 | 1.00 | 156.26 | O |
| ATOM | 846 | CB | ASP | A | 24 | −7.542 | 15.468 | −54.915 | 1.00 | 156.26 | C |
| ATOM | 847 | CG | ASP | A | 24 | −9.020 | 15.682 | −55.147 | 1.00 | 156.26 | C |
| ATOM | 848 | OD1 | ASP | A | 24 | −9.760 | 15.861 | −54.147 | 1.00 | 156.26 | O |
| ATOM | 849 | OD2 | ASP | A | 24 | −9.441 | 15.693 | −56.328 | 1.00 | 156.26 | O + 1 |
| ATOM | 850 | N | TYR | A | 25 | −4.752 | 15.336 | −52.966 | 1.00 | 157.45 | N |
| ATOM | 851 | CA | TYR | A | 25 | −3.499 | 14.637 | −52.735 | 1.00 | 157.45 | C |
| ATOM | 852 | C | TYR | A | 25 | −2.916 | 14.997 | −51.375 | 1.00 | 157.45 | C |
| ATOM | 853 | O | TYR | A | 25 | −1.769 | 14.674 | −51.072 | 1.00 | 157.45 | O |
| ATOM | 854 | CB | TYR | A | 25 | −2.519 | 14.975 | −53.848 | 1.00 | 157.45 | C |
| ATOM | 855 | CG | TYR | A | 25 | −2.914 | 14.402 | −55.190 | 1.00 | 157.45 | C |
| ATOM | 856 | CD1 | TYR | A | 25 | −3.432 | 15.216 | −56.193 | 1.00 | 157.45 | C |
| ATOM | 857 | CD2 | TYR | A | 25 | −2.744 | 13.050 | −55.472 | 1.00 | 157.45 | C |
| ATOM | 858 | CE1 | TYR | A | 25 | −3.726 | 14.711 | −57.460 | 1.00 | 157.45 | C |
| ATOM | 859 | CE2 | TYR | A | 25 | −3.033 | 12.533 | −56.736 | 1.00 | 157.45 | C |
| ATOM | 860 | CZ | TYR | A | 25 | −3.530 | 13.367 | −57.726 | 1.00 | 157.45 | C |
| ATOM | 861 | OH | TYR | A | 25 | −3.826 | 12.873 | −58.973 | 1.00 | 157.45 | O |
| ATOM | 862 | N | LEU | A | 26 | −3.719 | 15.635 | −50.539 | 1.00 | 161.33 | N |
| ATOM | 863 | CA | LEU | A | 26 | −3.274 | 16.036 | −49.222 | 1.00 | 161.33 | C |
| ATOM | 864 | C | LEU | A | 26 | −3.778 | 15.147 | −48.125 | 1.00 | 161.33 | C |
| ATOM | 865 | O | LEU | A | 26 | −4.883 | 14.587 | −48.193 | 1.00 | 161.33 | O |
| ATOM | 866 | CB | LEU | A | 26 | −3.739 | 17.464 | −48.912 | 1.00 | 161.33 | C |
| ATOM | 867 | CG | LEU | A | 26 | −2.907 | 18.595 | −49.467 | 1.00 | 161.33 | C |
| ATOM | 868 | CD1 | LEU | A | 26 | −3.439 | 19.914 | −48.993 | 1.00 | 161.33 | C |
| ATOM | 869 | CD2 | LEU | A | 26 | −1.481 | 18.497 | −49.018 | 1.00 | 161.33 | C |
| ATOM | 870 | N | LEU | A | 27 | −2.946 | 15.057 | −47.093 | 1.00 | 164.65 | N |
| ATOM | 871 | CA | LEU | A | 27 | −3.244 | 14.462 | −45.810 | 1.00 | 164.65 | C |
| ATOM | 872 | C | LEU | A | 27 | −3.843 | 15.607 | −45.017 | 1.00 | 164.65 | C |
| ATOM | 873 | O | LEU | A | 27 | −3.119 | 16.489 | −44.541 | 1.00 | 164.65 | O |
| ATOM | 874 | CB | LEU | A | 27 | −1.971 | 13.896 | −45.173 | 1.00 | 164.65 | C |
| ATOM | 875 | CG | LEU | A | 27 | −1.357 | 12.683 | −45.865 | 1.00 | 164.65 | C |
| ATOM | 876 | CD1 | LEU | A | 27 | −0.112 | 12.234 | −45.177 | 1.00 | 164.65 | C |
| ATOM | 877 | CD2 | LEU | A | 27 | −2.318 | 11.530 | −45.914 | 1.00 | 164.65 | C |
| ATOM | 878 | N | GLN | A | 28 | −5.176 | 15.647 | −44.972 | 1.00 | 166.78 | N |
| ATOM | 879 | CA | GLN | A | 28 | −6.001 | 16.720 | −44.421 | 1.00 | 166.78 | C |
| ATOM | 880 | C | GLN | A | 28 | −5.640 | 17.254 | −43.026 | 1.00 | 166.78 | C |
| ATOM | 881 | O | GLN | A | 28 | −5.956 | 18.412 | −42.751 | 1.00 | 166.78 | O |
| ATOM | 882 | CB | GLN | A | 28 | −7.459 | 16.270 | −44.378 | 1.00 | 166.78 | C |
| ATOM | 883 | CG | GLN | A | 28 | −8.310 | 16.740 | −45.541 | 1.00 | 166.78 | C |
| ATOM | 884 | CD | GLN | A | 28 | −7.764 | 16.314 | −46.871 | 1.00 | 166.78 | C |
| ATOM | 885 | NE2 | GLN | A | 28 | −7.570 | 17.290 | −47.753 | 1.00 | 166.78 | N |
| ATOM | 886 | OE1 | GLN | A | 28 | −7.489 | 15.126 | −47.107 | 1.00 | 166.78 | O |
| ATOM | 887 | N | ASP | A | 29 | −5.034 | 16.453 | −42.142 | 1.00 | 168.93 | N |
| ATOM | 888 | CA | ASP | A | 29 | −4.747 | 16.968 | −40.801 | 1.00 | 168.93 | C |
| ATOM | 889 | C | ASP | A | 29 | −3.297 | 17.389 | −40.629 | 1.00 | 168.93 | C |
| ATOM | 890 | O | ASP | A | 29 | −2.814 | 17.482 | −39.491 | 1.00 | 168.93 | O |
| ATOM | 891 | CB | ASP | A | 29 | −5.156 | 15.964 | −39.706 | 1.00 | 168.93 | C |
| ATOM | 892 | CG | ASP | A | 29 | −4.336 | 14.679 | −39.658 | 1.00 | 168.93 | C |
| ATOM | 893 | OD1 | ASP | A | 29 | −3.656 | 14.357 | −40.669 | 1.00 | 168.93 | O |
| ATOM | 894 | OD2 | ASP | A | 29 | −4.395 | 13.977 | −38.623 | 1.00 | 168.93 | O + 1 |
| ATOM | 895 | N | TYR | A | 30 | −2.605 | 17.654 | −41.748 | 1.00 | 166.35 | N |
| ATOM | 896 | CA | TYR | A | 30 | −1.235 | 18.132 | −41.683 | 1.00 | 166.35 | C |
| ATOM | 897 | C | TYR | A | 30 | −1.263 | 19.426 | −40.884 | 1.00 | 166.35 | C |
| ATOM | 898 | O | TYR | A | 30 | −2.086 | 20.292 | −41.187 | 1.00 | 166.35 | O |
| ATOM | 899 | CB | TYR | A | 30 | −0.652 | 18.339 | −43.084 | 1.00 | 166.35 | C |
| ATOM | 900 | CG | TYR | A | 30 | 0.729 | 18.961 | −43.084 | 1.00 | 166.35 | C |
| ATOM | 901 | CD1 | TYR | A | 30 | 1.865 | 18.180 | −43.254 | 1.00 | 166.35 | C |
| ATOM | 902 | CD2 | TYR | A | 30 | 0.898 | 20.334 | −42.927 | 1.00 | 166.35 | C |
| ATOM | 903 | CE1 | TYR | A | 30 | 3.133 | 18.747 | −43.289 | 1.00 | 166.35 | C |
| ATOM | 904 | CE2 | TYR | A | 30 | 2.165 | 20.907 | −42.913 | 1.00 | 166.35 | C |
| ATOM | 905 | CZ | TYR | A | 30 | 3.280 | 20.109 | −43.111 | 1.00 | 166.35 | C |
| ATOM | 906 | OH | TYR | A | 30 | 4.538 | 20.657 | −43.130 | 1.00 | 166.35 | O |
| ATOM | 907 | N | PRO | A | 31 | −0.410 | 19.602 | −39.863 | 1.00 | 162.90 | N |
| ATOM | 908 | CA | PRO | A | 31 | −0.506 | 20.824 | −39.065 | 1.00 | 162.90 | C |
| ATOM | 909 | C | PRO | A | 31 | 0.118 | 22.071 | −39.698 | 1.00 | 162.90 | C |
| ATOM | 910 | O | PRO | A | 31 | 1.309 | 22.131 | −40.006 | 1.00 | 162.90 | O |
| ATOM | 911 | CB | PRO | A | 31 | 0.174 | 20.453 | −37.757 | 1.00 | 162.90 | C |
| ATOM | 912 | CG | PRO | A | 31 | 1.141 | 19.407 | −38.127 | 1.00 | 162.90 | C |
| ATOM | 913 | CD | PRO | A | 31 | 0.618 | 18.689 | −39.334 | 1.00 | 162.90 | C |
| ATOM | 914 | N | VAL | A | 32 | −0.733 | 23.082 | −39.866 | 1.00 | 161.19 | N |
| ATOM | 915 | CA | VAL | A | 32 | −0.391 | 24.423 | −40.331 | 1.00 | 161.19 | C |
| ATOM | 916 | C | VAL | A | 32 | −0.826 | 25.356 | −39.203 | 1.00 | 161.19 | C |
| ATOM | 917 | O | VAL | A | 32 | −1.238 | 24.871 | −38.147 | 1.00 | 161.19 | O |
| ATOM | 918 | CB | VAL | A | 32 | −1.059 | 24.783 | −41.685 | 1.00 | 161.19 | C |
| ATOM | 919 | CG1 | VAL | A | 32 | −0.493 | 23.959 | −42.832 | 1.00 | 161.19 | C |
| ATOM | 920 | CG2 | VAL | A | 32 | −2.572 | 24.642 | −41.605 | 1.00 | 161.19 | C |
| ATOM | 921 | N | THR | A | 33 | −0.772 | 26.675 | −39.418 | 1.00 | 159.72 | N |
| ATOM | 922 | CA | THR | A | 33 | −1.244 | 27.643 | −38.430 | 1.00 | 159.72 | C |
| ATOM | 923 | C | THR | A | 33 | −2.101 | 28.693 | −39.085 | 1.00 | 159.72 | C |
| ATOM | 924 | O | THR | A | 33 | −2.040 | 28.901 | −40.294 | 1.00 | 159.72 | O |
| ATOM | 925 | CB | THR | A | 33 | −0.103 | 28.323 | −37.655 | 1.00 | 159.72 | C |
| ATOM | 926 | CG2 | THR | A | 33 | 0.831 | 27.336 | −37.008 | 1.00 | 159.72 | C |
| ATOM | 927 | OG1 | THR | A | 33 | 0.620 | 29.210 | −38.511 | 1.00 | 159.72 | O |
| ATOM | 928 | N | VAL | A | 34 | −2.884 | 29.367 | −38.273 | 1.00 | 161.95 | N |
| ATOM | 929 | CA | VAL | A | 34 | −3.754 | 30.459 | −38.675 | 1.00 | 161.95 | C |
| ATOM | 930 | C | VAL | A | 34 | −3.652 | 31.500 | −37.599 | 1.00 | 161.95 | C |
| ATOM | 931 | O | VAL | A | 34 | −3.471 | 31.125 | −36.436 | 1.00 | 161.95 | O |
| ATOM | 932 | CB | VAL | A | 34 | −5.215 | 29.999 | −38.871 | 1.00 | 161.95 | C |
| ATOM | 933 | CG1 | VAL | A | 34 | −5.400 | 29.296 | −40.201 | 1.00 | 161.95 | C |
| ATOM | 934 | CG2 | VAL | A | 34 | −5.683 | 29.124 | −37.719 | 1.00 | 161.95 | C |
| ATOM | 935 | N | ALA | A | 35 | −3.775 | 32.789 | −37.939 | 1.00 | 164.70 | N |
| ATOM | 936 | CA | ALA | A | 35 | −3.743 | 33.798 | −36.888 | 1.00 | 164.70 | C |
| ATOM | 937 | C | ALA | A | 35 | −4.999 | 33.645 | −36.068 | 1.00 | 164.70 | C |
| ATOM | 938 | O | ALA | A | 35 | −6.080 | 33.479 | −36.636 | 1.00 | 164.70 | O |
| ATOM | 939 | CB | ALA | A | 35 | −3.645 | 35.191 | −37.476 | 1.00 | 164.70 | C |
| ATOM | 940 | N | SER | A | 36 | −4.857 | 33.632 | −34.746 | 1.00 | 168.33 | N |
| ATOM | 941 | CA | SER | A | 36 | −5.993 | 33.466 | −33.862 | 1.00 | 168.33 | C |
| ATOM | 942 | C | SER | A | 36 | −6.612 | 34.817 | −33.475 | 1.00 | 168.33 | C |
| ATOM | 943 | O | SER | A | 36 | −7.810 | 34.885 | −33.250 | 1.00 | 168.33 | O |
| ATOM | 944 | CB | SER | A | 36 | −5.588 | 32.680 | −32.618 | 1.00 | 168.33 | C |
| ATOM | 945 | OG | SER | A | 36 | −4.652 | 33.383 | −31.817 | 1.00 | 168.33 | O |
| ATOM | 946 | N | ASN | A | 37 | −5.827 | 35.895 | −33.428 | 1.00 | 171.90 | N |
| ATOM | 947 | CA | ASN | A | 37 | −6.369 | 37.179 | −32.986 | 1.00 | 171.90 | C |
| ATOM | 948 | C | ASN | A | 37 | −6.231 | 38.305 | −34.016 | 1.00 | 171.90 | C |
| ATOM | 949 | O | ASN | A | 37 | −5.745 | 39.394 | −33.684 | 1.00 | 171.90 | O |
| ATOM | 950 | CB | ASN | A | 37 | −5.685 | 37.584 | −31.681 | 1.00 | 171.90 | C |
| ATOM | 951 | CG | ASN | A | 37 | −4.222 | 37.915 | −31.850 | 1.00 | 171.90 | C |
| ATOM | 952 | ND2 | ASN | A | 37 | −3.735 | 38.845 | −31.050 | 1.00 | 171.90 | N |
| ATOM | 953 | OD1 | ASN | A | 37 | −3.538 | 37.385 | −32.731 | 1.00 | 171.90 | O |
| ATOM | 954 | N | LEU | A | 38 | −6.685 | 38.066 | −35.248 | 1.00 | 174.92 | N |
| ATOM | 955 | CA | LEU | A | 38 | −6.642 | 39.116 | −36.258 | 1.00 | 174.92 | C |
| ATOM | 956 | C | LEU | A | 38 | −7.794 | 40.052 | −36.024 | 1.00 | 174.92 | C |
| ATOM | 957 | O | LEU | A | 38 | −8.932 | 39.599 | −35.894 | 1.00 | 174.92 | O |
| ATOM | 958 | CB | LEU | A | 38 | −6.690 | 38.565 | −37.692 | 1.00 | 174.92 | C |
| ATOM | 959 | CG | LEU | A | 38 | −5.363 | 38.247 | −38.370 | 1.00 | 174.92 | C |
| ATOM | 960 | CD1 | LEU | A | 38 | −5.607 | 37.620 | −39.691 | 1.00 | 174.92 | C |
| ATOM | 961 | CD2 | LEU | A | 38 | −4.504 | 39.487 | −38.554 | 1.00 | 174.92 | C |
| ATOM | 962 | N | GLN | A | 39 | −7.500 | 41.353 | −35.936 | 1.00 | 178.71 | N |
| ATOM | 963 | CA | GLN | A | 39 | −8.503 | 42.387 | −35.740 | 1.00 | 178.71 | C |
| ATOM | 964 | C | GLN | A | 39 | −9.610 | 42.223 | −36.757 | 1.00 | 178.71 | C |
| ATOM | 965 | O | GLN | A | 39 | −9.341 | 41.848 | −37.898 | 1.00 | 178.71 | O |
| ATOM | 966 | CB | GLN | A | 39 | −7.850 | 43.771 | −35.854 | 1.00 | 178.71 | C |
| ATOM | 967 | CG | GLN | A | 39 | −8.828 | 44.923 | −36.003 | 1.00 | 178.71 | C |
| ATOM | 968 | CD | GLN | A | 39 | −8.116 | 46.202 | −36.325 | 1.00 | 178.71 | C |
| ATOM | 969 | NE2 | GLN | A | 39 | −8.197 | 46.655 | −37.573 | 1.00 | 178.71 | N |
| ATOM | 970 | OE1 | GLN | A | 39 | −7.473 | 46.798 | −35.456 | 1.00 | 178.71 | O |
| ATOM | 971 | N | ASP | A | 40 | −10.856 | 42.459 | −36.340 | 1.00 | 183.25 | N |
| ATOM | 972 | CA | ASP | A | 40 | −11.986 | 42.376 | −37.260 | 1.00 | 183.25 | C |
| ATOM | 973 | C | ASP | A | 40 | −11.959 | 43.592 | −38.160 | 1.00 | 183.25 | C |
| ATOM | 974 | O | ASP | A | 40 | −12.146 | 44.718 | −37.692 | 1.00 | 183.25 | O |
| ATOM | 975 | CB | ASP | A | 40 | −13.320 | 42.258 | −36.510 | 1.00 | 183.25 | C |
| ATOM | 976 | CG | ASP | A | 40 | −13.605 | 40.867 | −35.960 | 1.00 | 183.25 | C |
| ATOM | 977 | OD1 | ASP | A | 40 | −12.921 | 39.906 | −36.379 | 1.00 | 183.25 | O |
| ATOM | 978 | OD2 | ASP | A | 40 | −14.516 | 40.738 | −35.108 | 1.00 | 183.25 | O + 1 |
| ATOM | 979 | N | ASP | A | 41 | −11.638 | 43.369 | −39.435 | 1.00 | 182.51 | N |
| ATOM | 980 | CA | ASP | A | 41 | −11.520 | 44.438 | −40.418 | 1.00 | 182.51 | C |
| ATOM | 981 | C | ASP | A | 41 | −12.039 | 43.939 | −41.751 | 1.00 | 182.51 | C |
| ATOM | 982 | O | ASP | A | 41 | −11.279 | 43.401 | −42.555 | 1.00 | 182.51 | O |
| ATOM | 983 | CB | ASP | A | 41 | −10.052 | 44.911 | −40.526 | 1.00 | 182.51 | C |
| ATOM | 984 | CG | ASP | A | 41 | −9.871 | 46.277 | −41.151 | 1.00 | 182.51 | C |
| ATOM | 985 | OD1 | ASP | A | 41 | −10.709 | 46.660 | −42.006 | 1.00 | 182.51 | O |
| ATOM | 986 | OD2 | ASP | A | 41 | −8.887 | 46.966 | −40.793 | 1.00 | 182.51 | O + 1 |
| ATOM | 987 | N | GLU | A | 42 | −13.322 | 44.165 | −42.011 | 1.00 | 179.48 | N |
| ATOM | 988 | CA | GLU | A | 42 | −13.994 | 43.701 | −43.221 | 1.00 | 179.48 | C |
| ATOM | 989 | C | GLU | A | 42 | −13.332 | 44.110 | −44.564 | 1.00 | 179.48 | C |
| ATOM | 990 | O | GLU | A | 42 | −13.758 | 43.622 | −45.614 | 1.00 | 179.48 | O |
| ATOM | 991 | CB | GLU | A | 42 | −15.433 | 44.185 | −43.200 | 1.00 | 179.48 | C |
| ATOM | 992 | N | LEU | A | 43 | −12.272 | 44.931 | −44.533 | 1.00 | 175.07 | N |
| ATOM | 993 | CA | LEU | A | 43 | −11.585 | 45.340 | −45.748 | 1.00 | 175.07 | C |
| ATOM | 994 | C | LEU | A | 43 | −10.243 | 44.614 | −45.884 | 1.00 | 175.07 | C |
| ATOM | 995 | O | LEU | A | 43 | −9.891 | 44.175 | −46.982 | 1.00 | 175.07 | O |
| ATOM | 996 | CB | LEU | A | 43 | −11.390 | 46.876 | −45.759 | 1.00 | 175.07 | C |
| ATOM | 997 | CG | LEU | A | 43 | −10.245 | 47.435 | −46.638 | 1.00 | 175.07 | C |
| ATOM | 998 | CD1 | LEU | A | 43 | −10.660 | 47.584 | −48.097 | 1.00 | 175.07 | C |
| ATOM | 999 | CD2 | LEU | A | 43 | −9.725 | 48.747 | −46.110 | 1.00 | 175.07 | C |
| ATOM | 1000 | N | CYS | A | 44 | −9.500 | 44.500 | −44.775 | 1.00 | 169.01 | N |
| ATOM | 1001 | CA | CYS | A | 44 | −8.171 | 43.907 | −44.779 | 1.00 | 169.01 | C |
| ATOM | 1002 | C | CYS | A | 44 | −8.166 | 42.453 | −44.337 | 1.00 | 169.01 | C |
| ATOM | 1003 | O | CYS | A | 44 | −7.234 | 41.730 | −44.673 | 1.00 | 169.01 | O |
| ATOM | 1004 | CB | CYS | A | 44 | −7.222 | 44.731 | −43.918 | 1.00 | 169.01 | C |
| ATOM | 1005 | SG | CYS | A | 44 | −7.014 | 46.447 | −44.467 | 1.00 | 169.01 | S |
| ATOM | 1006 | N | GLY | A | 45 | −9.176 | 42.050 | −43.576 | 1.00 | 162.29 | N |
| ATOM | 1007 | CA | GLY | A | 45 | −9.323 | 40.705 | −43.018 | 1.00 | 162.29 | C |
| ATOM | 1008 | C | GLY | A | 45 | −8.874 | 39.527 | −43.855 | 1.00 | 162.29 | C |
| ATOM | 1009 | O | GLY | A | 45 | −7.887 | 38.869 | −43.521 | 1.00 | 162.29 | O |
| ATOM | 1010 | N | GLY | A | 46 | −9.599 | 39.261 | −44.929 | 1.00 | 154.64 | N |
| ATOM | 1011 | CA | GLY | A | 46 | −9.285 | 38.162 | −45.829 | 1.00 | 154.64 | C |
| ATOM | 1012 | C | GLY | A | 46 | −7.910 | 38.298 | −46.442 | 1.00 | 154.64 | C |
| ATOM | 1013 | O | GLY | A | 46 | −7.202 | 37.305 | −46.612 | 1.00 | 154.64 | O |
| ATOM | 1014 | N | LEU | A | 47 | −7.522 | 39.544 | −46.744 | 1.00 | 146.30 | N |
| ATOM | 1015 | CA | LEU | A | 47 | −6.234 | 39.899 | −47.324 | 1.00 | 146.30 | C |
| ATOM | 1016 | C | LEU | A | 47 | −5.087 | 39.544 | −46.370 | 1.00 | 146.30 | C |
| ATOM | 1017 | O | LEU | A | 47 | −4.068 | 39.027 | −46.810 | 1.00 | 146.30 | O |
| ATOM | 1018 | CB | LEU | A | 47 | −6.234 | 41.397 | −47.644 | 1.00 | 146.30 | C |
| ATOM | 1019 | CG | LEU | A | 47 | −5.251 | 41.889 | −48.673 | 1.00 | 146.30 | C |
| ATOM | 1020 | CD1 | LEU | A | 47 | −5.491 | 41.236 | −49.997 | 1.00 | 146.30 | C |
| ATOM | 1021 | CD2 | LEU | A | 47 | −5.366 | 43.378 | −48.831 | 1.00 | 146.30 | C |
| ATOM | 1022 | N | TRP | A | 48 | −5.263 | 39.795 | −45.075 | 1.00 | 141.37 | N |
| ATOM | 1023 | CA | TRP | A | 48 | −4.254 | 39.481 | −44.074 | 1.00 | 141.37 | C |
| ATOM | 1024 | C | TRP | A | 48 | −4.027 | 37.985 | −43.999 | 1.00 | 141.37 | C |
| ATOM | 1025 | O | TRP | A | 48 | −2.891 | 37.526 | −43.953 | 1.00 | 141.37 | O |
| ATOM | 1026 | CB | TRP | A | 48 | −4.700 | 40.004 | −42.702 | 1.00 | 141.37 | C |
| ATOM | 1027 | CG | TRP | A | 48 | −4.556 | 41.486 | −42.482 | 1.00 | 141.37 | C |
| ATOM | 1028 | CD1 | TRP | A | 48 | −3.685 | 42.330 | −43.104 | 1.00 | 141.37 | C |
| ATOM | 1029 | CD2 | TRP | A | 48 | −5.274 | 42.283 | −41.525 | 1.00 | 141.37 | C |
| ATOM | 1030 | CE2 | TRP | A | 48 | −4.788 | 43.602 | −41.632 | 1.00 | 141.37 | C |
| ATOM | 1031 | CE3 | TRP | A | 48 | −6.282 | 42.007 | −40.583 | 1.00 | 141.37 | C |
| ATOM | 1032 | NE1 | TRP | A | 48 | −3.823 | 43.603 | −42.605 | 1.00 | 141.37 | N |
| ATOM | 1033 | CZ2 | TRP | A | 48 | −5.279 | 44.646 | −40.840 | 1.00 | 141.37 | C |
| ATOM | 1034 | CZ3 | TRP | A | 48 | −6.770 | 43.041 | −39.804 | 1.00 | 141.37 | C |
| ATOM | 1035 | CH2 | TRP | A | 48 | −6.277 | 44.343 | −39.940 | 1.00 | 141.37 | C |
| ATOM | 1036 | N | ARG | A | 49 | −5.118 | 37.228 | −43.998 | 1.00 | 139.14 | N |
| ATOM | 1037 | CA | ARG | A | 49 | −5.087 | 35.774 | −43.911 | 1.00 | 139.14 | C |
| ATOM | 1038 | C | ARG | A | 49 | −4.531 | 35.159 | −45.190 | 1.00 | 139.14 | C |
| ATOM | 1039 | O | ARG | A | 49 | −3.867 | 34.127 | −45.123 | 1.00 | 139.14 | O |
| ATOM | 1040 | CB | ARG | A | 49 | −6.487 | 35.219 | −43.608 | 1.00 | 139.14 | C |
| ATOM | 1041 | CG | ARG | A | 49 | −7.300 | 36.057 | −42.612 | 1.00 | 139.14 | C |
| ATOM | 1042 | CD | ARG | A | 49 | −8.808 | 35.810 | −42.650 | 1.00 | 139.14 | C |
| ATOM | 1043 | NE | ARG | A | 49 | −9.224 | 34.727 | −41.747 | 1.00 | 139.14 | N |
| ATOM | 1044 | CZ | ARG | A | 49 | −9.348 | 34.841 | −40.421 | 1.00 | 139.14 | C |
| ATOM | 1045 | NH1 | ARG | A | 49 | −9.073 | 35.993 | −39.816 | 1.00 | 139.14 | N + 1 |
| ATOM | 1046 | NH2 | ARG | A | 49 | −9.729 | 33.797 | −39.690 | 1.00 | 139.14 | N |
| ATOM | 1047 | N | LEU | A | 50 | −4.802 | 35.784 | −46.348 | 1.00 | 134.28 | N |
| ATOM | 1048 | CA | LEU | A | 50 | −4.278 | 35.308 | −47.622 | 1.00 | 134.28 | C |
| ATOM | 1049 | C | LEU | A | 50 | −2.779 | 35.487 | −47.647 | 1.00 | 134.28 | C |
| ATOM | 1050 | O | LEU | A | 50 | −2.069 | 34.635 | −48.158 | 1.00 | 134.28 | O |
| ATOM | 1051 | CB | LEU | A | 50 | −4.914 | 36.043 | −48.799 | 1.00 | 134.28 | C |
| ATOM | 1052 | CG | LEU | A | 50 | −6.263 | 35.524 | −49.279 | 1.00 | 134.28 | C |
| ATOM | 1053 | CD1 | LEU | A | 50 | −6.772 | 36.364 | −50.412 | 1.00 | 134.28 | C |
| ATOM | 1054 | CD2 | LEU | A | 50 | −6.181 | 34.075 | −49.716 | 1.00 | 134.28 | C |
| ATOM | 1055 | N | VAL | A | 51 | −2.295 | 36.580 | −47.067 | 1.00 | 130.97 | N |
| ATOM | 1056 | CA | VAL | A | 51 | −0.864 | 36.853 | −46.971 | 1.00 | 130.97 | C |
| ATOM | 1057 | C | VAL | A | 51 | −0.209 | 35.763 | −46.139 | 1.00 | 130.97 | C |
| ATOM | 1058 | O | VAL | A | 51 | 0.855 | 35.267 | −46.491 | 1.00 | 130.97 | O |
| ATOM | 1059 | CB | VAL | A | 51 | −0.634 | 38.245 | −46.343 | 1.00 | 130.97 | C |
| ATOM | 1060 | CG1 | VAL | A | 51 | 0.827 | 38.454 | −45.967 | 1.00 | 130.97 | C |
| ATOM | 1061 | CG2 | VAL | A | 51 | −1.109 | 39.351 | −47.268 | 1.00 | 130.97 | C |
| ATOM | 1062 | N | LEU | A | 52 | −0.854 | 35.408 | −45.032 | 1.00 | 129.47 | N |
| ATOM | 1063 | CA | LEU | A | 52 | −0.382 | 34.386 | −44.115 | 1.00 | 129.47 | C |
| ATOM | 1064 | C | LEU | A | 52 | −0.461 | 33.016 | −44.749 | 1.00 | 129.47 | C |
| ATOM | 1065 | O | LEU | A | 52 | 0.484 | 32.235 | −44.642 | 1.00 | 129.47 | O |
| ATOM | 1066 | CB | LEU | A | 52 | −1.204 | 34.416 | −42.826 | 1.00 | 129.47 | C |
| ATOM | 1067 | CG | LEU | A | 52 | −1.102 | 35.682 | −41.979 | 1.00 | 129.47 | C |
| ATOM | 1068 | CD1 | LEU | A | 52 | −2.122 | 35.672 | −40.865 | 1.00 | 129.47 | C |
| ATOM | 1069 | CD2 | LEU | A | 52 | 0.289 | 35.858 | −41.401 | 1.00 | 129.47 | C |
| ATOM | 1070 | N | ALA | A | 53 | −1.591 | 32.720 | −45.412 | 1.00 | 131.94 | N |
| ATOM | 1071 | CA | ALA | A | 53 | −1.811 | 31.453 | −46.120 | 1.00 | 131.94 | C |
| ATOM | 1072 | C | ALA | A | 53 | −0.776 | 31.322 | −47.202 | 1.00 | 131.94 | C |
| ATOM | 1073 | O | ALA | A | 53 | −0.227 | 30.245 | −47.412 | 1.00 | 131.94 | O |
| ATOM | 1074 | CB | ALA | A | 53 | −3.210 | 31.415 | −46.721 | 1.00 | 131.94 | C |
| ATOM | 1075 | N | GLN | A | 54 | −0.481 | 32.449 | −47.861 | 1.00 | 134.96 | N |
| ATOM | 1076 | CA | GLN | A | 54 | 0.523 | 32.530 | −48.908 | 1.00 | 134.96 | C |
| ATOM | 1077 | C | GLN | A | 54 | 1.883 | 32.183 | −48.350 | 1.00 | 134.96 | C |
| ATOM | 1078 | O | GLN | A | 54 | 2.618 | 31.428 | −48.981 | 1.00 | 134.96 | O |
| ATOM | 1079 | CB | GLN | A | 54 | 0.546 | 33.923 | −49.536 | 1.00 | 134.96 | C |
| ATOM | 1080 | CG | GLN | A | 54 | 1.219 | 33.972 | −50.906 | 1.00 | 134.96 | C |
| ATOM | 1081 | CD | GLN | A | 54 | 2.727 | 33.973 | −50.832 | 1.00 | 134.96 | C |
| ATOM | 1082 | NE2 | GLN | A | 54 | 3.352 | 33.388 | −51.829 | 1.00 | 134.96 | N |
| ATOM | 1083 | OE1 | GLN | A | 54 | 3.342 | 34.471 | −49.879 | 1.00 | 134.96 | O |
| ATOM | 1084 | N | ARG | A | 55 | 2.229 | 32.731 | −47.176 | 1.00 | 139.94 | N |
| ATOM | 1085 | CA | ARG | A | 55 | 3.505 | 32.423 | −46.551 | 1.00 | 139.94 | C |
| ATOM | 1086 | C | ARG | A | 55 | 3.595 | 30.934 | −46.256 | 1.00 | 139.94 | C |
| ATOM | 1087 | O | ARG | A | 55 | 4.688 | 30.385 | −46.295 | 1.00 | 139.94 | O |
| ATOM | 1088 | CB | ARG | A | 55 | 3.715 | 33.246 | −45.284 | 1.00 | 139.94 | C |
| ATOM | 1089 | CG | ARG | A | 55 | 4.449 | 34.557 | −45.586 | 1.00 | 139.94 | C |
| ATOM | 1090 | CD | ARG | A | 55 | 5.092 | 35.149 | −44.341 | 1.00 | 139.94 | C |
| ATOM | 1091 | NE | ARG | A | 55 | 5.426 | 36.565 | −44.501 | 1.00 | 139.94 | N |
| ATOM | 1092 | N | TRP | A | 56 | 2.449 | 30.273 | −46.025 | 1.00 | 143.91 | N |
| ATOM | 1093 | CA | TRP | A | 56 | 2.419 | 28.848 | −45.748 | 1.00 | 143.91 | C |
| ATOM | 1094 | C | TRP | A | 56 | 2.637 | 28.007 | −46.991 | 1.00 | 143.91 | C |
| ATOM | 1095 | O | TRP | A | 56 | 3.250 | 26.950 | −46.891 | 1.00 | 143.91 | O |
| ATOM | 1096 | CB | TRP | A | 56 | 1.111 | 28.450 | −45.079 | 1.00 | 143.91 | C |
| ATOM | 1097 | CG | TRP | A | 56 | 1.248 | 28.192 | −43.607 | 1.00 | 143.91 | C |
| ATOM | 1098 | CD1 | TRP | A | 56 | 0.677 | 28.906 | −42.590 | 1.00 | 143.91 | C |
| ATOM | 1099 | CD2 | TRP | A | 56 | 1.993 | 27.130 | −42.984 | 1.00 | 143.91 | C |
| ATOM | 1100 | CE2 | TRP | A | 56 | 1.832 | 27.269 | −41.584 | 1.00 | 143.91 | C |
| ATOM | 1101 | CE3 | TRP | A | 56 | 2.787 | 26.077 | −43.474 | 1.00 | 143.91 | C |
| ATOM | 1102 | NE1 | TRP | A | 56 | 1.030 | 28.365 | −41.371 | 1.00 | 143.91 | N |
| ATOM | 1103 | CZ2 | TRP | A | 56 | 2.444 | 26.399 | −40.668 | 1.00 | 143.91 | C |
| ATOM | 1104 | CZ3 | TRP | A | 56 | 3.385 | 25.214 | −42.567 | 1.00 | 143.91 | C |
| ATOM | 1105 | CH2 | TRP | A | 56 | 3.204 | 25.372 | −41.183 | 1.00 | 143.91 | C |
| ATOM | 1106 | N | MET | A | 57 | 2.156 | 28.463 | −48.158 | 1.00 | 147.65 | N |
| ATOM | 1107 | CA | MET | A | 57 | 2.321 | 27.736 | −49.426 | 1.00 | 147.65 | C |
| ATOM | 1108 | C | MET | A | 57 | 3.776 | 27.617 | −49.802 | 1.00 | 147.65 | C |
| ATOM | 1109 | O | MET | A | 57 | 4.164 | 26.641 | −50.434 | 1.00 | 147.65 | O |
| ATOM | 1110 | CB | MET | A | 57 | 1.562 | 28.408 | −50.574 | 1.00 | 147.65 | C |
| ATOM | 1111 | CG | MET | A | 57 | 0.071 | 28.290 | −50.458 | 1.00 | 147.65 | C |
| ATOM | 1112 | SD | MET | A | 57 | −0.497 | 26.589 | −50.265 | 1.00 | 147.65 | S |
| ATOM | 1113 | CE | MET | A | 57 | −2.159 | 26.913 | −49.638 | 1.00 | 147.65 | C |
| ATOM | 1114 | N | GLU | A | 58 | 4.579 | 28.603 | −49.406 | 1.00 | 151.17 | N |
| ATOM | 1115 | CA | GLU | A | 58 | 6.011 | 28.638 | −49.672 | 1.00 | 151.17 | C |
| ATOM | 1116 | C | GLU | A | 58 | 6.733 | 27.657 | −48.776 | 1.00 | 151.17 | C |
| ATOM | 1117 | O | GLU | A | 58 | 7.556 | 26.886 | −49.268 | 1.00 | 151.17 | O |
| ATOM | 1118 | CB | GLU | A | 58 | 6.554 | 30.049 | −49.453 | 1.00 | 151.17 | C |
| ATOM | 1119 | CG | GLU | A | 58 | 5.965 | 31.072 | −50.416 | 1.00 | 151.17 | C |
| ATOM | 1120 | CD | GLU | A | 58 | 6.548 | 31.121 | −51.820 | 1.00 | 151.17 | C |
| ATOM | 1121 | OE1 | GLU | A | 58 | 5.945 | 31.808 | −52.678 | 1.00 | 151.17 | O |
| ATOM | 1122 | OE2 | GLU | A | 58 | 7.594 | 30.479 | −52.068 | 1.00 | 151.17 | O + 1 |
| ATOM | 1123 | N | ARG | A | 59 | 6.425 | 27.682 | −47.458 | 1.00 | 153.37 | N |
| ATOM | 1124 | CA | ARG | A | 59 | 7.018 | 26.780 | −46.468 | 1.00 | 153.37 | C |
| ATOM | 1125 | C | ARG | A | 59 | 6.733 | 25.357 | −46.851 | 1.00 | 153.37 | C |
| ATOM | 1126 | O | ARG | A | 59 | 7.619 | 24.518 | −46.766 | 1.00 | 153.37 | O |
| ATOM | 1127 | CB | ARG | A | 59 | 6.470 | 27.053 | −45.058 | 1.00 | 153.37 | C |
| ATOM | 1128 | CG | ARG | A | 59 | 7.163 | 28.187 | −44.312 | 1.00 | 153.37 | C |
| ATOM | 1129 | CD | ARG | A | 59 | 6.652 | 28.358 | −42.875 | 1.00 | 153.37 | C |
| ATOM | 1130 | NE | ARG | A | 59 | 5.251 | 28.817 | −42.806 | 1.00 | 153.37 | N |
| ATOM | 1131 | CZ | ARG | A | 59 | 4.858 | 30.080 | −42.613 | 1.00 | 153.37 | C |
| ATOM | 1132 | NH1 | ARG | A | 59 | 5.753 | 31.050 | −42.467 | 1.00 | 153.37 | N + 1 |
| ATOM | 1133 | NH2 | ARG | A | 59 | 3.565 | 30.379 | −42.572 | 1.00 | 153.37 | N |
| ATOM | 1134 | N | LEU | A | 60 | 5.501 | 25.092 | −47.307 | 1.00 | 152.61 | N |
| ATOM | 1135 | CA | LEU | A | 60 | 5.060 | 23.761 | −47.700 | 1.00 | 152.61 | C |
| ATOM | 1136 | C | LEU | A | 60 | 5.768 | 23.234 | −48.934 | 1.00 | 152.61 | C |
| ATOM | 1137 | O | LEU | A | 60 | 6.030 | 22.033 | −49.004 | 1.00 | 152.61 | O |
| ATOM | 1138 | CB | LEU | A | 60 | 3.557 | 23.734 | −47.943 | 1.00 | 152.61 | C |
| ATOM | 1139 | CG | LEU | A | 60 | 2.652 | 23.798 | −46.728 | 1.00 | 152.61 | C |
| ATOM | 1140 | CD1 | LEU | A | 60 | 1.233 | 23.606 | −47.146 | 1.00 | 152.61 | C |
| ATOM | 1141 | CD2 | LEU | A | 60 | 3.033 | 22.772 | −45.688 | 1.00 | 152.61 | C |
| ATOM | 1142 | N | LYS | A | 61 | 6.064 | 24.107 | −49.908 | 1.00 | 151.58 | N |
| ATOM | 1143 | CA | LYS | A | 61 | 6.736 | 23.710 | −51.141 | 1.00 | 151.58 | C |
| ATOM | 1144 | C | LYS | A | 61 | 8.106 | 23.111 | −50.863 | 1.00 | 151.58 | C |
| ATOM | 1145 | O | LYS | A | 61 | 8.499 | 22.183 | −51.563 | 1.00 | 151.58 | O |
| ATOM | 1146 | CB | LYS | A | 61 | 6.873 | 24.899 | −52.081 | 1.00 | 151.58 | C |
| ATOM | 1147 | CG | LYS | A | 61 | 5.619 | 25.174 | −52.895 | 1.00 | 151.58 | C |
| ATOM | 1148 | CD | LYS | A | 61 | 5.623 | 26.572 | −53.486 | 1.00 | 151.58 | C |
| ATOM | 1149 | CE | LYS | A | 61 | 6.668 | 26.739 | −54.558 | 1.00 | 151.58 | C |
| ATOM | 1150 | NZ | LYS | A | 61 | 6.908 | 28.171 | −54.874 | 1.00 | 151.58 | N + 1 |
| ATOM | 1151 | N | THR | A | 62 | 8.811 | 23.605 | −49.824 | 1.00 | 150.95 | N |
| ATOM | 1152 | CA | THR | A | 62 | 10.164 | 23.158 | −49.457 | 1.00 | 150.95 | C |
| ATOM | 1153 | C | THR | A | 62 | 10.227 | 21.734 | −48.896 | 1.00 | 150.95 | C |
| ATOM | 1154 | O | THR | A | 62 | 11.280 | 21.100 | −48.961 | 1.00 | 150.95 | O |
| ATOM | 1155 | CB | THR | A | 62 | 10.788 | 24.126 | −48.456 | 1.00 | 150.95 | C |
| ATOM | 1156 | CG2 | THR | A | 62 | 10.873 | 25.547 | −49.002 | 1.00 | 150.95 | C |
| ATOM | 1157 | OG1 | THR | A | 62 | 10.038 | 24.115 | −47.242 | 1.00 | 150.95 | O |
| ATOM | 1158 | N | VAL | A | 63 | 9.125 | 21.243 | −48.337 | 1.00 | 150.93 | N |
| ATOM | 1159 | CA | VAL | A | 63 | 9.066 | 19.906 | −47.744 | 1.00 | 150.93 | C |
| ATOM | 1160 | C | VAL | A | 63 | 8.102 | 19.013 | −48.522 | 1.00 | 150.93 | C |
| ATOM | 1161 | O | VAL | A | 63 | 7.673 | 17.969 | −48.024 | 1.00 | 150.93 | O |
| ATOM | 1162 | CB | VAL | A | 63 | 8.671 | 19.984 | −46.253 | 1.00 | 150.93 | C |
| ATOM | 1163 | CG1 | VAL | A | 63 | 9.609 | 20.905 | −45.488 | 1.00 | 150.93 | C |
| ATOM | 1164 | CG2 | VAL | A | 63 | 7.223 | 20.435 | −46.090 | 1.00 | 150.93 | C |
| ATOM | 1165 | N | ALA | A | 64 | 7.748 | 19.437 | −49.732 | 1.00 | 150.82 | N |
| ATOM | 1166 | CA | ALA | A | 64 | 6.825 | 18.708 | −50.586 | 1.00 | 150.82 | C |
| ATOM | 1167 | C | ALA | A | 64 | 7.562 | 17.895 | −51.616 | 1.00 | 150.82 | C |
| ATOM | 1168 | O | ALA | A | 64 | 8.649 | 18.281 | −52.051 | 1.00 | 150.82 | O |
| ATOM | 1169 | CB | ALA | A | 64 | 5.892 | 19.678 | −51.279 | 1.00 | 150.82 | C |
| ATOM | 1170 | N | GLY | A | 65 | 6.958 | 16.782 | −52.012 | 1.00 | 152.23 | N |
| ATOM | 1171 | CA | GLY | A | 65 | 7.521 | 15.909 | −53.031 | 1.00 | 152.23 | C |
| ATOM | 1172 | C | GLY | A | 65 | 7.427 | 16.552 | −54.390 | 1.00 | 152.23 | C |
| ATOM | 1173 | O | GLY | A | 65 | 6.631 | 17.474 | −54.581 | 1.00 | 152.23 | O |
| ATOM | 1174 | N | SER | A | 66 | 8.229 | 16.077 | −55.341 | 1.00 | 154.59 | N |
| ATOM | 1175 | CA | SER | A | 66 | 8.268 | 16.613 | −56.705 | 1.00 | 154.59 | C |
| ATOM | 1176 | C | SER | A | 66 | 6.882 | 16.951 | −57.277 | 1.00 | 154.59 | C |
| ATOM | 1177 | O | SER | A | 66 | 6.698 | 18.042 | −57.810 | 1.00 | 154.59 | O |
| ATOM | 1178 | CB | SER | A | 66 | 8.960 | 15.629 | −57.636 | 1.00 | 154.59 | C |
| ATOM | 1179 | OG | SER | A | 66 | 8.174 | 14.458 | −57.776 | 1.00 | 154.59 | O |
| ATOM | 1180 | N | LYS | A | 67 | 5.912 | 16.043 | −57.141 | 1.00 | 157.07 | N |
| ATOM | 1181 | CA | LYS | A | 67 | 4.582 | 16.272 | −57.699 | 1.00 | 157.07 | C |
| ATOM | 1182 | C | LYS | A | 67 | 3.669 | 17.100 | −56.774 | 1.00 | 157.07 | C |
| ATOM | 1183 | O | LYS | A | 67 | 2.771 | 17.773 | −57.271 | 1.00 | 157.07 | O |
| ATOM | 1184 | CB | LYS | A | 67 | 3.911 | 14.951 | −58.069 | 1.00 | 157.07 | C |
| ATOM | 1185 | CG | LYS | A | 67 | 4.657 | 14.178 | −59.149 | 1.00 | 157.07 | C |
| ATOM | 1186 | CD | LYS | A | 67 | 3.895 | 12.903 | −59.526 | 1.00 | 157.07 | C |
| ATOM | 1187 | CE | LYS | A | 67 | 4.588 | 12.025 | −60.550 | 1.00 | 157.07 | C |
| ATOM | 1188 | NZ | LYS | A | 67 | 3.905 | 10.696 | −60.680 | 1.00 | 157.07 | N + 1 |
| ATOM | 1189 | N | MET | A | 68 | 3.891 | 17.066 | −55.451 | 1.00 | 156.90 | N |
| ATOM | 1190 | CA | MET | A | 68 | 3.101 | 17.862 | −54.510 | 1.00 | 156.90 | C |
| ATOM | 1191 | C | MET | A | 68 | 3.512 | 19.305 | −54.652 | 1.00 | 156.90 | C |
| ATOM | 1192 | O | MET | A | 68 | 2.708 | 20.202 | −54.436 | 1.00 | 156.90 | O |
| ATOM | 1193 | CB | MET | A | 68 | 3.301 | 17.364 | −53.068 | 1.00 | 156.90 | C |
| ATOM | 1194 | CG | MET | A | 68 | 2.567 | 18.168 | −51.999 | 1.00 | 156.90 | C |
| ATOM | 1195 | SD | MET | A | 68 | 0.915 | 18.725 | −52.446 | 1.00 | 156.90 | S |
| ATOM | 1196 | CE | MET | A | 68 | −0.063 | 17.270 | −52.105 | 1.00 | 156.90 | C |
| ATOM | 1197 | N | GLN | A | 69 | 4.769 | 19.525 | −55.040 | 1.00 | 159.73 | N |
| ATOM | 1198 | CA | GLN | A | 69 | 5.320 | 20.854 | −55.260 | 1.00 | 159.73 | C |
| ATOM | 1199 | C | GLN | A | 69 | 4.539 | 21.598 | −56.337 | 1.00 | 159.73 | C |
| ATOM | 1200 | O | GLN | A | 69 | 4.331 | 22.805 | −56.202 | 1.00 | 159.73 | O |
| ATOM | 1201 | CB | GLN | A | 69 | 6.785 | 20.763 | −55.664 | 1.00 | 159.73 | C |
| ATOM | 1202 | CG | GLN | A | 69 | 7.747 | 20.754 | −54.492 | 1.00 | 159.73 | C |
| ATOM | 1203 | CD | GLN | A | 69 | 9.160 | 21.056 | −54.935 | 1.00 | 159.73 | C |
| ATOM | 1204 | NE2 | GLN | A | 69 | 10.057 | 21.240 | −53.974 | 1.00 | 159.73 | N |
| ATOM | 1205 | OE1 | GLN | A | 69 | 9.472 | 21.115 | −56.136 | 1.00 | 159.73 | O |
| ATOM | 1206 | N | GLY | A | 70 | 4.105 | 20.872 | −57.375 | 1.00 | 158.09 | N |
| ATOM | 1207 | CA | GLY | A | 70 | 3.327 | 21.426 | −58.480 | 1.00 | 158.09 | C |
| ATOM | 1208 | C | GLY | A | 70 | 1.935 | 21.847 | −58.060 | 1.00 | 158.09 | C |
| ATOM | 1209 | O | GLY | A | 70 | 1.450 | 22.895 | −58.486 | 1.00 | 158.09 | O |
| ATOM | 1210 | N | LEU | A | 71 | 1.297 | 21.044 | −57.201 | 1.00 | 153.93 | N |
| ATOM | 1211 | CA | LEU | A | 71 | −0.042 | 21.333 | −56.706 | 1.00 | 153.93 | C |
| ATOM | 1212 | C | LEU | A | 71 | −0.016 | 22.509 | −55.760 | 1.00 | 153.93 | C |
| ATOM | 1213 | O | LEU | A | 71 | −0.942 | 23.316 | −55.781 | 1.00 | 153.93 | O |
| ATOM | 1214 | CB | LEU | A | 71 | −0.636 | 20.113 | −56.013 | 1.00 | 153.93 | C |
| ATOM | 1215 | CG | LEU | A | 71 | −0.800 | 18.872 | −56.872 | 1.00 | 153.93 | C |
| ATOM | 1216 | CD1 | LEU | A | 71 | −1.197 | 17.694 | −56.021 | 1.00 | 153.93 | C |
| ATOM | 1217 | CD2 | LEU | A | 71 | −1.817 | 19.103 | −57.984 | 1.00 | 153.93 | C |
| ATOM | 1218 | N | LEU | A | 72 | 1.049 | 22.613 | −54.938 | 1.00 | 149.29 | N |
| ATOM | 1219 | CA | LEU | A | 72 | 1.231 | 23.713 | −53.994 | 1.00 | 149.29 | C |
| ATOM | 1220 | C | LEU | A | 72 | 1.489 | 24.969 | −54.754 | 1.00 | 149.29 | C |
| ATOM | 1221 | O | LEU | A | 72 | 0.788 | 25.957 | −54.565 | 1.00 | 149.29 | O |
| ATOM | 1222 | CB | LEU | A | 72 | 2.391 | 23.446 | −53.030 | 1.00 | 149.29 | C |
| ATOM | 1223 | CG | LEU | A | 72 | 2.190 | 22.369 | −51.973 | 1.00 | 149.29 | C |
| ATOM | 1224 | CD1 | LEU | A | 72 | 3.449 | 22.141 | −51.217 | 1.00 | 149.29 | C |
| ATOM | 1225 | CD2 | LEU | A | 72 | 1.088 | 22.744 | −51.001 | 1.00 | 149.29 | C |
| ATOM | 1226 | N | GLU | A | 73 | 2.475 | 24.917 | −55.648 | 1.00 | 144.34 | N |
| ATOM | 1227 | CA | GLU | A | 73 | 2.836 | 26.042 | −56.485 | 1.00 | 144.34 | C |
| ATOM | 1228 | C | GLU | A | 73 | 1.634 | 26.561 | −57.269 | 1.00 | 144.34 | C |
| ATOM | 1229 | O | GLU | A | 73 | 1.454 | 27.772 | −57.365 | 1.00 | 144.34 | O |
| ATOM | 1230 | CB | GLU | A | 73 | 3.967 | 25.665 | −57.442 | 1.00 | 144.34 | C |
| ATOM | 1231 | CG | GLU | A | 73 | 4.363 | 26.770 | −58.413 | 1.00 | 144.34 | C |
| ATOM | 1232 | CD | GLU | A | 73 | 4.584 | 28.151 | −57.809 | 1.00 | 144.34 | C |
| ATOM | 1233 | OE1 | GLU | A | 73 | 4.193 | 29.150 | −58.455 | 1.00 | 144.34 | O |
| ATOM | 1234 | OE2 | GLU | A | 73 | 5.123 | 28.239 | −56.683 | 1.00 | 144.34 | O + 1 |
| ATOM | 1235 | N | ARG | A | 74 | 0.805 | 25.660 | −57.802 | 1.00 | 137.64 | N |
| ATOM | 1236 | CA | ARG | A | 74 | −0.373 | 26.048 | −58.571 | 1.00 | 137.64 | C |
| ATOM | 1237 | C | ARG | A | 74 | −1.432 | 26.737 | −57.711 | 1.00 | 137.64 | C |
| ATOM | 1238 | O | ARG | A | 74 | −2.233 | 27.506 | −58.235 | 1.00 | 137.64 | O |
| ATOM | 1239 | CB | ARG | A | 74 | −0.977 | 24.837 | −59.270 | 1.00 | 137.64 | C |
| ATOM | 1240 | CG | ARG | A | 74 | −0.178 | 24.413 | −60.494 | 1.00 | 137.64 | C |
| ATOM | 1241 | CD | ARG | A | 74 | −0.639 | 23.076 | −61.076 | 1.00 | 137.64 | C |
| ATOM | 1242 | NE | ARG | A | 74 | −1.875 | 23.169 | −61.853 | 1.00 | 137.64 | N |
| ATOM | 1243 | N | VAL | A | 75 | −1.455 | 26.459 | −56.410 | 1.00 | 133.57 | N |
| ATOM | 1244 | CA | VAL | A | 75 | −2.382 | 27.113 | −55.481 | 1.00 | 133.57 | C |
| ATOM | 1245 | C | VAL | A | 75 | −1.716 | 28.381 | −55.031 | 1.00 | 133.57 | C |
| ATOM | 1246 | O | VAL | A | 75 | −2.380 | 29.365 | −54.731 | 1.00 | 133.57 | O |
| ATOM | 1247 | CB | VAL | A | 75 | −2.751 | 26.207 | −54.293 | 1.00 | 133.57 | C |
| ATOM | 1248 | CG1 | VAL | A | 75 | −3.424 | 26.994 | −53.180 | 1.00 | 133.57 | C |
| ATOM | 1249 | CG2 | VAL | A | 75 | −3.645 | 25.068 | −54.745 | 1.00 | 133.57 | C |
| ATOM | 1250 | N | ASN | A | 76 | −0.387 | 28.347 | −54.989 | 1.00 | 132.81 | N |
| ATOM | 1251 | CA | ASN | A | 76 | 0.428 | 29.480 | −54.613 | 1.00 | 132.81 | C |
| ATOM | 1252 | C | ASN | A | 76 | 0.221 | 30.598 | −55.612 | 1.00 | 132.81 | C |
| ATOM | 1253 | O | ASN | A | 76 | 0.076 | 31.740 | −55.205 | 1.00 | 132.81 | O |
| ATOM | 1254 | CB | ASN | A | 76 | 1.899 | 29.089 | −54.536 | 1.00 | 132.81 | C |
| ATOM | 1255 | CG | ASN | A | 76 | 2.783 | 30.166 | −53.979 | 1.00 | 132.81 | C |
| ATOM | 1256 | ND2 | ASN | A | 76 | 4.009 | 30.236 | −54.460 | 1.00 | 132.81 | N |
| ATOM | 1257 | OD1 | ASN | A | 76 | 2.389 | 30.940 | −53.108 | 1.00 | 132.81 | O |
| ATOM | 1258 | N | THR | A | 77 | 0.152 | 30.265 | −56.911 | 1.00 | 134.70 | N |
| ATOM | 1259 | CA | THR | A | 77 | −0.052 | 31.237 | −57.994 | 1.00 | 134.70 | C |
| ATOM | 1260 | C | THR | A | 77 | −1.394 | 31.967 | −57.875 | 1.00 | 134.70 | C |
| ATOM | 1261 | O | THR | A | 77 | −1.492 | 33.144 | −58.236 | 1.00 | 134.70 | O |
| ATOM | 1262 | CB | THR | A | 77 | 0.023 | 30.562 | −59.364 | 1.00 | 134.70 | C |
| ATOM | 1263 | CG2 | THR | A | 77 | 1.385 | 29.982 | −59.657 | 1.00 | 134.70 | C |
| ATOM | 1264 | OG1 | THR | A | 77 | −0.983 | 29.552 | −59.445 | 1.00 | 134.70 | O |
| ATOM | 1265 | N | GLU | A | 78 | −2.421 | 31.260 | −57.372 | 1.00 | 137.89 | N |
| ATOM | 1266 | CA | GLU | A | 78 | −3.762 | 31.804 | −57.205 | 1.00 | 137.89 | C |
| ATOM | 1267 | C | GLU | A | 78 | −3.809 | 32.909 | −56.166 | 1.00 | 137.89 | C |
| ATOM | 1268 | O | GLU | A | 78 | −4.723 | 33.735 | −56.215 | 1.00 | 137.89 | O |
| ATOM | 1269 | CB | GLU | A | 78 | −4.750 | 30.694 | −56.817 | 1.00 | 137.89 | C |
| ATOM | 1270 | CG | GLU | A | 78 | −4.938 | 29.641 | −57.887 | 1.00 | 137.89 | C |
| ATOM | 1271 | CD | GLU | A | 78 | −5.387 | 30.202 | −59.217 | 1.00 | 137.89 | C |
| ATOM | 1272 | OE1 | GLU | A | 78 | −6.094 | 31.236 | −59.209 | 1.00 | 137.89 | O |
| ATOM | 1273 | OE2 | GLU | A | 78 | −5.031 | 29.613 | −60.264 | 1.00 | 137.89 | O + 1 |
| ATOM | 1274 | N | ILE | A | 79 | −2.849 | 32.918 | −55.217 | 1.00 | 139.92 | N |
| ATOM | 1275 | CA | ILE | A | 79 | −2.817 | 33.885 | −54.115 | 1.00 | 139.92 | C |
| ATOM | 1276 | C | ILE | A | 79 | −1.478 | 34.611 | −53.998 | 1.00 | 139.92 | C |
| ATOM | 1277 | O | ILE | A | 79 | −1.269 | 35.315 | −53.015 | 1.00 | 139.92 | O |
| ATOM | 1278 | CB | ILE | A | 79 | −3.163 | 33.181 | −52.779 | 1.00 | 139.92 | C |
| ATOM | 1279 | CG1 | ILE | A | 79 | −2.137 | 32.100 | −52.437 | 1.00 | 139.92 | C |
| ATOM | 1280 | CG2 | ILE | A | 79 | −4.565 | 32.607 | −52.816 | 1.00 | 139.92 | C |
| ATOM | 1281 | CD1 | ILE | A | 79 | −2.145 | 31.687 | −51.010 | 1.00 | 139.92 | C |
| ATOM | 1282 | N | HIS | A | 80 | −0.583 | 34.459 | −54.981 | 1.00 | 146.78 | N |
| ATOM | 1283 | CA | HIS | A | 80 | 0.753 | 35.074 | −54.952 | 1.00 | 146.78 | C |
| ATOM | 1284 | C | HIS | A | 80 | 0.704 | 36.572 | −55.125 | 1.00 | 146.78 | C |
| ATOM | 1285 | O | HIS | A | 80 | 1.663 | 37.247 | −54.756 | 1.00 | 146.78 | O |
| ATOM | 1286 | CB | HIS | A | 80 | 1.621 | 34.476 | −56.076 | 1.00 | 146.78 | C |
| ATOM | 1287 | CG | HIS | A | 80 | 3.113 | 34.479 | −55.871 | 1.00 | 146.78 | C |
| ATOM | 1288 | CD2 | HIS | A | 80 | 4.001 | 33.464 | −56.023 | 1.00 | 146.78 | C |
| ATOM | 1289 | ND1 | HIS | A | 80 | 3.813 | 35.642 | −55.594 | 1.00 | 146.78 | N |
| ATOM | 1290 | CE1 | HIS | A | 80 | 5.086 | 35.283 | −55.513 | 1.00 | 146.78 | C |
| ATOM | 1291 | NE2 | HIS | A | 80 | 5.247 | 33.983 | −55.768 | 1.00 | 146.78 | N |
| ATOM | 1292 | N | PHE | A | 81 | −0.385 | 37.100 | −55.696 | 1.00 | 150.80 | N |
| ATOM | 1293 | CA | PHE | A | 81 | −0.520 | 38.534 | −55.943 | 1.00 | 150.80 | C |
| ATOM | 1294 | C | PHE | A | 81 | −0.376 | 39.385 | −54.656 | 1.00 | 150.80 | C |
| ATOM | 1295 | O | PHE | A | 81 | −0.010 | 40.553 | −54.753 | 1.00 | 150.80 | O |
| ATOM | 1296 | CB | PHE | A | 81 | −1.858 | 38.834 | −56.628 | 1.00 | 150.80 | C |
| ATOM | 1297 | CG | PHE | A | 81 | −3.081 | 38.501 | −55.799 | 1.00 | 150.80 | C |
| ATOM | 1298 | CD1 | PHE | A | 81 | −3.520 | 39.361 | −54.799 | 1.00 | 150.80 | C |
| ATOM | 1299 | CD2 | PHE | A | 81 | −3.806 | 37.341 | −56.034 | 1.00 | 150.80 | C |
| ATOM | 1300 | CE1 | PHE | A | 81 | −4.640 | 39.051 | −54.028 | 1.00 | 150.80 | C |
| ATOM | 1301 | CE2 | PHE | A | 81 | −4.942 | 37.042 | −55.272 | 1.00 | 150.80 | C |
| ATOM | 1302 | CZ | PHE | A | 81 | −5.345 | 37.893 | −54.270 | 1.00 | 150.80 | C |
| ATOM | 1303 | N | VAL | A | 82 | −0.650 | 38.804 | −53.468 | 1.00 | 152.56 | N |
| ATOM | 1304 | CA | VAL | A | 82 | −0.557 | 39.515 | −52.188 | 1.00 | 152.56 | C |
| ATOM | 1305 | C | VAL | A | 82 | 0.855 | 40.035 | −51.925 | 1.00 | 152.56 | C |
| ATOM | 1306 | O | VAL | A | 82 | 1.035 | 40.946 | −51.121 | 1.00 | 152.56 | O |
| ATOM | 1307 | CB | VAL | A | 82 | −1.015 | 38.648 | −51.003 | 1.00 | 152.56 | C |
| ATOM | 1308 | CG1 | VAL | A | 82 | −2.481 | 38.265 | −51.131 | 1.00 | 152.56 | C |
| ATOM | 1309 | CG2 | VAL | A | 82 | −0.129 | 37.420 | −50.837 | 1.00 | 152.56 | C |
| ATOM | 1310 | N | THR | A | 83 | 1.852 | 39.450 | −52.586 | 1.00 | 158.41 | N |
| ATOM | 1311 | CA | THR | A | 83 | 3.247 | 39.852 | −52.424 | 1.00 | 158.41 | C |
| ATOM | 1312 | C | THR | A | 83 | 3.580 | 41.038 | −53.325 | 1.00 | 158.41 | C |
| ATOM | 1313 | O | THR | A | 83 | 4.684 | 41.579 | −53.251 | 1.00 | 158.41 | O |
| ATOM | 1314 | CB | THR | A | 83 | 4.177 | 38.674 | −52.686 | 1.00 | 158.41 | C |
| ATOM | 1315 | CG2 | THR | A | 83 | 3.971 | 37.548 | −51.695 | 1.00 | 158.41 | C |
| ATOM | 1316 | OG1 | THR | A | 83 | 3.976 | 38.192 | −54.013 | 1.00 | 158.41 | O |
| ATOM | 1317 | N | LYS | A | 84 | 2.625 | 41.449 | −54.170 | 1.00 | 164.53 | N |
| ATOM | 1318 | CA | LYS | A | 84 | 2.794 | 42.613 | −55.021 | 1.00 | 164.53 | C |
| ATOM | 1319 | C | LYS | A | 84 | 2.790 | 43.845 | −54.134 | 1.00 | 164.53 | C |
| ATOM | 1320 | O | LYS | A | 84 | 3.229 | 44.910 | −54.559 | 1.00 | 164.53 | O |
| ATOM | 1321 | CB | LYS | A | 84 | 1.675 | 42.687 | −56.063 | 1.00 | 164.53 | C |
| ATOM | 1322 | N | CYS | A | 85 | 2.307 | 43.684 | −52.884 | 1.00 | 170.17 | N |
| ATOM | 1323 | CA | CYS | A | 85 | 2.205 | 44.745 | −51.890 | 1.00 | 170.17 | C |
| ATOM | 1324 | C | CYS | A | 85 | 3.190 | 44.533 | −50.758 | 1.00 | 170.17 | C |
| ATOM | 1325 | O | CYS | A | 85 | 3.366 | 43.409 | −50.271 | 1.00 | 170.17 | O |
| ATOM | 1326 | CB | CYS | A | 85 | 0.780 | 44.850 | −51.360 | 1.00 | 170.17 | C |
| ATOM | 1327 | SG | CYS | A | 85 | −0.451 | 45.319 | −52.605 | 1.00 | 170.17 | S |
| ATOM | 1328 | N | ALA | A | 86 | 3.784 | 45.645 | −50.305 | 1.00 | 170.29 | N |
| ATOM | 1329 | CA | ALA | A | 86 | 4.766 | 45.689 | −49.225 | 1.00 | 170.29 | C |
| ATOM | 1330 | C | ALA | A | 86 | 4.126 | 45.488 | −47.836 | 1.00 | 170.29 | C |
| ATOM | 1331 | O | ALA | A | 86 | 4.149 | 46.392 | −46.989 | 1.00 | 170.29 | O |
| ATOM | 1332 | CB | ALA | A | 86 | 5.524 | 47.006 | −49.273 | 1.00 | 170.29 | C |
| ATOM | 1333 | N | PHE | A | 87 | 3.581 | 44.283 | −47.602 | 1.00 | 170.15 | N |
| ATOM | 1334 | CA | PHE | A | 87 | 2.976 | 43.930 | −46.321 | 1.00 | 170.15 | C |
| ATOM | 1335 | C | PHE | A | 87 | 4.050 | 43.874 | −45.250 | 1.00 | 170.15 | C |
| ATOM | 1336 | O | PHE | A | 87 | 5.083 | 43.233 | −45.454 | 1.00 | 170.15 | O |
| ATOM | 1337 | CB | PHE | A | 87 | 2.251 | 42.582 | −46.420 | 1.00 | 170.15 | C |
| ATOM | 1338 | CG | PHE | A | 87 | 0.876 | 42.655 | −47.032 | 1.00 | 170.15 | C |
| ATOM | 1339 | CD1 | PHE | A | 87 | 0.685 | 42.401 | −48.386 | 1.00 | 170.15 | C |
| ATOM | 1340 | CD2 | PHE | A | 87 | −0.232 | 42.953 | −46.252 | 1.00 | 170.15 | C |
| ATOM | 1341 | CE1 | PHE | A | 87 | −0.588 | 42.460 | −48.948 | 1.00 | 170.15 | C |
| ATOM | 1342 | CE2 | PHE | A | 87 | −1.502 | 43.008 | −46.814 | 1.00 | 170.15 | C |
| ATOM | 1343 | CZ | PHE | A | 87 | −1.672 | 42.763 | −48.158 | 1.00 | 170.15 | C |
| ATOM | 1344 | N | GLN | A | 88 | 3.822 | 44.559 | −44.124 | 1.00 | 172.23 | N |
| ATOM | 1345 | CA | GLN | A | 88 | 4.768 | 44.587 | −43.014 | 1.00 | 172.23 | C |
| ATOM | 1346 | C | GLN | A | 88 | 4.770 | 43.263 | −42.263 | 1.00 | 172.23 | C |
| ATOM | 1347 | O | GLN | A | 88 | 3.745 | 42.579 | −42.239 | 1.00 | 172.23 | O |
| ATOM | 1348 | CB | GLN | A | 88 | 4.443 | 45.726 | −42.048 | 1.00 | 172.23 | C |
| ATOM | 1349 | N | PRO | A | 89 | 5.908 | 42.884 | −41.633 | 1.00 | 174.22 | N |
| ATOM | 1350 | CA | PRO | A | 89 | 5.948 | 41.613 | −40.899 | 1.00 | 174.22 | C |
| ATOM | 1351 | C | PRO | A | 89 | 4.923 | 41.586 | −39.774 | 1.00 | 174.22 | C |
| ATOM | 1352 | O | PRO | A | 89 | 4.578 | 42.649 | −39.237 | 1.00 | 174.22 | O |
| ATOM | 1353 | CB | PRO | A | 89 | 7.386 | 41.552 | −40.362 | 1.00 | 174.22 | C |
| ATOM | 1354 | CG | PRO | A | 89 | 7.853 | 42.954 | −40.363 | 1.00 | 174.22 | C |
| ATOM | 1355 | CD | PRO | A | 89 | 7.215 | 43.567 | −41.569 | 1.00 | 174.22 | C |
| ATOM | 1356 | N | PRO | A | 90 | 4.409 | 40.378 | −39.433 | 1.00 | 174.34 | N |
| ATOM | 1357 | CA | PRO | A | 90 | 3.402 | 40.289 | −38.371 | 1.00 | 174.34 | C |
| ATOM | 1358 | C | PRO | A | 90 | 3.832 | 40.981 | −37.070 | 1.00 | 174.34 | C |
| ATOM | 1359 | O | PRO | A | 90 | 5.010 | 40.922 | −36.696 | 1.00 | 174.34 | O |
| ATOM | 1360 | CB | PRO | A | 90 | 3.223 | 38.774 | −38.170 | 1.00 | 174.34 | C |
| ATOM | 1361 | CG | PRO | A | 90 | 3.594 | 38.172 | −39.472 | 1.00 | 174.34 | C |
| ATOM | 1362 | CD | PRO | A | 90 | 4.700 | 39.041 | −40.005 | 1.00 | 174.34 | C |
| ATOM | 1363 | N | PRO | A | 91 | 2.873 | 41.669 | −36.397 | 1.00 | 174.64 | N |
| ATOM | 1364 | CA | PRO | A | 91 | 3.181 | 42.334 | −35.118 | 1.00 | 174.64 | C |
| ATOM | 1365 | C | PRO | A | 91 | 3.372 | 41.314 | −33.991 | 1.00 | 174.64 | C |
| ATOM | 1366 | O | PRO | A | 91 | 2.948 | 40.158 | −34.098 | 1.00 | 174.64 | O |
| ATOM | 1367 | CB | PRO | A | 91 | 1.959 | 43.223 | −34.877 | 1.00 | 174.64 | C |
| ATOM | 1368 | CG | PRO | A | 91 | 0.856 | 42.537 | −35.585 | 1.00 | 174.64 | C |
| ATOM | 1369 | CD | PRO | A | 91 | 1.448 | 41.823 | −36.760 | 1.00 | 174.64 | C |
| ATOM | 1370 | N | SER | A | 92 | 4.024 | 41.746 | −32.912 | 1.00 | 174.69 | N |
| ATOM | 1371 | CA | SER | A | 92 | 4.305 | 40.916 | −31.740 | 1.00 | 174.69 | C |
| ATOM | 1372 | C | SER | A | 92 | 3.030 | 40.374 | −31.104 | 1.00 | 174.69 | C |
| ATOM | 1373 | O | SER | A | 92 | 3.046 | 39.289 | −30.518 | 1.00 | 174.69 | O |
| ATOM | 1374 | CB | SER | A | 92 | 5.075 | 41.731 | −30.704 | 1.00 | 174.69 | C |
| ATOM | 1375 | OG | SER | A | 92 | 4.359 | 42.903 | −30.338 | 1.00 | 174.69 | O |
| ATOM | 1376 | N | CYS | A | 93 | 1.935 | 41.146 | −31.217 | 1.00 | 172.90 | N |
| ATOM | 1377 | CA | CYS | A | 93 | 0.635 | 40.834 | −30.633 | 1.00 | 172.90 | C |
| ATOM | 1378 | C | CYS | A | 93 | −0.060 | 39.672 | −31.328 | 1.00 | 172.90 | C |
| ATOM | 1379 | O | CYS | A | 93 | −0.990 | 39.104 | −30.749 | 1.00 | 172.90 | O |
| ATOM | 1380 | CB | CYS | A | 93 | −0.263 | 42.069 | −30.630 | 1.00 | 172.90 | C |
| ATOM | 1381 | SG | CYS | A | 93 | −0.591 | 42.777 | −32.275 | 1.00 | 172.90 | S |
| ATOM | 1382 | N | LEU | A | 94 | 0.331 | 39.358 | −32.577 | 1.00 | 173.60 | N |
| ATOM | 1383 | CA | LEU | A | 94 | −0.321 | 38.318 | −33.363 | 1.00 | 173.60 | C |
| ATOM | 1384 | C | LEU | A | 94 | −0.002 | 36.916 | −32.870 | 1.00 | 173.60 | C |
| ATOM | 1385 | O | LEU | A | 94 | 1.156 | 36.486 | −32.897 | 1.00 | 173.60 | O |
| ATOM | 1386 | CB | LEU | A | 94 | 0.041 | 38.458 | −34.840 | 1.00 | 173.60 | C |
| ATOM | 1387 | CG | LEU | A | 94 | −0.938 | 37.872 | −35.853 | 1.00 | 173.60 | C |
| ATOM | 1388 | CD1 | LEU | A | 94 | −2.379 | 38.218 | −35.505 | 1.00 | 173.60 | C |
| ATOM | 1389 | CD2 | LEU | A | 94 | −0.611 | 38.373 | −37.243 | 1.00 | 173.60 | C |
| ATOM | 1390 | N | ARG | A | 95 | −1.053 | 36.215 | −32.419 | 1.00 | 174.62 | N |
| ATOM | 1391 | CA | ARG | A | 95 | −0.976 | 34.848 | −31.918 | 1.00 | 174.62 | C |
| ATOM | 1392 | C | ARG | A | 95 | −1.397 | 33.882 | −32.995 | 1.00 | 174.62 | C |
| ATOM | 1393 | O | ARG | A | 95 | −2.395 | 34.116 | −33.675 | 1.00 | 174.62 | O |
| ATOM | 1394 | CB | ARG | A | 95 | −1.868 | 34.671 | −30.683 | 1.00 | 174.62 | C |
| ATOM | 1395 | CG | ARG | A | 95 | −1.189 | 33.912 | −29.552 | 1.00 | 174.62 | C |
| ATOM | 1396 | CD | ARG | A | 95 | −1.214 | 34.665 | −28.221 | 1.00 | 174.62 | C |
| ATOM | 1397 | NE | ARG | A | 95 | −0.992 | 36.117 | −28.342 | 1.00 | 174.62 | N |
| ATOM | 1398 | CZ | ARG | A | 95 | 0.201 | 36.711 | −28.414 | 1.00 | 174.62 | C |
| ATOM | 1399 | NH1 | ARG | A | 95 | 1.317 | 35.988 | −28.401 | 1.00 | 174.62 | N + 1 |
| ATOM | 1400 | NH2 | ARG | A | 95 | 0.286 | 38.032 | −28.512 | 1.00 | 174.62 | N |
| ATOM | 1401 | N | PHE | A | 96 | −0.656 | 32.787 | −33.144 | 1.00 | 172.75 | N |
| ATOM | 1402 | CA | PHE | A | 96 | −0.978 | 31.775 | −34.141 | 1.00 | 172.75 | C |
| ATOM | 1403 | C | PHE | A | 96 | −1.472 | 30.518 | −33.453 | 1.00 | 172.75 | C |
| ATOM | 1404 | O | PHE | A | 96 | −1.036 | 30.216 | −32.341 | 1.00 | 172.75 | O |
| ATOM | 1405 | CB | PHE | A | 96 | 0.234 | 31.482 | −35.035 | 1.00 | 172.75 | C |
| ATOM | 1406 | CG | PHE | A | 96 | 0.683 | 32.679 | −35.837 | 1.00 | 172.75 | C |
| ATOM | 1407 | CD1 | PHE | A | 96 | 0.093 | 32.980 | −37.056 | 1.00 | 172.75 | C |
| ATOM | 1408 | CD2 | PHE | A | 96 | 1.688 | 33.516 | −35.365 | 1.00 | 172.75 | C |
| ATOM | 1409 | CE1 | PHE | A | 96 | 0.496 | 34.106 | −37.785 | 1.00 | 172.75 | C |
| ATOM | 1410 | CE2 | PHE | A | 96 | 2.095 | 34.638 | −36.098 | 1.00 | 172.75 | C |
| ATOM | 1411 | CZ | PHE | A | 96 | 1.498 | 34.924 | −37.303 | 1.00 | 172.75 | C |
| ATOM | 1412 | N | VAL | A | 97 | −2.397 | 29.802 | −34.100 | 1.00 | 171.81 | N |
| ATOM | 1413 | CA | VAL | A | 97 | −2.965 | 28.578 | −33.554 | 1.00 | 171.81 | C |
| ATOM | 1414 | C | VAL | A | 97 | −2.858 | 27.457 | −34.588 | 1.00 | 171.81 | C |
| ATOM | 1415 | O | VAL | A | 97 | −3.121 | 27.682 | −35.773 | 1.00 | 171.81 | O |
| ATOM | 1416 | CB | VAL | A | 97 | −4.417 | 28.803 | −33.060 | 1.00 | 171.81 | C |
| ATOM | 1417 | CG1 | VAL | A | 97 | −5.426 | 28.793 | −34.204 | 1.00 | 171.81 | C |
| ATOM | 1418 | CG2 | VAL | A | 97 | −4.793 | 27.781 | −32.002 | 1.00 | 171.81 | C |
| ATOM | 1419 | N | GLN | A | 98 | −2.472 | 26.255 | −34.135 | 1.00 | 170.10 | N |
| ATOM | 1420 | CA | GLN | A | 98 | −2.321 | 25.089 | −35.007 | 1.00 | 170.10 | C |
| ATOM | 1421 | C | GLN | A | 98 | −3.656 | 24.580 | −35.544 | 1.00 | 170.10 | C |
| ATOM | 1422 | O | GLN | A | 98 | −4.581 | 24.317 | −34.771 | 1.00 | 170.10 | O |
| ATOM | 1423 | CB | GLN | A | 98 | −1.626 | 23.965 | −34.263 | 1.00 | 170.10 | C |
| ATOM | 1424 | CG | GLN | A | 98 | −1.301 | 22.781 | −35.138 | 1.00 | 170.10 | C |
| ATOM | 1425 | CD | GLN | A | 98 | −0.012 | 22.241 | −34.632 | 1.00 | 170.10 | C |
| ATOM | 1426 | NE2 | GLN | A | 98 | 1.088 | 22.872 | −35.039 | 1.00 | 170.10 | N |
| ATOM | 1427 | OE1 | GLN | A | 98 | 0.011 | 21.320 | −33.821 | 1.00 | 170.10 | O |
| ATOM | 1428 | N | THR | A | 99 | −3.742 | 24.405 | −36.865 | 1.00 | 165.60 | N |
| ATOM | 1429 | CA | THR | A | 99 | −4.961 | 23.931 | −37.506 | 1.00 | 165.60 | C |
| ATOM | 1430 | C | THR | A | 99 | −4.615 | 23.004 | −38.656 | 1.00 | 165.60 | C |
| ATOM | 1431 | O | THR | A | 99 | −3.489 | 23.029 | −39.133 | 1.00 | 165.60 | O |
| ATOM | 1432 | CB | THR | A | 99 | −5.803 | 25.132 | −37.974 | 1.00 | 165.60 | C |
| ATOM | 1433 | CG2 | THR | A | 99 | −5.071 | 26.004 | −38.977 | 1.00 | 165.60 | C |
| ATOM | 1434 | OG1 | THR | A | 99 | −7.003 | 24.643 | −38.556 | 1.00 | 165.60 | O |
| ATOM | 1435 | N | ASN | A | 100 | −5.587 | 22.205 | −39.110 | 1.00 | 163.03 | N |
| ATOM | 1436 | CA | ASN | A | 100 | −5.428 | 21.301 | −40.243 | 1.00 | 163.03 | C |
| ATOM | 1437 | C | ASN | A | 100 | −5.253 | 22.079 | −41.525 | 1.00 | 163.03 | C |
| ATOM | 1438 | O | ASN | A | 100 | −5.892 | 23.114 | −41.718 | 1.00 | 163.03 | O |
| ATOM | 1439 | CB | ASN | A | 100 | −6.633 | 20.383 | −40.364 | 1.00 | 163.03 | C |
| ATOM | 1440 | CG | ASN | A | 100 | −6.664 | 19.293 | −39.327 | 1.00 | 163.03 | C |
| ATOM | 1441 | ND2 | ASN | A | 100 | −7.724 | 18.477 | −39.364 | 1.00 | 163.03 | N |
| ATOM | 1442 | OD1 | ASN | A | 100 | −5.730 | 19.137 | −38.516 | 1.00 | 163.03 | O |
| ATOM | 1443 | N | ILE | A | 101 | −4.400 | 21.570 | −42.416 | 1.00 | 160.68 | N |
| ATOM | 1444 | CA | ILE | A | 101 | −4.154 | 22.195 | −43.713 | 1.00 | 160.68 | C |
| ATOM | 1445 | C | ILE | A | 101 | −5.452 | 22.249 | −44.523 | 1.00 | 160.68 | C |
| ATOM | 1446 | O | ILE | A | 101 | −5.597 | 23.101 | −45.393 | 1.00 | 160.68 | O |
| ATOM | 1447 | CB | ILE | A | 101 | −3.041 | 21.461 | −44.485 | 1.00 | 160.68 | C |
| ATOM | 1448 | CG1 | ILE | A | 101 | −2.555 | 22.318 | −45.661 | 1.00 | 160.68 | C |
| ATOM | 1449 | CG2 | ILE | A | 101 | −3.519 | 20.086 | −44.957 | 1.00 | 160.68 | C |
| ATOM | 1450 | CD1 | ILE | A | 101 | −1.404 | 21.763 | −46.393 | 1.00 | 160.68 | C |
| ATOM | 1451 | N | SER | A | 102 | −6.383 | 21.335 | −44.230 | 1.00 | 158.66 | N |
| ATOM | 1452 | CA | SER | A | 102 | −7.678 | 21.300 | −44.878 | 1.00 | 158.66 | C |
| ATOM | 1453 | C | SER | A | 102 | −8.465 | 22.530 | −44.468 | 1.00 | 158.66 | C |
| ATOM | 1454 | O | SER | A | 102 | −9.235 | 23.061 | −45.268 | 1.00 | 158.66 | O |
| ATOM | 1455 | CB | SER | A | 102 | −8.430 | 20.039 | −44.483 | 1.00 | 158.66 | C |
| ATOM | 1456 | OG | SER | A | 102 | −8.880 | 20.109 | −43.138 | 1.00 | 158.66 | O |
| ATOM | 1457 | N | ARG | A | 103 | −8.276 | 22.973 | −43.205 | 1.00 | 157.48 | N |
| ATOM | 1458 | CA | ARG | A | 103 | −8.936 | 24.161 | −42.676 | 1.00 | 157.48 | C |
| ATOM | 1459 | C | ARG | A | 103 | −8.363 | 25.385 | −43.361 | 1.00 | 157.48 | C |
| ATOM | 1460 | O | ARG | A | 103 | −9.120 | 26.250 | −43.806 | 1.00 | 157.48 | O |
| ATOM | 1461 | CB | ARG | A | 103 | −8.783 | 24.264 | −41.154 | 1.00 | 157.48 | C |
| ATOM | 1462 | CG | ARG | A | 103 | −9.149 | 25.652 | −40.622 | 1.00 | 157.48 | C |
| ATOM | 1463 | CD | ARG | A | 103 | −10.421 | 25.702 | −39.811 | 1.00 | 157.48 | C |
| ATOM | 1464 | NE | ARG | A | 103 | −10.251 | 26.650 | −38.710 | 1.00 | 157.48 | N |
| ATOM | 1465 | CZ | ARG | A | 103 | −11.142 | 26.894 | −37.752 | 1.00 | 157.48 | C |
| ATOM | 1466 | NH1 | ARG | A | 103 | −10.860 | 27.755 | −36.783 | 1.00 | 157.48 | N + 1 |
| ATOM | 1467 | NH2 | ARG | A | 103 | −12.316 | 26.270 | −37.750 | 1.00 | 157.48 | N |
| ATOM | 1468 | N | LEU | A | 104 | −7.030 | 25.442 | −43.465 | 1.00 | 155.21 | N |
| ATOM | 1469 | CA | LEU | A | 104 | −6.343 | 26.551 | −44.101 | 1.00 | 155.21 | C |
| ATOM | 1470 | C | LEU | A | 104 | −6.762 | 26.694 | −45.556 | 1.00 | 155.21 | C |
| ATOM | 1471 | O | LEU | A | 104 | −7.037 | 27.811 | −46.008 | 1.00 | 155.21 | O |
| ATOM | 1472 | CB | LEU | A | 104 | −4.827 | 26.368 | −44.000 | 1.00 | 155.21 | C |
| ATOM | 1473 | CG | LEU | A | 104 | −3.969 | 27.497 | −44.582 | 1.00 | 155.21 | C |
| ATOM | 1474 | CD1 | LEU | A | 104 | −4.161 | 28.802 | −43.810 | 1.00 | 155.21 | C |
| ATOM | 1475 | CD2 | LEU | A | 104 | −2.510 | 27.118 | −44.606 | 1.00 | 155.21 | C |
| ATOM | 1476 | N | LEU | A | 105 | −6.820 | 25.567 | −46.282 | 1.00 | 156.56 | N |
| ATOM | 1477 | CA | LEU | A | 105 | −7.196 | 25.555 | −47.698 | 1.00 | 156.56 | C |
| ATOM | 1478 | C | LEU | A | 105 | −8.632 | 26.004 | −47.909 | 1.00 | 156.56 | C |
| ATOM | 1479 | O | LEU | A | 105 | −8.922 | 26.717 | −48.875 | 1.00 | 156.56 | O |
| ATOM | 1480 | CB | LEU | A | 105 | −7.009 | 24.162 | −48.307 | 1.00 | 156.56 | C |
| ATOM | 1481 | CG | LEU | A | 105 | −5.597 | 23.683 | −48.572 | 1.00 | 156.56 | C |
| ATOM | 1482 | CD1 | LEU | A | 105 | −5.618 | 22.647 | −49.636 | 1.00 | 156.56 | C |
| ATOM | 1483 | CD2 | LEU | A | 105 | −4.701 | 24.811 | −48.999 | 1.00 | 156.56 | C |
| ATOM | 1484 | N | GLN | A | 106 | −9.526 | 25.574 | −47.009 | 1.00 | 158.45 | N |
| ATOM | 1485 | CA | GLN | A | 106 | −10.932 | 25.909 | −47.078 | 1.00 | 158.45 | C |
| ATOM | 1486 | C | GLN | A | 106 | −11.130 | 27.394 | −46.871 | 1.00 | 158.45 | C |
| ATOM | 1487 | O | GLN | A | 106 | −11.867 | 28.032 | −47.625 | 1.00 | 158.45 | O |
| ATOM | 1488 | CB | GLN | A | 106 | −11.730 | 25.097 | −46.049 | 1.00 | 158.45 | C |
| ATOM | 1489 | CG | GLN | A | 106 | −13.231 | 25.384 | −46.045 | 1.00 | 158.45 | C |
| ATOM | 1490 | CD | GLN | A | 106 | −13.967 | 25.121 | −47.357 | 1.00 | 158.45 | C |
| ATOM | 1491 | NE2 | GLN | A | 106 | −13.249 | 24.881 | −48.458 | 1.00 | 158.45 | N |
| ATOM | 1492 | OE1 | GLN | A | 106 | −15.204 | 25.153 | −47.410 | 1.00 | 158.45 | O |
| ATOM | 1493 | N | GLU | A | 107 | −10.457 | 27.941 | −45.862 | 1.00 | 158.86 | N |
| ATOM | 1494 | CA | GLU | A | 107 | −10.541 | 29.358 | −45.546 | 1.00 | 158.86 | C |
| ATOM | 1495 | C | GLU | A | 107 | −9.980 | 30.195 | −46.686 | 1.00 | 158.86 | C |
| ATOM | 1496 | O | GLU | A | 107 | −10.599 | 31.188 | −47.072 | 1.00 | 158.86 | O |
| ATOM | 1497 | CB | GLU | A | 107 | −9.808 | 29.662 | −44.240 | 1.00 | 158.86 | C |
| ATOM | 1498 | CG | GLU | A | 107 | −10.650 | 29.399 | −42.999 | 1.00 | 158.86 | C |
| ATOM | 1499 | CD | GLU | A | 107 | −9.917 | 29.556 | −41.677 | 1.00 | 158.86 | C |
| ATOM | 1500 | OE1 | GLU | A | 107 | −8.832 | 30.185 | −41.668 | 1.00 | 158.86 | O |
| ATOM | 1501 | OE2 | GLU | A | 107 | −10.432 | 29.059 | −40.647 | 1.00 | 158.86 | O + 1 |
| ATOM | 1502 | N | THR | A | 108 | −8.834 | 29.769 | −47.254 | 1.00 | 154.65 | N |
| ATOM | 1503 | CA | THR | A | 108 | −8.209 | 30.464 | −48.377 | 1.00 | 154.65 | C |
| ATOM | 1504 | C | THR | A | 108 | −9.202 | 30.598 | −49.523 | 1.00 | 154.65 | C |
| ATOM | 1505 | O | THR | A | 108 | −9.304 | 31.671 | −50.121 | 1.00 | 154.65 | O |
| ATOM | 1506 | CB | THR | A | 108 | −6.956 | 29.734 | −48.829 | 1.00 | 154.65 | C |
| ATOM | 1507 | CG2 | THR | A | 108 | −6.447 | 30.243 | −50.161 | 1.00 | 154.65 | C |
| ATOM | 1508 | OG1 | THR | A | 108 | −5.941 | 29.903 | −47.843 | 1.00 | 154.65 | O |
| ATOM | 1509 | N | SER | A | 109 | −9.930 | 29.510 | −49.828 | 1.00 | 153.07 | N |
| ATOM | 1510 | CA | SER | A | 109 | −10.930 | 29.525 | −50.889 | 1.00 | 153.07 | C |
| ATOM | 1511 | C | SER | A | 109 | −11.982 | 30.566 | −50.567 | 1.00 | 153.07 | C |
| ATOM | 1512 | O | SER | A | 109 | −12.272 | 31.424 | −51.402 | 1.00 | 153.07 | O |
| ATOM | 1513 | CB | SER | A | 109 | −11.569 | 28.152 | −51.053 | 1.00 | 153.07 | C |
| ATOM | 1514 | OG | SER | A | 109 | −12.182 | 28.019 | −52.325 | 1.00 | 153.07 | O |
| ATOM | 1515 | N | GLU | A | 110 | −12.505 | 30.528 | −49.332 | 1.00 | 153.58 | N |
| ATOM | 1516 | CA | GLU | A | 110 | −13.522 | 31.462 | −48.863 | 1.00 | 153.58 | C |
| ATOM | 1517 | C | GLU | A | 110 | −13.068 | 32.910 | −48.980 | 1.00 | 153.58 | C |
| ATOM | 1518 | O | GLU | A | 110 | −13.833 | 33.756 | −49.453 | 1.00 | 153.58 | O |
| ATOM | 1519 | CB | GLU | A | 110 | −13.881 | 31.166 | −47.417 | 1.00 | 153.58 | C |
| ATOM | 1520 | CG | GLU | A | 110 | −14.620 | 29.858 | −47.210 | 1.00 | 153.58 | C |
| ATOM | 1521 | CD | GLU | A | 110 | −14.851 | 29.494 | −45.752 | 1.00 | 153.58 | C |
| ATOM | 1522 | OE1 | GLU | A | 110 | −14.426 | 30.265 | −44.859 | 1.00 | 153.58 | O |
| ATOM | 1523 | OE2 | GLU | A | 110 | −15.463 | 28.429 | −45.503 | 1.00 | 153.58 | O + 1 |
| ATOM | 1524 | N | GLN | A | 111 | −11.823 | 33.194 | −48.567 | 1.00 | 151.63 | N |
| ATOM | 1525 | CA | GLN | A | 111 | −11.265 | 34.544 | −48.619 | 1.00 | 151.63 | C |
| ATOM | 1526 | C | GLN | A | 111 | −11.137 | 35.047 | −50.041 | 1.00 | 151.63 | C |
| ATOM | 1527 | O | GLN | A | 111 | −11.334 | 36.238 | −50.282 | 1.00 | 151.63 | O |
| ATOM | 1528 | CB | GLN | A | 111 | −9.904 | 34.593 | −47.944 | 1.00 | 151.63 | C |
| ATOM | 1529 | CG | GLN | A | 111 | −9.926 | 34.256 | −46.454 | 1.00 | 151.63 | C |
| ATOM | 1530 | CD | GLN | A | 111 | −8.597 | 33.690 | −45.970 | 1.00 | 151.63 | C |
| ATOM | 1531 | NE2 | GLN | A | 111 | −7.524 | 33.862 | −46.741 | 1.00 | 151.63 | N |
| ATOM | 1532 | OE1 | GLN | A | 111 | −8.506 | 33.081 | −44.895 | 1.00 | 151.63 | O |
| ATOM | 1533 | N | LEU | A | 112 | −10.818 | 34.149 | −50.983 | 1.00 | 146.44 | N |
| ATOM | 1534 | CA | LEU | A | 112 | −10.707 | 34.532 | −52.380 | 1.00 | 146.44 | C |
| ATOM | 1535 | C | LEU | A | 112 | −12.075 | 34.901 | −52.927 | 1.00 | 146.44 | C |
| ATOM | 1536 | O | LEU | A | 112 | −12.200 | 35.884 | −53.652 | 1.00 | 146.44 | O |
| ATOM | 1537 | CB | LEU | A | 112 | −10.078 | 33.415 | −53.207 | 1.00 | 146.44 | C |
| ATOM | 1538 | CG | LEU | A | 112 | −8.564 | 33.318 | −53.140 | 1.00 | 146.44 | C |
| ATOM | 1539 | CD1 | LEU | A | 112 | −8.096 | 32.055 | −53.771 | 1.00 | 146.44 | C |
| ATOM | 1540 | CD2 | LEU | A | 112 | −7.905 | 34.503 | −53.831 | 1.00 | 146.44 | C |
| ATOM | 1541 | N | VAL | A | 113 | −13.102 | 34.143 | −52.542 | 1.00 | 143.55 | N |
| ATOM | 1542 | CA | VAL | A | 113 | −14.473 | 34.379 | −52.973 | 1.00 | 143.55 | C |
| ATOM | 1543 | C | VAL | A | 113 | −14.960 | 35.734 | −52.505 | 1.00 | 143.55 | C |
| ATOM | 1544 | O | VAL | A | 113 | −15.475 | 36.498 | −53.319 | 1.00 | 143.55 | O |
| ATOM | 1545 | CB | VAL | A | 113 | −15.413 | 33.259 | −52.486 | 1.00 | 143.55 | C |
| ATOM | 1546 | CG1 | VAL | A | 113 | −16.881 | 33.678 | −52.572 | 1.00 | 143.55 | C |
| ATOM | 1547 | CG2 | VAL | A | 113 | −15.181 | 31.982 | −53.282 | 1.00 | 143.55 | C |
| ATOM | 1548 | N | ALA | A | 114 | −14.808 | 36.026 | −51.203 | 1.00 | 140.81 | N |
| ATOM | 1549 | CA | ALA | A | 114 | −15.258 | 37.284 | −50.615 | 1.00 | 140.81 | C |
| ATOM | 1550 | C | ALA | A | 114 | −14.487 | 38.483 | −51.168 | 1.00 | 140.81 | C |
| ATOM | 1551 | O | ALA | A | 114 | −15.045 | 39.572 | −51.306 | 1.00 | 140.81 | O |
| ATOM | 1552 | CB | ALA | A | 114 | −15.117 | 37.226 | −49.108 | 1.00 | 140.81 | C |
| ATOM | 1553 | N | LEU | A | 115 | −13.222 | 38.272 | −51.504 | 1.00 | 139.88 | N |
| ATOM | 1554 | CA | LEU | A | 115 | −12.341 | 39.313 | −52.002 | 1.00 | 139.88 | C |
| ATOM | 1555 | C | LEU | A | 115 | −12.509 | 39.591 | −53.489 | 1.00 | 139.88 | C |
| ATOM | 1556 | O | LEU | A | 115 | −12.216 | 40.698 | −53.939 | 1.00 | 139.88 | O |
| ATOM | 1557 | CB | LEU | A | 115 | −10.908 | 38.875 | −51.720 | 1.00 | 139.88 | C |
| ATOM | 1558 | CG | LEU | A | 115 | −9.811 | 39.911 | −51.641 | 1.00 | 139.88 | C |
| ATOM | 1559 | CD1 | LEU | A | 115 | −10.218 | 41.104 | −50.806 | 1.00 | 139.88 | C |
| ATOM | 1560 | CD2 | LEU | A | 115 | −8.595 | 39.298 | −51.027 | 1.00 | 139.88 | C |
| ATOM | 1561 | N | LYS | A | 116 | −12.971 | 38.593 | −54.247 | 1.00 | 142.54 | N |
| ATOM | 1562 | CA | LYS | A | 116 | −13.135 | 38.666 | −55.700 | 1.00 | 142.54 | C |
| ATOM | 1563 | C | LYS | A | 116 | −13.928 | 39.908 | −56.180 | 1.00 | 142.54 | C |
| ATOM | 1564 | O | LYS | A | 116 | −13.472 | 40.573 | −57.122 | 1.00 | 142.54 | O |
| ATOM | 1565 | CB | LYS | A | 116 | −13.778 | 37.380 | −56.238 | 1.00 | 142.54 | C |
| ATOM | 1566 | N | PRO | A | 117 | −15.078 | 40.268 | −55.571 | 1.00 | 147.62 | N |
| ATOM | 1567 | CA | PRO | A | 117 | −15.813 | 41.438 | −56.084 | 1.00 | 147.62 | C |
| ATOM | 1568 | C | PRO | A | 117 | −15.214 | 42.797 | −55.694 | 1.00 | 147.62 | C |
| ATOM | 1569 | O | PRO | A | 117 | −15.630 | 43.819 | −56.248 | 1.00 | 147.62 | O |
| ATOM | 1570 | CB | PRO | A | 117 | −17.208 | 41.272 | −55.479 | 1.00 | 147.62 | C |
| ATOM | 1571 | CG | PRO | A | 117 | −17.008 | 40.444 | −54.261 | 1.00 | 147.62 | C |
| ATOM | 1572 | CD | PRO | A | 117 | −15.825 | 39.584 | −54.490 | 1.00 | 147.62 | C |
| ATOM | 1573 | N | TRP | A | 118 | −14.239 | 42.821 | −54.776 | 1.00 | 150.16 | N |
| ATOM | 1574 | CA | TRP | A | 118 | −13.698 | 44.088 | −54.289 | 1.00 | 150.16 | C |
| ATOM | 1575 | C | TRP | A | 118 | −12.227 | 44.326 | −54.560 | 1.00 | 150.16 | C |
| ATOM | 1576 | O | TRP | A | 118 | −11.784 | 45.468 | −54.451 | 1.00 | 150.16 | O |
| ATOM | 1577 | CB | TRP | A | 118 | −13.929 | 44.179 | −52.782 | 1.00 | 150.16 | C |
| ATOM | 1578 | CG | TRP | A | 118 | −15.369 | 44.005 | −52.424 | 1.00 | 150.16 | C |
| ATOM | 1579 | CD1 | TRP | A | 118 | −16.006 | 42.835 | −52.135 | 1.00 | 150.16 | C |
| ATOM | 1580 | CD2 | TRP | A | 118 | −16.368 | 45.025 | −52.404 | 1.00 | 150.16 | C |
| ATOM | 1581 | CE2 | TRP | A | 118 | −17.590 | 44.404 | −52.068 | 1.00 | 150.16 | C |
| ATOM | 1582 | CE3 | TRP | A | 118 | −16.346 | 46.417 | −52.610 | 1.00 | 150.16 | C |
| ATOM | 1583 | NE1 | TRP | A | 118 | −17.340 | 43.066 | −51.912 | 1.00 | 150.16 | N |
| ATOM | 1584 | CZ2 | TRP | A | 118 | −18.781 | 45.123 | −51.950 | 1.00 | 150.16 | C |
| ATOM | 1585 | CZ3 | TRP | A | 118 | −17.526 | 47.128 | −52.505 | 1.00 | 150.16 | C |
| ATOM | 1586 | CH2 | TRP | A | 118 | −18.731 | 46.479 | −52.209 | 1.00 | 150.16 | C |
| ATOM | 1587 | N | ILE | A | 119 | −11.472 | 43.285 | −54.903 | 1.00 | 157.85 | N |
| ATOM | 1588 | CA | ILE | A | 119 | −10.031 | 43.388 | −55.118 | 1.00 | 157.85 | C |
| ATOM | 1589 | C | ILE | A | 119 | −9.574 | 44.574 | −56.009 | 1.00 | 157.85 | C |
| ATOM | 1590 | O | ILE | A | 119 | −8.466 | 45.092 | −55.805 | 1.00 | 157.85 | O |
| ATOM | 1591 | CB | ILE | A | 119 | −9.477 | 42.067 | −55.676 | 1.00 | 157.85 | C |
| ATOM | 1592 | CG1 | ILE | A | 119 | −7.945 | 42.040 | −55.583 | 1.00 | 157.85 | C |
| ATOM | 1593 | CG2 | ILE | A | 119 | −9.978 | 41.786 | −57.110 | 1.00 | 157.85 | C |
| ATOM | 1594 | CD1 | ILE | A | 119 | −7.387 | 42.230 | −54.214 | 1.00 | 157.85 | C |
| ATOM | 1595 | N | THR | A | 120 | −10.409 | 44.992 | −56.978 | 1.00 | 164.58 | N |
| ATOM | 1596 | CA | THR | A | 120 | −10.030 | 46.057 | −57.904 | 1.00 | 164.58 | C |
| ATOM | 1597 | C | THR | A | 120 | −10.697 | 47.394 | −57.618 | 1.00 | 164.58 | C |
| ATOM | 1598 | O | THR | A | 120 | −10.336 | 48.386 | −58.254 | 1.00 | 164.58 | O |
| ATOM | 1599 | CB | THR | A | 120 | −10.334 | 45.641 | −59.331 | 1.00 | 164.58 | C |
| ATOM | 1600 | CG2 | THR | A | 120 | −9.574 | 44.398 | −59.733 | 1.00 | 164.58 | C |
| ATOM | 1601 | OG1 | THR | A | 120 | −11.742 | 45.439 | −59.463 | 1.00 | 164.58 | O |
| ATOM | 1602 | N | ARG | A | 121 | −11.649 | 47.433 | −56.670 | 1.00 | 170.17 | N |
| ATOM | 1603 | CA | ARG | A | 121 | −12.373 | 48.664 | −56.330 | 1.00 | 170.17 | C |
| ATOM | 1604 | C | ARG | A | 121 | −11.789 | 49.381 | −55.105 | 1.00 | 170.17 | C |
| ATOM | 1605 | O | ARG | A | 121 | −12.009 | 50.580 | −54.920 | 1.00 | 170.17 | O |
| ATOM | 1606 | CB | ARG | A | 121 | −13.854 | 48.354 | −56.074 | 1.00 | 170.17 | C |
| ATOM | 1607 | CG | ARG | A | 121 | −14.703 | 48.161 | −57.320 | 1.00 | 170.17 | C |
| ATOM | 1608 | CD | ARG | A | 121 | −14.844 | 46.697 | −57.647 | 1.00 | 170.17 | C |
| ATOM | 1609 | NE | ARG | A | 121 | −15.695 | 46.486 | −58.814 | 1.00 | 170.17 | N |
| ATOM | 1610 | CZ | ARG | A | 121 | −15.829 | 45.321 | −59.442 | 1.00 | 170.17 | C |
| ATOM | 1611 | NH1 | ARG | A | 121 | −15.165 | 44.249 | −59.020 | 1.00 | 170.17 | N + 1 |
| ATOM | 1612 | NH2 | ARG | A | 121 | −16.623 | 45.220 | −60.503 | 1.00 | 170.17 | N |
| ATOM | 1613 | N | GLN | A | 122 | −11.056 | 48.648 | −54.274 | 1.00 | 173.38 | N |
| ATOM | 1614 | CA | GLN | A | 122 | −10.527 | 49.201 | −53.045 | 1.00 | 173.38 | C |
| ATOM | 1615 | C | GLN | A | 122 | −9.061 | 49.516 | −53.105 | 1.00 | 173.38 | C |
| ATOM | 1616 | O | GLN | A | 122 | −8.293 | 48.928 | −53.871 | 1.00 | 173.38 | O |
| ATOM | 1617 | CB | GLN | A | 122 | −10.768 | 48.236 | −51.870 | 1.00 | 173.38 | C |
| ATOM | 1618 | CG | GLN | A | 122 | −12.215 | 47.778 | −51.692 | 1.00 | 173.38 | C |
| ATOM | 1619 | CD | GLN | A | 122 | −13.138 | 48.858 | −51.152 | 1.00 | 173.38 | C |
| ATOM | 1620 | NE2 | GLN | A | 122 | −12.575 | 49.958 | −50.641 | 1.00 | 173.38 | N |
| ATOM | 1621 | OE1 | GLN | A | 122 | −14.377 | 48.722 | −51.182 | 1.00 | 173.38 | O |
| ATOM | 1622 | N | ASN | A | 123 | −8.698 | 50.472 | −52.261 | 1.00 | 174.37 | N |
| ATOM | 1623 | CA | ASN | A | 123 | −7.342 | 50.891 | −51.966 | 1.00 | 174.37 | C |
| ATOM | 1624 | C | ASN | A | 123 | −6.968 | 50.146 | −50.680 | 1.00 | 174.37 | C |
| ATOM | 1625 | O | ASN | A | 123 | −7.594 | 50.332 | −49.631 | 1.00 | 174.37 | O |
| ATOM | 1626 | CB | ASN | A | 123 | −7.247 | 52.417 | −51.830 | 1.00 | 174.37 | C |
| ATOM | 1627 | CG | ASN | A | 123 | −5.859 | 52.925 | −51.513 | 1.00 | 174.37 | C |
| ATOM | 1628 | ND2 | ASN | A | 123 | −5.671 | 54.228 | −51.555 | 1.00 | 174.37 | N |
| ATOM | 1629 | OD1 | ASN | A | 123 | −4.940 | 52.164 | −51.235 | 1.00 | 174.37 | O |
| ATOM | 1630 | N | PHE | A | 124 | −5.984 | 49.263 | −50.775 | 1.00 | 172.43 | N |
| ATOM | 1631 | CA | PHE | A | 124 | −5.616 | 48.409 | −49.653 | 1.00 | 172.43 | C |
| ATOM | 1632 | C | PHE | A | 124 | −4.347 | 48.853 | −48.973 | 1.00 | 172.43 | C |
| ATOM | 1633 | O | PHE | A | 124 | −3.703 | 48.060 | −48.285 | 1.00 | 172.43 | O |
| ATOM | 1634 | CB | PHE | A | 124 | −5.471 | 46.972 | −50.150 | 1.00 | 172.43 | C |
| ATOM | 1635 | CG | PHE | A | 124 | −6.758 | 46.416 | −50.702 | 1.00 | 172.43 | C |
| ATOM | 1636 | CD1 | PHE | A | 124 | −7.025 | 46.458 | −52.069 | 1.00 | 172.43 | C |
| ATOM | 1637 | CD2 | PHE | A | 124 | −7.714 | 45.870 | −49.856 | 1.00 | 172.43 | C |
| ATOM | 1638 | CE1 | PHE | A | 124 | −8.219 | 45.942 | −52.578 | 1.00 | 172.43 | C |
| ATOM | 1639 | CE2 | PHE | A | 124 | −8.905 | 45.355 | −50.364 | 1.00 | 172.43 | C |
| ATOM | 1640 | CZ | PHE | A | 124 | −9.149 | 45.389 | −51.721 | 1.00 | 172.43 | C |
| ATOM | 1641 | N | SER | A | 125 | −4.009 | 50.131 | −49.126 | 1.00 | 170.68 | N |
| ATOM | 1642 | CA | SER | A | 125 | −2.811 | 50.710 | −48.542 | 1.00 | 170.68 | C |
| ATOM | 1643 | C | SER | A | 125 | −2.758 | 50.545 | −47.018 | 1.00 | 170.68 | C |
| ATOM | 1644 | O | SER | A | 125 | −1.673 | 50.323 | −46.491 | 1.00 | 170.68 | O |
| ATOM | 1645 | CB | SER | A | 125 | −2.699 | 52.184 | −48.911 | 1.00 | 170.68 | C |
| ATOM | 1646 | OG | SER | A | 125 | −2.619 | 52.347 | −50.318 | 1.00 | 170.68 | O |
| ATOM | 1647 | N | ARG | A | 126 | −3.907 | 50.608 | −46.321 | 1.00 | 168.27 | N |
| ATOM | 1648 | CA | ARG | A | 126 | −3.945 | 50.488 | −44.864 | 1.00 | 168.27 | C |
| ATOM | 1649 | C | ARG | A | 126 | −3.897 | 49.032 | −44.362 | 1.00 | 168.27 | C |
| ATOM | 1650 | O | ARG | A | 126 | −3.842 | 48.799 | −43.153 | 1.00 | 168.27 | O |
| ATOM | 1651 | CB | ARG | A | 126 | −5.195 | 51.187 | −44.327 | 1.00 | 168.27 | C |
| ATOM | 1652 | N | CYS | A | 127 | −3.901 | 48.065 | −45.277 | 1.00 | 168.87 | N |
| ATOM | 1653 | CA | CYS | A | 127 | −3.865 | 46.641 | −44.941 | 1.00 | 168.87 | C |
| ATOM | 1654 | C | CYS | A | 127 | −2.447 | 46.164 | −44.735 | 1.00 | 168.87 | C |
| ATOM | 1655 | O | CYS | A | 127 | −2.234 | 45.062 | −44.222 | 1.00 | 168.87 | O |
| ATOM | 1656 | CB | CYS | A | 127 | −4.547 | 45.833 | −46.036 | 1.00 | 168.87 | C |
| ATOM | 1657 | SG | CYS | A | 127 | −6.252 | 46.321 | −46.353 | 1.00 | 168.87 | S |
| ATOM | 1658 | N | LEU | A | 128 | −1.474 | 46.978 | −45.155 | 1.00 | 167.63 | N |
| ATOM | 1659 | CA | LEU | A | 128 | −0.057 | 46.638 | −45.105 | 1.00 | 167.63 | C |
| ATOM | 1660 | C | LEU | A | 128 | 0.444 | 46.384 | −43.691 | 1.00 | 167.63 | C |
| ATOM | 1661 | O | LEU | A | 128 | 1.410 | 45.641 | −43.515 | 1.00 | 167.63 | O |
| ATOM | 1662 | CB | LEU | A | 128 | 0.765 | 47.720 | −45.801 | 1.00 | 167.63 | C |
| ATOM | 1663 | CG | LEU | A | 128 | 0.467 | 47.893 | −47.303 | 1.00 | 167.63 | C |
| ATOM | 1664 | CD1 | LEU | A | 128 | 1.602 | 48.589 | −47.992 | 1.00 | 167.63 | C |
| ATOM | 1665 | CD2 | LEU | A | 128 | 0.222 | 46.551 | −47.993 | 1.00 | 167.63 | C |
| ATOM | 1666 | N | GLU | A | 129 | −0.240 | 46.948 | −42.689 | 1.00 | 165.82 | N |
| ATOM | 1667 | CA | GLU | A | 129 | 0.091 | 46.711 | −41.294 | 1.00 | 165.82 | C |
| ATOM | 1668 | C | GLU | A | 129 | −0.970 | 45.811 | −40.711 | 1.00 | 165.82 | C |
| ATOM | 1669 | O | GLU | A | 129 | −2.124 | 46.228 | −40.590 | 1.00 | 165.82 | O |
| ATOM | 1670 | CB | GLU | A | 129 | 0.197 | 48.028 | −40.519 | 1.00 | 165.82 | C |
| ATOM | 1671 | N | LEU | A | 130 | −0.599 | 44.558 | −40.405 | 1.00 | 164.53 | N |
| ATOM | 1672 | CA | LEU | A | 130 | −1.519 | 43.585 | −39.822 | 1.00 | 164.53 | C |
| ATOM | 1673 | C | LEU | A | 130 | −1.851 | 44.018 | −38.421 | 1.00 | 164.53 | C |
| ATOM | 1674 | O | LEU | A | 130 | −0.956 | 44.435 | −37.694 | 1.00 | 164.53 | O |
| ATOM | 1675 | CB | LEU | A | 130 | −0.909 | 42.183 | −39.828 | 1.00 | 164.53 | C |
| ATOM | 1676 | CG | LEU | A | 130 | −1.122 | 41.349 | −41.090 | 1.00 | 164.53 | C |
| ATOM | 1677 | CD1 | LEU | A | 130 | −0.316 | 41.888 | −42.277 | 1.00 | 164.53 | C |
| ATOM | 1678 | CD2 | LEU | A | 130 | −0.760 | 39.901 | −40.842 | 1.00 | 164.53 | C |
| ATOM | 1679 | N | GLN | A | 131 | −3.125 | 43.965 | −38.044 | 1.00 | 167.56 | N |
| ATOM | 1680 | CA | GLN | A | 131 | −3.537 | 44.430 | −36.723 | 1.00 | 167.56 | C |
| ATOM | 1681 | C | GLN | A | 131 | −4.198 | 43.337 | −35.884 | 1.00 | 167.56 | C |
| ATOM | 1682 | O | GLN | A | 131 | −4.838 | 42.443 | −36.426 | 1.00 | 167.56 | O |
| ATOM | 1683 | CB | GLN | A | 131 | −4.482 | 45.626 | −36.853 | 1.00 | 167.56 | C |
| ATOM | 1684 | CG | GLN | A | 131 | −3.775 | 46.940 | −37.188 | 1.00 | 167.56 | C |
| ATOM | 1685 | CD | GLN | A | 131 | −4.663 | 47.913 | −37.939 | 1.00 | 167.56 | C |
| ATOM | 1686 | NE2 | GLN | A | 131 | −4.749 | 49.141 | −37.443 | 1.00 | 167.56 | N |
| ATOM | 1687 | OE1 | GLN | A | 131 | −5.254 | 47.593 | −38.980 | 1.00 | 167.56 | O |
| ATOM | 1688 | N | CYS | A | 132 | −4.041 | 43.420 | −34.549 | 1.00 | 172.25 | N |
| ATOM | 1689 | CA | CYS | A | 132 | −4.608 | 42.469 | −33.571 | 1.00 | 172.25 | C |
| ATOM | 1690 | C | CYS | A | 132 | −5.870 | 43.095 | −32.905 | 1.00 | 172.25 | C |
| ATOM | 1691 | O | CYS | A | 132 | −5.887 | 44.313 | −32.721 | 1.00 | 172.25 | O |
| ATOM | 1692 | CB | CYS | A | 132 | −3.564 | 42.049 | −32.533 | 1.00 | 172.25 | C |
| ATOM | 1693 | SG | CYS | A | 132 | −1.950 | 41.573 | −33.218 | 1.00 | 172.25 | S |
| ATOM | 1694 | N | GLN | A | 133 | −6.982 | 42.324 | −32.687 | 1.00 | 169.42 | N |
| ATOM | 1695 | CA | GLN | A | 133 | −8.271 | 42.876 | −32.175 | 1.00 | 169.42 | C |
| ATOM | 1696 | C | GLN | A | 133 | −8.228 | 43.303 | −30.707 | 1.00 | 169.42 | C |
| ATOM | 1697 | O | GLN | A | 133 | −9.029 | 44.145 | −30.296 | 1.00 | 169.42 | O |
| ATOM | 1698 | CB | GLN | A | 133 | −9.437 | 41.891 | −32.372 | 1.00 | 169.42 | C |
| END | | | | | | | | | | | |

1. A method of identifying a ligand which modulates Flt3 signaling, comprising the step of employing a three dimensional structure represented by a set of atomic coordinates presented in Table 3, or a subset thereof, or atomic coordinates which deviate from those in Table 3, or a subset thereof, by RMSD over protein backbone atoms by no more than 3 Å.

2. The method according to claim 1, wherein said subset comprises at least 5 consecutive amino acid residues of amino acid residues 245-345 of Flt3 and/or at least 5 consecutive amino acid residues of amino acid residues 5-20 of FL or wherein said subset comprises at least 5 consecutive amino acid residues of amino acid residues 279-311 of Flt3 and/or at least 5 consecutive amino acid residues of amino acid residues 5-20 of FL.

3. (canceled)

4. The method according to claim 1, further comprising the step of structure-based identification of a ligand based on the interaction of said ligand with the 3D structure represented by said atomic coordinates, or said subset thereof.

5. The method according to claim 1, which is a computer-implemented method, said computer comprising an inputting device, a processor, a user interface, and an outputting device, wherein said method comprises the steps of:
a) generating a three-dimensional structure of said atomic coordinates, or said subset thereof;
b) fitting the structure of step a) with the structure of a candidate ligand by computational modeling; and
c) selecting a ligand that possesses energetically favorable interactions with the structure of step a).

6-7. (canceled)

8. The method according to claim 5, wherein said ligand of step c) can bind to at least 1 amino acid residue of the structure of step a) without steric interference.

9. A method for identifying a ligand which modulates Flt3 signaling, comprising the steps of:
a) providing a candidate ligand;
b1) providing a polypeptide comprising a region of at least 5 consecutive amino acid residues of amino acid residues 245-345 of Flt3; or
b2) providing a polypeptide comprising a region of at least 5 consecutive amino acid residues of amino acid residues 5-20 of FL;
c) contacting said candidate ligand with said polypeptide of step b1) or step b2);
d) determining the binding of said candidate ligand with said region of step b1) or step b2); and
e) identifying said candidate ligand as a ligand which modulates Flt3 signaling if binding between said candidate ligand and said region of step b1) or step b2) is detected.

10. An in vitro method for modulating Flt3 signaling, comprising the steps of:
a) providing a composition comprising an Flt3 polyprotein; and
b) contacting said composition with a ligand as identified according to claim 9.

11-13. (canceled)

14. The method according to claim 9, wherein said ligand is an agonist or an antagonist, selected from the group consisting of an Alphabody™, a Nanobody®, an antibody, or a small molecule.

15. An Alphabody™, a Nanobody®, or an antibody which binds to the region comprised within amino acid residues 279-311 of Flt3, or which binds to the region comprised within amino acid residues 5-20 of FL.

16. A polypeptide consisting of at least 5 consecutive amino acid residues of amino acid residues 279-311 of Flt3 or at least 5 consecutive amino acid residues of amino acid residues 5-20 of FL, optionally wherein one or more of amino acids 279, 280, 281, 301, 302, 303, 307, 309, or 311 is mutated.

17-27. (canceled)

28. A computer system comprising:
a) a database containing the atomic coordinates or a subset thereof as defined in claim 1 stored on a computer readable storage medium; and
b) a user interface to view the information.

29-34. (canceled)

35. A method for identifying a ligand of Flt3 which binds to a region of Flt3 comprised within amino acid residues 279-311, the method comprising the steps of:
a) providing a candidate ligand;
b) providing an Flt3 polypeptide comprising said region,
c) contacting said candidate ligand with said polypeptide;
d) determining the binding of said candidate ligand with said region; and
e) identifying said candidate ligand as a ligand of Flt3 if binding is detected.

36. A method for identifying a ligand of Flt3, comprising the steps of
a) providing a candidate ligand;
b1) providing a first polypeptide comprising a region of at least 5 consecutive amino acid residues of amino acid residues 279-311 of Flt3;
b2) providing a second polypeptide comprising a region of at least 5 consecutive amino acid residues of amino acid residues 279-311 of Flt3 wherein at least one amino acid residue of amino acid residues 279-311 is mutated;
c) contacting said candidate ligand with said polypeptide of step b1) or step b2);
d) determining the binding of said candidate ligand with said region of step b1) and step b2); and
e) identifying said candidate ligand as a ligand of Flt3 if binding between said candidate ligand and said polypeptide of step b1) is detected and if no binding between said candidate ligand and said polypeptide of step b2) is detected.

37-40. (canceled)

41. The method according to claim 9, wherein step b1) comprises providing a polypeptide comprising a region of at least 5 consecutive amino acid residues of amino acid residues 279-311 of Flt3.

42. A method of modulating Flt3 signaling using a ligand as identified according to claim 1.

43. A method of modulating Flt3 signaling using a ligand as identified according to claim 9.

44. A method of modulating Flt3 signaling using a ligand as identified according to claim 35.

45. A method of modulating Flt3 signaling using a ligand as identified according to claim 36.

46. The method according to claim 9, wherein said candidate ligand is a ligand which modulates Flt3 signaling.

47. The method according to claim 43, wherein the ligand is an Alphabody™, a Nanobody®, an antibody, or a small molecule.
